SEQUENCE LISTING (64 sequences)

SEQ ID NO: 1 - protein, 469 aa, Artificial Sequence
  HER2(C5)-CD28TM,ICD-CD3Z CAR
MTRAMDWIWRILFLVGAATGAHSQVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARYAPDSSGYLVAFDIWGQGTMVTVSSGGGGSGGGGSGGGGSQTVVTQEPSFSVSPGGTVTLTCGLSSGSVSTGYYPSWYQQTPGQAPRTLIYSTNSRSSGVPDRFSGSILGNKAALTITGAQADDESDYYCVLYMGSGISVFGGGTKLTVLGSEPKSCDKTHTCPTRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 467 aa, Artificial Sequence
  HER2(E4)-CD28TM,ICD-CD3Z CAR
MTRAMDWIWRILFLVGAATGAHSQVQLQQWGAGLLKPSETLSLTCAVYGGSFSGYYWSWIRQPPGKGLEWIGEINHSGSTNYNPSLKSRVTISVDTSKNQFSLKLSSVTTADTAVYYCARMGINSGGYLYGMDVWGQGTTVTVSSGGGGSGGGGSGGGGSQTVVTQEPSFSVSPGGTVTLTCGLSSGSVSTSYYPSWYQQIPGQAPRTLIYTTNIRSSGVPDRFGGSILGNKAALTITGAQAEDESDYYCMLYMGSGIWVFGGGTKLTVLGSEPKSCDKTHTCPTRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 3 - protein, 468 aa, Artificial Sequence
  HER2(F1)-CD28TM,ICD-CD3Z CAR
MTRAMDWIWRILFLVGAATGAHSQVQLVESGPGLVKPSGTLSLTCAVSGGSISSSNWWSWVRQPPGKGLEWIGEIYHSGSTNYNPSLKSRVTISVDTSKNQFSLKLSSVTAADTAVYYCARMGANSGGYLYGMDVWGQGTTVTVSSGGGGSGGGGSGGGGSQTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGGGTKLTVLGSEPKSCDKTHTCPTRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 27 aa, Artificial Sequence
  CD28 TMD
FWVLVVVGGVLACYSLLVTVAFIIFWV

SEQ ID NO: 5 - protein, 41 aa, Artificial Sequence
  CD28 ICD
RSKRSRLLHSDYMNMTPRRPGPTRKHYQPYAPPRDFAAYRS

SEQ ID NO: 6 - protein, 113 aa, Artificial Sequence
  CD3Z ICD
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 15 aa, Artificial Sequence
  (G4S)3 linker
GGGGSGGGGSGGGGS

SEQ ID NO: 8 - protein, 19 aa, Artificial Sequence
  huIgG H leader
MDWIWRILFLVGAATGAHS

SEQ ID NO: 9 - protein, 14 aa, Artificial Sequence
  Hinge
EPKSCDKTHTCPTR

SEQ ID NO: 10 - protein, 14 aa, Artificial Sequence
  HER2(C5) LC-CDR1
GLSSGSVSTGYYPS

SEQ ID NO: 11 - protein, 7 aa, Artificial Sequence
  HER2(C5) LC-CDR2
STNSRSS

SEQ ID NO: 12 - protein, 10 aa, Artificial Sequence
  HER2(C5) LC-CDR3
VLYMGSGISV

SEQ ID NO: 13 - protein, 7 aa, Artificial Sequence
  HER2(C5) HC-CDR1
SSSYYWG

SEQ ID NO: 14 - protein, 16 aa, Artificial Sequence
  HER2(C5) HC-CDR2
SIYYSGSTYYNPSLKS

SEQ ID NO: 15 - protein, 14 aa, Artificial Sequence
  HER2(C5) HC-CDR3
YAPDSSGYLVAFDI

SEQ ID NO: 16 - protein, 112 aa, Artificial Sequence
  HER2(C5) VL
QTVVTQEPSFSVSPGGTVTLTCGLSSGSVSTGYYPSWYQQTPGQAPRTLIYSTNSRSSGVPDRFSGSILGNKAALTITGAQADDESDYYCVLYMGSGISVFGGGTKLTVLGS

SEQ ID NO: 17 - protein, 124 aa, Artificial Sequence
  HER2(C5) VH
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARYAPDSSGYLVAFDIWGQGTMVTVSS

SEQ ID NO: 18 - protein, 14 aa, Artificial Sequence
  HER2(E4) LC-CDR1
GLSSGSVSTSYYPS

SEQ ID NO: 19 - protein, 7 aa, Artificial Sequence
  HER2(E4) LC-CDR2
TTNIRSS

SEQ ID NO: 20 - protein, 10 aa, Artificial Sequence
  HER2(E4) LC-CDR3
MLYMGSGIWV

SEQ ID NO: 21 - protein, 6 aa, Artificial Sequence
  HER2(E4) HC-CDR1
SGYYWS

SEQ ID NO: 22 - protein, 16 aa, Artificial Sequence
  HER2(E4) HC-CDR2
EINHSGSTNYNPSLKS

SEQ ID NO: 23 - protein, 14 aa, Artificial Sequence
  HER2(E4) HC-CDR3
MGINSGGYLYGMDV

SEQ ID NO: 24 - protein, 112 aa, Artificial Sequence
  HER2(E4) VL
QTVVTQEPSFSVSPGGTVTLTCGLSSGSVSTSYYPSWYQQIPGQAPRTLIYTTNIRSSGVPDRFGGSILGNKAALTITGAQAEDESDYYCMLYMGSGIWVFGGGTKLTVLGS

SEQ ID NO: 25 - protein, 122 aa, Artificial Sequence
  HER2(E4) VH
QVQLQQWGAGLLKPSETLSLTCAVYGGSFSGYYWSWIRQPPGKGLEWIGEINHSGSTNYNPSLKSRVTISVDTSKNQFSLKLSSVTTADTAVYYCARMGINSGGYLYGMDVWGQGTTVTVSS

SEQ ID NO: 26 - protein, 14 aa, Artificial Sequence
  HER2(F1) LC-CDR1
GLSSGSVSTSYYPS

SEQ ID NO: 27 - protein, 7 aa, Artificial Sequence
  HER2(F1) LC-CDR2
STNTRSS

SEQ ID NO: 28 - protein, 10 aa, Artificial Sequence
  HER2(F1) LC-CDR3
VLYMGSGIWV

SEQ ID NO: 29 - protein, 6 aa, Artificial Sequence
  HER2(F1) HC-CDR1
SSNWWS

SEQ ID NO: 30 - protein, 16 aa, Artificial Sequence
  HER2(F1) HC-CDR2
EIYHSGSTNYNPSLKS

SEQ ID NO: 31 - protein, 14 aa, Artificial Sequence
  HER2(F1) HC-CDR3
MGANSGGYLYGMDV

SEQ ID NO: 32 - protein, 112 aa, Artificial Sequence
  HER2(F1) VL
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGGGTKLTVLGS

SEQ ID NO: 33 - protein, 123 aa, Artificial Sequence
  HER2(F1) VH
QVQLVESGPGLVKPSGTLSLTCAVSGGSISSSNWWSWVRQPPGKGLEWIGEIYHSGSTNYNPSLKSRVTISVDTSKNQFSLKLSSVTAADTAVYYCARMGANSGGYLYGMDVWGQGTTVTVSS

SEQ ID NO: 34 - protein, 281 aa, Artificial Sequence
  Ad2 E1Adelta24
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 35 - protein, 536 aa, Artificial Sequence
  huIL-12p70
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - protein, 376 aa, Artificial Sequence
  HSV1 TK
MASYPGHQHASAFDQAARSRGHSNRRTALRPRRQQEATEVRPEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMIQTHVTTPGSIPTICDLARTFAREMGEAN

SEQ ID NO: 37 - protein, 9 aa, Artificial Sequence
  HA tag
YPYDVPDYA

SEQ ID NO: 38 - protein, 534 aa, Artificial Sequence
  PD-L1(H12_gl) minibody
MDWIWRILFLVGAATGAHSEVQLVQSGAEVKKPGASVKVSCKASGGTFSSYAISWVRQAPGQGLEWMGRIIPILGIANYAQKFQGRVTITADKSTSTAYMELSSLRSEDTAVYYCARSGHGYSYGAFDYWGQGTLVTVSSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVVFGGGTKLTVLEAKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSYPYDVPDYAGYPYDVPDYAGYPYDVPDYA

SEQ ID NO: 39 - protein, 14 aa, Artificial Sequence
  PD-L1(H12_gl) LC-CDR1
TGSSSNIGAGYDVH

SEQ ID NO: 40 - protein, 7 aa, Artificial Sequence
  PD-L1(H12_gl) LC-CDR2
GNSNRPS

SEQ ID NO: 41 - protein, 13 aa, Artificial Sequence
  PD-L1(H12_gl) LC-CDR3
QSYDSSLSGSYVV

SEQ ID NO: 42 - protein, 5 aa, Artificial Sequence
  PD-L1(H12_gl) HC-CDR1
SYAIS

SEQ ID NO: 43 - protein, 17 aa, Artificial Sequence
  PD-L1(H12_gl) HC-CDR2
RIIPILGIANYAQKFQG

SEQ ID NO: 44 - protein, 12 aa, Artificial Sequence
  PD-L1(H12_gl) HC-CDR3
SGHGYSYGAFDY

SEQ ID NO: 45 - protein, 113 aa, Artificial Sequence
  PD-L1(H12_gl) VL
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVVFGGGTKLTVL

SEQ ID NO: 46 - protein, 121 aa, Artificial Sequence
  PD-L1(H12_gl) VH
EVQLVQSGAEVKKPGASVKVSCKASGGTFSSYAISWVRQAPGQGLEWMGRIIPILGIANYAQKFQGRVTITADKSTSTAYMELSSLRSEDTAVYYCARSGHGYSYGAFDYWGQGTLVTVSS

SEQ ID NO: 47 - DNA, 3750 bp, Artificial Sequence
  HER2(C5)-CD28TM,ICD-CD3Z CAR
aagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatgactcgagccatggattggatctggcgcatcctgtttctcgtgggagctgccacaggcgcccattctcaggttcagctgcaagagtctggccctggcctggtcaagcctagcgaaacactgagcctgacctgtaccgtgtctggcggcagcatcagcagcagctcttactactggggctggatcagacagcctcctggcaaaggcctggaatggatcggctccatctactacagcggcagcacctactacaaccccagcctgaagtccagagtgaccatcagcgtggacaccagcaagaaccagttctccctgaagctgagcagcgtgacagccgccgatacagccgtgtactactgtgccagatacgcccctgatagcagcggctacctggtggcctttgatatctggggccagggcacaatggtcaccgtttctagcggaggcggaggttctggtggcggaggaagtggcggcggaggatctcagacagtggtcacacaagagcccagcttctccgtgtctcctggcggaacagtgaccctgacatgtggccttagctctggctctgtgtccaccggctactaccccagctggtatcagcagacacctggacaggcccctcggacactgatctacagcaccaacagcagatccagcggcgtgcccgatagattcagcggctctatcctgggcaacaaggccgcactgacaatcacaggcgctcaggccgatgacgagagcgactactactgcgtgctgtacatgggcagcggcatctccgtttttggcggaggcacaaagctgaccgtgctgggatccgaaccaaagagttgcgacaaaacacacacctgccctacgcgtttttgggtgctcgtggtggtgggtggcgtgctcgcttgctactcacttctggtgaccgtagcgtttatcattttttgggtcaggagcaagcgatcccgcctattgcacagcgactacatgaacatgaccccccggcgccccgggccaacccggaagcactaccagccatatgcgcctccccgcgatttcgcagcgtatcggtcccgggtcaaattttcacggtccgctgacgccccggcctatcaacagggccagaatcagctgtataatgaattaaacctcggtagacgcgaggagtacgacgtcctcgacaagagaagggggcgcgacccagagatgggaggcaaaccgcagcgcaggaagaatccacaggagggcctgtacaacgaattacagaaggacaagatggcagaggcctacagcgagataggaatgaagggtgaaaggcgtcgtggaaagggccacgatgggctttaccagggcctaagtactgccacaaaagatacgtatgacgcgctgcatatgcaagccctcccccccaggtaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttc

SEQ ID NO: 48 - DNA, 3744 bp, Artificial Sequence
  HER2(E4)-CD28TM,ICD-CD3Z CAR
aagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatgactcgagccatggattggatctggcgcatcctgtttctcgtgggagctgccacaggcgcccattctcaggttcagctgcaacagtggggagccggactgctgaagcctagcgaaacactgagcctgacctgtgccgtgtacggcggcagctttagcggctactactggtcctggatcagacagcctcctggcaaaggcctggaatggatcggcgagatcaatcacagcggcagcaccaactacaaccccagcctgaagtccagagtgaccatcagcgtggacaccagcaagaaccagttctccctgaagctgagcagcgtgaccacagccgataccgccgtgtactactgtgcccggatgggcatcaatagcggcggctacctgtacggcatggatgtgtggggacagggcaccaccgtgacagtttctagcggaggcggaggttctggtggcggaggaagtggcggcggaggatctcagacagtggtcacacaagagcccagcttctccgtgtctcctggcggaacagtgaccctgacatgtggccttagcagcggctctgtgtccaccagctactaccctagctggtatcagcagatccccggacaggcccctcggacactgatctacaccaccaacatcagatccagcggcgtgcccgatagattcggcggatctatcctgggcaacaaggccgcactgacaatcacaggtgcccaggccgaggacgagtccgactactactgcatgctgtacatgggcagcggcatctgggttttcggcggaggcacaaagctgaccgttctgggatccgaaccaaagagttgcgacaaaacacacacctgccctacgcgtttttgggtgctcgtggtggtgggtggcgtgctcgcttgctactcacttctggtgaccgtagcgtttatcattttttgggtcaggagcaagcgatcccgcctattgcacagcgactacatgaacatgaccccccggcgccccgggccaacccggaagcactaccagccatatgcgcctccccgcgatttcgcagcgtatcggtcccgggtcaaattttcacggtccgctgacgccccggcctatcaacagggccagaatcagctgtataatgaattaaacctcggtagacgcgaggagtacgacgtcctcgacaagagaagggggcgcgacccagagatgggaggcaaaccgcagcgcaggaagaatccacaggagggcctgtacaacgaattacagaaggacaagatggcagaggcctacagcgagataggaatgaagggtgaaaggcgtcgtggaaagggccacgatgggctttaccagggcctaagtactgccacaaaagatacgtatgacgcgctgcatatgcaagccctcccccccaggtaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttc

SEQ ID NO: 49 - DNA, 3747 bp, Artificial Sequence
  HER2(F1)-CD28TM,ICD-CD3Z CAR
aagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatgactcgagccatggattggatctggcgcatcctgtttctcgtgggagctgccacaggcgcccattctcaggttcagctggtggaatctggccctggcctggttaagcctagcggcacactgtctctgacctgtgctgtgtctggcggcagcatcagcagcagcaattggtggtcttgggtccgacagcctcctggcaaaggcctggaatggatcggcgagatctaccacagcggcagcaccaactacaaccccagcctgaagtccagagtgaccatcagcgtggacaccagcaagaaccagttctccctgaagctgagcagcgtgacagccgccgatacagccgtgtactactgtgccagaatgggagccaatagcggcggctacctgtacggcatggatgtgtggggacagggcaccaccgtgacagtttctagcggaggcggaggttctggtggcggaggaagtggcggcggaggatctcagacagtggtcacacaagagcccagcttctccgtgtctcctggcggaacagtgaccctgacatgtggccttagcagcggctctgtgtctaccagctactacccctcctggtatcagcagacccctggacaggctccccggacactgatctactccaccaacaccagatccagcggcgtgcccgatagattctccggctctatcctgggcaacaaggccgcactgacaatcacaggcgctcaggccgatgacgagagcgactactactgcgtgctgtacatgggcagcggcatctgggttttcggcggaggcacaaagctgaccgttctgggatccgaaccaaagagttgcgacaaaacacacacctgccctacgcgtttttgggtgctcgtggtggtgggtggcgtgctcgcttgctactcacttctggtgaccgtagcgtttatcattttttgggtcaggagcaagcgatcccgcctattgcacagcgactacatgaacatgaccccccggcgccccgggccaacccggaagcactaccagccatatgcgcctccccgcgatttcgcagcgtatcggtcccgggtcaaattttcacggtccgctgacgccccggcctatcaacagggccagaatcagctgtataatgaattaaacctcggtagacgcgaggagtacgacgtcctcgacaagagaagggggcgcgacccagagatgggaggcaaaccgcagcgcaggaagaatccacaggagggcctgtacaacgaattacagaaggacaagatggcagaggcctacagcgagataggaatgaagggtgaaaggcgtcgtggaaagggccacgatgggctttaccagggcctaagtactgccacaaaagatacgtatgacgcgctgcatatgcaagccctcccccccaggtaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttc

SEQ ID NO: 50 - DNA, 30590 bp, Artificial Sequence
  HDAdIL12p70_TK_aPD-L1
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgcagttagggataacagggtaatacgaccatggcatgtcctctagactcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggaggggctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttattagctaggagtttcagaaaagggggcctgagtggccccttttttcaacttaattaacctgcagggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgtcatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggtcgcacgtcatgaatctagaaccatggcttcgtaccccggccatcagcacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgccttgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatgatccagacccacgtcaccaccccaggctccataccgacgatctgcgacctggcgcgcacgtttgcccgggagatgggggaggctaactgagtataccctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagctgcctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagttaccggcggaaacggtctcgggttgagaggtcacccgagggacaggcagctgctgaaccaataggaccggcgcacagggcggatgctgcccctcattggcggccgttgagagtgaccaagagccaatgagtcagcccggggggcgtagcagtgacgtaagttgcggaggaggccgcttcgaatcggcagcggccagcttggtggcatggaccaatcagcgtcctccaacgaggagcgccttcgccaatcggaggcctccacgacggggctggggggagggtatataagccgagtcggcggcggcgcgctccacacgggccgagaccacagcgacgggagcgtctgcctctgcggggccgagaggtaagcgccgcggcctgccctttccaggccaactcggagcccgtctcgtggctccgcctgatcgggggctcctgtcgccctcagatcggtcggaacgccgtcgcgctccgggactacaagcctgttgctgggcccggagactgccgaaggaccgctgagcactgtcctcagcgccggcaccatggattggatctggcggatcctgttccttgtgggagctgccacaggcgcccattctgaagttcagctggttcagtctggcgccgaagtgaagaaacctggcgcctctgtgaaggtgtcctgcaaagcttctggcggcaccttcagcagctacgccatctcttgggttcgacaggcccctggacaaggcctggaatggatgggcagaatcatccccatcctgggaatcgccaactacgcccagaaattccagggcagagtgaccatcaccgccgacaagagcacaagcaccgcctacatggaactgagcagcctgagaagcgaggacaccgccgtgtactactgtgccagaagcggccacggctacagctacggcgcctttgattattggggccagggcaccctggtcaccgtttctagcggaggcggaggtagtggtggcggaggttcaggcggcggaggatctcaatctgtgctgacacagcctccaagcgtgtcaggtgctcctggccagagagtgacaatcagctgtacaggcagcagcagcaacatcggagccggctatgacgtgcactggtatcagcagctgcctggcacagcccctaaactgctgatctacggcaacagcaacagacccagcggcgtgcccgatagattttccggctctaagagcggcacaagcgccagcctggctattactggactgcaggccgaggacgaggccgactactactgtcagagctacgacagcagcctgtccggcagctacgttgtgtttggcggcggaacaaagctgaccgtgctggaagccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggacccagaagtgaagttcaattggtacgtggacggcgtggaagtgcacaacgccaagaccaagcctagagaggaacagtacaacagcacctacagagtggtgtccgtgctgacagtgctgcaccaggattggctgaacggcaaagagtacaagtgcaaggtgtccaacaaggccctgcctgctcctatcgagaaaaccatcagcaaggccaagggccagcctagggaaccccaggtttacacactgccacctagcagggacgagctgaccaagaatcaggtgtccctgacctgcctggtcaagggcttctacccttccgatatcgccgtggaatgggagagcaatggccagccagagaacaactacaagacaacccctcctgtgctggacagcgacggctcattcttcctgtactccaagctgactgtggacaagagccggtggcagcagggcaatgtgttcagctgtagcgtgatgcacgaggccctgcacaaccactacacacagaagtccctgtctctgagccccggaaaaggtggcggtggctcttacccttacgacgtgccagattacgccggctatccctacgatgtgcctgactatgctggctacccctatgacgtccccgactacgcttaactagctacggaattccggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcagggttcgaaatcgataagcttggatccggagagctcccaacgcgtcggctagctagtagggataacagggtaataagcgtcgacggcgcgcccctaggggccggccttaattaaatcaagcttatcgataccgtcgaacctcgagggggggcatcactccgccctaaaacctacgtcacccgccccgttcccacgccccgcgccacgtcacaaactccaccccctcattatcatattggcttcaatccaaaataaggtatattattgatgatgttt

SEQ ID NO: 51 - DNA, 39752 bp, Artificial Sequence
  ICOSTAT
  misc_feature (5915)..(5917): n is a, c, g, or t
  misc_feature (32912)..(32912): n is a, c, g, or t
  misc_feature (34601)..(34601): n is a, c, g, or t
  misc_feature (38163)..(38168): n is a, c, g, or t
taacatcatcaattataccttccattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgtacgtcggcggctcgtggctcttccgggaaaaggattctcggaaagtggttcgagtacgtcggcggctcgtggctcttccgggaaaaggattctcggaaagtggttcgaagtacgtcgaccacaaaccccgcccagcgtcttgtcattggcgtcgacgctgtacggggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagactnnngttttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgacctagggataacagggtaataagcaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaataaactctaaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcaagtgatattaataaactccccgggcagctcacttaagttcatgtcgctgtccagctgctgagccacaggctgctgtccaacttgcggttgcttaacgggcggcgaaggagaagtccacgcctacatggggggagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgcttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgccccgccgggntatacactgcagggaaccgggacttggacaatgacaagtgggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatatttaagtccgggccattgtaaaaaatttggctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcagtccggagtcataatgtaagactcggtaaacacatcaggttgattcacatcggtcagtgttaaaaagcgaccgaaatagccngggggaatacaatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacggtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaacatgcatggatcctcgtctcgacgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttggttgnnnnnnaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaat

SEQ ID NO: 52 - protein, 8 aa, Artificial Sequence
  Amino acids 121-128 of Ad E1A protein
LTCHEACF

SEQ ID NO: 53 - protein, 9 aa, Artificial Sequence
  STAT1 binding site (1)
TTCCGGGAA

SEQ ID NO: 54 - protein, 9 aa, Artificial Sequence
  STAT1 binding site (2)
TTCTCGGAA

SEQ ID NO: 55 - DNA, 35010 bp, Artificial Sequence
  Ad5/3Ad2E1Adelta24
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtcggtgatagagtggtgggtttggtgtggtaatttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgttcaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcaccttctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggttacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggatggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgacctagggataacagggtaataagcaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaataaactctaaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaat

SEQ ID NO: 56 - protein, 464 aa, Artificial Sequence
  HER2(A3)-CD28TM,ICD-CD3Z CAR
MTRAMDWIWRILFLVGAATGAHSEVQLVQSGTEVKKPGASVRVSCKSSGYTFTSYYIHWVRQAPGQGLEWMAIINPGNGDTNYAQRFQGRVTMTRDTSTSTVYMELRSLRSDDTAVYFCAREIASYSGSYYDYWGQGTLVTVSSGGGGSGGGGSGGGGSQAVVLQEPSLSVSPGGTVTLTCGLSSGSVSTGHYASWYQQTPGQAPRTLFYNTNTRSSGVPDRFSGSIVGNKAALTITGAQADDESDYYCVLYVGDGIWVFGGGTKLTVLEPKSCDKTHTCPTRFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - protein, 14 aa, Artificial Sequence
  HER2(A3) LC-CDR1
GLSSGSVSTGHYAS

SEQ ID NO: 58 - protein, 7 aa, Artificial Sequence
  HER2(A3) LC-CDR2
NTNTRSS

SEQ ID NO: 59 - protein, 10 aa, Artificial Sequence
  HER2(A3) LC-CDR3
VLYVGDGIWV

SEQ ID NO: 60 - protein, 10 aa, Artificial Sequence
  HER2(A3) HC-CDR1
SYYIHWVRQA

SEQ ID NO: 61 - protein, 17 aa, Artificial Sequence
  HER2(A3) HC-CDR2
IINPGNGDTNYAQRFQG

SEQ ID NO: 62 - protein, 12 aa, Artificial Sequence
  HER2(A3) HC-CDR3
EIASYSGSYYDY

SEQ ID NO: 63 - protein, 110 aa, Artificial Sequence
  HER2(A3) VL
QAVVLQEPSLSVSPGGTVTLTCGLSSGSVSTGHYASWYQQTPGQAPRTLFYNTNTRSSGVPDRFSGSIVGNKAALTITGAQADDESDYYCVLYVGDGIWVFGGGTKLTVL

SEQ ID NO: 64 - protein, 121 aa, Artificial Sequence
  HER2(A3) VH
EVQLVQSGTEVKKPGASVRVSCKSSGYTFTSYYIHWVRQAPGQGLEWMAIINPGNGDTNYAQRFQGRVTMTRDTSTSTVYMELRSLRSDDTAVYFCAREIASYSGSYYDYWGQGTLVTVSS

(19) United States
(12) Patent Application Publication
(10) Pub. No.: US 2022/0387530 A1
(43) Pub. Date: Dec. 8, 2022

(54) ONCOLYTIC VIROTHERAPY AND IMMUNOTHERAPY

(71) Applicant: Baylor College of Medicine, Houston, TX (US)

(72) Inventors: Masataka Suzuki, Houston, TX (US); Amanda Rosewell Shaw, Pearland, TX (US); Caroline Elaine Porter, Houston, TX (US); Norihiro Watanabe, Houston, TX (US); Malcolm K. Brenner, Bellaire, TX (US); Andrew Sikora, Houston, TX (US)

(73) Assignee: Baylor College of Medicine, Houston, TX (US)

(21) Appl. No.: 17/775,203

(22) PCT Filed: Nov. 7, 2019
(86) PCT No.: PCT/US2019/060250

§ 371 Date: May 6, 2022

(51) Int. Cl. A61K 35/768; A61P 35/00; A61K 38/20; A61K 39/395; A61K 35/17; C07K 14/005; A61K 38/17

(52) U.S. Cl. CPC A61K 35/768; A61P 35/00; A61K 38/208; A61K 39/39558; A61K 35/17; C07K 14/005; A61K 38/1774; A61K 2039/545

(57) ABSTRACT

The present disclosure concerns combination therapy for cancer thatutilizes (i) an oncolytic virus; (ii) a virus comprising nucleic acid encoding an immunomodulatory factor; and (iii) at least one cell comprising a chimeric antigen receptor (CAR) specific for a cancer cell antigen. In particular embodiments, the virus comprises nucleic acid encoding an immunomodulatory factor comprises nucleic acid encoding IL-12 and/or antagonist anti-PD-L1 antibody.

### TECHNICAL FIELD

The present disclosure relates at least to the fields of cell biology, molecular biology, immunology, virology, and medicine, including cancer therapy. In particular embodiments the disclosure relates to combination treatments involving the use of oncolytic virotherapy and immunotherapy.

### BACKGROUND

Oncolytic virotherapy for squamous cell carcinoma of the head and neck (HNSCC) HNSCC is the sixth leading cancer by incidence worldwide. Treatment of locally advanced, recurrent and metastatic HNSCC is often limited by an unfavorable efficacy to toxicity ratio and median survival for patients with metastatic disease remains less than one year (Zandberg and Strome, Oral Oncology (2014) 50: 627-632). Since HNSCC is a locoregional disease that presents at or close to the surface of the body, it is amenable to initial intratumoral injection of adenoviral vectors (Ads) to prompt a loco-regional and even a systemic anti-tumor immune response (Liu et al., Nature Clinical Practice Oncology (2007) 4: 101-117). Several clinical trials of conditionally-replicating Ads (OncAds) or replication-deficient Ads encoding a therapeutic transgene have demonstrated the safety and feasibility of Ad gene therapy for HNSCC, but failed to show improved overall survival since intensive local treatment, even when combined with chemo/radiotherapy, did not prevent metastasis to distant sites (Liu et al., supra). OncAds are generally administered intratumorally, and poorly re-target to metastasized tumors (Koksi et al., Molecular Therapy: The Journal of the American Society of Gene Therapy (2015) 23:1641-1652).

OncAd with Helper-Dependent Ad (HDAd) Expressing Immunomodulatory Molecules

Adenoviral-based vectors (Ads) can infect a range of malignant cells and express high levels of lytic antigens and immunogenic transgenes, making them attractive as agents for cancer gene therapy (Cerullo et al., Advances in Cancer Research (2012) 115, 265-318). OncAds selectively replicate in cancer cells and are commonly used Ad-based vectors in clinical trials for cancer gene therapy. However, OncAds have a limited coding capacity for transgenes (˜1.5 kb). Helper-dependent Ads (HDAds) are devoid of viral coding sequences, enabling a cargo capacity of up to 34 kb for insertion of multiple transgenes in a single vector (Suzuki et al., Human Gene Therapy (2010) 21; 120-126). Since HDAd vector DNA encodes packaging signals, the OncAd replication machinery acts in trans to replicate and package both OncAd and HDAd within infected tumor cells, leading to multiple cycles of production and release of both the oncolytic virus and the transgenes encoded by the HDAd (combinatorial adenoviral vectors: CAd-VEC; Farzad et al., Molecular Therapy—Oncolytics (2014) 1, 14008).

CAR T-Cell Therapy

The use of T-cells as agents for cancer therapy has recently been facilitated by the expression of cancer cell antigen-directed chimeric antigen receptors (CARs; reviewed in Kershaw et al., Nature (2013) 13: 525-541). CAR-modified T-cells have shown promise for the treatment of hematological malignancies (Garfall et al., The New England Journal of Medicine (2015) 373:1040-1047), but have been less effective in treating solid tumors, which may in part be a consequence of the highly immunosuppressive nature of the solid tumor microenvironment (Quail et al., Nature Medicine (2013) 19:1423-1437). Due to immunosuppressive mechanisms at tumor site CAR T-cells fail to expand and persist long term despite the expression of one or two costimulatory endodomains.

The present disclosure provides a solution to a long-felt need for effective cancer therapies, including combinatorial cancer therapies.

### BRIEF SUMMARY

In one aspect, the present disclosure provides a method of treating a cancer, comprising administering to a subject:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is a combination of (i) an oncolytic virus, (ii) a virus comprising nucleic acid encoding an immunomodulatory factor, and (iii) at least one cell comprising a chimeric antigen receptor (CAR) specific for a cancer cell antigen, for use in a method of treating a cancer.

Also provided is the use of (i) an oncolytic virus, (ii) a virus comprising nucleic acid encoding an immunomodulatory factor, and (iii) at least one cell comprising a chimeric antigen receptor (CAR) specific for a cancer cell antigen, in the manufacture of a medicament for use in a method of treating a cancer.

In some embodiments the methods, compositions and uses of present disclosure comprise administering and/or providing (i) and (ii) in separate compositions. In some embodiments the methods, compositions and uses of present disclosure comprise administering and/or providing (i) and (ii) in the same composition.

Also provided is a method of treating a cancer, comprising administering to a subject:
(i) an oncolytic virus; and
(ii) at least one cell comprising a chimeric antigen receptor (CAR) specific for a cancer cell antigen.

Also provided is a combination of (i) an oncolytic virus, and (ii) at least one cell comprising a chimeric antigen receptor (CAR) specific for a cancer cell antigen, for use in a method of treating a cancer.

Also provided is the use of (i) an oncolytic virus, and (ii) at least one cell comprising a chimeric antigen receptor (CAR) specific for a cancer cell antigen, in the manufacture of a medicament for use in a method of treating a cancer.

Also provided is an oncolytic virus for use in a method of treating a cancer, the method comprising separate or simultaneous administration to a subject of:
(i) an oncolytic virus; and
(ii) at least one cell comprising a chimeric antigen receptor (CAR) specific for a cancer cell antigen.

Also provided is at least one cell comprising a chimeric antigen receptor (CAR) specific for a cancer cell antigen for use in a method of treating a cancer, the method comprising separate or simultaneous administration to a subject of:
(i) an oncolytic virus; and
(ii) at least one cell comprising a chimeric antigen receptor (CAR) specific for a cancer cell antigen.

Also provided is the use of an oncolytic virus in the manufacture of a medicament for use in a method comprising separate or simultaneous administration to a subject of:
(i) an oncolytic virus; and
(ii) at least one cell comprising a chimeric antigen receptor (CAR) specific for a cancer cell antigen.

Also provided is the use of at least one cell comprising a chimeric antigen receptor (CAR) specific for a cancer cell antigen in the manufacture of a medicament for use in a method comprising separate or simultaneous administration to a subject of:
(i) an oncolytic virus; and
(ii) at least one cell comprising a chimeric antigen receptor (CAR) specific for a cancer cell antigen.

In some embodiments the cell comprising a CAR is specific for the oncolytic virus.

Also provided is a method of treating a cancer, comprising administering to a subject:
(i) an oncolytic virus; and
(ii) at least one immune cell specific for the oncolytic virus.

Also provided is a combination of (i) an oncolytic virus, and (ii) at least one immune cell specific for the oncolytic virus, for use in a method of treating a cancer.

Also provided is the use of (i) an oncolytic virus, and (ii) at least one immune cell specific for the oncolytic virus, in the manufacture of a medicament for use in a method of treating a cancer.

Also provided is an oncolytic virus for use in a method of treating a cancer, the method comprising separate or simultaneous administration to a subject of:
(i) an oncolytic virus; and
(ii) at least one immune cell specific for the oncolytic virus.

Also provided is at least one immune cell specific for an oncolytic virus for use in a method of treating a cancer, the method comprising separate or simultaneous administration to a subject of:
(i) an oncolytic virus; and
(ii) at least one immune cell specific for the oncolytic virus.

Also provided is the use of an oncolytic virus in the manufacture of a medicament for use in a method comprising separate or simultaneous administration to a subject of:
(i) an oncolytic virus; and
(ii) at least one immune cell specific for the oncolytic virus.

Also provided is the use of at least one immune cell specific for an oncolytic virus in the manufacture of a medicament for use in a method comprising separate or simultaneous administration to a subject of:
(i) an oncolytic virus; and
(ii) at least one immune cell specific for the oncolytic virus.

In some embodiments the oncolytic virus and virus comprising nucleic acid encoding an immunomodulatory factor are administered at a total dose of 1×10¹⁰, 1×10¹¹, or 1×10¹² viral particles. In some embodiments the cells comprising a chimeric antigen receptor (CAR) specific for a cancer cell antigen and/or immune cells specific for an oncolytic virus are administered at a dose of 1×10⁶, 1×10⁷, or 1×10⁸ cells.

In some embodiments the oncolytic virus and/or virus comprising nucleic acid encoding an immunomodulatory factor are administered (i.e. on day 1), and cells comprising a CAR specific for a cancer cell antigen and/or immune cells specific for an oncolytic virus are administered three days later (i.e. on day 4).

In some embodiments the oncolytic virus and/or virus comprising nucleic acid encoding an immunomodulatory factor are administered via intratumoral administration (e.g. intratumoral injection). In some embodiments the cells comprising a CAR specific for a cancer cell antigen and/or immune cells specific for an oncolytic virus are administered via intravenous administration (e.g. intravenuous injection/infusion).

In some embodiments, the oncolytic virus and the virus comprising nucleic acid encoding an immunomodulatory factor are administered at a viral particle ratio of oncolytic virus:virus comprising nucleic acid encoding an immunomodulatory factor of from 1:0.5 (i.e. 2:1) to 1:25. In some embodiments, the oncolytic virus and the virus comprising nucleic acid encoding an immunomodulatory factor are administered at a viral particle ratio of oncolytic virus:virus comprising nucleic acid encoding an immunomodulatory factor of 1:1.

In some embodiments, the oncolytic virus is an oncolytic adenovirus (OncAd). In some embodiments, the oncolytic virus is derived from adenovirus 5 (Ad5). In some embodiments, the oncolytic virus encodes an E1A protein which displays reduced binding to Rb protein as compared to E1A protein encoded by Ad5. In some embodiments, the oncolytic virus encodes an E1A protein lacking the amino acid sequence LTCHEACF (SEQ ID NO:52). In some embodiments, the oncolytic virus encodes an E1A protein comprising, or consisting of or consisting essentially of, the amino acid sequence SEQ ID NO:34. In some embodiments, the oncolytic virus comprises nucleic acid having one or more binding sites for one or more transcription factors. In some embodiments, the oncolytic virus comprises nucleic acid having one or more binding sites for STAT1.

In some embodiments, the virus comprising nucleic acid encoding an immunomodulatory factor is a helper-dependent adenovirus (HDAd). In some embodiments, the immunomodulatory factor is selected from: an agonist of an effector immune response or antagonist of an immunoregulatory response. In some embodiments, the virus comprising nucleic acid encoding an immunomodulatory factor comprises nucleic acid encoding IL-12 and/or antagonist anti-PD-L1 antibody. In some embodiments, the virus comprising nucleic acid encoding an immunomodulatory factor comprises nucleic acid encoding an enzyme capable of catalysing conversion of a non-toxic factor to a cytotoxic form. In some embodiments, the enzyme is selected from: thymidine kinase, cytosine deaminase, nitroreductase, cytochrome P450, carboxypeptidase G2, purine nucleoside phosphorylase, horseradish peroxidase and carboxylesterase. In some embodiments, the virus comprising nucleic acid encoding an immunomodulatory factor comprises nucleic acid encoding a thymidine kinase.

In some embodiments, the at least one cell comprising a CAR specific for a cancer cell antigen is a T cell. In some embodiments, the CAR comprises an antigen binding domain capable of specific binding to HER2. In some embodiments, the CAR comprises an antigen binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;

and a VH domain comprising:
HC-CRD1: SEQ ID NO:21;
HC-CRD2: SEQ ID NO:22;
HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

In some embodiments, the CAR comprises an antigen binding domain comprising:
a VL comprising, or consisting of or consisting essentially of, an amino acid sequence having at least 75%, 80%, 85%, 90%, 95% or greater sequence identity to SEQ ID NO:16 and a VH comprising, or consisting of or consisting essentially of, an amino acid sequence having at least 75%, 80%, 85%, 90%, 95% or greater sequence identity to SEQ ID NO:17;

or
a VL comprising, or consisting of or consisting essentially of, an amino acid sequence having at least 75%, 80%, 85%, 90%, 95% or greater sequence identity to SEQ ID NO:24 and a VH comprising, or consisting of or consisting essentially of, an amino acid sequence having at least 75%, 80%, 85%, 90%, 95% or greater sequence identity to SEQ ID NO:25;

or
a VL comprising, or consisting of or consisting essentially of, an amino acid sequence having at least 75%, 80%, 85%, 90%, 95% or greater sequence identity to SEQ ID NO:32 and a VH comprising, or consisting of or consisting essentially of, an amino acid sequence having at least 75%, 80%, 85%, 90%, 95% or greater sequence identity to SEQ ID NO:33;

or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

In some embodiments, the method additionally comprises:
(a) isolating at least one cell from a subject, and in specific embodiments the cell is an immune cell;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject; in specific embodiments the modified cell upon administration is provided to the subject with one or more other agents for cancer therapy.

In some embodiments, the method of treating a cancer comprises:
(a) isolating at least one cell from a subject;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject.

In some embodiments, the method of treating a cancer comprises:
(a) isolating immune cells from a subject;
(b) generating or expanding a population of immune cells specific for an oncolytic virus by a method comprising: stimulating the immune cells by culture in the presence of antigen presenting cells (APCs) presenting a peptide of the oncolytic virus, and;
(c) administering at least one immune cell specific for the oncolytic virus to a subject.

In some embodiments, the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

The present disclosure also provides an oncolytic adenovirus (OncAd) encoding an E1A protein comprising, or consisting of or consisting essentially of, the amino acid sequence SEQ ID NO:34.

The present disclosure also provides an oncolytic adenovirus (OncAd) comprising nucleic acid having one or more binding sites for STAT1. In some embodiments, the OncAd comprises a nucleic acid sequence having at least 60%, 65%, 70%, 75%, 80%, 85%, 90%, 95% or greater sequence identity to SEQ ID NO:51 or an equivalent sequence as a result of codon degeneracy.

Also provided is an OncAd comprising a nucleic acid sequence having at least 60%, 65%, 70%, 75%, 80%, 85%, 90%, 95% or greater sequence identity to SEQ ID NO:55 or an equivalent sequence as a result of codon degeneracy. In some embodiments the OncAd encodes an E1A protein comprising, or consisting of or consisting essentially of, the amino acid sequence SEQ ID NO:34.

The present disclosure also provides a helper-dependent adenovirus (HDAd) comprising nucleic acid encoding IL-12 and/or antagonist anti-PD-L1 antibody. In some embodiments the HDAd additionally comprises nucleic acid encoding an enzyme capable of catalysing conversion of a non-toxic factor to a cytotoxic form. In some embodiments the enzyme is selected from: thymidine kinase, cytosine deaminase, nitroreductase, cytochrome P450, carboxypeptidase G2, purine nucleoside phosphorylase, horseradish peroxidase and carboxylesterase.

In some embodiments, the HDAd additionally comprises nucleic acid encoding a thymidine kinase. In cases wherein the HDAd nucleic acid encodes IL-12 and anti-PD-L1 antibody, the respective expression sequences may or may not be regulated by the same regulatory sequence.

In such cases wherein the HDAd nucleic acid encodes both IL-12 and anti-PD-L1 antibody, the positioning on the HDAd nucleic acid may be of any suitable configuration, such as in a 5′ to 3′ direction the nucleic acid region encoding IL-12 being either upstream or downstream of the nucleic acid region encoding anti-PD-L1 antibody.

The present disclosure also provides a chimeric antigen receptor (CAR) comprising an antigen binding domain comprising:
a VL domain comprising:
LC-CRD1: SEQ ID NO:10;
LC-CRD2: SEQ ID NO:11;
LC-CRD3: SEQ ID NO:12;
and a VH domain comprising:
HC-CRD1: SEQ ID NO:13;
HC-CRD2: SEQ ID NO:14;
HC-CRD3: SEQ ID NO:15;

or
a VL domain comprising:
LC-CRD1: SEQ ID NO:18;
LC-CRD2: SEQ ID NO:19;
LC-CRD3: SEQ ID NO:20;
and a VH domain comprising:
HC-CRD1: SEQ ID NO:21;
HC-CRD2: SEQ ID NO:22;
HC-CRD3: SEQ ID NO:23;

or
a VL domain comprising:
LC-CRD1: SEQ ID NO:26;
LC-CRD2: SEQ ID NO:27;
LC-CRD3: SEQ ID NO:28;
and a VH domain comprising:
HC-CRD1: SEQ ID NO:29;
HC-CRD2: SEQ ID NO:30;
HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

In some embodiments, the CAR comprises an antigen binding domain comprising:
a VL comprising, or consisting of or consisting essentially of, an amino acid sequence having at least 75%, 80%, 85%, 90%, 95% or greater sequence identity to SEQ ID NO:16 and a VH comprising, or consisting of or consisting essentially of, an amino acid sequence having at least 75%, 80%, 85%, 90%, 95% or greater sequence identity to SEQ ID NO:17;

or
a VL comprising, or consisting of, an amino acid sequence having at least 75%, 80%, 85%, 90%, 95% or greater sequence identity to SEQ ID NO:24 and a VH comprising, or consisting of or consisting essentially of, an amino acid sequence having at least 75%, 80%, 85%, 90%, 95% or greater sequence identity to SEQ ID NO:25;

or
a VL comprising, or consisting of, an amino acid sequence having at least 75%, 80%, 85%, 90%, 95% or greater sequence identity to SEQ ID NO:32 and a VH comprising, or consisting of or consisting essentially of, an amino acid sequence having at least 75%, 80%, 85%, 90%, 95% sequence identity to SEQ ID NO:33;

or
a VL comprising, or consisting of, an amino acid sequence having at least 75%, 80%, 85%, 90%, 95% or greater sequence identity to SEQ ID NO:63 and a VH comprising, or consisting of or consisting essentially of, an amino acid sequence having at least 75%, 80%, 85%, 90%, 95% sequence identity to SEQ ID NO:64.

The present disclosure also provides a nucleic acid, or a plurality of nucleic acids, optionally isolated, encoding the oncolytic adenovirus (OncAd), the helper-dependent adenovirus (HDAd), and/or the chimeric antigen receptor (CAR) according to the present disclosure.

The present disclosure also provides a cell comprising the oncolytic adenovirus (OncAd), the helper-dependent adenovirus (HDAd), the chimeric antigen receptor (CAR), and/or the nucleic acid or plurality of nucleic acids according to the present disclosure.

The present disclosure also provides a nucleic acid, or a plurality of nucleic acids, optionally isolated, encoding the oncolytic adenovirus (OncAd) and/or the chimeric antigen receptor (CAR) according to the present disclosure. The present disclosure also provides a cell comprising the oncolytic adenovirus (OncAd), the chimeric antigen receptor (CAR), and/or the nucleic acid or plurality of nucleic acids according to the present disclosure. Also provided is a cell comprising the oncolytic adenovirus (OncAd) and the chimeric antigen receptor (CAR) according to the present disclosure.

The present disclosure also provides a pharmaceutical composition comprising the oncolytic adenovirus (OncAd), the helper-dependent adenovirus (HDAd), the chimeric antigen receptor (CAR); the nucleic acid or plurality of nucleic acids or the cell according to the present disclosure may be associated with or comprised in a pharmaceutically acceptable carrier, diluent, excipient or adjuvant.

The present disclosure also provides a method of treating cancer comprising administering to a subject the oncolytic adenovirus (OncAd), the helper-dependent adenovirus (HDAd), the chimeric antigen receptor (CAR), the nucleic acid or plurality of nucleic acids, the cell or the pharmaceutical composition according to the present disclosure.

The present disclosure also provides the oncolytic adenovirus (OncAd), the helper-dependent adenovirus (HDAd), the chimeric antigen receptor (CAR), the nucleic acid or plurality of nucleic acids, the cell or the pharmaceutical composition according to the present disclosure for use in a method of treating a cancer.

The present disclosure also provides the use of the oncolytic adenovirus (OncAd), the helper-dependent adenovirus (HDAd), the chimeric antigen receptor (CAR), the nucleic acid or plurality of nucleic acids, the cell or the pharmaceutical composition according to the present disclosure in the manufacture of a medicament for treating a cancer.

In some embodiments in accordance with various aspects of the present disclosure, the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

The present disclosure also provides a kit of parts comprising a predetermined quantity of the oncolytic adenovirus (OncAd), the helper-dependent adenovirus (HDAd), the chimeric antigen receptor (CAR), the nucleic acid or plurality of nucleic acids, the cell and/or the pharmaceutical composition according to the present disclosure.

### DETAILED DESCRIPTION

The present disclosure is concerned with the combined use of multiple therapeutic agents for the treatment of cancer. In particular, (i) oncolytic virus, (ii) virus providing immunomodulatory factor(s) and (iii) CAR-bearing immune cells (such as T cells) specific for a cancer cell antigen are used in combination as a cancer therapy. The therapeutic agents are combined to provide an improved treatment effect as compared to the effect seen when any one of the agents is used alone. In certain embodiments, at least two of the three therapeutic agents act in an additive manner to treat the cancer, whereas in other embodiments at least two of the three different therapeutic agents act synergistically to treat the cancer.

Local treatment with oncolytic virus is ineffective in patients suffering from diffuse/metastatic cancer, e.g. head and neck squamous cell carcinoma, because locally injected virus tends to have limited distribution to metastasised tumours (Kanerva, A., et al., Clin Cancer Res, 2013. 19(10): p. 2734-44). Separately, oncolytic virus and HER2.CAR T cells have been shown to effectively kill HNSCC cell lines in vitro, but neither treatment showed substantial anti-tumour effects on HNSCC tumours in vivo when used alone or in combination, consistent with their limited activity in clinical trials for glioblastoma and sarcoma (Ahmed N., et al., JAMA Oncol 2017. 3(8): p. 1094-1101; Ahmed et al., J Clin Oncol. 2015 May 20; 33(15):1688-96).

Without wishing to be bound by any particular theory, the improved treatment effect is thought to be achieved by combining the advantageous features of oncolytic virotherapy (e.g. effective treatment of solid tumours) and CAR-T cell therapy (e.g. effective treatment of diffuse/metastatic cancer), in conjunction with providing a favourable immune environment for CAR-T cell proliferation and activity. The CAR-T cells activated and expanded at primary tumor sites can persist, re-circulate and target metastasized tumors.

Oncolytic Virus

The present disclosure employs oncolytic virus. Oncolytic viruses and their use to treat cancer is reviewed, for example, in Chiocca and Rabkin Cancer Immunol Res (2014) 2(4): 295-300, which is hereby incorporated by reference in its entirety.

Oncolytic viruses replicate in, and cause lysis of, cancer cells. Often they are selective for cancer cells over non-cancerous cells; for example, oncolytic viruses commonly replicate in dividing cells in preference to non-dividing cells. Oncolytic viruses are therefore useful to selectively kill cancer cells and destroy tumours, without causing substantial damage to normal, non-cancerous cells/tissue.

Oncolytic virotherapy is associated with several advantages features. Oncolytic viruses often target several oncogenic pathways and use multiple mechanisms for cytotoxicity, minimising the chances of resistance arising. As noted above, because oncolytic viruses replicate selectively in tumours and are non-pathogenic they display minimal toxicity. Virus dose in the tumour also increases overtime due to replication of the virus, and the oncolytic viruses can also be manipulated genetically to improve safety, e.g. by engineering sensitivity to a drug.

There are two main classes of oncolytic virus:
(i) viruses that naturally replicate preferentially in cancer cells, and which are non-pathogenic in humans often due to elevated sensitivity to innate antiviral signalling or dependence on oncogenic signalling pathways, including autonomous parvoviruses, myxoma virus (MYXV; poxvirus), Newcastle disease virus (NDV; paramyxovirus), reovirus, and Seneca valley virus (SVV; picornavirus); and
(ii) viruses that are genetically-manipulated, e.g. with mutations/deletions in genes required for replication in normal, but not cancer cells, including adenovirus (Ad), herpes simplex virus (HSV), vaccinia virus (VV), and vesicular stomatitis virus (VSV; rhabdovirus); or viruses that are genetically-manipulated for use as vaccine vectors including measles virus (MV; paramyxovirus), poliovirus (PV; picornavirus), and VV (poxvirus).

Genetic manipulation can include insertion/alteration of functional sequences to provide enhanced selectivity for cancer cells, safety, and/or to modify virus tropism.

For example, oncolytic virus may by genetically engineered to introduce tissue-specific internal ribosome entry sites (IRESs) only permitting viral translation in target cells, and/or to introduce miRNAs/miRNA response elements (MREs); differential miRNA expression between healthy cells or certain tissues vs. tumor cells allows viruses to be detargeted from healthy cells/tissues. Oncolytic virus may also by engineered to place transcription of the viral genome under the control of a cell- or tissue-specific regulatory region, such as promoter/enhancers (e.g. tumour cell-specific promoter). In some embodiments, the oncolytic virus according to the present disclosure may comprise one or more modifications for such purpose.

Virus may also be modified for transductional targeting, e.g. through modification of virus receptors/coat proteins to target tumour cells and/or detarget healthy cells/tissues.

Oncolytic viruses may be administered in such a way as to minimise anti-oncolytic virus responses (e.g. neutralisation by anti-virus antibodies) in the subject and sequestration in the liver, and to maximise tumour delivery, as described in Chiocca and Rabkin, supra. For example, oncolytic virus may be administered in a cell carrier, e.g. in mesenchymal stromal cells, myeloid-derived suppresser cells (MDSCs), neural stem cells, T cells, cytokine-induced killer cells, or irradiated tumor cells, or can be coated in nanoparticles.

In some embodiments, the oncolytic virus of the present disclosure is, or is derived from, an adenovirus (Ad), herpes simplex virus (HSV), vaccinia virus (VV), vesicular stomatitis virus (VSV); autonomous parvovirus, myxoma virus (MYXV), Newcastle disease virus (NDV), reovirus, Seneca valley virus (SVV) morbillivirus virus, retrovirus, influenza virus, Sindbis virus (SINV) or poxvirus, as examples. In some embodiments, the oncolytic virus is not vaccinia virus. In some embodiments, the oncolytic virus is not vaccinia virus JX-594.

As used herein, an oncolytic virus which is “derived from” a reference virus comprises a nucleic acid sequence or amino acid sequence which is possessed by the reference virus. In some embodiments an oncolytic virus which is “derived from” a reference virus comprises one or more genes possessed by the reference virus. In some embodiments an oncolytic virus which is “derived from” encodes one or more proteins encoded by the reference virus.

In some embodiments, an oncolytic virus which is derived from a reference virus may comprise nucleic acid sequence encoding one or more functional elements of the reference virus. A “functional element” may e.g. be a transcriptional regulator (e.g. a promoter/enhancer), a regulator of post-transcriptional processing, a translational regulator, a regulator of post-transcriptional processing, a response element, a repeat sequence, or a viral protein. In some embodiments, an oncolytic virus which is derived from a reference virus may comprise one or more genes of, or proteins encoded by, the reference virus.

In some embodiments the oncolytic virus of the present disclosure is, or is derived from, an adenovirus (OncAd). OncAds are reviewed e.g. in Larson et al., Oncotarget. (2015) 6(24): 19976-19989, which is hereby incorporated by reference in its entirety.

In some embodiments the OncAd is, or is derived from, a species A, B, C, D, E, F or G human adenovirus (i.e. HAdV-A, HAdV-B, HAdV-C, HAdV-D, HAdV-E, HAdV-F or HAdV-G). In some embodiments the OncAd is, or is derived from, a species C human adenovirus. In some embodiments the OncAd is, or is derived from, Ad5, Ad2, Ad1, Ad6 or Ad57.

In some embodiments the OncAd is a conditionally replicating adenovirus (or CRAd).

In some embodiments the OncAd has reduced ability to infect, replicate in and/or lyse non-cancerous cells (as compared to the ability to infect/replicate in and/or lyse equivalent cancerous cells), for example as a consequence of a genetic modification of the adenovirus from which the OncAd is derived.

In some embodiments the oncolytic virus comprises a modification to one or more protein encoding sequences. In some embodiments, the modification alters the production or activity of the encoded protein. In some embodiments, the modification is a truncation or deletion of the protein.

In some embodiments, the OncAd comprises modification to an adenovirus early protein. In some embodiments, the modification is to the region encoding E1A protein. In some embodiments, the OncAd encodes an E1A protein having reduced ability to bind to Rb protein as compared to wildtype E1A protein (e.g. E1A encoded by the adenovirus from which the OncAd is derived). In some embodiments the OncAd encodes an E1A protein lacking the amino acid sequence LTCHEACF (SEQ ID NO:52). An example of an OncAd comprising encodes an E1A protein lacking the amino acid sequence LTCHEACF (SEQ ID NO:52) is Onc5/3Ad2E1Δ24 shown in SEQ ID NO:55.

In some embodiments the oncolytic virus encodes an E1A protein comprising, or consisting of or consisting essentially of, an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:34.

In some embodiments, the oncolytic virus comprises a nucleic acid sequence providing one or more binding sites for one or more transcription factors. In some embodiments, the transcription factor is an activating transcription factor (i.e. a transcriptional activator). The one or more binding sites for one or more transcription factors are preferably provided upstream of (i.e. 5′ to) to nucleic acid sequence encoding one or more functional elements (e.g. viral proteins).

In some embodiments, the transcription factor is a transcription factor having increased expression, or increased activity, in cancerous cells as compared to comparable non-cancerous cells (e.g. non-cancerous cells derived from the same tissue/cell type).

Herein, “expression” may refer to gene expression or protein expression. Gene expression can be measured by various means known to those skilled in the art, for example by measuring levels of mRNA by quantitative real-time PCR (qRT-PCR), or by reporter-based methods. Similarly, protein expression can be measured by various methods well known in the art, e.g. by antibody-based methods, for example by western blot, immunohistochemistry, immunocytochemistry, flow cytometry, ELISA, ELISPOT, or reporter-based methods.

An example of an OncAd comprising one or more binding sites for one or more transcription factors is ICOVIR15 described in Rojas et al. 2010 Mol Ther 18 1960-1971, which is hereby incorporated by reference its entirety. ICOVIR15 comprises 8 binding sites for the transcription factor E2F.

In some embodiments the oncolytic virus comprises one or more binding sites for a transcription factor whose gene or protein expression, or activity in a cell, is upregulated in response to a factor produced or expressed by an immune cell. In some embodiments, a factor produced or expressed by an immune cell may at least one cytokine/chemokine produced by, or a protein expressed at the cell surface of, an effector immune cell, e.g. CD8+ cytotoxic T lymphocyte (CTL), CD4+ T helper 1 (T_{H}1) cell, natural killer (NK) cell or natural killer T (NKT) cell.

In some embodiments, the oncolytic virus of the present disclosure comprises one or more binding sites for a STAT transcription factor. In some embodiments, the oncolytic virus comprises one or more binding sites for a STAT1. An ICOSTAT OncAd described herein possesses 8 binding sites for STAT1, and STAT1 is known to be upregulated by IFNγ. In particular embodiments, ICOSTAT is a particularly effective treatment for a cancer because the host's immune response to the cancer cells will promote the replication of the oncolytic virus in situ.

In some embodiments, the oncolytic virus comprises more than one binding site for a STAT1, e.g. at least 2, 3, 4, 5, 6, 7, 8, 9, or 10 binding sites for STAT1. In some embodiments, a binding site for STAT1 may comprise or consist of or consist essentially of the sequence TTCCGGGAA (SEQ ID NO:53), or TTCTCGGAA (SEQ ID NO:54). In some embodiments, the oncolytic virus of the present disclosure comprises one or more copies of the sequence TTCCGGGAA (SEQ ID NO:53) or TTCTCGGAA (SEQ ID NO:54).

In some embodiments the oncolytic virus according to the present disclosure comprises, or consists of, or consists essentially of, a nucleic acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:51 or an equivalent sequence as a result of codon degeneracy.

In some embodiments the oncolytic virus according to the present disclosure comprises, or consists of, or consists essentially of, a nucleic acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:55 or an equivalent sequence as a result of codon degeneracy.

In some embodiments the oncolytic virus according to the present disclosure encodes the same proteins as the proteins encoded by an oncolytic virus comprising, consisting of, or consisting essentially of, the nucleic acid shown in SEQ ID NO:55. In some embodiments the oncolytic virus according to the present disclosure encodes the same proteins as the proteins encoded by an oncolytic virus comprising, consisting of, or consisting essentially of, the nucleic acid shown in SEQ ID NO:51.

Virus Comprising Nucleic Acid Encoding an Immunomodulatory Factor

The present disclosure employs a virus comprising nucleic acid encoding an immunomodulatory factor. The virus acts as a vector for delivering the immunomodulatory factor. In certain embodiments, the virus comprises nucleic acid encoding more than one immunomodulatory factor(s).

Any virus capable of introducing the nucleic acid encoding an immunomodulatory factor into a cell (e.g. a primary human immune cell) may be used. Suitable viruses include gammaretrovirus (e.g. murine Leukemia virus (MLV)-derived vectors), lentivirus, adenovirus, adeno-associated virus, vaccinia virus and herpesvirus, e.g. as described in Maus et al., Annu Rev Immunol (2014) 32:189-225 or Morgan and Boyerinas, Biomedicines 2016 4, 9, which are both hereby incorporated by reference in its entirety. In some embodiments, the virus comprising nucleic acid encoding an immunomodulatory factor is, or is derived from, an adenovirus, lentivirus, retrovirus, or herpesvirus.

In some embodiments, the virus comprising nucleic acid encoding at least one immunomodulatory factor is an oncolytic virus comprising nucleic acid encoding at least one immunomodulatory factor.

An immunomodulatory factor(s) encoded by the virus comprising nucleic acid encoding the immunomodulatory factor(s) according to the present disclosure are preferably selected to facilitate the immune response to a cancer in a subject, in particular the cell-mediated immune response. In one embodiment, the immunomodulatory factor(s) provide favourable conditions for the activation, recruitment, proliferation, activity and/or survival of effector immune cells (e.g. CTLs, T_{H}1 cells, NK cells or NKT cells).

In some embodiments, the immunomodulatory factor may be an agonist of an effector immune response, e.g. a cytokine or chemokine promoting activation, recruitment, proliferation, activity and/or survival of effector immune cells (e.g. IL-2, IL-7, IL-17, IL-12, IL-21, IL-15, MIP-1a or RANTES), agonist antibody for a costimulatory receptor (e.g. 4-1 BB, OX40, CD28, CD27, ICOS, CD30 or GITR), or ligand for a costimulatory receptor (e.g. 4-1 BBL, OX40L, CD80, CD86, CD70, ICOSL, CD30L or GITRL). In some embodiments, the agonist of an effector immune response may be an antagonist of an immune checkpoint inhibitor, or an antagonist of ligand for immune checkpoint inhibitor, e.g. antagonist antibody to PD-L1, PD-L2, PD-1, CTLA-4, LAG-3, TIM-3, Gal-9, TIGIT, VISTA or BTLA, or an antagonist of a cytokine/chemokine which is an antagonist of an effector immune response, e.g. TGFβ (i.e. antagonist anti-TGFβ antibody or soluble/decoy TGFβ receptor). In some embodiments, an agonist of an effector immune response may be a molecule for engaging and co-opting bystander effector immune cells such as T cells and NK cells.

In some embodiments, the immunomodulatory factor may be an antagonist of an immunoregulatory response, e.g. an antagonist of a cytokine/chemokine promoting activation, recruitment, proliferation, activity and/or survival of immunoregulatory cells such as regulatory T cells (Tregs) and/or myeloid-derived suppressor cells (MDSCs), e.g. CCL9, CXCL10, CCL20, CCL22.

In some embodiments the virus comprising nucleic acid encoding an immunomodulatory factor may additionally comprise nucleic acid encoding further functional sequence(s). For example, the virus may comprise nucleic acid encoding a protein(s) for reducing growth/proliferation/survival of infected cells, or protein(s) for rendering infected cells sensitive to treatment with a given agent, or protein(s) for disrupting tumour structure (e.g. enzymes for digesting tumour matrix) to facilitate immune cell infiltration.

In some embodiments the virus comprising nucleic acid encoding an immunomodulatory factor additionally comprises nucleic acid encoding an enzyme capable of catalysing conversion of a non-toxic factor to a cytotoxic form. The enzyme may catalyse conversion of a non-toxic prodrug into its active, cytotoxic form.

Enzyme/prodrug systems are well known in the art and include those described in Malekshah et al. Curr Pharmacol Rep. (2016) 2(6): 299-308 which is hereby incorporated by reference in its entirety. Examples of non-toxic prodrugs, their active cytotoxic forms and enzymes capable of catalysing conversion of the non-toxic prodrugs to their active cytotoxic forms are shown in FIG. 2 of Malekshah et al.

In some embodiments the virus comprising nucleic acid encoding an immunomodulatory factor additionally comprises nucleic acid encoding a thymidine kinase, cytosine deaminase, nitroreductase, cytochrome P450, carboxypeptidase G2, purine nucleoside phosphorylase, horseradish peroxidase and/or carboxylesterase.

For example, the virus may comprise nucleic acid encoding thymidine kinase for rendering cells expressing the virus sensitive to treatment with ganciclovir (GCV), aciclovir (ACV) and/or valaciclovir. The virus may comprise nucleic acid encoding cytosine deaminase for rendering cells expressing the virus sensitive to treatment with 5-fluorocytosine (5-FC), which is converted by cytosine deaminase to 5-fluorouracil (5-FU). The virus may comprise nucleic acid encoding nitroreductase for rendering cells expressing the virus sensitive to treatment with CB1954, nitro-CBI-DEI and/or PR-104A. The virus may comprise nucleic acid encoding cytochrome P450 for rendering cells expressing the virus sensitive to treatment with oxazaphosphorine (e.g. cyclophosphamide or ifosfamide). The virus may comprise nucleic acid encoding carboxypeptidase G2 for rendering cells expressing the virus sensitive to treatment with nitrogen mustard based drugs (e.g. CMDA or ZD2767P). The virus may comprise nucleic acid encoding purine nucleoside phosphorylase for rendering cells expressing the virus sensitive to treatment with 6-methylpurine 2-deoxyriboside and/or fludarabine (e.g. 6-methylpurine-2′-deoxyriboside (MeP-dR), 2-F-2′-deoxyadenosine (F-dAdo) or arabinofuranosyl-2-F-adenine monophosphate (F-araAMP). The virus may comprise nucleic acid encoding horseradish peroxidase for rendering cells expressing the virus sensitive to treatment with indole-3-acetic acid (IAA). The virus may comprise nucleic acid encoding carboxylesterase for rendering cells expressing the virus sensitive to treatment with irinotecan.

In some embodiments the virus may comprise nucleic acid encoding antagonist of a growth factor.

In some embodiments, the virus may be a helper-dependent adenovirus (HDAd). HDAds are reviewed, for example, in Rosewell et al., J Genet Syndr Gene Ther (2011) Suppl 5:001, which is hereby incorporated by reference in its entirety.

HDAds are devoid of viral protein coding sequences, and therefore possess a large capacity (up to 37 Kb) for transduction of a coding sequence of interest. HDAds are non-integrating, and are able to efficiently transduce a wide variety of cell types independently of the cell cycle, and mediate long-term transgene expression without chronic toxicity.

HDAds comprise only the cis acting viral elements required for genomic replication (inverted terminal repeats (ITRs)) and encapsidation (ty), and are therefore dependent on helper virus for propagation. When a cell is infected with both the helper virus and the HDAd, the helper virus replication machinery acts in trans to replicate and package HDAd.

In particular embodiments of the present disclosure, the oncolytic virus is an OncAd and the virus comprising nucleic acid encoding an immunomodulatory factor is a HDAd, and the OncAd and HDAd are able to co-infect and replicate in cells of a cancer.

Dependence of the HDAd on help from the OncAd provides highly localised expression of the immunomodulatory factor(s). That is, because the HDAd is only able to propagate in cells co-infected with the OncAd, and in turn because the OncAd is selective for replication in cancerous cells, expression of the factor(s) encoded by the HDAd is restricted to cancerous cells/tissue, minimising side effects.

Furthermore, because the OncAd and HDAd efficiently target and infect tumour cells, expression of the immunomodulatory factor(s) in those cells can change the normally immunosuppressive tumour microenvironment to provide conditions promoting the activation, recruitment (i.e. tumour penetration/infiltration), proliferation, activity and/or survival of effector immune cells.

In particular, in the context of the present disclosure wherein the methods of treatment employ the use of CAR-T cells, expression of the immunomodulatory factor(s) encoded by the HDAd provide for enhanced activation, recruitment, proliferation, activity and/or survival of the CAR-T cells.

In particular embodiments herein the virus comprising nucleic acid encoding an immunomodulatory factor is a HDAd comprising nucleic acid encoding IL-12p70, HSV-1 thymidine kinase and an antagonist anti-PD-L1 minibody.

In some embodiments, the virus comprising nucleic acid encoding an immunomodulatory factor according to the present disclosure encodes IL-12. In some embodiments the virus comprising nucleic acid encoding an immunomodulatory factor comprises nucleic acid encoding an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:35.

In some embodiments, the virus comprising nucleic acid encoding an immunomodulatory factor according to the present disclosure encodes an antagonist of PD-1/PD-L1 signalling. In some embodiments the antagonist of PD-1/PD-L1 signalling is an anti-PD-L1 antibody.

In some embodiments the anti-PD-L1 antibody comprises an antigen binding domain comprising a VL domain comprising:
LC-CRD1: SEQ ID NO:39;
LC-CRD2: SEQ ID NO:40;
LC-CRD3: SEQ ID NO:41;

and a VH domain comprising:
HC-CRD1: SEQ ID NO:42;
HC-CRD2: SEQ ID NO:43;
HC-CRD3: SEQ ID NO:44.

In some embodiments the anti-PD-L1 antibody comprises a VL comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:45 and a VH comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:46.

In some embodiments the virus comprising nucleic acid encoding an immunomodulatory factor comprises nucleic acid encoding an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:38.

In some embodiments, the virus comprising nucleic acid encoding an immunomodulatory factor according to the present disclosure comprises an amino acid sequence encoding a thymidine kinase. In some embodiments the virus comprising nucleic acid encoding an immunomodulatory factor comprises nucleic acid encoding an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:36.

In some embodiments, the virus comprising nucleic acid encoding an immunomodulatory factor according to the present disclosure comprises, or consists of or consist essentially of, a nucleic acid sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:50 or an equivalent sequence as a result of codon degeneracy.

Chimeric Antigen Receptors (CARs) and CAR-Expressing Cells

The present disclosure employs immune cells comprising a chimeric antigen receptor (CAR).

Chimeric Antigen Receptors (CARs) are recombinant receptors that provide both antigen-binding and immune cell activating functions. CAR structure and engineering is reviewed, for example, in Dotti et al., Immunol Rev (2014) 257(1), hereby incorporated by reference in its entirety. CARs comprise an antigen-binding region linked to a cell membrane anchor region and a signaling region. An optional hinge region may provide separation between the antigen-binding region and cell membrane anchor region, and may act as a flexible linker.

The antigen-binding region of a CAR may be based on the antigen-binding region of an antibody which is specific for the antigen to which the CAR is targeted, or other agent capable of binding to the target. For example, the antigen-binding domain of a CAR may comprise amino acid sequences for the complementarity-determining regions (CDRs) or complete light chain and heavy chain variable region amino acid sequences of an antibody which binds specifically to the target protein. Antigen-binding domains of CARs may target antigen based on other protein:protein interaction, such as ligand:receptor binding; for example an IL-13Ra2-targeted CAR has been developed using an antigen-binding domain based on IL-13 (see e.g. Kahlon et al. 2004 Cancer Res 64(24): 9160-9166).

The CAR of the present disclosure comprises an antigen-binding region specific for a cancer cell antigen. The antigen binding region of the CAR may be provided with any suitable format, e.g. scFv, Fab, etc. In some embodiments, the antigen binding region of the CAR comprises or consists of a cancer cell antigen binding scFv.

A cancer cell antigen is an antigen which is expressed by a cancer cell. A cancer cell antigen may be any peptide/polypeptide, glycoprotein, lipoprotein, glycan, glycolipid, lipid, or fragment thereof. A cancer cell antigen's expression may be associated with a cancer. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response.

In some embodiments, the antigen is expressed at the cell surface of the cancer cell (i.e. the cancer cell antigen is a cancer cell surface antigen). In some embodiments, the part of the antigen which is bound by the bispecific antigen binding polypeptide of the present disclosure is displayed on the external surface of the cancer cell (i.e. is extracellular). In some embodiments, the antigen is anchored to the cell membrane, e.g. via a transmembrane domain or other membrane anchor (e.g. a lipid anchor such as a GPI anchor). In some embodiments, the cancer cell antigen is expressed at the cell surface (i.e. is expressed in or at the cell membrane) of a cancerous cell, but may be expressed inside the cell (i.e. is expressed inside comparable non-cancerous cells).

The cancer cell antigen may be a cancer-associated antigen. In some embodiments the cancer cell antigen is an antigen whose expression is associated with the development, progression and/or severity of symptoms of a cancer. The cancer-associated antigen may be associated with the cause or pathology of the cancer, or may be expressed abnormally as a consequence of the cancer. In some embodiments, the antigen is an antigen whose expression is upregulated (e.g. at the RNA and/or protein level) by cells of a cancer, e.g. as compared to the level of expression of by comparable non-cancerous cells (e.g. non-cancerous cells derived from the same tissue/cell type).

In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer cell antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6th edition. Hamilton (ON): BC Decker; 2003. Cancer cell antigens include oncofetal antigens: CEA, Immature laminin receptor, TAG-72; oncoviral antigens such as HPV E6 and E7; overexpressed proteins: BING-4, calcium-activated chloride channel 2, cyclin-B1, 9D7, Ep-CAM, EphA3, HER2/neu, telomerase, mesothelin, SAP-1, surviving; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-1/2, MC1R, prostate specific antigen; mutated antigens: β-catenin, BRCA1/2, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B.

In some embodiments, the cancer cell antigen is HER2. In some embodiments, the CAR of the present disclosure comprises an antigen binding domain capable of specific binding to HER2. In some embodiments, the CAR comprises an antigen binding domain comprising the CDRs of an antibody capable of specific binding to HER2. In some embodiments, the CAR comprises an antigen binding domain comprising the VL and VH regions of an antibody capable of specific binding to HER2.

In particular embodiments, the cell expressing the CAR comprises two, separate CARs each that target different cancer cell antigens, and in particular aspects at least one of the CARs targets HER2. In some cases, the CAR is bispecific for two different cancer cell antigens, one of which may be HER2.

In some embodiments the CAR comprises an antigen binding domain comprising a VL domain comprising:
LC-CRD1: SEQ ID NO:10, SEQ ID NO:18, SEQ ID NO:26 or SEQ ID NO: 57;
LC-CRD2: SEQ ID NO:11, SEQ ID NO:19, SEQ ID NO:27 or SEQ ID NO: 58;
LC-CRD3: SEQ ID NO:12, SEQ ID NO:20, SEQ ID NO:28 or SEQ ID NO: 59;

and a VH domain comprising:
HC-CRD1: SEQ ID NO:13, SEQ ID NO:21, SEQ ID NO:29 or SEQ ID NO: 60;
HC-CRD2: SEQ ID NO:14, SEQ ID NO:22, SEQ ID NO:30 or SEQ ID NO: 61;
HC-CRD3: SEQ ID NO:15, SEQ ID NO:23, SEQ ID NO:31 or SEQ ID NO: 62.

In some embodiments the CAR comprises an antigen binding domain comprising a VL domain comprising:
LC-CRD1: SEQ ID NO:10;
LC-CRD2: SEQ ID NO:11;
LC-CRD3: SEQ ID NO:12;

and a VH domain comprising:
HC-CRD1: SEQ ID NO:13;
HC-CRD2: SEQ ID NO:14;
HC-CRD3: SEQ ID NO:15.

In some embodiments the CAR comprises an antigen binding domain comprising a VL domain comprising:
LC-CRD1: SEQ ID NO:18;
LC-CRD2: SEQ ID NO:19;
LC-CRD3: SEQ ID NO:20;

and a VH domain comprising:
HC-CRD1: SEQ ID NO:21;
HC-CRD2: SEQ ID NO:22;
HC-CRD3: SEQ ID NO:23.

In some embodiments the CAR comprises an antigen binding domain comprising a VL domain comprising:
LC-CRD1: SEQ ID NO:26;
LC-CRD2: SEQ ID NO:27;
LC-CRD3: SEQ ID NO:28;

and a VH domain comprising:
HC-CRD1: SEQ ID NO:29;
HC-CRD2: SEQ ID NO:30;
HC-CRD3: SEQ ID NO:31.

In some embodiments the CAR comprises an antigen binding domain comprising a VL domain comprising:
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

In some embodiments the CAR comprises an antigen binding domain comprising a light chain variable region (VL) comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:16, 24, 32 or 63.

In some embodiments the CAR comprises an antigen binding domain comprising a heavy chain variable region (VH) comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:17, 25, 33 or 64.

In some embodiments the CAR comprises an antigen binding domain comprising a VL comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:16 and a VH comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:17. In some embodiments the CAR comprises an antigen binding domain comprising a VL comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:24 and a VH comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:25. In some embodiments the CAR comprises an antigen binding domain comprising a VL comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:32 and a VH comprising, or consisting of or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:33. In some embodiments the CAR comprises an antigen binding domain comprising a VL comprising, consisting of, or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:63 and a VH comprising, consisting of: or consisting essentially of, an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:64.

In some embodiments, the CAR of the present disclosure comprises an antigen binding region which comprises or consists of or consists essentially of an antibody/antigen binding fragment according to the present disclosure.

The cell membrane anchor region is provided between the antigen-binding region and the signalling region of the CAR. The cell membrane anchor region provides for anchoring the CAR to the cell membrane of a cell expressing a CAR, with the antigen-binding region in the extracellular space, and signalling region inside the cell. Suitable transmembrane domains include transmembrane region derived from CD28, CD3-ζ, CD4 or CD8.

In some embodiments the cell membrane anchor region comprises, or consists of or consists essentially of, an amino acid sequence having at least 80%, 85%, 90%, 91%, 92%, 93%, 94%, 95%, 96%, 97%, 98%, 99% or 100% sequence identity to SEQ ID NO:4.

The signalling region of a CAR allows for activation of the T cell. The CAR signalling regions may comprise the amino acid sequence of the intracellular domain of CD3-ζ, which provides immunoreceptor tyrosine-based activation motifs (ITAMs) for phosphorylation and activation of the CAR-expressing T cell. Signalling regions comprising sequences of other ITAM-containing proteins have also been employed in CARs, such as domains comprising the ITAM containing region of FcγRI (Haynes et al., 2001 J Immunol 166(1):182-187). CARs comprising a signalling region derived from the intracellular domain of CD3-(are often referred to as first generation CARs.

In some embodiments the cell membrane anchor region comprises, or consists of or consists essentially of, an amino acid sequence having at least 80%, 85%, 90%, 91%, 92%, 93%, 94%, 95%, 96%, 97%, 98%, 99% or 100% sequence identity to SEQ ID NO:6.

Signalling regions of CARs may also comprise co-stimulatory sequences derived from the signalling region of co-stimulatory molecules, to facilitate activation of CAR-expressing T cells upon binding to the target protein. Suitable co-stimulatory molecules include at least CD28, OX40, 4-1 BB, ICOS and CD27. CARs having a signalling region including additional co-stimulatory sequences are often referred to as second generation CARs.

In some cases CARs are engineered to provide for co-stimulation of different intracellular signalling pathways. For example, signalling associated with CD28 costimulation preferentially activates the phosphatidylinositol 3-kinase (P13K) pathway, whereas the 4-1 BB-mediated signalling is through TNF receptor associated factor (TRAF) adaptor proteins. Signalling regions of CARs therefore sometimes contain co-stimulatory sequences derived from signalling regions of more than one co-stimulatory molecule. CARs comprising a signalling region with multiple co-stimulatory sequences are often referred to as third generation CARs.

In some embodiments, the CAR of the present disclosure comprises one or more co-stimulatory sequences comprising or consisting of or consisting essentially of an amino acid sequence which comprises, consists of or consists essentially of, or is derived from, the amino acid sequence of the intracellular domain of one or more of CD28, OX40, 4-1 BB, ICOS and CD27.

In some embodiments the cell membrane anchor region comprises, or consists of or consists essentially of, an amino acid sequence having at least 80%, 85%, 90%, 91%, 92%, 93%, 94%, 95%, 96%, 97%, 98%, 99% or 100% sequence identity to SEQ ID NO:5.

An optional hinge region may provide separation between the antigen-binding domain and the transmembrane domain, and may act as a flexible linker. Hinge regions may be flexible domains allowing the binding moiety to orient in different directions. Hinge regions may be derived from IgG1 or the CH₂CH₃ region of immunoglobulin. In some embodiments, the CAR of the present disclosure comprises a hinge region comprising or consisting of or consisting essentially of an amino acid sequence which comprises, consists of or consists essentially of, or is derived from, the amino acid sequence of the hinge region of IgG1 or the CH₂CH₃ region of immunoglobulin. In some embodiments the cell membrane anchor region comprises, or consists of or consists essentially of, an amino acid sequence having at least 85%, 90%, 91%, 92%, 93%, 94%, 95%, 96%, 97%, 98%, 99% or 100% sequence identity to SEQ ID NO:9.

In some embodiments the CAR comprises, or consists of or consists essentially of, an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or having 100% sequence identity to SEQ ID NO:1, 2, 3 or 56.

The present disclosure also provides a cell comprising or expressing a CAR according to the present disclosure. Also provided is a cell comprising or expressing a nucleic acid encoding a CAR according to the disclosure. Engineering of CARs into T cells may be performed during culture, in vitro, for transduction and expansion, such as happens during expansion of T cells for adoptive T cell therapy. Methods for engineering immune cells to express CARs are known to the skilled person and are described e.g. in Wang and Riviere Mol Ther Oncolytics. (2016) 3:16015, which is hereby incorporated by reference in its entirety. It will be appreciated that “at least one cell” encompasses plural cells, e.g. populations of such cells.

The cell comprising or expressing a CAR according to the present disclosure may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell may be from, or may have been obtained from, a human subject. Where the CAR-expressing cell is to be used in the treatment of a subject, the cell may be from the subject to be treated with the CAR-expressing cell (i.e. the cell may be autologous), or the cell may be from a different subject (i.e. the cell may be allogeneic).

The cell may be an immune cell. The cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ε CD3ζ or CD3δ), TCR polypeptides (TCRα or TCRβ), CD27, CD28, CD4 or CD8.

In some embodiments, the cell is a T cell. In some embodiments, the T cell is a CD3+ T cell. In some embodiments, the T cell is a CD3+, CD8+ T cell. In some embodiments, the T cell is a cytotoxic T cell (e.g. a cytotoxic T lymphocyte (CTL)).

The use of CAR T-cells is associated with advantages that they can be systemically administered, and will home to both primary and metastasized tumors (Manzo et al., Human Molecular Genetics (2015) R67-73).

In some embodiments, the cell is an antigen-specific T cell. In embodiments herein, an “antigen-specific” T cell is a cell which displays certain functional properties of a T cell in response to the antigen for which the T cell is specific, or a cell expressing said antigen. In some embodiments, the properties are functional properties associated with effector T cells, e.g. cytotoxic T cells.

In some embodiments, an antigen-specific T cell may display one or more of the following properties: cytotoxicity, e.g. to a cell comprising/expressing antigen for which the T cell is specific; proliferation, IFNγ expression, CD107a expression, IL-2 expression, TNFα expression, perforin expression, granzyme expression, granulysin expression, and/or FAS ligand (FASL) expression, e.g. in response to antigen for which the T cell is specific or a cell comprising/expressing antigen for which the T cell is specific. Antigen-specific T cells comprise a TCR capable of recognising a peptide of the antigen for which the T cell is specific when presented by the appropriate MHC molecule. Antigen-specific T cells may be CD4+ T cells and/or CD8+ T cells.

In some embodiments, the antigen for which the T cell is specific may be a peptide or polypeptide of a virus, e.g. Adenovirus, Cytomegalovius (CMV), Epstein-Barr virus (EBV), human papilloma virus (HPV), influenza virus, measles virus, hepatitis B virus (HBV), hepatitis C virus (HCV), human immunodeficiency virus (HIV), lymphocytic choriomeningitis virus (LCMV), or herpes simplex virus (HSV).

A T cell which is specific for an antigen of a virus may be referred to herein as a virus-specific T cell (VST). VSTs may be CD4+ T cells (e.g. T_{H} cells) and/or CD8+ T cells (e.g. CTLs). A T cell which is specific for an antigen of a particular virus may be described as being specific for the relevant virus; for example, a T cell which is specific for an antigen of an Adenovirus may be referred to as an Adenovirus-specific T cell, or “AdVST”. The use of virus-specific T cells for the generation of CAR-T cells is associated with the advantage that whilst naïve T cells may have limited long-term persistence after infusion, virus-specific T-cells (VSTs) derived from the memory compartment, and genetically-modified VSTs have been shown to persist for over 10 years after infusion in stem cell transplant recipients (Cruz et al., Cytotherapy (2010) 12:743-749). For example, VSTs expressing GD2.CARs have been shown to persist long-term after infusion and produce complete tumor responses in patients with low tumor burden (Sun et al., Journal for Immunotherapy of Cancer (2015) 3:5 and Pule et al., Nature Medicine (2008) 14: 1264-1270).

In some embodiments the cell comprising/expressing the CAR is a virus-specific T cell (VST, e.g. a virus-specific CD4+ T cell (e.g. T_{H} cell) and/or a virus-specific CD8+ T cell (e.g. CTL). In some embodiments the CAR-expressing cell is an Adenovirus-specific T cell (AdVST), Cytomegalovius-specific T cell (CMVST), Epstein-Barr virus-specific T cell (EBVST), influenza virus-specific T cell, measles virus-specific T cell, hepatitis B virus-specific T cell (HBVST), hepatitis C virus-specific T cell (HCVST), human immunodeficiency virus-specific T cell (HIVST), lymphocytic choriomeningitis virus-specific T cell (LCMVST), Herpes simplex virus-specific T cell (HSVST) or human papilloma virus (HPVST).

In some embodiments the cell comprising/expressing the CAR is an oncolytic virus-specific immune cell (e.g. an oncolytic virus-specific T cell), e.g. as described herein.

Any cells of the disclosure may be included in an isolated population of cells that may or may not be homogeneous. In specific embodiments, the cell population has a majority of cells that are immune cells specific for an oncolytic virus and/or that express a CAR. The cells in the cell population may comprise an oncolytic adenovirus (OncAd), a helper-dependent adenovirus (HDAd), a chimeric antigen receptor (CAR) and/or nucleic acid or plurality of nucleic acids that encodes one or more of the OncAd, HDAd, and/or CAR. In particular embodiments, the cell population has at least 70, 75, 80, 85, 90, 91, 92, 93, 94, 95, 96, 97, 98, or 99% of cells that comprise an oncolytic adenovirus (OncAd), a helper-dependent adenovirus (HDAd), a chimeric antigen receptor (CAR) and/or nucleic acid or plurality of nucleic acids that encodes one or more of the OncAd, HDAd, and/or CAR.

In any embodiments provided herein the cell comprising/expressing the CAR is an activated immune cell, e.g. an activated T cell (ATC). Activated immune cells may be prepared as described herein, e.g. in Example 8. The activated immune cell may be a virus-specific immune cell e.g. an oncolytic virus-specific T cell such as those described herein.

Oncolytic Virus-Specific Immune Cells

Aspects of the present disclosure provide oncolytic virus-specific immune cells (also referred to herein as immune cells specific for an oncolytic virus). Oncolytic virus-specific immune cells express/comprise a receptor capable of recognising a peptide of an antigen of an oncolytic virus (e.g. when presented by an MHC molecule). The immune cell may express/comprise such a receptor as a result of expression of endogenous nucleic acid encoding such antigen receptor, or as a result of having been engineered to express such a receptor.

In some embodiments an oncolytic virus-specific immune cell may be a cell of hematopoietic origin, e.g. a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. The cell may express e.g. CD3 polypeptides (e.g. CD3γ CD3ε CD3ζ or CD3δ), TCR polypeptides (TCRα or TCRβ), CD27, CD28, CD4 or CD8. In some embodiments, the oncolytic virus-specific immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (T_{H} cell)). In some embodiments, the T cell is a cytotoxic T cell (e.g. a cytotoxic T lymphocyte (CTL)).

The oncolytic virus-specific immune cell (e.g. oncolytic virus-specific T cell) may be specific for an oncolytic virus as described herein. That is to say, the oncolytic virus-specific immune cell may be specific for one or more antigens of an oncolytic virus described herein.

Methods for generating/expanding populations of immune cells specific for antigen(s) of interest and/or a virus of interest are well known in the art, and are described e.g. in Wang and Riviere Cancer Gene Ther. (2015) 22(2):85-94, which is hereby incorporated by reference in its entirety.

Such methods may involve contacting heterogeneous populations of immune cells (e.g. peripheral blood mononuclear cells (PBMCs), peripheral blood lymphocytes (PBLs) tumor-infiltrating lymphocytes (TILs)) with one or more peptides of the antigen(s) of interest, or cells comprising/expressing the antigen(s)/peptides. Cells comprising/expressing the antigen(s)/peptides may do so as a consequence of infection with the virus comprising/encoding the antigen(s), uptake by the cell of the antigen(s)/peptides thereof or expression of the antigen(s)/peptides thereof. The presentation is typically in the context of an MHC molecule at the cell surface of the antigen-presenting cell.

Cells comprising/expressing the antigen(s)/peptides may have been contacted (“pulsed”) with peptides of the antigen(s) according to methods well known to the skilled person. Antigenic peptides may be provided in a library of peptide mixtures (corresponding to one or more antigens), which may be referred to as pepmixes. Peptides of pepmixes may e.g. be overlapping peptides of 8-20 amino acids in length, and may cover all or part of the amino acid sequence of the relevant antigen.

Cells within the population of immune cells comprising receptors specific for the peptide(s) may be activated (and stimulated to proliferate), following recognition of peptide(s) of the antigen(s) presented by antigen-presenting cells (APCs) in the context of appropriate costimulatory signals. It will be appreciated that “an immune cell specific for an oncolytic virus” encompasses plural cells, e.g. populations of such cells. Such populations may be generated/expanded in vitro and/or ex vivo.

In some embodiments, an immune cell specific for an oncolytic virus is specific for an oncolytic adenovirus (OncAd), e.g. an OncAd as described herein. In some embodiments, an immune cell specific for an oncolytic virus is specific for an antigen of an OncAd. In some embodiments, the antigen is, or is derived from, an OncAd protein, e.g. a protein encoded by an early gene (e.g. E1 (e.g. E1A, E1B), E2 (e.g. E2A, E2B), E3 or E4), a protein encoded by a late gene (e.g. L1, L2, L3, L4 or L5), a protein encoded by IX, or a protein encoded by IVa2. In some embodiments, the antigen is, or is derived from, an OncAd hexon and/or penton.

In some embodiments in accordance with various aspects of the present disclosure an immune cell specific for a virus may be generated/expanded (or may have been generated/expanded) by a method comprising: stimulating a population of immune cells by culture in the presence of antigen presenting cells (APCs) presenting a peptide of the virus.

In some embodiments an immune cell specific for an oncolytic virus according to the present disclosure is prepared by a method employing a PepMix comprising a mixture of overlapping peptides corresponding to Human Adenovirus 3 hexon and/or a PepMix comprising a mixture of overlapping peptides corresponding to Human Adenovirus 5 penton.

In some embodiments the oncolytic virus-specific immune cell expresses/comprises a CAR, e.g. a CAR as described herein. The oncolytic virus-specific immune cell may be engineered to express a CAR e.g. by transfection/transduction of the oncolytic virus-specific immune cell with nucleic acid encoding a CAR.

Combinations of the Disclosure

Aspects of the present invention include compositions and methods comprising/employing (i) an oncolytic virus; (ii) a virus comprising nucleic acid encoding an immunomodulatory factor; and (iii) at least one cell comprising a chimeric antigen receptor (CAR) specific for a cancer cell antigen.

Also provided is an oncolytic virus for use in a method of treating a cancer, the method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is a virus comprising nucleic acid encoding an immunomodulatory factor for use in a method of treating a cancer, the method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is at least one cell comprising a chimeric antigen receptor (CAR) specific for a cancer cell antigen for use in a method of treating a cancer, the method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is the use of an oncolytic virus in the manufacture of a medicament for use in a method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is the use of a virus comprising nucleic acid encoding an immunomodulatory factor in the manufacture of a medicament for use in a method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

Also provided is the use of at least one cell comprising a chimeric antigen receptor (CAR) specific for a cancer cell antigen in the manufacture of a medicament for use in a method comprising separate, simultaneous or sequential administration to a subject of:
(i) an oncolytic virus;
(ii) a virus comprising nucleic acid encoding an immunomodulatory factor; and
(iii) at least one cell comprising a chimeric antigen receptor (CAR) specific for a cancer cell antigen.

The present disclosure also provides compositions and methods comprising/employing (i) an oncolytic virus and a virus comprising nucleic acid encoding an immunomodulatory factor, e.g. as described herein; and (ii) at least one cell comprising a chimeric antigen receptor (CAR) specific for a cancer cell antigen, e.g. as described herein.

Provided is a method of treating a cancer comprising separate, simultaneous or sequential administration to a subject of:
(i) an oncolytic virus and a virus comprising nucleic acid encoding an immunomodulatory factor; and, optionally,
(ii) at least one cell comprising a chimeric antigen receptor (CAR) specific for a cancer cell antigen.

Also provided is an oncolytic virus and/or a virus comprising nucleic acid encoding an immunomodulatory factor for use in a method of treating a cancer, the method comprising separate, simultaneous or sequential administration to a subject of:
(i) an oncolytic virus and a virus comprising nucleic acid encoding an immunomodulatory factor; and
(ii) at least one cell comprising a chimeric antigen receptor (CAR) specific for a cancer cell antigen.

Also provided is at least one cell comprising a chimeric antigen receptor (CAR) specific for a cancer cell antigen for use in a method of treating a cancer, the method comprising separate, simultaneous or sequential administration to a subject of:
(i) an oncolytic virus and a virus comprising nucleic acid encoding an immunomodulatory factor; and
(ii) at least one cell comprising a chimeric antigen receptor (CAR) specific for a cancer cell antigen.

Also provided is the use of an oncolytic virus and/or a virus comprising nucleic acid encoding an immunomodulatory factor in the manufacture of a medicament for use in a method comprising separate, simultaneous or sequential administration to a subject of:
(i) an oncolytic virus and a virus comprising nucleic acid encoding an immunomodulatory factor; and
(ii) at least one cell comprising a chimeric antigen receptor (CAR) specific for a cancer cell antigen.

Also provided is the use of at least one cell comprising a chimeric antigen receptor (CAR) specific for a cancer cell antigen in the manufacture of a medicament for use in a method comprising separate, simultaneous or sequential administration to a subject of:
(i) an oncolytic virus and a virus comprising nucleic acid encoding an immunomodulatory factor; and
(ii) at least one cell comprising a chimeric antigen receptor (CAR) specific for a cancer cell antigen.

In some embodiments the oncolytic virus and the virus comprising nucleic acid encoding an immunomodulatory factor are provided separately. In some embodiments the oncolytic virus and the virus comprising nucleic acid encoding an immunomodulatory factor are provided in separate compositions. In some embodiments the oncolytic virus and the virus comprising nucleic acid encoding an immunomodulatory factor are provided simultaneously. In some embodiments the oncolytic virus and the virus comprising nucleic acid encoding an immunomodulatory factor are provided in a single composition.

Also provided are compositions and methods comprising/employing (i) an oncolytic virus; and (ii) at least one cell comprising a chimeric antigen receptor (CAR) specific for a cancer cell antigen (i.e. without necessarily also employing a virus comprising nucleic acid encoding an immunomodulatory factor).

Also provided are compositions and methods comprising/employing (i) an oncolytic virus; and (ii) an immune cell specific for the oncolytic virus. In some embodiments, the compositions and methods may comprise/employ a virus comprising nucleic acid encoding an immunomodulatory factor, as described herein.

In some embodiments, the compositions and methods provided herein do not comprise a virus (or the step of administration of a virus to a subject) comprising nucleic acid encoding an immunomodulatory factor, e.g. as described herein. For example, the compositions and methods comprising/employing (i) an oncolytic virus; and (ii) at least one cell comprising a chimeric antigen receptor (CAR) specific for a cancer cell antigen or an immune cell specific for the oncolytic virus; do not comprise a virus (or the step of administration of a virus to a subject) comprising nucleic acid encoding an immunomodulatory factor.

In some embodiments in accordance with various aspects described herein the cell comprising/expressing the CAR is specific for the oncolytic virus employed (e.g. comprises antigen receptor (e.g. TCR) specific for an antigen of the oncolytic virus). That is to say, in some embodiments the oncolytic virus and the specificity of the cell comprising/expressing the CAR are matched. By way of example, in some embodiments the oncolytic virus is an adenovirus, and the CAR-expressing cell comprising/expressing a CAR is an Adenovirus-specific T cell.

Similarly, in various aspects described herein an oncolytic virus is employed in combination with an immune cell specific for the oncolytic virus (i.e. the same oncolytic virus).

“Combinations” as referred to herein encompass products and compositions (e.g. pharmaceutical compositions) comprising the components of the combination. “Combinations” also encompass therapeutic regimens employing the components of the combination.

In some embodiments the components of a combination are provided in separate compositions. In some embodiments more than one component of a combination is provided in a composition. In some embodiments the components of a combination are provided in one composition.

Similarly, in some embodiments the components of a combination are administered separately. In some embodiments a component of a combination is administered with another component of the combination. In some embodiments the components of a combination are administered together.

By way of illustration, in the example of a combination comprising an oncolytic virus, a virus comprising nucleic acid encoding an immunomodulatory factor and at least one cell comprising a CAR specific for a cancer cell antigen, the oncolytic virus and the virus comprising nucleic acid encoding an immunomodulatory factor may be administered together, and the at least one cell comprising a CAR specific for a cancer cell antigen may be administered separately (e.g. subsequently).

Where components of a combination are administered together administration may be simultaneous administration as described hereinbelow. Where components of a combination are administered separately, administration may be simultaneous administration or sequential administration, as described hereinbelow. In cases wherein components of a combination are administered separately, the administration of the separate components may or may not be administered via the same administration routes

Functional Properties

The agents of the present disclosure may be defined by reference to one of more functional properties. The agents may be evaluated for the functional properties, for example, by analysis as described in the experimental examples. Similarly, the combinations and methods of the present disclosure may be defined by reference to one or more functional properties and/or effects, and may be evaluated for such properties/effects e.g. by analysis as described in the experimental examples.

In some embodiments, an oncolytic virus according to the present disclosure, e.g. administered at a dosage described herein and/or in accordance with a schedule described herein, may possess one or more of the following functional properties:
ability to replicate in, and/or cause cell killing of, cancer cells;
reduced ability to replicate in and/or cause cell killing of, non-cancerous cells as compared to the ability to replicate in, and/or cause cell killing of, cancer cells;
comparable or improved ability to cause cell killing of cancer cells as compared to the ability of one or more oncolytic viruses known in the art;
ability to help replication of helper-dependent adenovirus (HDAd);
comparable or improved ability to replicate in cancer cells as compared to the ability of one or more oncolytic viruses known in the art.

In some embodiments, a cell comprising a chimeric antigen receptor (CAR) specific for a cancer cell antigen according to the present disclosure, e.g. administered at a dosage described herein and/or in accordance with a schedule described herein, may possess one or more of the following functional properties:
ability to bind to HER2;
ability to bind to HER2-expressing cells;
ability to cause cell killing of HER2-expressing cells;
reduced ability to cause cell killing of cell not expressing HER2 as compared to the ability to cause cell killing of HER2-expressing cells.

In some embodiments the combination of an oncolytic virus, a virus comprising nucleic acid encoding an immunomodulatory factor and at least one cell comprising a CAR specific for a cancer cell antigen, e.g. wherein each virus and/or the cell are each administered at a dosage described herein and/or in accordance with a schedule described herein, may possess one or more of the following functional properties:
improved ability to cause cell killing of cancer cells as compared to the ability to cause cell killing of cancer cells by any one of the components (i.e. components of the combination) used alone, or by any two of the components used in combination.
ability to cause cell killing of cancer cells which is synergistic (i.e. super-additive) as compared to the ability to cause cell killing of cancer cells by the components used alone.

In some embodiments the combination of an oncolytic virus, a virus comprising nucleic acid encoding an immunomodulatory factor and at least one cell comprising a CAR specific for a cancer cell antigen, e.g. wherein each virus and/or the cell are each administered at a dosage described herein and/or in accordance with a schedule described herein, is associated with improved targeting of a cell comprising a CAR specific for a cancer cell antigen to a secondary tumour site (i.e. a tumour site other than the primary tumour site) as compared to targeting of such cells to such tumour sites observed when a cell comprising a CAR specific for a cancer cell antigen is used alone, or in combination with an oncolytic virus or a virus comprising nucleic acid encoding an immunomodulatory factor.

A primary tumour site refers to the tumour-containing region into which the oncolytic virus and/or the virus comprising nucleic acid encoding an immunomodulatory factor and/or a cell comprising a CAR are administered, e.g. as described herein. A secondary tumour site refers to a tumour-containing region which has not received direct administration of oncolytic virus and/or virus comprising nucleic acid encoding an immunomodulatory factor and/or a cell comprising a CAR. In some embodiments the combination of an oncolytic virus, a virus comprising nucleic acid encoding an immunomodulatory factor and at least one cell comprising a CAR specific for a cancer cell antigen, e.g. wherein each virus and/or the cell are each administered at a dosage described herein and/or in accordance with a schedule described herein, is associated with improved targeting of a cell comprising a CAR specific for a cancer cell antigen to a metastatic tumour site. In some embodiments said combination is associated with improved treatment of metastatic tumours.

In some embodiments the combination of an oncolytic virus and at least one cell comprising a CAR specific for a cancer cell antigen, e.g. wherein the virus and/or cell are administered at a dosage described herein and/or in accordance with a schedule described herein, may possess one or more of the following functional properties:
improved ability to cause cell killing of cancer cells as compared to the ability to cause cell killing of cancer cells by either component used alone.
ability to cause cell killing of cancer cells which is synergistic (i.e. super-additive) as compared to the ability to cause cell killing of cancer cells by the components used alone.

In some embodiments the combination of an oncolytic virus and an immune cell specific for the oncolytic virus, e.g. wherein the virus and/or cell are administered at a dosage described herein and/or in accordance with a schedule described herein, may possess one or more of the following functional properties:
improved ability to cause cell killing of cancer cells as compared to the ability to cause cell killing of cancer cells by either component used alone.
ability to cause cell killing of cancer cells which is synergistic (i.e. super-additive) as compared to the ability to cause cell killing of cancer cells the components used alone.

Analysis of the ability to cause cell killing of cancer cells may be assessed e.g. in vitro, by analysis of number/viability of cancer cells. Analysis of the ability to cause cell killing of cancer cells may also be analysed in vivo in an appropriate model, e.g. by analysis of number of cancer cells, tumor size/volume and/or some other correlate of the number of cancer cells (e.g. disease progression, severity of symptoms of the cancer etc.).

In some embodiments, response to treatment in accordance with the present disclosure can be characterised by reference to tumour/lesion responses. In some embodiments, tumour/lesion responses are evaluated in accordance with the response evaluation criteria in solid tumours (RECIST) criteria, e.g. the RECIST 1.1 criteria as described in Eisenhauer et al., Eur J Cancer. 2009 January; 45(2):228-47, which is hereby incorporated by reference in its entirety.

In some embodiments treatment of a subject with the combination of an oncolytic virus, a virus comprising nucleic acid encoding an immunomodulatory factor, and at least one cell comprising a CAR specific for a cancer cell antigen and/or the immune cell specific for an oncolytic virus, e.g. wherein the viruses and/or the cell(s) are administered to a subject at a dosage and/or in accordance with a schedule described herein, may be associated with one or more of the following (as assessed in accordance with the RECIST 1.1 criteria):
A complete response (CR). A CR refers to a complete macroscopic disappearance of all target and/or non-target tumours. A CR may involve normalisation of tumour marker level.
An increased proportion of subjects displaying a complete response (CR), as compared to the proportion of untreated subjects displaying a CR.
An increased proportion of subjects displaying a complete response (CR), as compared to the proportion of subjects displaying a CR associated with treatment with any component (i.e. any component of the combination) used alone, or by any two of the components used in combination.
Increased overall survival (OS), e.g. as compared to untreated subjects. OS is defined as the time from the start of treatment to death due to any cause.
Increased overall survival (OS) as compared to the OS of subjects associated with treatment with any component used alone, or by any two of the components used in combination.
Increased progression-free survival (PFS), e.g. as compared to untreated subjects. PFS refers to the time from start of treatment to disease progression or death.
Increased progression-free survival (PFS) as compared to the PFS of subjects associated with treatment with any component used alone, or by any two of the components used in combination.
A partial response (PR). PR refers to a reduction of at least 30% in the sum of all target tumour diameters compared to baseline sum diameters calculated before treatment.
An increased proportion of subjects displaying a partial response (PR), as compared to the proportion of untreated subjects displaying a PR.
An increased proportion of subjects displaying a partial response (PR), as compared to the proportion of subjects displaying a PR associated with treatment with any component used alone, or by any two of the components used in combination.
A mixed response (MR). MR refers to one or more tumour lesions fulfilling the criteria for PR and other tumour lesion(s) fulfilling the criteria for progressive disease (at least a 20% increase in the sum of all tumour diameters from the smallest tumour size and/or the appearance of a new tumour lesion).
An increased proportion of subjects displaying a mixed response (MR), as compared to the proportion of untreated subjects displaying a MR.
An increased proportion of subjects displaying a mixed response (MR), as compared to the proportion of subjects displaying a MR associated with treatment with any component used alone, or by any two of the components used in combination.
Stable disease (SD). SD refers to neither partial response nor progressive disease compared to the tumour burden at the start of treatment.
An increased proportion of subjects displaying stable disease (SD), as compared to the proportion of untreated subjects displaying SD.
An increased proportion of subjects displaying stable disease (SD), as compared to the proportion of subjects displaying SD associated with treatment with any component used alone, or by any two of the components used in combination.
An improved overall response (OR), as compared to the OR of untreated subjects.
An improved overall response (OR), as compared to the OR associated with treatment with any component used alone, or by any two of the components used in combination.
An increased overall response rate (ORR) as compared to the ORR of untreated subjects. ORR is defined as the number of patients experiencing PR or better (i.e. PR+CR) divided by the number evaluable for efficacy.
An increased overall response rate (ORR) as compared to the ORR associated with treatment with any component used alone, or by any two of the components used in combination.
An increased disease control rate (DCR) as compared to the DCR of untreated subjects. DCR is defined as the number of patients experiencing SD or better (i.e. SD+PR+CR) divided by the number evaluable for efficacy.
An increased disease control rate (DCR) as compared to the DCR associated with treatment with any component used alone, or by any two of the components used in combination.

Tumour responses can be evaluated using the appropriate imaging technique according to the tumour and its location, e.g. CT scan, MRI scan and FDG-PET. Appropriate techniques will be familiar to the skilled person and are described in Eisenhauer et al, supra.

Tumour lesions may be macroscopic or microscopic. Tumour lesions may be measurable or non-measurable. Measurable tumour lesions must have a longest diameter with minimum size of 10 mm by CT scan (scan thickness no greater than 5 mm) or MRI, 10 mm caliper measurement by clinical exam, or 20 mm by chest X-ray. Non-measurable lesions include small lesions (<10 mm) and pathological lymph nodes with ≥10 to <15 mm (short axis).

In some embodiments, an oncolytic virus according to the present disclosure, e.g. administered at a dosage described herein and/or in accordance with a schedule described herein e.g. in combination with a virus comprising nucleic acid encoding an immunomodulatory factor and at least one cell comprising a CAR specific for a cancer cell antigen and/or an immune cell specific for an oncolytic virus, may possess one or more of the following functional properties:
ability to induce a host immune response;
ability to induce cytokine production, e.g. Th1-type cytokines;
ability to induce anti-adenoviral antibodies.

Therapeutic Applications

Aspects of the present disclosure are concerned in particular with the use of an oncolytic virus, a virus comprising nucleic acid encoding an immunomodulatory factor and at least one T cell comprising a chimeric antigen receptor (CAR) specific for a cancer cell antigen, in the treatment of a cancer in a subject.

Accordingly, the present disclosure provides a method of treating a cancer, comprising administering to a subject: an oncolytic virus; a virus comprising nucleic acid encoding an immunomodulatory factor; and at least one T cell comprising a chimeric antigen receptor (CAR) specific for a cancer cell antigen.

The present disclosure also provides an oncolytic virus; a virus comprising nucleic acid encoding an immunomodulatory factor; and at least one T cell comprising a chimeric antigen receptor (CAR) specific for a cancer cell antigen; for use in a method of treating a cancer. Also provided is the use of an oncolytic virus; a virus comprising nucleic acid encoding an immunomodulatory factor; and at least one T cell comprising a chimeric antigen receptor (CAR) specific for a cancer cell antigen; in the manufacture of a medicament for treating a cancer.

The present disclosure also provides a method of treating a cancer, comprising administering to a subject: (i) an oncolytic virus; and (ii) at least one cell comprising a chimeric antigen receptor (CAR) specific for a cancer cell antigen. Also provided is (i) an oncolytic virus; and (ii) at least one cell comprising a chimeric antigen receptor (CAR) specific for a cancer cell antigen for use in a method of treating a cancer. Also provided is the use of (i) an oncolytic virus; and (ii) at least one cell comprising a chimeric antigen receptor (CAR) specific for a cancer cell antigen in the manufacture of a medicament for use in a method of treating a cancer.

The present disclosure also provides a method of treating a cancer, comprising administering to a subject: (i) an oncolytic virus; and (ii) an immune cell specific for the oncolytic virus. Also provided is (i) an oncolytic virus; and (ii) an immune cell specific for the oncolytic virus for use in a method of treating a cancer. Also provided is the use of (i) an oncolytic virus; and (ii) an immune cell specific for the oncolytic virus in the manufacture of a medicament for use in a method of treating a cancer.

Also provided are methods for treating cancer comprising administering the OncAds, HDAds, CARs, nucleic acids/plurality of nucleic acids, cells and pharmaceutical compositions of the present disclosure to a subject. Also provided are the OncAds, HDAds, CARs, nucleic acids/plurality of nucleic acids, cells and pharmaceutical compositions of the present disclosure for use in methods for treating cancer. Also provided are the use of the OncAds, HDAds, CARs, nucleic acids/plurality of nucleic acids, cells and pharmaceutical compositions of the present disclosure in the manufacture of a medicament for treating cancer.

‘Treatment’ may, for example, be reduction in the development or progression of a cancer, alleviation of the symptoms of a cancer or reduction in the pathology of a cancer. Treatment or alleviation of a cancer may be effective to prevent progression of the cancer, e.g. to prevent worsening of the condition or to slow the rate of development of a more severe disease state. In some embodiments treatment or alleviation may lead to an improvement in the cancer, e.g. a reduction in the symptoms of the cancer or reduction in some other correlate of the severity/activity of the cancer. Prevention of a cancer may refer to prevention of a worsening of the condition or prevention of the development of the cancer, e.g preventing an early stage cancer developing to a later stage.

In some embodiments, the treatment may be aimed at reducing the number of cells of the cancer or the amount of tissue comprising cancerous cells in the subject. In some embodiments, the treatment may be aimed at reducing the size of and/or preventing the growth of at least one tumor in the subject.

In some embodiments, the treatment comprises administering an oncolytic virus according to the present disclosure to the subject. In some embodiments, the treatment may comprise administering to a subject a cell or population of cells comprising or encoding an oncolytic virus according to the present disclosure. In some embodiments, the treatment comprises administering an oncolytic virus and a virus encoding an immunomodulatory factor according to the present disclosure to the subject. In some embodiments, the treatment may comprise administering to a subject a cell or population of cells comprising or encoding an oncolytic virus and/or virus encoding an immunomodulatory factor according to the present disclosure.

In some embodiments, the treatment may comprise modifying a cell or population of cells to comprise/express a CAR according to the present disclosure. In some embodiments, the treatment may comprise administering to a subject a cell or population of cells modified to comprise/express a CAR of the present disclosure. In some embodiments, the treatment is aimed at providing the subject with an immune cell or population of immune cells which having specificity for a cancer cell antigen, e.g. by administering a CAR-expressing cell according to the present disclosure, or generating a CAR-expressing cell according to the present disclosure.

In some embodiments, the treatment may comprise administering to a subject an immune cell/population of immune cells specific for an oncolytic virus according to the present disclosure. In some embodiments, the treatment is aimed at providing the subject with an immune cell/population of immune cells having specificity for an oncolytic virus. In some embodiments, the treatment may comprise generating/expanding a population of immune cells specific for an oncolytic virus according to the present disclosure.

In some embodiments, the treatment may comprise administering to a subject an immune cell/population of immune cells specific for an oncolytic virus according to the present disclosure, modified to comprise/express a CAR according to the present disclosure. In some embodiments, the treatment is aimed at providing the subject with an immune cell/population of immune cells having specificity for an oncolytic virus also having specificity for a cancer cell antigen. In some embodiments, the treatment may comprise generating/expanding a population of immune cells specific for an oncolytic virus according to the present disclosure, and modifying a cell or cells of the population to comprise/express a CAR according to the present disclosure.

The subject to be treated may be any animal or human. The subject is preferably mammalian, more preferably human. The subject may be a non-human mammal, but is more preferably human. The subject may be male or female or of any gender. The subject may be a patient. A subject may have been diagnosed with a cancer requiring treatment, may be suspected of having such a cancer, or may be at risk of developing such a cancer.

In some embodiments the subject to be treated:
Has a histologically-confirmed HER2-positive solid tumor, which may e.g. be head and neck squamous cell carcinoma; cancer of the salivary glands; lung cancer; breast cancer;
bladder cancer; gastric cancer; esophageal cancer; colorectal cancer; or pancreatic adenocarcinoma.
   E.g. wherein HER2 positivity is defined as ≥2+ staining by IHC with the FDA-approved 4B5 antibody (Ventana) or CB11 antibody (Abcam), which refers to greater than weak-to-moderate staining intensity in >10% tumor cells.
Has disease unsuitable for curative surgery, radiotherapy, systemic therapy or any combination of such modalities.
Has disease which has progressed after standard first line therapy, or without available effective treatment options.
Has at least one tumor site appropriate for intratumoral injection.
Has radiographically measurable disease as per RECIST 1.1.
Has adequate organ function within 7 days prior to consent for treatment as indicated by following measures:
   Absolute neutrophil count (ANC) ≥1.0×10\hat{ }9/l.
   Hemoglobin ≥9 g/dl.
   Platelet count ≥100×10\hat{ }9/l.
   PT or PTT ≤1.5×ULN (unless the subject is receiving anticoagulation).
   Bilirubin <2 ULN.
   AST and ALT <3 ULN.
   Serum creatinine <2× the ULN or creatinine clearance >30 mL/min.
Eastern Cooperative Oncology Group (ECOG) performance status 2 or less.
Is not pregnant; and
Is ≥18 years of age, and able to understand and given informed consent.

In some embodiments, the cancer to be treated comprises cells expressing a cancer cell antigen, e.g. a cancer cell antigen as described herein (e.g. HER2). In some embodiments, the cells express the cancer cell antigen (e.g. HER2) at the cell surface.

In some embodiments, the cancer to be treated comprises cells expressing a cancer cell antigen for which the CAR is specific. In some embodiments, the CAR comprises a cancer cell antigen binding domain, and the cancer to be treated comprises cells expressing the cancer cell antigen, e.g. cells expressing the cancer cell antigen at the cell surface.

In some embodiments, the cancer over-expresses the cancer cell antigen. Overexpression of a cancer cell antigen can be determined by detection of a level of expression of the cancer cell antigen which is greater than the level of expression by equivalent non-cancerous cells/non-tumor tissue.

In some embodiments the cancer is a cancer expressing HER2, e.g. a cancer expressing HER2 at the cell surface. In some embodiments, the cancer over-expresses HER2. Overexpression of HER2 can be determined by detection of a level of expression of HER2 which is greater than the level of expression of HER2 by equivalent non-cancerous cells/non-tumor tissue.

In some embodiments, the subject to be treated according to the present disclosure is selected for treatment on the basis of detection of expression/overexpression of the cancer cell antigen by a cancer cell or tumour obtained from the subject.

In some embodiments the subject to be treated according to the present disclosure is selected for treatment on the basis of having a HER2-positive cancer. In some embodiments the subject to be treated according to the present disclosure is selected for treatment on the basis of detection of a HER2-positive cancer. In some embodiments the cancer is a HER2-positive solid tumour. The tumour may be an advanced refractory HER2-positive tumour. The subject may have failed first line standard-of-care therapy for their cancer/tumour type.

In some embodiments the methods provided herein comprise a step of determining whether a subject has a HER2-positive cancer. A subject who is determined to have a HER2-positive cancer, e.g. a HER2-positive tumour, may be treated according to the present disclosure.

Expression of a given cancer cell antigen may be determined by any suitable means. Expression may be gene expression or protein expression. Gene expression can be determined e.g. by detection of mRNA encoding the cancer cell antigen, for example by quantitative real-time PCR (qRT-PCR). Protein expression can be determined e.g. by detection of the cancer cell antigen, for example by antibody-based methods, for example by western blot, immunohistochemistry, immunocytochemistry, flow cytometry, or ELISA.

In some embodiments the cancer/tumour is a HER2-expressing or HER2-overexpressing cancer/tumour. Analysis of HER2 expression may be performed e.g. by immunohistochemistry (IHC), or fluorescence in situ hybridisation (FISH).

In some embodiments the cancer/tumour is determined to be a HER2-expressing or HER2-overexpressing cancer/tumour by IHC-based analysis, e.g. by HercepTest (Dako), or Pathway (Ventana). In some embodiments the subject has a tumour with a HER2 status of >2+ as determined by IHC staining with the FDA-approved 4B5 antibody (Ventana) or CB11 antibody (Abcam) (i.e. greater than weak-to-moderate staining intensity in >10% tumor cells).

The cancer to be treated/prevented in accordance with the present disclosure may be any unwanted cell proliferation (or any disease manifesting itself by unwanted cell proliferation), neoplasm or tumor. The cancer may be benign or malignant and may be primary or secondary (metastatic). The cancer may be resistant (initially or following treatment) and/or the cancer may be recurring. A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

The cancer to be treated/prevented may be any kind of cancer, including any one of an acute lymphoblastic leukemia (ALL), acute myeloid leukemia (AML), adrenocortical carcinoma, AIDS-related cancer (e.g. Kaposi sarcoma, AIDS-related lymphoma, primary CNS lymphoma), anal cancer, appendix cancer, astrocytoma, basal cell carcinoma of the skin, bile duct cancer (e.g. cholangiocarcinoma), bladder cancer, bone cancer (e.g. Ewing sarcoma, osteosarcoma, malignant fibrous histiocytoma), brain tumor, breast cancer, bronchial tumor, Burkitt lymphoma, carcinoid tumor, carcinoma of unknown primary, cardiac tumor, central nervous system cancer (e.g. atypical teratoid/rhabdoid tumor, embryonal tumor, germ cell tumor, primary CNS lymphoma), cervical cancer, chordoma, chronic lymphocytic leukemia (CLL), chronic myelogenous leukemia (CML), chronic myeloproliferative neoplasm, colorectal cancer, craniopharyngioma, cutaneous T-cell lymphoma (e.g. mycosis fungoides, Sezary syndrome), ductal carcinoma in situ (DCIS), endometrial cancer (uterine cancer), ependymoma, esophageal cancer, esthesioneuroblastoma, extracranial germ cell tumor, extragonadal germ cell tumor, eye cancer (e.g. intraocular melanoma, retinoblastoma) fallopian tube cancer, malignant fibrous histiocytoma of bone, gallbladder cancer, gastric (stomach) cancer, gastrointestinal carcinoid tumor, gastrointestinal stromal tumor (GIST), ovarian germ cell tumor, testicular cancer, gestational trophoblastic disease, hairy cell leukemia, head and neck cancer, heart tumor, hepatocellular (liver) cancer, histiocytosis, Langerhans cell, Hodgkin lymphoma, hypopharyngeal cancer, islet cell tumor (pancreatic neuroendocrine tumor), kidney (renal cell) cancer, laryngeal cancer, papillomatosis, leukemia, lip and oral cavity cancer, lung cancer (non-small cell lung cancer (NSCLC) and small cell lung cancer (SCLC)) lymphoma, male breast cancer, melanoma, Merkel cell carcinoma, mesothelioma, metastatic cancer, metastatic squamous neck cancer with occult primary, midline tract carcinoma involving NUT gene, mouth cancer, multiple endocrine neoplasia syndromes, multiple myeloma/plasma cell neoplasms, mycosis fungoides, myelodysplastic syndrome, myelodysplastic/myeloproliferative neoplasm, myelogenous leukemia, chronic myeloid leukemia, acute myeloid leukemia (AML), nasal cavity and paranasal sinus cancer, nasopharyngeal cancer, neuroblastoma, non-Hodgkin lymphoma, oral cancer, lip and oral cavity cancer, oropharyngeal cancer, osteosarcoma, ovarian cancer, pancreatic cancer, papillomatosis, paraganglioma, paranasal sinus cancer, nasal cavity cancer, parathyroid cancer, penile cancer, pharyngeal cancer, pheochromocytoma, pituitary tumor, plasma cell neoplasm/multiple myeloma, pleuropulmonary blastoma, pregnancy and breast cancer, primary peritoneal cancer, prostate cancer, rectal cancer, recurrent cancer, retinoblastoma, rhabdomyosarcoma, salivary gland cancer, vascular tumor, uterine sarcoma, skin cancer, small intestine cancer, squamous cell carcinoma of the skin, T-cell lymphoma, throat cancer, thymoma, thymic carcinoma, thyroid cancer, transitional cell cancer of the renal pelvis and ureter, urethral cancer, vaginal cancer, vulvar cancer or Wilms tumor.

In some embodiments, the cancer to be treated is one or more of nasopharyngeal carcinoma (NPC; e.g. Epstein-Barr Virus (EBV)-positive NPC), cervical carcinoma (CC; e.g. human papillomavirus (HPV)-positive CC), oropharyngeal carcinoma (OPC; e.g. HPV-positive OPC), gastric carcinoma (GC; e.g. EBV-positive GC), hepatocellular carcinoma (HCC; e.g. Hepatitis B Virus (HBV)-positive HCC), lung cancer (e.g. non-small cell lung cancer (NSCLC)) and head and neck cancer (e.g. cancer originating from tissues of the lip, mouth, nose, sinuses, pharynx or larynx, e.g. head and neck squamous cell carcinoma (HNSCC)).

In some embodiments the cancer is associated with, or caused by, a virus. In some embodiments the cancer is an EBV-positive cancer. In some embodiments the cancer is an HPV-positive cancer.

In some embodiments, the cancer is one of a head and neck cancer, nasopharyngeal carcinoma (NPC), oropharyngeal cancer (OPC), cervical cancer (CC), gastric/stomach cancer, gastric carcinoma or lung cancer.

In some embodiments the cancer is one of bladder cancer, head and neck cancer (e.g. HNSCC), cancer of the salivary gland, lung cancer, breast cancer, gastric cancer, oesophageal cancer, colorectal cancer and pancreatic adenocarcinoma.

Methods of medical treatment may also involve in vivo, ex vivo, and adoptive immunotherapies, including those using autologous and/or heterologous cells or immortalized cell lines.

Administration

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the condition to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins. In some embodiments an oncolytic virus is administered at a viral particle dose of from 1×10⁵ particles to 1×10¹⁵ particles per administration, e.g. one of 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ particles. In some embodiments an oncolytic virus is administered at a viral particle dose of from 1×10⁵ particles to 1×10¹⁵ particles, 1×10⁶ particles to 1×10¹⁴ particles, 1×10⁷ particles to 1×10¹³ particles, 1×10⁸ particles to 1×10¹² particles, or 1×10⁹ particles to 1×10¹¹ particles.

In some embodiments a virus comprising nucleic acid encoding an immunomodulatory factor is administered at a viral particle dose of from 1×10⁵ particles to 1×10¹⁵ particles per administration, e.g. one of 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ particles. In some embodiments a virus comprising nucleic acid encoding an immunomodulatory factor is administered at a viral particle dose of from 1×10⁵ particles to 1×10¹⁵ particles, 1×10⁶ particles to 1×10¹⁴ particles, 1×10⁷ particles to 1×10¹³ particles, 1×10⁸ particles to 1×10¹² particles, or 1×10⁹ particles to 1×10¹¹ particles.

In some embodiments an oncolytic virus and a virus comprising nucleic acid encoding an immunomodulatory factor are administered together. In some embodiments, the an oncolytic virus and a virus comprising nucleic acid encoding an immunomodulatory factor are administered in the form of a composition comprising viral particles of an oncolytic virus and viral particles of a virus comprising nucleic acid encoding an immunomodulatory factor.

In some embodiments an oncolytic virus and a virus comprising nucleic acid encoding an immunomodulatory factor are administered at a viral particle dose (i.e. a total viral particle dose) of from 1×10⁵ particles to 1×10¹⁵ particles per administration, e.g. one of 1×10⁵, 1×10⁶, 1×10⁷, 1×10⁸, 1×10⁹, 1×10¹⁰, 1×10¹¹, 1×10¹², 1×10¹³, 1×10¹⁴, or 1×10¹⁵ particles. In some embodiments an oncolytic virus and a virus comprising nucleic acid encoding an immunomodulatory factor are administered at a viral particle dose, e.g. a total viral particle dose, of from 1×10⁵ to 1×10¹⁵ particles, 1×10⁶ to 1×10¹⁴ particles, 1×10⁷ to 1×10¹³ particles, 1×10⁸ to 1×10¹² particles, or 1×10⁹ to 1×10¹¹ particles. In some embodiments an oncolytic virus and a virus comprising nucleic acid encoding an immunomodulatory factor are administered at a viral particle dose, e.g. a total viral particle dose, of 1×10¹⁰ to 1×10¹² particles.

In some embodiments an oncolytic virus and a virus comprising nucleic acid encoding an immunomodulatory factor are administered at a viral particle dose, e.g. a total viral particle dose, of 1×10¹⁰ particles. In some embodiments an oncolytic virus and a virus comprising nucleic acid encoding an immunomodulatory factor are administered at a viral particle dose, e.g. a total viral particle dose, of 1×10¹¹ particles. In some embodiments an oncolytic virus and a virus comprising nucleic acid encoding an immunomodulatory factor are administered at a viral particle dose, e.g. a total viral particle dose, of 1×10¹² particles.

In some embodiments an oncolytic virus and a virus comprising nucleic acid encoding an immunomodulatory factor are administered at a viral particle ratio of oncolytic virus:virus comprising nucleic acid encoding an immunomodulatory factor of from 1:1 to 1:25, e.g. one of 1:1, 1:2, 1:3, 1:4, 1:5, 1:7, 1:10, 1:12, 1:15, 1:17, 1:20, 1:22 or 1:25. In some embodiments an oncolytic virus and a virus comprising nucleic acid encoding an immunomodulatory factor are administered at a viral particle ratio of oncolytic virus:virus comprising nucleic acid encoding an immunomodulatory factor of from 1:1 to 1:25, 1:3 to 1:25, 1:7 to 1:22, 1:10 to 1:20, or 1:12 to 1:17. In some embodiments an oncolytic virus and a virus comprising nucleic acid encoding an immunomodulatory factor are administered at a viral particle ratio of oncolytic virus:virus comprising nucleic acid encoding an immunomodulatory factor of from 1:0.5 (2:1) to 1:10, 1:1 to 1:10, or 1:1 to 1:5. In some embodiments an oncolytic virus and a virus comprising nucleic acid encoding an immunomodulatory factor are administered at a viral particle ratio of oncolytic virus:virus comprising nucleic acid encoding an immunomodulatory factor of 1:1.

In some embodiments: (i) an oncolytic virus is administered at a viral particle dose of 5×10⁹ particles, and (ii) a virus comprising nucleic acid encoding an immunomodulatory factor is administered at a viral particle dose of 5×10⁹ particles (e.g. a total viral particle dose of 1×10¹⁰ at a ratio of oncolytic virus:virus comprising nucleic acid encoding an immunomodulatory factor of 1:1).

In some embodiments: (i) an oncolytic virus is administered at a viral particle dose of 5×10¹⁰ particles, and (ii) a virus comprising nucleic acid encoding an immunomodulatory factor is administered at a viral particle dose of 5×10¹⁰ particles (e.g. a total viral particle dose of 1×10¹¹ at a ratio of oncolytic virus:virus comprising nucleic acid encoding an immunomodulatory factor of 1:1).

In some embodiments: (i) an oncolytic virus is administered at a viral particle dose of 5×10¹¹ particles, and (ii) a virus comprising nucleic acid encoding an immunomodulatory factor is administered at a viral particle dose of 5×10¹¹ particles (e.g. a total viral particle dose of 1×10¹² at a ratio of oncolytic virus:virus comprising nucleic acid encoding an immunomodulatory factor of 1:1).

In some embodiments: (i) an oncolytic virus is administered at a viral particle dose of 2×10⁹ to 5×10⁹ particles, and (ii) a virus comprising nucleic acid encoding an immunomodulatory factor is administered at a viral particle dose of 5×10⁹ particles to 8×10⁹ particles (e.g. a total viral particle dose of 1×10¹⁰ at a ratio of oncolytic virus:virus comprising nucleic acid encoding an immunomodulatory factor of 1:1 to 1:5).

In some embodiments: (i) an oncolytic virus is administered at a viral particle dose of 2×10¹⁰ to 5×10¹⁰ particles, and (ii) a virus comprising nucleic acid encoding an immunomodulatory factor is administered at a viral particle dose of 5×10¹⁰ particles to 8×10¹⁰ particles (e.g. a total viral particle dose of 1×10¹¹ at a ratio of oncolytic virus:virus comprising nucleic acid encoding an immunomodulatory factor of 1:1 to 1:5).

In some embodiments: (i) an oncolytic virus is administered at a viral particle dose of 2×10¹¹ to 5×10¹¹ particles, and (ii) a virus comprising nucleic acid encoding an immunomodulatory factor is administered at a viral particle dose of 5×10¹¹ particles to 8×10¹¹ particles (e.g. a total viral particle dose of 1×10¹² at a ratio of oncolytic virus:virus comprising nucleic acid encoding an immunomodulatory factor of 1:1 to 1:5).

In some embodiments: (i) an oncolytic virus is administered at a viral particle dose of 1×10⁹ to 5×10⁹ particles, and (ii) a virus comprising nucleic acid encoding an immunomodulatory factor is administered at a viral particle dose of 5×10⁹ particles to 9×10⁹ particles (e.g. a total viral particle dose of 1×10¹⁰ at a ratio of oncolytic virus:virus comprising nucleic acid encoding an immunomodulatory factor of 1:1 to 1:10).

In some embodiments: (i) an oncolytic virus is administered at a viral particle dose of 1×10¹⁰ to 5×10¹⁰ particles, and (ii) a virus comprising nucleic acid encoding an immunomodulatory factor is administered at a viral particle dose of 5×10¹⁰ particles to 9×10¹⁰ particles (e.g. a total viral particle dose of 1×10¹¹ at a ratio of oncolytic virus:virus comprising nucleic acid encoding an immunomodulatory factor of 1:1 to 1:10).

In some embodiments: (i) an oncolytic virus is administered at a viral particle dose of 1×10¹¹ to 5×10¹¹ particles, and (ii) a virus comprising nucleic acid encoding an immunomodulatory factor is administered at a viral particle dose of 5×10¹¹ particles to 9×10¹¹ particles (e.g. a total viral particle dose of 1×10¹² at a ratio of oncolytic virus:virus comprising nucleic acid encoding an immunomodulatory factor of 1:1 to 1:10).

In some embodiments: (i) an oncolytic virus is administered at a viral particle dose of 5×10⁸ particles to 1×10⁹ particles, and (ii) a virus comprising nucleic acid encoding an immunomodulatory factor is administered at a viral particle dose of 9×10⁹ particles to 9.5×10⁹ particles (e.g. a total viral particle dose of 1×10¹⁰ at a ratio of oncolytic virus:virus comprising nucleic acid encoding an immunomodulatory factor of 1:10 to 1:20).

In some embodiments: (i) an oncolytic virus is administered at a viral particle dose of 5×10⁹ particles to 1×10¹⁰ particles, and (ii) a virus comprising nucleic acid encoding an immunomodulatory factor is administered at a viral particle dose of 9×10¹⁰ particles to 9.5×10¹⁰ particles (e.g. a total viral particle dose of 1×10¹¹ at a ratio of oncolytic virus:virus comprising nucleic acid encoding an immunomodulatory factor of 1:10 to 1:20).

In some embodiments: (i) an oncolytic virus is administered at a viral particle dose of 5×10¹⁰ particles to 1×10¹¹ particles, and (ii) a virus comprising nucleic acid encoding an immunomodulatory factor is administered at a viral particle dose of 9×10¹¹ particles to 9.5×10¹¹ particles (e.g. a total viral particle dose of 1×10¹² at a ratio of oncolytic virus:virus comprising nucleic acid encoding an immunomodulatory factor of 1:10 to 1:20).

In some embodiments: (i) an oncolytic virus is administered at a viral particle dose of 1×10⁹ particles, and (ii) a virus comprising nucleic acid encoding an immunomodulatory factor is administered at a viral particle dose of 9×10⁹ particles (e.g. a total viral particle dose of 1×10¹⁰ at a ratio of oncolytic virus:virus comprising nucleic acid encoding an immunomodulatory factor of 1:10).

In some embodiments: (i) an oncolytic virus is administered at a viral particle dose of 5×10⁸ particles, and (ii) a virus comprising nucleic acid encoding an immunomodulatory factor is administered at a viral particle dose of 9.5×10⁹ particles (e.g. a total viral particle dose of 1×10¹⁰ at a ratio of oncolytic virus:virus comprising nucleic acid encoding an immunomodulatory factor of 1:20).

In some embodiments: (i) an oncolytic virus is administered at a viral particle dose of 1×10¹⁰ particles, and (ii) a virus comprising nucleic acid encoding an immunomodulatory factor is administered at a viral particle dose of 9×10¹⁰ particles (e.g. a total viral particle dose of 1×10¹¹ at a ratio of oncolytic virus:virus comprising nucleic acid encoding an immunomodulatory factor of 1:10).

In some embodiments: (i) an oncolytic virus is administered at a viral particle dose of 5×10⁹ particles, and (ii) a virus comprising nucleic acid encoding an immunomodulatory factor is administered at a viral particle dose of 9.5×10¹⁰ particles (e.g. a total viral particle dose of 1×10¹¹ at a ratio of oncolytic virus:virus comprising nucleic acid encoding an immunomodulatory factor of 1:20).

In some embodiments: (i) an oncolytic virus is administered at a viral particle dose of 1×10¹¹ particles, and (ii) a virus comprising nucleic acid encoding an immunomodulatory factor is administered at a viral particle dose of 9×10¹¹ particles (e.g. a total viral particle dose of 1×10¹² at a ratio of oncolytic virus:virus comprising nucleic acid encoding an immunomodulatory factor of 1:10).

In some embodiments: (i) an oncolytic virus is administered at a viral particle dose of 5×10¹⁰ particles, and (ii) a virus comprising nucleic acid encoding an immunomodulatory factor is administered at a viral particle dose of 9.5×10¹¹ particles (e.g. a total viral particle dose of 1×10¹² at a ratio of oncolytic virus:virus comprising nucleic acid encoding an immunomodulatory factor of 1:20).

In some embodiments cells comprising a chimeric antigen receptor specific for a cancer cell antigen and/or immune cells specific for an oncolytic virus are administered at a dose of from 1×10⁴ to 1×10¹⁰ cells per administration, e.g. one of 1×10⁴, 1×10⁵, 1×10⁶, 1×10⁷, 1×10⁸, 1×10⁹ or 1×10¹⁰ cells. In some embodiments cells comprising a chimeric antigen receptor specific for a cancer cell antigen and/or immune cells specific for an oncolytic virus are administered at a dose of from 1 ×10⁴ to 1×10¹⁰ cells, 1×10⁵ to 1×10⁹ cells, or 1×10⁶ to 1×10⁸ cells.

In some embodiments cells comprising a chimeric antigen receptor specific for a cancer cell antigen and/or immune cells specific for an oncolytic virus are administered at a dose of 1×10⁶ cells. In some embodiments cells comprising a chimeric antigen receptor specific for a cancer cell antigen and/or immune cells specific for an oncolytic virus are administered at a dose of 1×10⁷ cells. In some embodiments cells comprising a chimeric antigen receptor specific for a cancer cell antigen and/or immune cells specific for an oncolytic virus are administered at a dose of 1×10⁸ cells.

In some embodiments, treatment in accordance with the present invention comprises administration of an oncolytic virus and a virus comprising nucleic acid encoding an immunomodulatory factor in accordance with an embodiment described herein, and administration of cells comprising a chimeric antigen receptor specific for a cancer cell antigen and/or immune cells specific for an oncolytic virus in accordance with an embodiment described herein.

In some embodiments: (i) an oncolytic virus is administered at a viral particle dose of 5×10⁹ particles, (ii) a virus comprising nucleic acid encoding an immunomodulatory factor is administered at a viral particle dose of 5×10⁹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 5×10¹⁰ particles, (ii) a virus comprising nucleic acid encoding an immunomodulatory factor is administered at a viral particle dose of 5×10¹⁰ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 5×10¹¹ particles, (ii) a virus comprising nucleic acid encoding an immunomodulatory factor is administered at a viral particle dose of 5×10¹¹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 1×10⁹ particles to 5×10⁹ particles, (ii) a virus comprising nucleic acid encoding an immunomodulatory factor is administered at a viral particle dose of 5×10⁹ particles to 9×10⁹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 1×10¹⁰ particles to 5×10¹⁰ particles, (ii) a virus comprising nucleic acid encoding an immunomodulatory factor is administered at a viral particle dose of 5×10¹⁰ particles to 9×10¹⁰ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 1×10¹¹ particles to 5×10¹¹ particles, (ii) a virus comprising nucleic acid encoding an immunomodulatory factor is administered at a viral particle dose of 5×10¹¹ particles to 9×10¹¹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 5×10⁸ particles to 1×10⁹ particles, (ii) a virus comprising nucleic acid encoding an immunomodulatory factor is administered at a viral particle dose of 9×10⁹ particles to 9.5×10⁹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 5×10⁹ particles to 1×10¹⁰ particles, (ii) a virus comprising nucleic acid encoding an immunomodulatory factor is administered at a viral particle dose of 9×10¹⁰ particles to 9.5×10¹⁰ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments: (i) an oncolytic virus is administered at a viral particle dose of 5×10¹⁰ particles to 1×10¹¹ particles, (ii) a virus comprising nucleic acid encoding an immunomodulatory factor is administered at a viral particle dose of 9×10¹¹ particles to 9.5×10¹¹ particles, and (iii) cells comprising a chimeric antigen receptor specific for a cancer cell antigen and/or immune cells specific for an oncolytic virus are administered at a dose of 1×10⁶ to 1×10⁸ cells.

In some embodiments, a combination of the oncolytic virus, virus comprising nucleic acid encoding an immunomodulatory factor and cells comprising a chimeric antigen receptor specific for a cancer cell antigen/immune cells specific for an oncolytic virus is effective at treating cancer when one, two or three components are administered at a dose of 0.5, 1, 2, 3, or 4 log lower than the effective dose required of any component used alone, or of any two of the components used in combination.

Viruses, CARs, nucleic acids, and cells according to the present disclosure may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration which may include injection or infusion. Suitable formulations may comprise the viruses, CARs, nucleic acids, or cells in sterile or isotonic medium. Medicaments and pharmaceutical compositions may be formulated in fluid, including gel, form. Fluid formulations may be formulated for administration by injection or infusion (e.g. via catheter) to a selected region of the human or animal body.

The oncolytic virus and/or the virus comprising nucleic acid encoding an immunomodulatory factor may be formulated for intratumoral administration. In some embodiments, the methods may comprise intratumoral administration of the oncolytic virus and/or the virus comprising nucleic acid encoding an immunomodulatory factor. In some embodiments the oncolytic virus and/or the virus comprising nucleic acid encoding an immunomodulatory factor is administered via a single application, e.g. a single intratumoral administration. In some embodiments the oncolytic virus and the virus comprising nucleic acid encoding an immunomodulatory factor are administered via a single injection. In some embodiments administration of the oncolytic virus and/or the virus comprising nucleic acid encoding an immunomodulatory factor is by direct injection of a tumour.

The cell comprising a CAR and/or the immune cell specific for an oncolytic virus may be formulated for intravascular (e.g. intravenous) administration, e.g. intravenous injection/infusion. In some embodiments, the methods may comprise intravenous administration of the cell comprising a CAR and/or the immune cell specific for an oncolytic virus.

Administration of the components of combinations of the present disclosure (e.g. oncolytic virus, virus comprising nucleic acid encoding an immunomodulatory factor; at least one T cell comprising a CAR specific for a cancer cell antigen; immune cell specific for an oncolytic virus in accordance with the present disclosure) may be simultaneous or sequential. The present disclosure also contemplates simultaneous or sequential administration of the OncAds, HDAds, CARs, nucleic acids/plurality of nucleic acids, cells and pharmaceutical compositions of the present disclosure.

Simultaneous administration refers to administration of the agents together, for example as a pharmaceutical composition containing the agents (i.e. a combined preparation), or immediately after each other and optionally via the same route of administration, e.g. to the same artery, vein or other blood vessel. In particular embodiments, the oncolytic virus and virus comprising nucleic acid encoding an immunomodulatory factor may be administered simultaneously in a combined preparation. In certain embodiments upon simultaneous administration the two or more of the agents may be administered via different routes of administration. In some embodiments simultaneous administration refers to administration at the same time, or within e.g. 1 hr, 2 hrs, 3 hrs, 4 hrs, 5 hrs, 6 hrs, 8 hrs, 12 hrs, 24 hrs, 36 hrs or 48 hrs.

Sequential administration refers to administration of one or more of the agents followed after a given time interval by separate administration of another of the agents. It is not required that the two agents are administered by the same route, although this is the case in some embodiments. The time interval may be any time interval, including hours, days, weeks, months, or years. In some embodiments sequential administration refers to administrations separated by a time interval of one of at least 10 min, 30 min, 1 hr, 6 hrs, 8 hrs, 12 hrs, 24 hrs, 36 hrs, 48 hrs, 3 days, 4 days, 5 days, 6 days, 1 week, 2 weeks, 3 weeks, 1 month, 6 weeks, 2 months, 3 months, 4 months, 5 months or 6 months.

In some embodiments, methods of the present disclosure comprise administering an oncolytic virus and/or a virus comprising nucleic acid encoding an immunomodulatory factor, or a composition comprising both, e.g. at a dosage provided herein, and administering cells comprising a CAR, e.g. at a dosage provided herein, on the same day as administration of the oncolytic virus and/or the virus comprising nucleic acid encoding an immunomodulatory factor. In some embodiments, methods of the present disclosure comprise administering an oncolytic virus and/or a virus comprising nucleic acid encoding an immunomodulatory factor, or a composition comprising both, e.g. at a dosage provided herein, and administering cells comprising a CAR, e.g. at a dosage provided herein, on a different day as administration of the oncolytic virus and/or the virus comprising nucleic acid encoding an immunomodulatory factor.

In some embodiments the methods comprise administering cells comprising a CAR (e.g. at a dosage provided herein) at 1 to 15 days, e.g. one of 1, 2, 3, 4, 5, 6, 7, 8, 9, 10, 11, 12, 13, 14 or 15 days after administration of the oncolytic virus and/or the virus comprising nucleic acid encoding an immunomodulatory factor. In some embodiments the methods comprise administering cells comprising a CAR at 1 to 10 days, 2 to 8 days, or 2 to 5 days after administration of the oncolytic virus and/or the virus comprising nucleic acid encoding an immunomodulatory factor. In some embodiments the methods comprise administering cells comprising a CAR 3 days after administration of the oncolytic virus and/or the virus comprising nucleic acid encoding an immunomodulatory factor.

In some embodiments the method comprises a step of administering an oncolytic virus and/or a virus comprising nucleic acid encoding an immunomodulatory factor, or a composition comprising both (i.e. on day 1). In some embodiments the method comprises a step of administering cells comprising a CAR, e.g. at a dosage described herein, on one of days 1, 2, 3, 4, 5, 6 or 7 following administration of the one or more viruses (on day 1). In some embodiments the method comprises administering cells comprising a CAR on day 4, following administration of the one or more viruses on day 1.

In some embodiments the methods comprise administering one or more doses of oncolytic virus and/or a virus comprising nucleic acid encoding an immunomodulatory factor; and one or more doses of cells comprising a CAR.

In some embodiments, the present invention comprises administration in accordance with one of dose levels 1 to 6 according to Example 14.6.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹⁰ to 1×10¹² viral particles (e.g. at a ratio of oncolytic virus:virus comprising nucleic acid encoding an immunomodulatory factor of 1:1); and
administering 1×10⁶ to 1×10⁸ cells comprising a CAR (e.g. T cells comprising a HER2-specific CAR, e.g. HER2-specific AdVSTs) 3 days later.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹⁰ viral particles (e.g. at a ratio of oncolytic virus:virus comprising nucleic acid encoding an immunomodulatory factor of 1:1).

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹⁰ viral particles (e.g. at a ratio of oncolytic virus:virus comprising nucleic acid encoding an immunomodulatory factor of 1:1); and
administering 1×10⁶ cells comprising a CAR (e.g. T cells comprising a HER2-specific CAR, e.g. HER2-specific CAR-AdVSTs) 3 days later.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹¹ viral particles (e.g. at a ratio of oncolytic virus:virus comprising nucleic acid encoding an immunomodulatory factor of 1:1); and
administering 1×10⁶ cells comprising a CAR (e.g. T cells comprising a HER2-specific CAR, e.g. HER2-specific CAR-AdVSTs) 3 days later.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹¹ viral particles (e.g. at a ratio of oncolytic virus:virus comprising nucleic acid encoding an immunomodulatory factor of 1:1); and
administering 1×10⁷ cells comprising a CAR (e.g. T cells comprising a HER2-specific CAR, e.g. HER2-specific CAR-AdVSTs) 3 days later.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹² viral particles (e.g. at a ratio of oncolytic virus:virus comprising nucleic acid encoding an immunomodulatory factor of 1:1); and
administering 1×10⁷ cells comprising a CAR (e.g. T cells comprising a HER2-specific CAR, e.g. HER2-specific CAR-AdVSTs) 3 days later.

In some embodiments, treatment comprises:
administering an oncolytic virus (e.g. an OncAd) and a virus comprising nucleic acid encoding an immunomodulatory factor (e.g. HDAd encoding IL-12 and antagonist anti-PD-L1 antibody) at a total viral particle dose of 1×10¹² viral particles (e.g. at a ratio of oncolytic virus:virus comprising nucleic acid encoding an immunomodulatory factor of 1:1); and
administering 1×10⁸ cells comprising a CAR (e.g. T cells comprising a HER2-specific CAR, e.g. HER2-specific CAR-AdVSTs) 3 days later.

It will be appreciated that features of administration described herein may be employed in the context of methods of treatment and medical uses described herein.

In some embodiments the oncolytic virus and the virus comprising nucleic acid encoding an immunomodulatory factor are provided separately. In some embodiments the oncolytic virus and the virus comprising nucleic acid encoding an immunomodulatory factor are provided in separate compositions. In some embodiments the oncolytic virus and the virus comprising nucleic acid encoding an immunomodulatory factor are provided simultaneously. In some embodiments the oncolytic virus and the virus comprising nucleic acid encoding an immunomodulatory factor are provided in the same composition.

In some embodiments cells comprising a CAR are administered separately to the oncolytic virus and/or the virus comprising nucleic acid encoding an immunomodulatory factor. In some embodiments the oncolytic virus, the virus comprising nucleic acid encoding an immunomodulatory factor and cells comprising a CAR are administered simultaneously. In some embodiments the oncolytic virus, the virus comprising nucleic acid encoding an immunomodulatory factor and cells comprising a CAR are provided in the same composition.

In some embodiments, the treatment may further comprise other therapeutic or prophylactic intervention, e.g. chemotherapy, immunotherapy, radiotherapy, surgery, vaccination and/or hormone therapy. Such other therapeutic or prophylactic intervention may occur before, during and/or after the therapies encompassed by the disclosure, and the deliveries of the other therapeutic or prophylactic interventions may occur via different administration routes as the therapies of the disclosure. Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, nucleic acid or peptide aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs and biologics may be selected from: alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-PD-1 antibodies, anti-PD-L1 antibodies, anti-TIM-3 antibodies, anti-CTLA-4, anti-4-1BB, anti-GITR, anti-CD27, anti-BLTA, anti-OX43, anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; EGFR inhibitors such as erlotinib, cetuximab and gefitinib; anti-angiogenic agents such as bevacizumab (Avastin®); cancer vaccines such as Sipuleucel-T (Provenge®).

Further chemotherapeutic drugs may be selected from: 13-cis-Retinoic Acid, 2-Chlorodeoxyadenosine, 5-Azacitidine 5-Fluorouracil, 6-Mercaptopurine, 6-Thioguanine, Abraxane, Accutane®, Actinomycin-D Adriamycin®, Adrucil®, Afinitor®, Agrylin®, Ala-Cort®, Aldesleukin, Alemtuzumab, ALIMTA, Alitretinoin, Alkaban-AQ®, Alkeran®, All-transretinoic Acid, Alpha Interferon, Altretamine, Amethopterin, Amifostine, Aminoglutethimide, Anagrelide, Anandron®, Anastrozole, Arabinosylcytosine, Aranesp®, Aredia®, Arimidex®, Aromasin®, Arranon®, Arsenic Trioxide, Asparaginase, ATRA Avastin®, Azacitidine, BCG, BCNU, Bendamustine, Bevacizumab, Bexarotene, BEXXAR®, Bicalutamide, BiCNU, Blenoxane®, Bleomycin, Bortezomib, Busulfan, Busulfex®, Calcium Leucovorin, Campath®, Camptosar®, Camptothecin-11, Capecitabine, Carac™, Carboplatin, Carmustine, Casodex®, CC-5013, CCI-779, CCNU, CDDP, CeeNU, Cerubidine®, Cetuximab, Chlorambucil, Cisplatin, Citrovorum Factor, Cladribine, Cortisone, Cosmegen®, CPT-11, Cyclophosphamide, Cytadren®, Cytarabine Cytosar-U®, Cytoxan®, Dacogen, Dactinomycin, Darbepoetin Alfa, Dasatinib, Daunomycin, Daunorubicin, Daunorubicin Hydrochloride, Daunorubicin Liposomal, DaunoXome®, Decadron, Decitabine, Delta-Cortef®, Deltasone®, Denileukin, Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil®, Doxorubicin, Doxorubicin Liposomal, Droxia™, DTIC, DTIC-Dome®, Duralone®, Eligard™, Ellence™, Eloxatin™, Elspar®, Emcyt®, Epirubicin, Epoetin Alfa, Erbitux, Erlotinib, Erwinia L-asparaginase, Estramustine, Ethyol Etopophos®, Etoposide, Etoposide Phosphate, Eulexin®, Everolimus, Evista®, Exemestane, Faslodex®, Femara®, Filgrastim, Floxuridine, Fludara®, Fludarabine, Fluoroplex®, Fluorouracil, Fluoxymesterone, Flutamide, Folinic Acid, FUDR®, Fulvestrant, Gefitinib, Gemcitabine, Gemtuzumab ozogamicin, Gleevec™, Gliadel® Wafer, Goserelin, Granulocyte—Colony Stimulating Factor, Granulocyte Macrophage Colony Stimulating Factor, Herceptin®, Hexadrol, Hexalen®, Hexamethylmelamine, HMM, Hycamtin®, Hydrea®, Hydrocort Acetate®, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin®, Idarubicin, Ifex®, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A® (interferon alfa-2b), Iressa®, Irinotecan, Isotretinoin, Ixabepilone, Ixempra™, Kidrolase, Lanacort®, Lapatinib, L-asparaginase, LCR, Lenalidomide, Letrozole, Leucovorin, Leukeran, Leukine™, Leuprolide, Leurocristine, Leustatin™, Liposomal Ara-C, Liquid Pred®, Lomustine, L-PAM, L-Sarcolysin, Lupron®, Lupron Depot®, Matulane®, Maxidex, Mechlorethamine, Mechlorethamine Hydrochloride, Medralone®, Medrol®, Megace®, Megestrol, Megestrol Acetate, Melphalan, Mercaptopurine, Mesna, Mesnex™, Methotrexate, Methotrexate Sodium, Methylprednisolone, Meticorten®, Mitomycin, Mitomycin-C, Mitoxantrone, M-Prednisol®, MTC, MTX, Mustargen®, Mustine, Mutamycin®, Myleran®, Mylocel™, Mylotarg®, Navelbine®, Nelarabine, Neosar®, Neulasta™, Neumega®, Neupogen®, Nexavar®, Nilandron®, Nilutamide, Nipent®, Nitrogen Mustard, Novaldex®, Novantrone®, Octreotide, Octreotide acetate, Oncospar®, Oncovin®, Ontak®, Onxal™, Oprevelkin, Orapred®, Orasone®, Oxaliplatin, Paclitaxel, Paclitaxel Protein-bound, Pamidronate, Panitumumab, Panretin®, Paraplatin®, Pediapred®, PEG Interferon, Pegaspargase, Pegfilgrastim, PEG-INTRON™, PEG-L-asparaginase, PEMETREXED, Pentostatin, Phenylalanine Mustard, Platinol®, Platinol-AQ®, Prednisolone, Prednisone, Prelone®, Procarbazine, PROCRIT®, Proleukin®, Prolifeprospan 20 with Carmustine Implant Purinethol®, Raloxifene, Revlimid®, Rheumatrex®, Rituxan®, Rituximab, Roferon-A® (Interferon Alfa-2a), Rubex®, Rubidomycin hydrochloride, Sandostatin® Sandostatin LAR®, Sargramostim, Solu-Cortef®, Solu-Medrol®, Sorafenib, SPRYCEL™, STI-571, Streptozocin, SU11248, Sunitinib, Sutent®, Tamoxifen, Tarceva®, Targretin®, Taxol®, Taxotere®, Temodar®, Temozolomide, Temsirolimus, Teniposide, TESPA, Thalidomide, Thalomid®, TheraCys®, Thioguanine, Thioguanine Tabloid®, Thiophosphoamide, Thioplex®, Thiotepa, TICE®, Toposar®, Topotecan, Toremifene, Torisel®, Tositumomab, Trastuzumab, Treanda®, Tretinoin, Trexall™, Trisenox®, TSPA, TYKERB®, VCR, Vectibix™, Velban®, Velcade®, VePesid®, Vesanoid®, Viadur™ Vidaza®, Vinblastine, Vinblastine Sulfate, Vincasar Pfs®, Vincristine, Vinorelbine, Vinorelbine tartrate, VLB, VM-26, Vorinostat, VP-16, Vumon®, Xeloda®, Zanosar®, Zevalin™, Zinecard®, Zoladex®, Zoledronic acid, Zolinza, Zometa®.

In embodiments of the present disclosure wherein a nucleic acid/virus encoding an enzyme capable of catalysing conversion of a non-toxic factor to a cytotoxic form is employed, the method may further comprise administration with a prodrug substrate for the enzyme. The prodrug may be administered simultaneously or sequentially to administration of the nucleic acid/virus encoding an enzyme capable of catalysing conversion of a non-toxic factor to a cytotoxic form.

In some embodiments the prodrug is selected from ganciclovir (GCV), aciclovir (ACV) and/or valaciclovir, e.g. where the nucleic acid/virus encodes a thymidine kinase. In some embodiments the prodrug is 5-fluorocytosine (5-FC), e.g. where the nucleic acid/virus encodes a cytosine deaminase. In some embodiments the prodrug is selected from CB1954, nitro-CBI-DEI and/or PR-104A, e.g. where the nucleic acid/virus encodes a nitroreductase. In some embodiments the prodrug is oxazaphosphorine (e.g. cyclophosphamide or ifosfamide), e.g. where the nucleic acid/virus encodes a cytochrome P450. In some embodiments the prodrug is a nitrogen mustard based drug (e.g. CMDA or ZD2767P), e.g. where the nucleic acid/virus encodes a carboxypeptidase G2. In some embodiments the prodrug is 6-methylpurine 2-deoxyriboside and/or fludarabine (e.g. 6-methylpurine-2′-deoxyriboside (MeP-dR), 2-F-2′-deoxyadenosine (F-dAdo) or arabinofuranosyl-2-F-adenine monophosphate (F-araAMP), e.g. where the nucleic acid/virus encodes a purine nucleoside phosphorylase. In some embodiments the prodrug is indole-3-acetic acid (IAA), e.g. where the nucleic acid/virus encodes a horseradish peroxidase. In some embodiments the prodrug is irinotecan, e.g. where the nucleic acid/virus encodes a carboxylesterase.

Multiple doses of the agents (e.g. viruses (OncAds, HdAds), CARs, nucleic acids/plurality of nucleic acids, vectors, cells, compositions, combinations, prodrugs) of the present disclosure may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, or more hours or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Adoptive Transfer

In embodiments of the present disclosure, the methods of treatment comprise adoptive transfer of immune cells. Adoptive cell transfer (ACT) generally refers to a process by which cells (e.g. immune cells) are obtained from a subject, typically by drawing a blood sample from which the cells are isolated. The cells are then typically treated or altered in some way, and then administered either to the same subject (adoptive transfer is of autologous cells) or to a different subject (adoptive transfer is of allogeneic cells). The treatment is typically aimed at providing population of cells with certain desired characteristics to a subject, or increasing the frequency of cells with such characteristics in that subject. In the present disclosure, adoptive transfer may be performed with the aim of introducing a cell or population of cells into a subject, and/or increasing the frequency of a cell or population of cells in a subject.

In some embodiments, the subject from which the cell is isolated is the subject administered with the modified cell (i.e., adoptive transfer is of autologous cells). In some embodiments, the subject from which the cell is isolated is a different subject to the subject to which the modified cell is administered (i.e., adoptive transfer is of allogeneic cells).

Adoptive transfer of T cells is described, for example, in Kalos and June 2013, Immunity 39(1): 49-60, which is hereby incorporated by reference in its entirety. Adoptive transfer of NK cells is described, for example, in Davis et al. 2015, Cancer J. 21(6): 486-491, which is hereby incorporated by reference in its entirety.

The cell may e.g. be a neutrophil, eosinophil, basophil, dendritic cell, lymphocyte, or monocyte. The lymphocyte may be e.g. a T cell, B cell, NK cell, NKT cell or innate lymphoid cell (ILC), or a precursor thereof. In some embodiments, the cell is a T cell. In some embodiments, the T cell is a CD3+ T cell. In some embodiments, the T cell is a CD3+, CD4+ T cell. In some embodiments, the T cell is a CD3+, CD8+ T cell. In some embodiments, the T cell is a T helper cell (T_{H} cell)). In some embodiments, the T cell is a cytotoxic T cell (e.g. a cytotoxic T lymphocyte (CTL)). In some embodiments, the T cell is a virus-specific T cell. In some embodiments, the T cell is specific for EBV, HPV, HBV, HCV or sHIV.

In some embodiments the cell is an immune cell specific for an oncolytic virus, as described herein. Accordingly, in some embodiments the methods comprise administration of at least one immune cell specific for an oncolytic virus to a subject. In some embodiments, the methods of the disclosure comprise generating/expanding a population of immune cells specific for an oncolytic virus, and administering at least one immune cell specific for the oncolytic virus to a subject.

In some embodiments, the methods comprise:
(a) isolating immune cells from a subject;
(b) generating or expanding a population of immune cells specific for an oncolytic virus by a method comprising: stimulating the immune cells by culture in the presence of antigen presenting cells (APCs) presenting a peptide of the oncolytic virus, and;
(c) administering at least one immune cell specific for the oncolytic virus to a subject.

In some embodiments the method steps for production of an immune cell specific for an oncolytic virus may comprise one or more of: taking a blood sample from a subject; isolating PBMCs from the blood sample; generating/expanding a population of immune cells specific for an oncolytic virus (e.g. by culturing PBMCs in the presence of cells (e.g. APCs) comprising/expressing antigen(s)/peptide(s) of the oncolytic virus); culturing immune cells specific for an oncolytic virus in in vitro or ex vivo cell culture; collecting immune cells specific for an oncolytic virus; mixing immune cells specific for an oncolytic virus with an adjuvant, diluent, or carrier; administering the modified cell to a subject.

The present disclosure also provides methods of treating a cancer in a subject comprising administering at least one cell comprising a chimeric antigen receptor (CAR) specific for a cancer cell antigen. In connection with this feature of the disclosure, in some embodiments, the method additionally comprises steps for production of the at least one cell comprising a chimeric antigen receptor (CAR) specific for a cancer cell antigen. The CAR may be a first generation, second generation or third or subsequent generation CAR. The CAR may comprise one, two, three, or more costimulatory domains, for example.

In some embodiments, the methods comprise modifying at least one cell obtained from a subject to express or comprise a CAR according to the disclosure, optionally expanding the modified at least one cell, and administering the modified at least one cell to a subject.

In some embodiments, the methods comprise:
(a) isolating at least one cell from a subject;
(b) modifying the at least one cell to express or comprise a CAR according to the present disclosure, or a nucleic acid encoding a CAR according to the present disclosure,
(c) optionally expanding the modified at least one cell, and;
(d) administering the modified at least one cell to a subject.

In some embodiments the cell comprising/expressing a CAR specific for a cancer cell antigen is an immune cell specific for an oncolytic virus, as described herein. In some embodiments, the methods comprise modifying an immune cell specific for an oncolytic virus to express or comprise a CAR according to the disclosure, optionally expanding the modified immune cell specific for an oncolytic virus, and administering the modified immune cell specific for an oncolytic virus to a subject.

In some embodiments, the methods comprise:
(a) isolating immune cells from a subject;
(b) generating or expanding a population of immune cells specific for an oncolytic virus by a method comprising: stimulating the immune cells by culture in the presence of antigen presenting cells (APCs) presenting a peptide of the oncolytic virus;
(c) modifying at least one immune cell specific for an oncolytic virus to express or comprise a CAR according to the present disclosure, or a nucleic acid encoding a CAR according to the present disclosure,
(d) optionally expanding the modified at least one immune cell specific for an oncolytic virus, and;
(e) administering the modified at least one immune cell specific for an oncolytic virus to a subject.

The at least one cell modified according to the present disclosure can be modified to comprise/express a CAR according to methods well known to the skilled person. The modification may comprise nucleic acid transfer for permanent or transient expression of the transferred nucleic acid. Any suitable genetic engineering platform may be used to modify a cell according to the present disclosure. Suitable methods for modifying a cell include the use of genetic engineering platforms such as gammaretroviral vectors, lentiviral vectors, adenovirus vectors, DNA transfection, transposon-based gene delivery and RNA transfection, for example as described in Maus et al., Annu Rev Immunol (2014) 32:189-225, incorporated by reference hereinabove.

In some embodiments the method steps for production of the at least one cell comprising a chimeric antigen receptor (CAR) specific for a cancer cell antigen may comprise one or more of: taking a blood sample from a subject; isolating and/or expanding at least one cell from the blood sample; culturing the at least one cell in in vitro or ex vivo cell culture; introducing into the at least one cell a CAR as described herein, or a nucleic acid encoding a CAR as described herein, thereby modifying the at least one cell; expanding the at least one modified cell; collecting the at least one modified cell; mixing the modified cell with an adjuvant, diluent, or carrier; administering the modified cell to a subject.

In some embodiments, the methods may additionally comprise treating the cell to induce/enhance expression of the CAR or nucleic acid encoding the CAR. For example, the nucleic acid may comprise a control element for inducible upregulation of expression of the CAR from the nucleic acid in response to treatment with a particular agent. In some embodiments, treatment may be in vivo by administration of the agent to a subject having been administered with a modified cell according to the disclosure. In some embodiments, treatment may be ex vivo or in vitro by administration of the agent to cells in culture ex vivo or in vitro.

The skilled person is able to determine appropriate reagents and procedures for adoptive transfer of cells according to the present disclosure, for example by reference to Dai et al., 2016 J Nat Cancer Inst 108(7): djv439, which is incorporated by reference in its entirety.

In a related aspect, the present disclosure provides a method of preparing a modified cell, the method comprising introducing into a cell a CAR according to the present disclosure or a nucleic acid encoding a CAR according to the present disclosure, thereby modifying the at least one cell. The method is preferably performed in vitro or ex vivo.

Compositions/Products/Kits

The present disclosure also provides an oncolytic virus as described herein, optionally isolated. Also provided is a nucleic acid encoding the oncolytic virus, optionally isolated. Also provided is a cell comprising the oncolytic virus, or comprising nucleic acid encoding the oncolytic virus, optionally isolated.

The present disclosure also provides a virus comprising nucleic acid encoding an immunomodulatory factor as described herein, optionally isolated. Also provided is a nucleic acid encoding the virus, optionally isolated. Also provided is a cell comprising the virus, or comprising nucleic acid encoding the virus, optionally isolated.

The present disclosure also provides a chimeric antigen receptor (CAR) as described herein, optionally isolated. Also provided is a nucleic acid encoding the CAR, optionally isolated. Also provided is a cell comprising the CAR, or comprising nucleic acid encoding the CAR, optionally isolated.

The present disclosure also provides compositions comprising an oncolytic virus, a virus comprising nucleic acid encoding an immunomodulatory factor, a chimeric antigen receptor, a nucleic acid/plurality of nucleic acids, and/or a cell according to the disclosure.

The oncolytic virus, virus comprising nucleic acid encoding an immunomodulatory factor, chimeric antigen receptor, nucleic acid/plurality of nucleic acids and/or cell according to the present disclosure may be formulated as pharmaceutical compositions for clinical use and may comprise a pharmaceutically acceptable carrier, diluent, excipient or adjuvant. Combinations of the present disclosure may be provided in a single composition, or may be provided as plural compositions comprising the components of the combination.

In some embodiments the oncolytic virus and the virus comprising nucleic acid encoding an immunomodulatory factor are provided in a single composition. The composition may be administered as described herein.

In accordance with the present disclosure methods are also provided for the production of pharmaceutically useful compositions, such methods of production may comprise one or more steps selected from: isolating an oncolytic virus, a virus comprising nucleic acid encoding an immunomodulatory factor, a chimeric antigen receptor, a nucleic acid/plurality of nucleic acids, or a cell as described herein; and/or mixing an oncolytic virus, a virus comprising nucleic acid encoding an immunomodulatory factor, a chimeric antigen receptor, a nucleic acid/plurality of nucleic acids, or a cell as described herein with a pharmaceutically acceptable carrier, adjuvant, excipient or diluent.

For example, a further aspect of the present disclosure relates to a method of formulating or producing a medicament or pharmaceutical composition for use in the treatment of a cancer, the method comprising formulating a pharmaceutical composition or medicament by mixing an oncolytic virus, a virus comprising nucleic acid encoding an immunomodulatory factor, a chimeric antigen receptor, a nucleic acid/plurality of nucleic acids, or a cell as described herein with a pharmaceutically acceptable carrier, adjuvant, excipient or diluent.

The present disclosure also provides a kit of parts comprising one or more of an oncolytic virus, a virus comprising nucleic acid encoding an immunomodulatory factor, a chimeric antigen receptor, a nucleic acid, a cell or a composition according to the present disclosure.

In some embodiments the kit may have at least one container having a predetermined quantity of an oncolytic virus, a virus comprising nucleic acid encoding an immunomodulatory factor, a chimeric antigen receptor, a nucleic acid/plurality of nucleic acids, or a cell according to the disclosure or a composition according to the present disclosure. The kit may have containers containing individual components of the combinations of the present disclosure, or may have containers containing combinations of the components of the combinations of the present disclosure.

In some embodiments the kit may have at least one container having a predetermined quantity of oncolytic virus and at least one container having a predetermined quantity of virus comprising nucleic acid encoding an immunomodulatory factor, and optionally at least one container having a predetermined quantity of cells described herein. In some embodiments the kit may have at least one container having a predetermined quantity of oncolytic virus and a predetermined quantity of virus comprising nucleic acid encoding an immunomodulatory factor.

The kit may provide the oncolytic virus, virus comprising nucleic acid encoding immunomodulatory factor, CAR, nucleic acid, cell or composition with instructions for administration to a patient in order to treat a specified cancer. The oncolytic virus, virus comprising nucleic acid encoding immunomodulatory factor, CAR, nucleic acid/plurality of nucleic acids, cell or composition may be formulated so as to be suitable for injection or infusion to a tumor or to the blood.

In some embodiments the kit may comprise materials for producing a cell according to the present disclosure. For example, the kit may comprise materials for modifying a cell to express or comprise a virus or an antigen/peptide thereof, CAR or nucleic acid/plurality of nucleic acids according to the present disclosure, or materials for introducing into a cell the virus or an antigen/peptide thereof or nucleic acid/plurality of nucleic acids according to the present disclosure. The kit may comprise materials for producing an immune cell specific for an oncolytic virus; for example, the kit may comprise pepmixes of one or more antigens of the oncolytic virus.

In some embodiments the kit may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the cancer. The therapeutic agent may also be formulated so as to be suitable for injection or infusion to a tumor or to the blood.

Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Sdding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | HER2(C5)- | MTRAMDWIWRILFLVGAATGAHSQVQLQESGPGLVKPSETLSLTCTVSGGSIS |
| | CD28TM, ICD- | SSSYYWGWIRQPPGKGLEWIGSIYYSGSTYYNPSLKSRVTISVDTSKNQFSLKL |
| | CD3Z CAR | SSVTAADTAVYYCARYAPDSSGYLVAFDIWGQGTMVTVSSGGGGSGGGGSG |
| | | GGGSQTVVTQEPSFSVSPGGTVTLTCGLSSGSVSTGYYPSWYQQTPGQAPR |
| | | TLIYSTNSRSSGVPDRFSGSILGNKAALTITGAQADDESDYYCVLYMGSGISVF |
| | | GGGTKLTVLGSEPKSCDKTHTCPTRFWVLVVVGGVLACYSLLVTVAFIIFWVRS |
| | | KRSRLLHSDYMNMTPRRPGPTRKHYQPYAPPRDFAAYRSRVKFSRSADAPAY |
| | | QQGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPPR |
| 2 | HER2(E4)- | MTRAMDWIWRILFLVGAATGAHSQVQLQQWGAGLLKPSETLSLTCAVYGGSF |
| | CD28TM, ICD- | SGYYWSWIRQPPGKGLEWIGEINHSGSTNYNPSLKSRVTISVDTSKNQFSLKL |
| | CD3Z CAR | SSVTTADTAVYYCARMGINSGGYLYGMDVWGQGTTVTVSSGGGGSGGGGS |
| | | GGGGSQTVVTQEPSFSVSPGGTVTLTCGLSSGSVSTSYYPSWYQQIPGQAPR |
| | | TLIYTTNIRSSGVPDRFGGSILGNKAALTITGAQAEDESDYYCMLYMGSGIWVF |
| | | GGGTKLTVLGSEPKSCDKTHTCPTRFWVLVVVGGVLACYSLLVTVAFIIFWVRS |
| | | KRSRLLHSDYMNMTPRRPGPTRKHYQPYAPPRDFAAYRSRVKFSRSADAPAY |
| | | QQGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPPR |
| 3 | HER2(F1)- | MTRAMDWIWRILFLVGAATGAHSQVQLVESGPGLVKPSGTLSLTCAVSGGSIS |
| | CD28TM, ICD- | SSNWWSWVRQPPGKGLEWIGEIYHSGSTNYNPSLKSRVTISVDTSKNQFSLKL |
| | CD3Z CAR | SSVTAADTAVYYCARMGANSGGYLYGMDVWGQGTTVTVSSGGGGSGGGGS |
| | | GGGGSQTVVTQEPSFSVSPGGTVTLTCGLSSGSVSTSYYPSWYQQTPGQAP |
| | | RTLIYSTNTRSSGVPDRFSGSILGNKAALTITGAQADDESDYYCVLYMGSGIWV |
| | | FGGGTKLTVLGSEPKSCDKTHTCPTRFWVLVVVGGVLACYSLLVTVAFIIFWVR |
| | | SKRSRLLHSDYMNMTPRRPGPTRKHYQPYAPPRDFAAYRSRVKFSRSADAPA |
| | | YQQGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPPR |
| 4 | CD28 TMD | FWVLVVVGGVLACYSLLVTVAFIIFWV |
| 5 | CD28 ICD | RSKRSRLLHSDYMNMTPRRPGPTRKHYQPYAPPRDFAAYRS |
| 6 | CD3Z ICD | RVKFSRSADAPAYQQGQNQLYNELNLGRREEYDVLDKRRGRDPEMGGKPQR |
| | | RKNPQEGLYNELQKDKMAEAYSEIGMKGERRRGKGHDGLYQGLSTATKDTYD |
| | | ALHMQALPPR |
| 7 | (G4S)3 linker | GGGGSGGGGSGGGGS |
| 8 | huIgG H | MDWIWRILFLVGAATGAHS |
| | leader | |
| 9 | Hinge | EPKSCDKTHTCPTR |
| 10 | HER2(C5) LC- | GLSSGSVSTGYYPS |
| | CDR1 | |
| 11 | HER2(C5) LC- | STNSRSS |
| | CDR2 | |
| 12 | HER2(C5) LC- | VLYMGSGISV |
| | CDR3 | |
| 13 | HER2(C5) HC- | SSSYYWG |
| | CDR1 | |
| 14 | HER2(C5) HC- | SIYYSGSTYYNPSLKS |
| | CDR2 | |
| 15 | HER2(C5) HC- | YAPDSSGYLVAFDI |
| | CDR3 | |
| 16 | HER2(C5) VL | QTVVTQEPSFSVSPGGTVTLTCGLSSGSVSTGYYPSWYQQTPGQAPRTLIYST |
| | | NSRSSGVPDRFSGSILGNKAALTITGAQADDESDYYCVLYMGSGISVFGGGTK |
| | | LTVLGS |
| 17 | HER2(C5) VH | QVQLQESGPGLVKPSETLSLTCTVSGGSISSSSYYWGWIRQPPGKGLEWIGSI |
| | | YYSGSTYYNPSLKSRVTISVDTSKNQFSLKLSSVTAADTAVYYCARYAPDSSGY |
| | | LVAFDIWGQGTMVTVSS |
| 18 | HER2(E4) LC- | GLSSGSVSTSYYPS |
| | CDR1 | |
| 19 | HER2(E4) LC- | TTNIRSS |
| | CDR2 | |
| 20 | HER2(E4) LC- | MLYMGSGIWV |
| | CDR3 | |
| 21 | HER2(E4) HC- | SGYYWS |
| | CDR1 | |
| 22 | HER2(E4) HC- | EINHSGSTNYNPSLKS |
| | CDR2 | |
| 23 | HER2(E4) HC- | MGINSGGYLYGMDV |
| | CDR3 | |
| 24 | HER2(E4) VL | QTVVTQEPSFSVSPGGTVTLTCGLSSGSVSTSYYPSWYQQIPGQAPRTLIYTT |
| | | NIRSSGVPDRFGGSILGNKAALTITGAQAEDESDYYCMLYMGSGIWVFGGGTK |
| | | LTVLGS |
| 25 | HER2(E4) VH | QVQLQQWGAGLLKPSETLSLTCAVYGGSFSGYYWSWIRQPPGKGLEWIGEIN |
| | | HSGSTNYNPSLKSRVTISVDTSKNQFSLKLSSVTTADTAVYYCARMGINSGGYL |
| | | YGMDVWGQGTTVTVSS |
| 26 | HER2(F1) LC- | GLSSGSVSTSYYPS |
| | CDR1 | |
| 27 | HER2(F1) LC- | STNTRSS |
| | CDR2 | |
| 28 | HER2(F1) LC- | VLYMGSGIWV |
| | CDR3 | |
| 29 | HER2(F1) HC- | SSNWWS |
| | CDR1 | |
| 30 | HER2(F1) HC- | EIYHSGSTNYNPSLKS |
| | CDR2 | |
| 31 | HER2(F1) HC- | MGANSGGYLYGMDV |
| | CDR3 | |
| 32 | HER2(F1) VL | QTVVTQEPSFSVSPGGTVTLTCGLSSGSVSTSYYPSWYQQTPGQAPRTLIYST |
| | | NTRSSGVPDRFSGSILGNKAALTITGAQADDESDYYCVLYMGSGIWVFGGGTK |
| | | LTVLGS |
| 33 | HER2(F1) VH | QVQLVESGPGLVKPSGTLSLTCAVSGGSISSSNWWSWVRQPPGKGLEWIGEI |
| | | YHSGSTNYNPSLKSRVTISVDTSKNQFSLKLSSVTAADTAVYYCARMGANSGG |
| | | YLYGMDVWGQGTTVTVSS |
| 34 | Ad2 E1AΔ24 | MRHIICHGGVITEEMAASLLDQLIEEVLADNLPPPSHFEPPTLHELYDLDVTAPE |
| | | DPNEEAVSQIFPESVMLAVQEGIDLFTFPPAPGSPEPPHLSRQPEQPEQRALG |
| | | PVSMPNLVPEVIDPPSDDEDEEGEEFVLDYVEHPGHGCRSCHYHRRNTGDPD |
| | | IMCSLCYMRTCGMFVYSPVSEPEPEPEPEPEPARPTRRPKLVPAILRRPTSPV |
| | | SRECNSSTDSCDSGPSNTPPEIHPVVPLCPIKPVAVRVGGRRQAVECIEDLLNE |
| | | SGQPLDLSCKRPRP |
| 35 | hulL-12p70 | MGHQQLVISWFSLVFLASPLVAIWELKKDVYVVELDWYPDAPGEMVVLTCDTP |
| | | EEDGITWTLDQSSEVLGSGKTLTIQVKEFGDAGQYTCHKGGEVLSHSLLLLHK |
| | | KEDGIWSTDILKDQKEPKNKTFLRCEAKNYSGRFTCWWLTTISTDLTFSVKSSR |
| | | GSSDPQGVTCGAATLSAERVRGDNKEYEYSVECQEDSACPAAEESLPIEVMV |
| | | DAVHKLKYENYTSSFFIRDIIKPDPPKNLQLKPLKNSRQVEVSWEYPDTWSTPH |
| | | SYFSLTFCVQVQGKSKREKKDRVFTDKTSATVICRKNASISVRAQDRYYSSSW |
| | | SEWASVPCSVPGVGVPGVGARNLPVATPDPGMFPCLHHSQNLLRAVSNMLQ |
| | | KARQTLEFYPCTSEEIDHEDITKDKTSTVEACLPLELTKNESCLNSRETSFITNG |
| | | SCLASRKTSFMMALCLSSIYEDLKMYQVEFKTMNAKLLMDPKRQIFLDQNMLA |
| | | VIDELMQALNFNSETVPQKSSLEEPDFYKTKIKLCILLHAFRIRAVTIDRVMSYLN |
| | | AS |
| 36 | HSV1 TK | MASYPGHQHASAFDQAARSRGHSNRRTALRPRRQQEATEVRPEQKMPTLLR |
| | | VYIDGPHGMGKTTTTQLLVALGSRDDIVYVPEPMTYWRVLGASETIANIYTTQH |
| | | RLDQGEISAGDAAVVMTSAQITMGMPYAVTDAVLAPHIGGEAGSSHAPPPALT |
| | | LIFDRHPIAALLCYPAARYLMGSMTPQAVLAFVALIPPTLPGTNIVLGALPEDRHI |
| | | DRLAKRQRPGERLDLAMLAAIRRVYGLLANTVRYLQGGGSWREDWGQLSGT |
| | | AVPPQGAEPQSNAGPRPHIGDTLFTLFRAPELLAPNGDLYNVFAWALDVLAKR |
| | | LRPMHVFILDYDQSPAGCRDALLQLTSGMIQTHVTTPGSIPTICDLARTFAREM |
| | | GEAN |
| 37 | HA tag | YPYDVPDYA |
| 38 | PD-L1(H12_gl) | MDWIWRILFLVGAATGAHSEVQLVQSGAEVKKPGASVKVSCKASGGTFSSYAI |
| | minibody | SWVRQAPGQGLEWMGRIIPILGIANYAQKFQGRVTITADKSTSTAYMELSSLRS |
| | | EDTAVYYCARSGHGYSYGAFDYWGQGTLVTVSSGGGGSGGGGSGGGGSQS |
| | | VLTQPPSVSGAPGQRVTISCTGSSSNIGAGYDVHWYQQLPGTAPKLLIYGNSN |
| | | RPSGVPDRFSGSKSGTSASLAITGLQAEDEADYYCQSYDSSLSGSYVVFGGG |
| | | TKLTVLEAKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLN |
| | | GKEYKCKVSNKALPAPIEKTISKAKGQPREPQVYTLPPSRDELTKNQVSLTCLV |
| | | KGFYPSDIAVEWESNGQPENNYKTTPPVLDSDGSFFLYSKLTVDKSRWQQGN |
| | | VFSCSVMHEALHNHYTQKSLSLSPGKGGGGSYPYDVPDYAGYPYDVPDYAG |
| | | YPYDVPDYA |
| 39 | PD-L1(H12_gl) | TGSSSNIGAGYDVH |
| | LC-CDR1 | |
| 40 | PD-L1(H12_gl) | GNSNRPS |
| | LC-CDR2 | |
| 41 | PD-L1(H12_gl) | QSYDSSLSGSYVV |
| | LC-CDR3 | |
| 42 | PD-L1(H12_gl) | SYAIS |
| | HC-CDR1 | |
| 43 | PD-L1(H12_gl) | RIIPILGIANYAQKFQG |
| | HC-CDR2 | |
| 44 | PD-L1(H12_gl) | SGHGYSYGAFDY |
| | HC-CDR3 | |
| 45 | PD-L1(H12_gl) | QSVLTQPPSVSGAPGQRVTISCTGSSSNIGAGYDVHWYQQLPGTAPKLLIYGN |
| | VL | SNRPSGVPDRFSGSKSGTSASLAITGLQAEDEADYYCQSYDSSLSGSYVVFG |
| | | GGTKLTVL |
| 46 | PD-L1(H12_gl) | EVQLVQSGAEVKKPGASVKVSCKASGGTFSSYAISWVRQAPGQGLEWMGRII |
| | VH | PILGIANYAQKFQGRVTITADKSTSTAYMELSSLRSEDTAVYYCARSGHGYSYG |
| | | AFDYWGQGTLVTVSS |
| 47 | HER2(C5)- | AAGTTCAGATCAAGGTCAGGAACAGATGGAACAGCTGAATATGGGCCAAAC |
| | CD28TM, ICD- | AGGATATCTGTGGTAAGCAGTTCCTGCCCCGGCTCAGGGCCAAGAACAGA |
| | CD3Z CAR | TGGAACAGCTGAATATGGGCCAAACAGGATATCTGTGGTAAGCAGTTCCTG |
| | | CCCCGGCTCAGGGCCAAGAACAGATGGTCCCCAGATGCGGTCCAGCCCTC |
| | | AGCAGTTTCTAGAGAACCATCAGATGTTTCCAGGGTGCCCCAAGGACCTGA |
| | | AATGACCCTGTGCCTTATTTGAACTAACCAATCAGTTCGCTTCTCGCTTCTG |
| | | TTCGCGCGCTTCTGCTCCCCGAGCTCAATAAAAGAGCCCACAACCCCTCAC |
| | | TCGGCGCGCCAGTCCTCCGATTGACTGAGTCGCCCGGGTACCCGTGTATC |
| | | CAATAAACCCTCTTGCAGTTGCATCCGACTTGTGGTCTCGCTGTTCCTTGG |
| | | GAGGGTCTCCTCTGAGTGATTGACTACCCGTCAGCGGGGGTCTTTCATTTG |
| | | GGGGCTCGTCCGGGATCGGGAGACCCCTGCCCAGGGACCACCGACCCAC |
| | | CACCGGGAGGTAAGCTGGCCAGCAACTTATCTGTGTCTGTCCGATTGTCTA |
| | | GTGTCTATGACTGATTTTATGCGCCTGCGTCGGTACTAGTTAGCTAACTAGC |
| | | TCTGTATCTGGCGGACCCGTGGTGGAACTGACGAGTTCGGAACACCCGGC |
| | | CGCAACCCTGGGAGACGTCCCAGGGACTTCGGGGGCCGTTTTTGTGGCCC |
| | | GACCTGAGTCCTAAAATCCCGATCGTTTAGGACTCTTTGGTGCACCCCCCT |
| | | TAGAGGAGGGATATGTGGTTCTGGTAGGAGACGAGAACCTAAAACAGTTCC |
| | | CGCCTCCGTCTGAATTTTTGCTTTCGGTTTGGGACCGAAGCCGCGCCGCG |
| | | CGTCTTGTCTGCTGCAGCATCGTTCTGTGTTGTCTCTGTCTGACTGTGTTTC |
| | | TGTATTTGTCTGAAAATATGGGCCCGGGCTAGCCTGTTACCACTCCCTTAA |
| | | GTTTGACCTTAGGTCACTGGAAAGATGTCGAGCGGATCGCTCACAACCAGT |
| | | CGGTAGATGTCAAGAAGAGACGTTGGGTTACCTTCTGCTCTGCAGAATGGC |
| | | CAACCTTTAACGTCGGATGGCCGCGAGACGGCACCTTTAACCGAGACCTC |
| | | ATCACCCAGGTTAAGATCAAGGTCTTTTCACCTGGCCCGCATGGACACCCA |
| | | GACCAGGTCCCCTACATCGTGACCTGGGAAGCCTTGGCTTTTGACCCCCCT |
| | | CCCTGGGTCAAGCCCTTTGTACACCCTAAGCCTCCGCCTCCTCTTCCTCCA |
| | | TCCGCCCCGTCTCTCCCCCTTGAACCTCCTCGTTCGACCCCGCCTCGATCC |
| | | TCCCTTTATCCAGCCCTCACTCCTTCTCTAGGCGCCCCCATATGGCCATAT |
| | | GAGATCTTATATGGGGCACCCCCGCCCCTTGTAAACTTCCCTGACCCTGAC |
| | | ATGACAAGAGTTACTAACAGCCCCTCTCTCCAAGCTCACTTACAGGCTCTCT |
| | | ACTTAGTCCAGCACGAAGTCTGGAGACCTCTGGCGGCAGCCTACCAAGAA |
| | | CAACTGGACCGACCGGTGGTACCTCACCCTTACCGAGTCGGCGACACAGT |
| | | GTGGGTCCGCCGACACCAGACTAAGAACCTAGAACCTCGCTGGAAAGGAC |
| | | CTTACACAGTCCTGCTGACCACCCCCACCGCCCTCAAAGTAGACGGCATC |
| | | GCAGCTTGGATACACGCCGCCCACGTGAAGGCTGCCGACCCCGGGGGTG |
| | | GACCATGACTCGAGCCATGGATTGGATCTGGCGCATCCTGTTTCTCGTGGG |
| | | AGCTGCCACAGGCGCCCATTCTCAGGTTCAGCTGCAAGAGTCTGGCCCTG |
| | | GCCTGGTCAAGCCTAGCGAAACACTGAGCCTGACCTGTACCGTGTCTGGC |
| | | GGCAGCATCAGCAGCAGCTCTTACTACTGGGGCTGGATCAGACAGCCTCC |
| | | TGGCAAAGGCCTGGAATGGATCGGCTCCATCTACTACAGCGGCAGCACCT |
| | | ACTACAACCCCAGCCTGAAGTCCAGAGTGACCATCAGCGTGGACACCAGC |
| | | AAGAACCAGTTCTCCCTGAAGCTGAGCAGCGTGACAGCCGCCGATACAGC |
| | | CGTGTACTACTGTGCCAGATACGCCCCTGATAGCAGCGGCTACCTGGTGG |
| | | CCTTTGATATCTGGGGCCAGGGCACAATGGTCACCGTTTCTAGCGGAGGC |
| | | GGAGGTTCTGGTGGCGGAGGAAGTGGCGGCGGAGGATCTCAGACAGTGG |
| | | TCACACAAGAGCCCAGCTTCTCCGTGTCTCCTGGCGGAACAGTGACCCTG |
| | | ACATGTGGCCTTAGCTCTGGCTCTGTGTCCACCGGCTACTACCCCAGCTGG |
| | | TATCAGCAGACACCTGGACAGGCCCCTCGGACACTGATCTACAGCACCAA |
| | | CAGCAGATCCAGCGGCGTGCCCGATAGATTCAGCGGCTCTATCCTGGGCA |
| | | ACAAGGCCGCACTGACAATCACAGGCGCTCAGGCCGATGACGAGAGCGAC |
| | | TACTACTGCGTGCTGTACATGGGCAGCGGCATCTCCGTTTTTGGCGGAGG |
| | | CACAAAGCTGACCGTGCTGGGATCCGAACCAAAGAGTTGCGACAAAACAC |
| | | ACACCTGCCCTACGCGTTTTTGGGTGCTCGTGGTGGTGGGTGGCGTGCTC |
| | | GCTTGCTACTCACTTCTGGTGACCGTAGCGTTTATCATTTTTTGGGTCAGGA |
| | | GCAAGCGATCCCGCCTATTGCACAGCGACTACATGAACATGACCCCCCGG |
| | | CGCCCCGGGCCAACCCGGAAGCACTACCAGCCATATGCGCCTCCCCGCG |
| | | ATTTCGCAGCGTATCGGTCCCGGGTCAAATTTTCACGGTCCGCTGACGCCC |
| | | CGGCCTATCAACAGGGCCAGAATCAGCTGTATAATGAATTAAACCTCGGTA |
| | | GACGCGAGGAGTACGACGTCCTCGACAAGAGAAGGGGGCGCGACCCAGA |
| | | GATGGGAGGCAAACCGCAGCGCAGGAAGAATCCACAGGAGGGCCTGTAC |
| | | AACGAATTACAGAAGGACAAGATGGCAGAGGCCTACAGCGAGATAGGAAT |
| | | GAAGGGTGAAAGGCGTCGTGGAAAGGGCCACGATGGGCTTTACCAGGGC |
| | | CTAAGTACTGCCACAAAAGATACGTATGACGCGCTGCATATGCAAGCCCTC |
| | | CCCCCCAGGTAAGCATGCAACCTCGATCCGGATTAGTCCAATTTGTTAAAG |
| | | ACAGGATATCAGTGGTCCAGGCTCTAGTTTTGACTCAACAATATCACCAGCT |
| | | GAAGCCTATAGAGTACGAGCCATAGATAAAATAAAAGATTTTATTTAGTCTC |
| | | CAGAAAAAGGGGGGAATGAAAGACCCCACCTGTAGGTTTGGCAAGCTAGC |
| | | TTAAGTAACGCCATTTTGCAAGGCATGGAAAAATACATAACTGAGAATAGAG |
| | | AAGTTCAGATCAAGGTCAGGAACAGATGGAACAGCTGAATATGGGCCAAAC |
| | | AGGATATCTGTGGTAAGCAGTTCCTGCCCCGGCTCAGGGCCAAGAACAGA |
| | | TGGAACAGCTGAATATGGGCCAAACAGGATATCTGTGGTAAGCAGTTCCTG |
| | | CCCCGGCTCAGGGCCAAGAACAGATGGTCCCCAGATGCGGTCCAGCCCTC |
| | | AGCAGTTTCTAGAGAACCATCAGATGTTTCCAGGGTGCCCCAAGGACCTGA |
| | | AATGACCCTGTGCCTTATTTGAACTAACCAATCAGTTCGCTTCTCGCTTCTG |
| | | TTCGCGCGCTTC |
| 48 | HER2(E4)- | AAGTTCAGATCAAGGTCAGGAACAGATGGAACAGCTGAATATGGGCCAAAC |
| | CD28TM, ICD- | AGGATATCTGTGGTAAGCAGTTCCTGCCCCGGCTCAGGGCCAAGAACAGA |
| | CD3Z CAR | TGGAACAGCTGAATATGGGCCAAACAGGATATCTGTGGTAAGCAGTTCCTG |
| | | CCCCGGCTCAGGGCCAAGAACAGATGGTCCCCAGATGCGGTCCAGCCCTC |
| | | AGCAGTTTCTAGAGAACCATCAGATGTTTCCAGGGTGCCCCAAGGACCTGA |
| | | AATGACCCTGTGCCTTATTTGAACTAACCAATCAGTTCGCTTCTCGCTTCTG |
| | | TTCGCGCGCTTCTGCTCCCCGAGCTCAATAAAAGAGCCCACAACCCCTCAC |
| | | TCGGCGCGCCAGTCCTCCGATTGACTGAGTCGCCCGGGTACCCGTGTATC |
| | | CAATAAACCCTCTTGCAGTTGCATCCGACTTGTGGTCTCGCTGTTCCTTGG |
| | | GAGGGTCTCCTCTGAGTGATTGACTACCCGTCAGCGGGGGTCTTTCATTTG |
| | | GGGGCTCGTCCGGGATCGGGAGACCCCTGCCCAGGGACCACCGACCCAC |
| | | CACCGGGAGGTAAGCTGGCCAGCAACTTATCTGTGTCTGTCCGATTGTCTA |
| | | GTGTCTATGACTGATTTTATGCGCCTGCGTCGGTACTAGTTAGCTAACTAGC |
| | | TCTGTATCTGGCGGACCCGTGGTGGAACTGACGAGTTCGGAACACCCGGC |
| | | CGCAACCCTGGGAGACGTCCCAGGGACTTCGGGGGCCGTTTTTGTGGCCC |
| | | GACCTGAGTCCTAAAATCCCGATCGTTTAGGACTCTTTGGTGCACCCCCCT |
| | | TAGAGGAGGGATATGTGGTTCTGGTAGGAGACGAGAACCTAAAACAGTTCC |
| | | CGCCTCCGTCTGAATTTTTGCTTTCGGTTTGGGACCGAAGCCGCGCCGCG |
| | | CGTCTTGTCTGCTGCAGCATCGTTCTGTGTTGTCTCTGTCTGACTGTGTTTC |
| | | TGTATTTGTCTGAAAATATGGGCCCGGGCTAGCCTGTTACCACTCCCTTAA |
| | | GTTTGACCTTAGGTCACTGGAAAGATGTCGAGCGGATCGCTCACAACCAGT |
| | | CGGTAGATGTCAAGAAGAGACGTTGGGTTACCTTCTGCTCTGCAGAATGGC |
| | | CAACCTTTAACGTCGGATGGCCGCGAGACGGCACCTTTAACCGAGACCTC |
| | | ATCACCCAGGTTAAGATCAAGGTCTTTTCACCTGGCCCGCATGGACACCCA |
| | | GACCAGGTCCCCTACATCGTGACCTGGGAAGCCTTGGCTTTTGACCCCCCT |
| | | CCCTGGGTCAAGCCCTTTGTACACCCTAAGCCTCCGCCTCCTCTTCCTCCA |
| | | TCCGCCCCGTCTCTCCCCCTTGAACCTCCTCGTTCGACCCCGCCTCGATCC |
| | | TCCCTTTATCCAGCCCTCACTCCTTCTCTAGGCGCCCCCATATGGCCATAT |
| | | GAGATCTTATATGGGGCACCCCCGCCCCTTGTAAACTTCCCTGACCCTGAC |
| | | ATGACAAGAGTTACTAACAGCCCCTCTCTCCAAGCTCACTTACAGGCTCTCT |
| | | ACTTAGTCCAGCACGAAGTCTGGAGACCTCTGGCGGCAGCCTACCAAGAA |
| | | CAACTGGACCGACCGGTGGTACCTCACCCTTACCGAGTCGGCGACACAGT |
| | | GTGGGTCCGCCGACACCAGACTAAGAACCTAGAACCTCGCTGGAAAGGAC |
| | | CTTACACAGTCCTGCTGACCACCCCCACCGCCCTCAAAGTAGACGGCATC |
| | | GCAGCTTGGATACACGCCGCCCACGTGAAGGCTGCCGACCCCGGGGGTG |
| | | GACCATGACTCGAGCCATGGATTGGATCTGGCGCATCCTGTTTCTCGTGGG |
| | | AGCTGCCACAGGCGCCCATTCTCAGGTTCAGCTGCAACAGTGGGGAGCCG |
| | | GACTGCTGAAGCCTAGCGAAACACTGAGCCTGACCTGTGCCGTGTACGGC |
| | | GGCAGCTTTAGCGGCTACTACTGGTCCTGGATCAGACAGCCTCCTGGCAA |
| | | AGGCCTGGAATGGATCGGCGAGATCAATCACAGCGGCAGCACCAACTACA |
| | | ACCCCAGCCTGAAGTCCAGAGTGACCATCAGCGTGGACACCAGCAAGAAC |
| | | CAGTTCTCCCTGAAGCTGAGCAGCGTGACCACAGCCGATACCGCCGTGTA |
| | | CTACTGTGCCCGGATGGGCATCAATAGCGGCGGCTACCTGTACGGCATGG |
| | | ATGTGTGGGGACAGGGCACCACCGTGACAGTTTCTAGCGGAGGCGGAGGT |
| | | TCTGGTGGCGGAGGAAGTGGCGGCGGAGGATCTCAGACAGTGGTCACAC |
| | | AAGAGCCCAGCTTCTCCGTGTCTCCTGGCGGAACAGTGACCCTGACATGT |
| | | GGCCTTAGCAGCGGCTCTGTGTCCACCAGCTACTACCCTAGCTGGTATCAG |
| | | CAGATCCCCGGACAGGCCCCTCGGACACTGATCTACACCACCAACATCAG |
| | | ATCCAGCGGCGTGCCCGATAGATTCGGCGGATCTATCCTGGGCAACAAGG |
| | | CCGCACTGACAATCACAGGTGCCCAGGCCGAGGACGAGTCCGACTACTAC |
| | | TGCATGCTGTACATGGGCAGCGGCATCTGGGTTTTCGGCGGAGGCACAAA |
| | | GCTGACCGTTCTGGGATCCGAACCAAAGAGTTGCGACAAAACACACACCTG |
| | | CCCTACGCGTTTTTGGGTGCTCGTGGTGGTGGGTGGCGTGCTCGCTTGCT |
| | | ACTCACTTCTGGTGACCGTAGCGTTTATCATTTTTTGGGTCAGGAGCAAGC |
| | | GATCCCGCCTATTGCACAGCGACTACATGAACATGACCCCCCGGCGCCCC |
| | | GGGCCAACCCGGAAGCACTACCAGCCATATGCGCCTCCCCGCGATTTCGC |
| | | AGCGTATCGGTCCCGGGTCAAATTTTCACGGTCCGCTGACGCCCCGGCCT |
| | | ATCAACAGGGCCAGAATCAGCTGTATAATGAATTAAACCTCGGTAGACGCG |
| | | AGGAGTACGACGTCCTCGACAAGAGAAGGGGGCGCGACCCAGAGATGGG |
| | | AGGCAAACCGCAGCGCAGGAAGAATCCACAGGAGGGCCTGTACAACGAAT |
| | | TACAGAAGGACAAGATGGCAGAGGCCTACAGCGAGATAGGAATGAAGGGT |
| | | GAAAGGCGTCGTGGAAAGGGCCACGATGGGCTTTACCAGGGCCTAAGTAC |
| | | TGCCACAAAAGATACGTATGACGCGCTGCATATGCAAGCCCTCCCCCCCAG |
| | | GTAAGCATGCAACCTCGATCCGGATTAGTCCAATTTGTTAAAGACAGGATAT |
| | | CAGTGGTCCAGGCTCTAGTTTTGACTCAACAATATCACCAGCTGAAGCCTA |
| | | TAGAGTACGAGCCATAGATAAAATAAAAGATTTTATTTAGTCTCCAGAAAAA |
| | | GGGGGGAATGAAAGACCCCACCTGTAGGTTTGGCAAGCTAGCTTAAGTAA |
| | | CGCCATTTTGCAAGGCATGGAAAAATACATAACTGAGAATAGAGAAGTTCA |
| | | GATCAAGGTCAGGAACAGATGGAACAGCTGAATATGGGCCAAACAGGATAT |
| | | CTGTGGTAAGCAGTTCCTGCCCCGGCTCAGGGCCAAGAACAGATGGAACA |
| | | GCTGAATATGGGCCAAACAGGATATCTGTGGTAAGCAGTTCCTGCCCCGG |
| | | CTCAGGGCCAAGAACAGATGGTCCCCAGATGCGGTCCAGCCCTCAGCAGT |
| | | TTCTAGAGAACCATCAGATGTTTCCAGGGTGCCCCAAGGACCTGAAATGAC |
| | | CCTGTGCCTTATTTGAACTAACCAATCAGTTCGCTTCTCGCTTCTGTTCGCG |
| | | CGCTTC |
| 49 | HER2(F1)- | AAGTTCAGATCAAGGTCAGGAACAGATGGAACAGCTGAATATGGGCCAAAC |
| | CD28TM, ICD- | AGGATATCTGTGGTAAGCAGTTCCTGCCCCGGCTCAGGGCCAAGAACAGA |
| | CD3Z CAR | TGGAACAGCTGAATATGGGCCAAACAGGATATCTGTGGTAAGCAGTTCCTG |
| | | CCCCGGCTCAGGGCCAAGAACAGATGGTCCCCAGATGCGGTCCAGCCCTC |
| | | AGCAGTTTCTAGAGAACCATCAGATGTTTCCAGGGTGCCCCAAGGACCTGA |
| | | AATGACCCTGTGCCTTATTTGAACTAACCAATCAGTTCGCTTCTCGCTTCTG |
| | | TTCGCGCGCTTCTGCTCCCCGAGCTCAATAAAAGAGCCCACAACCCCTCAC |
| | | TCGGCGCGCCAGTCCTCCGATTGACTGAGTCGCCCGGGTACCCGTGTATC |
| | | CAATAAACCCTCTTGCAGTTGCATCCGACTTGTGGTCTCGCTGTTCCTTGG |
| | | GAGGGTCTCCTCTGAGTGATTGACTACCCGTCAGCGGGGGTCTTTCATTTG |
| | | GGGGCTCGTCCGGGATCGGGAGACCCCTGCCCAGGGACCACCGACCCAC |
| | | CACCGGGAGGTAAGCTGGCCAGCAACTTATCTGTGTCTGTCCGATTGTCTA |
| | | GTGTCTATGACTGATTTTATGCGCCTGCGTCGGTACTAGTTAGCTAACTAGC |
| | | TCTGTATCTGGCGGACCCGTGGTGGAACTGACGAGTTCGGAACACCCGGC |
| | | CGCAACCCTGGGAGACGTCCCAGGGACTTCGGGGGCCGTTTTTGTGGCCC |
| | | GACCTGAGTCCTAAAATCCCGATCGTTTAGGACTCTTTGGTGCACCCCCCT |
| | | TAGAGGAGGGATATGTGGTTCTGGTAGGAGACGAGAACCTAAAACAGTTCC |
| | | CGCCTCCGTCTGAATTTTTGCTTTCGGTTTGGGACCGAAGCCGCGCCGCG |
| | | CGTCTTGTCTGCTGCAGCATCGTTCTGTGTTGTCTCTGTCTGACTGTGTTTC |
| | | TGTATTTGTCTGAAAATATGGGCCCGGGCTAGCCTGTTACCACTCCCTTAA |
| | | GTTTGACCTTAGGTCACTGGAAAGATGTCGAGCGGATCGCTCACAACCAGT |
| | | CGGTAGATGTCAAGAAGAGACGTTGGGTTACCTTCTGCTCTGCAGAATGGC |
| | | CAACCTTTAACGTCGGATGGCCGCGAGACGGCACCTTTAACCGAGACCTC |
| | | ATCACCCAGGTTAAGATCAAGGTCTTTTCACCTGGCCCGCATGGACACCCA |
| | | GACCAGGTCCCCTACATCGTGACCTGGGAAGCCTTGGCTTTTGACCCCCCT |
| | | CCCTGGGTCAAGCCCTTTGTACACCCTAAGCCTCCGCCTCCTCTTCCTCCA |
| | | TCCGCCCCGTCTCTCCCCCTTGAACCTCCTCGTTCGACCCCGCCTCGATCC |
| | | TCCCTTTATCCAGCCCTCACTCCTTCTCTAGGCGCCCCCATATGGCCATAT |
| | | GAGATCTTATATGGGGCACCCCCGCCCCTTGTAAACTTCCCTGACCCTGAC |
| | | ATGACAAGAGTTACTAACAGCCCCTCTCTCCAAGCTCACTTACAGGCTCTCT |
| | | ACTTAGTCCAGCACGAAGTCTGGAGACCTCTGGCGGCAGCCTACCAAGAA |
| | | CAACTGGACCGACCGGTGGTACCTCACCCTTACCGAGTCGGCGACACAGT |
| | | GTGGGTCCGCCGACACCAGACTAAGAACCTAGAACCTCGCTGGAAAGGAC |
| | | CTTACACAGTCCTGCTGACCACCCCCACCGCCCTCAAAGTAGACGGCATC |
| | | GCAGCTTGGATACACGCCGCCCACGTGAAGGCTGCCGACCCCGGGGGTG |
| | | GACCATGACTCGAGCCATGGATTGGATCTGGCGCATCCTGTTTCTCGTGGG |
| | | AGCTGCCACAGGCGCCCATTCTCAGGTTCAGCTGGTGGAATCTGGCCCTG |
| | | GCCTGGTTAAGCCTAGCGGCACACTGTCTCTGACCTGTGCTGTGTCTGGC |
| | | GGCAGCATCAGCAGCAGCAATTGGTGGTCTTGGGTCCGACAGCCTCCTGG |
| | | CAAAGGCCTGGAATGGATCGGCGAGATCTACCACAGCGGCAGCACCAACT |
| | | ACAACCCCAGCCTGAAGTCCAGAGTGACCATCAGCGTGGACACCAGCAAG |
| | | AACCAGTTCTCCCTGAAGCTGAGCAGCGTGACAGCCGCCGATACAGCCGT |
| | | GTACTACTGTGCCAGAATGGGAGCCAATAGCGGCGGCTACCTGTACGGCA |
| | | TGGATGTGTGGGGACAGGGCACCACCGTGACAGTTTCTAGCGGAGGCGGA |
| | | GGTTCTGGTGGCGGAGGAAGTGGCGGCGGAGGATCTCAGACAGTGGTCA |
| | | CACAAGAGCCCAGCTTCTCCGTGTCTCCTGGCGGAACAGTGACCCTGACA |
| | | TGTGGCCTTAGCAGCGGCTCTGTGTCTACCAGCTACTACCCCTCCTGGTAT |
| | | CAGCAGACCCCTGGACAGGCTCCCCGGACACTGATCTACTCCACCAACAC |
| | | CAGATCCAGCGGCGTGCCCGATAGATTCTCCGGCTCTATCCTGGGCAACA |
| | | AGGCCGCACTGACAATCACAGGCGCTCAGGCCGATGACGAGAGCGACTAC |
| | | TACTGCGTGCTGTACATGGGCAGCGGCATCTGGGTTTTCGGCGGAGGCAC |
| | | AAAGCTGACCGTTCTGGGATCCGAACCAAAGAGTTGCGACAAAACACACAC |
| | | CTGCCCTACGCGTTTTTGGGTGCTCGTGGTGGTGGGTGGCGTGCTCGCTT |
| | | GCTACTCACTTCTGGTGACCGTAGCGTTTATCATTTTTTGGGTCAGGAGCAA |
| | | GCGATCCCGCCTATTGCACAGCGACTACATGAACATGACCCCCCGGCGCC |
| | | CCGGGCCAACCCGGAAGCACTACCAGCCATATGCGCCTCCCCGCGATTTC |
| | | GCAGCGTATCGGTCCCGGGTCAAATTTTCACGGTCCGCTGACGCCCCGGC |
| | | CTATCAACAGGGCCAGAATCAGCTGTATAATGAATTAAACCTCGGTAGACG |
| | | CGAGGAGTACGACGTCCTCGACAAGAGAAGGGGGCGCGACCCAGAGATG |
| | | GGAGGCAAACCGCAGCGCAGGAAGAATCCACAGGAGGGCCTGTACAACG |
| | | AATTACAGAAGGACAAGATGGCAGAGGCCTACAGCGAGATAGGAATGAAG |
| | | GGTGAAAGGCGTCGTGGAAAGGGCCACGATGGGCTTTACCAGGGCCTAAG |
| | | TACTGCCACAAAAGATACGTATGACGCGCTGCATATGCAAGCCCTCCCCCC |
| | | CAGGTAAGCATGCAACCTCGATCCGGATTAGTCCAATTTGTTAAAGACAGG |
| | | ATATCAGTGGTCCAGGCTCTAGTTTTGACTCAACAATATCACCAGCTGAAGC |
| | | CTATAGAGTACGAGCCATAGATAAAATAAAAGATTTTATTTAGTCTCCAGAA |
| | | AAAGGGGGGAATGAAAGACCCCACCTGTAGGTTTGGCAAGCTAGCTTAAGT |
| | | AACGCCATTTTGCAAGGCATGGAAAAATACATAACTGAGAATAGAGAAGTTC |
| | | AGATCAAGGTCAGGAACAGATGGAACAGCTGAATATGGGCCAAACAGGATA |
| | | TCTGTGGTAAGCAGTTCCTGCCCCGGCTCAGGGCCAAGAACAGATGGAAC |
| | | AGCTGAATATGGGCCAAACAGGATATCTGTGGTAAGCAGTTCCTGCCCCGG |
| | | CTCAGGGCCAAGAACAGATGGTCCCCAGATGCGGTCCAGCCCTCAGCAGT |
| | | TTCTAGAGAACCATCAGATGTTTCCAGGGTGCCCCAAGGACCTGAAATGAC |
| | | CCTGTGCCTTATTTGAACTAACCAATCAGTTCGCTTCTCGCTTCTGTTCGCG |
| | | CGCTTC |
| 50 | HDAdlL12p70_ | AAACATCATCAATAATATACCTTATTTTGGATTGAAGCCAATATGATAATGAG |
| | TK_aPD-L1 | GGGGTGGAGTTTGTGACGTGGCGCGGGGCGTGGGAACGGGGCGGGTGA |
| | | CGTAGTAGTGTGGCGGAAGTGTGATGTTGCAAGTGTGGCGGAACACATGT |
| | | AAGCGACGGATGTGGCAAAAGTGACGTTTTTGGTGTGCGCCGGTGTACAC |
| | | AGGAAGTGACAATTTTCGCGCGGTTTTAGGCGGATGTTGTAGTAAATTTGG |
| | | GCGTAACCGAGTAAGATTTGGCCATTTTCGCGGGAAAACTGAATAAGAGGA |
| | | AGTGAAATCTGAATAATTTTGTGTTACTCATAGCGCGTAATATTTGTCTAGG |
| | | GCCGCGGGGACTTTGACCGTTTACGTGGAGACTCGCCCAGGTGTTTTTCTC |
| | | AGGTGTTTTCCGCGTTCCGGGTCAAAGTTGGCGTTTTGATATCAAGCTTATC |
| | | GATACCGTAAACAAGTCTTTAATTCAAGCAAGACTTTAACAAGTTAAAAGGA |
| | | GCTTATGGGTAGGAAGTAGTGTTATGATGTATGGGCATAAAGGGTTTTAAT |
| | | GGGATAGTGAAAATGTCTATAATAATACTTAAATGGCTGCCCAATCACCTAC |
| | | AGGATTGATGTAAACATGGAAAAGGTCAAAAACTTGGGTCACTAAAATAGAT |
| | | GATTAATGGAGAGGATGAGGTTGATAGTTAAATGTAGATAAGTGGTCTTATT |
| | | CTCAATAAAAATGTGAACATAAGGCGAGTTTCTACAAAGATGGACAGGACT |
| | | CATTCATGAAACAGCAAAAACTGGACATTTGTTCTAATCTTTGAAGAGTATG |
| | | AAAAATTCCTATTTTAAAGGTAAAACAGTAACTCACAGGAAATACCAACCCA |
| | | ACATAAAATCAGAAACAATAGTCTAAAGTAATAAAAATCAAACGTTTGCACG |
| | | ATCAAATTATGAATGAAATTCACTACTAAAATTCACACTGATTTTGTTTCATC |
| | | CACAGTGTCAATGTTGTGATGCATTTCAATTGTGTGACACAGGCAGACTGT |
| | | GGATCAAAAGTGGTTTCTGGTGCGACTTACTCTCTTGAGTATACCTGCAGT |
| | | CCCCTTTCTTAAGTGTGTTAAAAAAAAAGGGGGATTTCTTCAATTCGCCAAT |
| | | ACTCTAGCTCTCCATGTGCTTTCTAGGAAACAAGTGTTAACCCACCTTATTT |
| | | GTCAAACCTAGCTCCAAAGGACTTTTGACTCCCCACAAACCGATGTAGCTC |
| | | AAGAGAGGGTATCTGTCACCAGTATGTATAGTGAAAAAAGTATCCCAAGTC |
| | | CCAACAGCAATTCCTAAAAGGAGTTTATTTAAAAAACCACACACACCTGTAA |
| | | AATAAGTATATATCCTCCAAGGTGACTAGTTTTAAAAAAACAGTATTGGCTTT |
| | | GATGTAAAGTACTAGTGAATATGTTAGAAAAATCTCACTGTAACCAAGTGAA |
| | | ATGAAAGCAAGTATGGTTTGCAGAGATTCAAAGAAAATATAAGAAAACCTAC |
| | | TGTTGCCACTAAAAAGAATCATATATTAAATATACTCACACAATAGCTCTTCA |
| | | GTCTGATAAAATCTACAGTCATAGGAATGGATCTATCACTATTTCTATTCAGT |
| | | GCTTTGATGTAATCCAGCAGGTCAGCAAAGAATTTATAGCCCCCCTTGAGC |
| | | ACACAGAGGGCTACAATGTGATGGCCTCCCATCTCCTTCATCACATCTCGA |
| | | GCAAGACGTTCAGTCCTACAGAAATAAAATCAGGAATTTAATAGAAAGTTTC |
| | | ATACATTAAACTTTATAACAAACACCTCTTAGTCATTAAACTTCCACACCAAC |
| | | CTGGGCAATATAGTGAGACCCCATGCCTGCAAAAAAAAAAAAATTAGCCAG |
| | | GCATGGTAGCATGTACCTGTAGTCCCAGCTACTTGAGAGGTGAGGTGGGA |
| | | AAATCACTTTAGTGCAGGATGTTGAGGCTGGAGTGAACTGTGATTGTGCCA |
| | | CTGCACTCCAGCCTGGACAATAGAGCAAGACCTTGTCTCAAAAAAATGCAT |
| | | TAAAAATTTTTTTTAAATCTTCCACGTATCACATCCTTTGCCCTCATGTTTCAT |
| | | AAGGTAAAAAATTTGATACCTTCAAAAAAACCAAGCATACCACTATCATAATT |
| | | TTTTTTAAATGCAAATAAAAACAAGATACCATTTTCACCTATCAGACTGGCAG |
| | | GTTCTGATTAAATGAAATTTTCTGGATAATATACAATATTAAGAGAGACTGTA |
| | | GAAACTGGGCCAGTGGCTCATGCCTGTAATCCCAGCACTTTGGGAGGCTG |
| | | GGTAACATGGCGAACCCTGTTTCTACAAAATAAAAATATTAGCTGGGAGTG |
| | | GTGGCGCACACCTATAGTCCCAGCTACTCAGGAGGCTGAGGTGGAAGGAT |
| | | CGCTTGAACCCAGGAGGTTGAGACTGCAGTGAACTGTGATCATTCTGCTGC |
| | | ACTGCACCCCAGCCTGGGCAACAGAGACCTTGTCTCAAAAAAAAAAAAAAA |
| | | AGAGACAAATTGTGAAGAGAAAGGTACTCTCATATAACATCAGGAGTATAAA |
| | | ATGATTCAACTTCTTAGAGGAAAATTTGGCAATACCAAAATATTCAATAAACT |
| | | CTTTCCCCTTGACCCAGAAATTCCACTTGAATAAAGCTGAACAAGTACCAAA |
| | | CATGTAAAAGAATGTTTCTTCTAGTACAGTCGGTAAGAACAAAATAGTGTCT |
| | | ATCAATAGTGGACTGGTTAAATCAGTTATGGTATCTCCATAAGACAGAATGC |
| | | TATGCAACCTTTAAAATATATTAGATAGCTCTAGACACACTAATATTAAAAGT |
| | | GTCCAATAACATTTAAAACTATACTCATACGTTAAAATATAAATGTATATATG |
| | | TACTTTTGCATATAGTATACATGCATAGGCCAGTGCTTGAGAAGAAATGTGT |
| | | ACAGAAGGCTGAAAGGAGAGAACTTTAGTCTTCTTGTTTATGGCCTCCATA |
| | | GTTAGAATATTTTATAACACAAATATTTTGATATTATAATTTTAAAATAAAAAC |
| | | ACAGAATAGCCAGACATACAATGCAAGCATTCAATACCAGGTAAGGTTTTTC |
| | | ACTGTAATTGACTTAACAGAAAATTTTCAAGCTAGATGTGCATAATAATAAAA |
| | | ATCTGACCTTGCCTTCATGTGATTCAGCCCCAGTCCATTACCCTGTTTAGGA |
| | | CTGAGAAATGCAAGACTCTGGCTAGAGTTCCTTCTTCCATCTCCCTTCAATG |
| | | TTTACTTTGTTCTGGTCCCTACAGAGTCCCACTATACCACAACTGATACTAA |
| | | GTAATTAGTAAGGCCCTCCTCTTTTATTTTTAATAAAGAAGATTTTAGAAAGC |
| | | ATCAGTTATTTAATAAGTTGGCCTAGTTTATGTTCAAATAGCAAGTACTCAGA |
| | | ACAGCTGCTGATGTTTGAAATTAACACAAGAAAAAGTAAAAAACCTCATTTT |
| | | AAGATCTTACTTACCTGTCCATAATTAGTCCATGAGGAATAAACACCCTTTC |
| | | CAAATCCTCAGCATAATGATTAGGTATGCAAAATAAATCAAGGTCATAACCT |
| | | GGTTCATCATCACTAATCTGAAAAAGAAATATAGCTGTTTCAATGAGAGCAT |
| | | TACAGGATACAAACATTTGATTGGATTAAGATGTTAAAAAATAACCTTAGTCT |
| | | ATCAGAGAAATTTAGGTGTAAGATGATATTAGTAACTGTTAACTTTGTAGGT |
| | | ATGATAATGAATTATGTAAGAAAACAACAGGCCGGGCGGGTTGGTTCACAC |
| | | GTGTAATCCCAGCACTTTGGGAGGCTGAGGCAGGCAGACTGCCTGAGCTC |
| | | AGGAGTTCGAGACCAGCCTGGGCAACACGGTGAAATCCCGTCTCTACTAAA |
| | | AATACAAAAAAATTAGCCGGGTGTGGTGACACATGCCTGTAGTCCCAGCTA |
| | | CTTGGGAGGCTGAGGCAGGAGAATCACTTGAACCTGGGAGGTGAAGGTTG |
| | | CAGTGAGCCAAGATGGCACCACTTCACTCCAGCCTGGGAAACAGAGCAAG |
| | | ACTCTGTCTCTGAGCTGAGATGGCACCACTTCACTCCAGCCTGGGAAACAG |
| | | AGCAAGACTCTGTCTCAAAAAAAACAAAACACACAAACAAAAAAACAGGCTG |
| | | GGCGCGGTGGCTCACGCCTGTAATCCCAGCACTTTGGGAGGCCGAGGCG |
| | | GGTGGATCACCTGAGGTCAGGAGTTCCAGACCAGCCTTGTCAACATGGTG |
| | | AAACCTCCCCCCGCCGTCTCTACTAAAAATACAAAAATTAGCCAGGCGTGG |
| | | TGGCAGGAGCCTGTAATCCCAGCTACTTGGGAGGCTGAGGCAGGAGAATC |
| | | GCTTGTACCCAGAAGGCAGAGGTTGCACTGAGCTGAGATGGCACCATTGC |
| | | ACTCCAGCCTGGGGGACAAGAGCGAGATTTCGTCTTTAAAAAACAAAAACA |
| | | AAACAAAAAACCATGTAACTATATGTCTTAGTCATCTTAGTCAAGAATGTAGA |
| | | AGTAAAGTGATAAGATATGGAATTTCCTTTAGGTCACAAAGAGAAAAAGAAA |
| | | AATTTTAAAGAGCTAAGACAAACGCAGCAAAATCTTTATATTTAATAATATTC |
| | | TAAACATGGGTGATGAACATACGGGTATTCATTATACTATTCTCTCCACTTTT |
| | | GAGTATGTTTGAAAATTTAGTAAAACAAGTTTTAACACACTGTAGTCTAACAA |
| | | GATAAAATATCACACTGAACAGGAAAAACTGGCATGGTGTGGTGGCTCACA |
| | | CTTGTAATCCCAGTGCTTTGGGAGGCTGAGACAGGAGAGTTGCTTGAGGC |
| | | CAGGAGTTCAAGACCGACATGGGGAATGTAGCAAGACCCCGTCCCTACAA |
| | | AAAACTTTGTAAAAATTTGCCAGGTATGGTGGTGCATACCTGTAGTCCCAGC |
| | | TACTCGGGAGGCGGAGGCAGAAGGAATCACTTGAGCCCAGGAGTTTGAGG |
| | | CTGCAGTGAGCTACGATCATACCACAGCACTCCAGCGTGGACAACAGAGTA |
| | | AGACCCTATCTCAAAAACAAAACAAAACAAAACAAACAAAAAAAACCACAAG |
| | | AAAAACTGCTGGCTGATGCAGCGGCTCATGCCTGTAATCCCAGTATTTTGG |
| | | GAGGCCCAGGTGGGCGTATCACCTGAGGTCAGGAGTTAGAGACCAGCCTG |
| | | GCCAACATGGTGAAACCCCATCTCTACTAAAAATACAAAATTAGCCAGGCAT |
| | | GTGGCACGCGCCTGTAGTCCCAGTTACTGGGAGGCTGAAGCAGGAGGATC |
| | | ACCTGAGCCCGGGAGGTGGAGGTTGCAGTGAGCCGAGATCACACCACTGC |
| | | ACTCCAGCCTGGGTGACACAGCAATACCCTACCTCAAAATAAAAAAGAAAA |
| | | AGAAAAGAAAAGTTGCTGTCCCCGCTACCCCAATCCCAAATCCAAACAGCC |
| | | TCTCTCATCTCACAGTAAGGGGGAAAAATCACCCAAAAAAGCTAAGTGATCT |
| | | TTTGAAAACCCAAACTCTTAGAAGTCTAAGATTATTATAGTCAACTCATGAAG |
| | | TGTCATCATAAAAGATACTCTAATATTATTTAAGTAGAACCACATATTGGTTG |
| | | TCTTGGTATGTCTAGCCCCTGGCATACAAAATATTTAATAACACTGATATGG |
| | | TACCTGTGATGTGAAAATGTACTATGAGTACAGCTTTATAAATACTATATATG |
| | | TACCTATATACAGAAAAAAATACAACAAAATCATAAAAGCACTTATCTTTGAA |
| | | AGAGGAGTTACAGCAATTTTATTTAGTTCTTTATTGCTTTGCTATATATTCTA |
| | | AATTTTTTTCAATGAATATATATCACTTTTAAAAAAATTCAATGGTCTTTCTTA |
| | | TAAATTATCTTTGGCAGCATGCGTTTTTATATATACATATAAAATGTATGGGA |
| | | AATTTTTAAAGGATACATTAAATTAAAGCAAAATATACAAACAAAAAATCAGA |
| | | ATACAAAAAGATAAAAAGATTGGGAAGGGAGGGAGGGAGTAAGGAGGAAG |
| | | GGTGGGTGGGTATAGAGAAATATACCAAATAATGGTAAGAAGTGGGGTCTT |
| | | GACACTTTCTACACTTTTTTTAAATAAAAAAAATTTTTTTCTCTCTCTTTTTTTT |
| | | TTTTAGAGACGAAGTCTCGCTATGTTGCCCAGGCTGGTCTTGAACTCCTGG |
| | | GATCAAGAGATCCTCCTGCCTCAGCCTCCCAAGGTGCTTGGATTACAGGTG |
| | | TGAGCCACCACGCCTGGTCACTTTCTACACTTTAATATATATATTTTTTCATT |
| | | TTCAATGTCATTTTTATTAGTTAATTTATAATACCCATTCACCATTATATTCAA |
| | | AGTCTATTTGAAGAAATAAACCAGAAAGAATGAAATACTCTAGCTCACATGC |
| | | TATTCAATACTAAATTACCTTTCAAATCACATTCAAGAAGCTGATGATTTAAG |
| | | CTTTGGCGGTTTCCAATAAATATTGGTCAAACCATAATTAAATCTCAATATAT |
| | | CAGTTAGTACCTATTGAGCATCTCCTTTTACAACCTAAGCATTGTATTAGGT |
| | | GCTTAAATACAAGCAGCTTGACTTTTAATACATTTAAAAATACATATTTAAGA |
| | | CTTAAAATCTTATTTATGGAATTCAGTTATATTTTGAGGTTTCCAGTGCTGAG |
| | | AAATTTGAGGTTTGTGCTGTCTTTCAGTCCCCAAAGCTCAGTTCTGAGTTCT |
| | | CAGACTTTGGTGGAACTTCATGTATTGTCAGGTTGGCCCGTAATACCTGTG |
| | | GGACAACTTCAGCCCCTGTGCACATGGCCAGGAGGCTGGTTGCAAACATTT |
| | | TCAGGTAGGTGGACCAGGACATGCCCCTGGTCATGGCCAGGTGGAGGCAT |
| | | AGTGCTATACAGCAGGCAGAAGTCAATATTGATTTGTTTTTAAAGAAACATG |
| | | TACTACTTTCATAAGCAGAAAAAATTTCTATTCTTGGGGGAAAAGATTATGC |
| | | CAGATCCTCTAGGATTAAATGCTGATGCATCTGCTAAACCTTCACATATCAG |
| | | AACATATTTACTATAGAAAGAATGAAAATGGGACATTTGTGTGTCACCTATG |
| | | TGAACATTCCAAAAATATTTTACAACAACTAAGTATTTTATAAATTTTATGAAC |
| | | TGAAATTTAGTTCAAGTTCTAGGAAAATACAAACCTTGCTAGATATTATAAAA |
| | | ATGATACAATATATATTCATTTCAGGCTCATCAGAATATATCTGTTATCACTT |
| | | GACAAGAATGAAAATGCACCATTTTGTAGTGCTTTAAAATCAGGAAGATCCA |
| | | GAGTACTAAAAATGACTTCTTCCTTGAAGCTTACTCACCAACTTCCTCCCAG |
| | | TTACTCACTGCTTCTGCCACAAGCATAAACTAGGACCCAGCCAGAACTCCC |
| | | TTGAAATATACACTTGCAACGATTACTGCATCTATCAAAATGGTTCAGTGCC |
| | | TGGCTACAGGTTCTGCAGATCGACTAAGAATTTGAAAAGTCTTGTTTATTTC |
| | | AAAGGAAGCCCATGTGAATTCTGCCCAGAGTTCATCCCAGATATGCAGTCT |
| | | AAGAATACAGACAGATCAGCAGAGATGTATTCTAAAACAGGAATTCTGGCA |
| | | ATATAACAAATTGATTTCCAATCAAAACAGATTTACATACCATACTTATGTCA |
| | | AGAAGTTGTTTTGTTTTATTGCATCCTAGATTTTATTTTTTTGATTTATGGTTT |
| | | ACTTTAAGCATAAAAAATTTGTCAATACAACTCTTCCCAAAAGGCATAAACAA |
| | | AAATTCATAAAACTTGCATCACTTGAGATACTTCAGGTATGAATTCACAACTT |
| | | TGTTACAACTTACTATATATATGCACACATATATATATATTTGGGTATATTGG |
| | | GGGGGTTCTAATTTAAGAAATGCATAATTGGCTATAGACAGACAGTTGTCTG |
| | | GAATGAAAATCAATACTTTTGCTATAATCGATTACTGAAATAATTTTACTTTC |
| | | CAGTAAAACTGGCATTATAATTTTTTTTAATTTTTAAAACTTCATAATTTTTTG |
| | | CCAGACTGACCCATGTAAACATACAAATTACTAATAATTATGCACGTCACAT |
| | | CTGTAATAATGGCCTTCATGTAAACATTTTTGTGGTTTACACATAAAATCTCT |
| | | AATTACAAAGCTATATTATCTAAAATTACAGTAAGCAAGAAAATTAATCCAAG |
| | | CTAAGACAATACTTGCAACATCAATTCATCATCTGTGACAAGGACTGCTTAA |
| | | GTCTCTTTGTGGTTAAAAAGGAAAAAAAAAAAAAAGACATGTTGGCCAGATG |
| | | CGGTGGCTCACACCTGTAATCCCAGCACTTTGGGAGGCTGAGGTGGGCGG |
| | | ATCACCCCTGGCCTGCCCAACATGGTGAAACCCCGTCTCTACTAAAAACAC |
| | | AAAAATTAGCTGGGCGTGGTGGCGGGCGCCTGTAATTCCAGCTACTCGGG |
| | | AGGCTGAGGCAGGAGAATTGCTAGAACCCAGGAGGCAGAGATTGCAGTGA |
| | | GCTGAGATTGCACCATTGCACTACAGTCTGGGCAACAAAAGTGAAACTCCA |
| | | TCTTAAAAAAAAAAAGACAATGTTCGTGGGTCCAAACAAGACTTAATGGAAG |
| | | TGAGTCTAAAAATGAGCTATGTGGGCCAGGCGTAGTGGCTCCCACCTGTAA |
| | | TCCCAGCACTTTGGGAGGCCGAAGCAGGCAGATCATGAGGTCAGGAGATG |
| | | GAGACCATCCTGGCCAACACGGTGAAATCCTGTCTCTACAAAAATTAGCTG |
| | | GGCGTGGTGGTGCCTGCCTGTAATCCCAGCTACTCAGAAGGCTCAGGCAG |
| | | GAGAATCGCTTGAACCAGGGAGTCGGTGGCTAGAGTGAGCCGAGATTTGC |
| | | ATCACTGCACTCCTGCCTGGTGACAGAGCAAGACTCCATCTCAAAAAAAAC |
| | | AAACAAAAATAAAAGATAAAAATGAGCTATGTGAATTAAAAGAGGTATAACA |
| | | ATAGATAAACCATATTTTATTTAATTCCTAGTAATGAGTAATATTTCCAAACTT |
| | | CTGGAATGGGCAGAAATTGCTAGTTGGCATATTTTTACCTTTTATATTCAGA |
| | | TACATTAAAATTCTCAAAAAAAAACACCTCAAAGCAGATGATCCGCCATCTC |
| | | CTTGGATAATTTGTGTTAACTCAGGATAACAGAAAACCAAAATTATGAGTTA |
| | | CTGATGCAATATTCCTAAATGTAAAAATAATTAAAGCTAATAGTAGATTCATC |
| | | TTCCAATTTCATATCAGTCTTACAAATAAACTACATATATAACTTGCTTGCCT |
| | | TCCCTTCTGAGGGATAAAGCTGTTAGAAGAATTAAAATCAGCATTCTTGACT |
| | | ATTCAACCAAGGGAGGGATAAATTATTACTCATTCTAGGGACATGGGCTCAT |
| | | AACTACTACATGTGTAAGGACATGAATTTACCCAATATTACAATTTTTCCTTT |
| | | TATTAGTGTGTACAGTGGAAGAATAGACATGTTCACTCTGGACAAAAAAAAA |
| | | ATTATACTTATCAGTTATCAGAAGCACAATGCTGAAGACAGTAGTTCCATAA |
| | | CAATTTGAAGTATGTGATCGAACTAGTAGATTATCTTAGTAGTAGTGAATTAT |
| | | TGTAAATGTTAGTAATTTGGCAGCCACTGGGCAGAAAAATAAGAATTGAGG |
| | | CTCAATATTGATATTAATGGTGGTGATTGACACATAAATTTTATCAAGTCTAC |
| | | ACAATATAAAATTACAGAAAGGTAGAAGAGTATACCAGTACAACTTCAACAT |
| | | ATCTTCACTACAAGGGAGTAAAATGACATGGCCTAGTTACTATCTAATGAAC |
| | | TGCAGAAAACTAAAAGAAAACTCCAAGGCAACTCTTCTCTGCTGATCTGGTT |
| | | GGTCCTTTTCCTACCTTTTGCAATACCCAGATACAAACAATGGATAGAAAAC |
| | | AAAGTAGACTTGTAGTATGCAGGTCACAGTGCTAAATTCACAGAAAGAAAC |
| | | CCCTGAACTGAACTGCTCTATTTCCTGGTGGTCACAAAGAGTAATTCTGGTT |
| | | TACACCTACAGATTGATGTCAATCTACACCCTGTTGATAACAGTGTGGCCAA |
| | | GGACAAAAAAAAGGTGCTCCGTTTTACCAATTCTGTAAAAAATTATTGGCAG |
| | | GGTAAGCTCGGCTAGGGCAGGATTACATTTCTAGGACTACCATCCCCGAAA |
| | | TTTAGAAGATATTATATCCACATAAAGCATATCTTTCACATTAATTTGCAAAA |
| | | ATCTAAAAGCTTTTTCTTAGCTCAAGTGTGTCCAAGTTTACCCTGGCAGTTT |
| | | AAAACGATAGTTACAAGCAGCATGGGTTGTATCAGACACATTTGAGGGCCA |
| | | ATTTCATGTAAGTGATATTGGGCAAGTTACTTCAACTATCTGTGCCTCCAAG |
| | | GTCATACTAGTGTTTATTTACCTAAAGGGTACCTGTTATGTAACTTTAGGGT |
| | | GTTTACATTAGATAATGCCTGCAAAATATTTACTTCAACGCCTAAAACATAGT |
| | | TAAGTATTCAATAAATACCTACTATTGTCACTACTAACTTAAAAGTTTAGAGA |
| | | TTAAGAGCAGAATCTGGGGTGAGACAAACTTAGGTTCAAATCCTAGTATTGT |
| | | TGGGTAATCTTGGGCAAGTTACTTAACCTCTCTGATTTGTGTAATTTAAAAAA |
| | | TTAGTTAATATACATAACAGGGCTTAGAAGAGTATCTAGCACATAGCACCAT |
| | | TTAAGCATTTGTTATTGCTAACATGCAAACAATTTAAGGGAAAGAAATTTTTT |
| | | AAAAAGGAAGAGGGATTTGCAAACTAAAAACAATGAGTATCTTATGTTCAAA |
| | | GAAAACTAACAAACAGCCAGCTCTAGCAATAATTAAATTCACTATATACTGG |
| | | GGCAGGCATCACACCCCAAAGCTAAAAGCGTCTACCTAGGCCAGGCACGG |
| | | TGGCTCATGCCTGTAATCCCAGCACTTTGGGAAGCAGAGGCGGGCAGATC |
| | | GCTTGAGCTCAGGAGTTCAAGACCAGCCTGGACAACATGGCAAAACACCAT |
| | | CTCTACAAAAAATACAAATATTAGGCCGGGCGCAGTGGCTCACGCCTGTAA |
| | | TCCCAGCACTTTGGGAGGCCAAGGCGGGTGGATCACCTGAGATCAGGAGT |
| | | TCGAGAGTAGCCTGGCCAACATGGTGAAACCTCGTCTCTATTAAAAATACA |
| | | AAAAATTAGCCAGGCATGGTGGCAGGCGCCTGTAATCCCAGCTACTCAGG |
| | | GGGATGAGGTAGGAGAATCGCTTGAACCCGGGAGGCAGAGGTTGCACTGA |
| | | GCCGAGATCATGCCACTGTACTCCAGCCCGGGCAACAAGAGCGAAACTCC |
| | | ATCTCAAAAAATAAATAAATAAATAAATAAAATAAAGTACAAATATTAGCCAG |
| | | GGATGGTGGTGCGCACCTGTAGTCCCAGCTACTTGGGAGGCTGAAGTGGG |
| | | AGAATCCCCTGAGCCTGGGGAGAATCACCCGAGCCCGGGAAGTCGAGGCT |
| | | GCAGTGAGCAGTGATTGTGCCACTGCACTCCATCCTAGGTGACAGAGTGA |
| | | GACCCTGTCTCAAAAAAAAGAAATTGGCAGAATTAAGTAAGTTGATGTTTAG |
| | | AGATGAAAAATCAACATTTTTTCCTCAGCAACTGAATAAAAACAACAGCCAC |
| | | TACCATTTTTTTGAGTACCTATTTGTAGCCTATTTTTTAACTGGTATTACTCG |
| | | AGAGAGAGAGAGCTAGGTTCGAGACAGAGCTCCTTCTCTTAATAACTGTAT |
| | | GACCTAGGGTATGTCTGTTAGCCTCTCTGAGGCTTCAAAGGTTCCTCATCT |
| | | GTAAAATGGTAATAATCATACCATTGCTACAGGGCTGTTTTGAAGACTAATT |
| | | AGGACTATGTAAGTAAACATGATGATGGCTATTATTACTGTTCCCCGCCAGG |
| | | GGCCATGCAAGGGTTGCTGATTCACATAGACTGTCTTATAATCCTCTCAATA |
| | | ACTCCAAGAGGTAGCCAGCACCTCAGATATACATAAAATGACTTAAGCCCA |
| | | GAGAGGTGAAGTAAGTTGCCCACAGCCACACAACTAGTAAATAGCCCAAAC |
| | | AAGCTGGATTCCCAGTTAGACTCCGTTAATAGCACTGCTCTTTACCTTAAGT |
| | | CATTACAATGCCTAATATGAAATAGAATCGCTTCTTTCTTAGGGTTCAAGTG |
| | | GTTAATTATTTAATGTATTCATTCAACAAACCATCATCGAGGACCTCTTACAA |
| | | GCCAAGTACTGTGCTAAGTGCTAGAGTTACGGCGGTGATTCCTGCCCTTAA |
| | | AAAGTTTTAGTGGGAGAAACAACAGGTAACCAGGTCATTGCCAAAACAACA |
| | | AAAATAATCATAATAAAGCAGGCTAAAGCATATTTAACTGGCCGGGGTTTTG |
| | | ACTATTTTAGCAAGCATGATCAGAACGGTTGAGGAGGGAGGCCAGCAGCTT |
| | | GGCCGGTTCAACAAACAAGAAAAAACCAGTGAGGGTGGAGCTAAGATACC |
| | | AGAGGCTGATTACGGTTAAGAATGTTCTTGAAGGTAAGGACCAGATTCTCA |
| | | TTTTCTATATCCTGGGGCATCGGTCAGCATGGAATCTGGATTCTAGCACAT |
| | | GTGAATTTCGGCTTGAAATGACCTAATGCCTTTTCCCTAGTTCCTTCGTGTG |
| | | TCAAATACGCATGGTTACCGCTACCAGAGCTGTAGTGGGGCTTCAATGAGG |
| | | CCATGAGCATCTCCATAAAGATGAACTACAGTGTGTGCAAAACTAAAGGCA |
| | | AAACCTGGTCCCCACACGCCCTCCCAGGTGGTCGCTTTCCGTGCCGAGGC |
| | | CCCTCCAGAGGTGCCCCGAGAACCTCACCATCGCACCCCAAACTTCCAGG |
| | | GAAGGGCCTCTCCCGAGAAAGCCCCCACGCCCCCACCCCGCGCCATCATT |
| | | CCCGAATCTGCCCTCGGCCCCTCCCCGCAGCACGCTCGCAGGCGGCACAT |
| | | GTCAACCAAAACGCCATTTCCACCTTCTCTTCCCACACGCAGTCCTCTTTTC |
| | | CCAGGGCTCCCCCGAGGAGGGACCCACCCCAAACCCCGCCATTCCGTCCT |
| | | CCCTGCCGCCCTCGCGTGACGTAAAGCCGAACCCGGGAAACTGGCCGCC |
| | | CCCGCCTGCGGGGTTCCCTGGGCCCGGCCGCTCTAGAACTAGTGGATCCC |
| | | AATTGAAGGCCTGGTCTAAATGACTCCAAAATCACCACTTAATTCAAGAGAC |
| | | TGATTTCCCTGAGTCAGGCCCCTTAAAGCAGCTATTTCAATGGGACAGGGA |
| | | AACAACCCTAGGATCTGGATTAGAATCACTTGGGGGCTGCCACACCCCCAG |
| | | GGCTCTGATCCTGCCCTTCTCCCACACGCACATTCACATACTGCTGCAGTG |
| | | ACCTTCCATTTCTAATGGGTTCCTGGGCCATCTGTCAGGTATAGGGAATGG |
| | | AAAAGGGGTTGGGGAGGCTCTGCTTCAGAAAGTTTGTGTCAGGGGCTCCC |
| | | AGAGCCTCCACAGATAGATAGCAGGGGTCCCCACCCTACCATGGCAGCTA |
| | | TAAATGTGATCAACATTTATTGGCCTAGGATACAGCAGTTAGCAAAATGCCT |
| | | GATGTAGTTCCCACTCCGTGGAGGTTGCAGGCTAGCTCTTTCCTAATGAGC |
| | | TTTACAGCAGAAGCTGTTTTATCGTTAAGTGCCCCACAGAGACACTTTACCA |
| | | GGAGGCTGGGAGAGTTCTCCAGATTTGGGAGAGGCGCAGAGACAGTGTGT |
| | | GAGCCGAGCCCTGTCTCAGCAATCCACCTGGAGGAGCTAGAGTATCCTCC |
| | | TCCCTTTACCATTCAGACCGAGAGAAAAAGCCCAGCTTGTGTGCACCCTCG |
| | | TGGGGTTAAGGCGAGCTGTTCCTGGTTTAAAGCCTTTCAGTATTTGTTTTGA |
| | | TGTAAGGCTCTGTGGTTTGGGGGGGAACATCTGTAAACATTATTAGTTGATT |
| | | TGGGGTTTGTCTTTGATGGTTTCTATCTGCAATTATCGTCATGTATATTTAAG |
| | | TGTCTGTTATAGAAAACCCACACCCACTGTCCTGTAAACTTTTCTCAGTGTC |
| | | CAGACTTTCTGTAATCACATTTTAATTGCCACCTCGTATTTCACCTCTACATT |
| | | TGAAATCTGGCGTCTGTTTCAAGCCAGTGTGTTTTTTCTTCGTTCTGTAATAA |
| | | ACAGCCAGGAGAAAAGTGCCTCTATGTTTTTATTTTTCAAGGGAGTATTCAG |
| | | TACCTACAAACCCAAGTCAGGAAGCCTGCTAGTGGCTTTGGTTCTTTCAGA |
| | | GGCTGCTCGATGCCTTGTGTGTCAGAAAGAAAGATTCAGCAGTTTTGCATC |
| | | ATGGCAAAGAAGCCTGTTATTTTGGGGCTCAGCCCCTCATTTTATAGAGGA |
| | | TGAAACAGAGGGGGATGGGAGGTCACAAAGACAACTGCCCCGGGAGCAG |
| | | GTGTGGGGGAGACTTGCCCTGAGGGTCTAGACGCTCTGCACCACCGTCCT |
| | | GTCTCCCTTGCTGAAGACCACACATGCCCTTCTTTGACCAGACCCTGCCAC |
| | | CTGATAGGCCAGGACCTGGTAGGCGGGTACCCAGGTTTCATGGATGGAAC |
| | | CACATCTCCCCAAAAGTGGGGAGGTAGCTACTGGGATGCACGCCTCCCGC |
| | | CATGTGCTATAGGAGAGCAGCTGAAGCAACAGTTGGGATCAGATGTAGTCA |
| | | CAATTGAATGCATCATCACATTTATCCCTCTAAGTGGCTGGGAGAGTTGATA |
| | | TCCTCATCCCTAAGGTACAAAATGTTCCAATTTGATCAGTGGCTTTCAGGAG |
| | | CTGAGAAAGGCATGTGCTCTGAGGCAGAGCTGTTATGTCCCGCAGAGCCT |
| | | AAAAATGCTCTAAGAACATGCTCCCTGCCAAAATTCTCAATGGCTGTGACAA |
| | | GGGACAACGATCGACCAATGGGGGTGGAAGCAGACCTCCGCAGTCCAGG |
| | | GGCCAGAGCTAGGACAGAGGGGTCGGAGAAAGAGTCATTTTCCCAACACT |
| | | CCAGCTCTTGGCCAGTCCTCACACAGTCCCCTCCTGCTTCCTGCTGAGAGA |
| | | GATATCCTCATAGGTCTGGGTAAAGTCCTTCAGTCAGCTTTCATTCCCTGTC |
| | | ACCAACTTTGTCTCTGTTCTCCCTGCCCGTCTCAGGCAGCACTCCTCAGGA |
| | | AACCTCTCCAAGAGCCAGCCTCACTGCAGCGCCCACTATTGTCCCTCTGCC |
| | | TCAAGTGTCCCATCCATGCCAGGCCCCAGGCAGGCTGCAGCTTTCCCTCA |
| | | GGGCCACACCAAAGCACTTGGGCTCAGCTGTGCTGTCCCCCTCCATCACT |
| | | GAGCTCAGGGGCAGCAGGGGTGGGGTGCCAGGAGGCCCATTCACCCTTC |
| | | TCTGGCTCTGTGTTGGACCCACCTGCCCAGCCACTGCTGCTTAGAACCTAC |
| | | CCGCTGGGAAAATGAAGCCCTCCCGGAGGGGCCACCTCAACCTGAGAGCC |
| | | TCACGGATCACAGTTGTCCCCACTCAGCTCTGCCAGCCCTCAGAGACCCAT |
| | | AGATAAAAGCTGAGCTTGGCTCGCAGAGCTGGTTCCATCTTCCATTCCCAG |
| | | AGGGTTCAACTTCCTACCCCAACCACACAGGGAACCTCAAGGCTGAGCCA |
| | | GTGTGGGCTGCAGTGCAGACCAGCTTCCTGGACACGTCCTGCCACCTGAC |
| | | CCCAGGCTGGCCTCACTGCCCCTGGCACTCCTGACCCTATCCTCATTCCTC |
| | | CTGGCAGTGCGTGTTCTGCCATTCCGCTTTCCCTTAGCTGTCCTCTCACTG |
| | | TACTGTCAGCTTCTCCTTTTCCAGGTGCCCCCCAGGGGCTTTCCACATGAC |
| | | CCTGTCACCCCACAGCCCATCCAGCACCAATTCCAGCTCTCTGCCACCCTT |
| | | CAAAGGAGTGACAGTGCCCTGCTTCACCTCCCACTCACCCCTCAACCCAGA |
| | | GCAATCTGGCTCCAGTCTTGCCTCCTTCCCCCTAAGTACTCTAGTCACAGTT |
| | | CCAAATTCCTCCTGGTCATAAAGCCAAATGAAGCTTCCTGGTCCTCAGCGG |
| | | ACTTGCCACTTCAGCAGTACTGGACTCTCTCCTCCCAGAAACCTGTTTCCC |
| | | CTTGGCTCCTGGAGCCCACACTCTGCTGGAATCCTTCTGCCTCTCTGGCCT |
| | | GTAGCCTGGCCCTCTCTCCCAACCTGAGGTCCATTCTCTCCTGCTCCTCCA |
| | | CAAGATGTTGCTCCTTCCATTACTTCCTCCCTCTCAACCAAAGCTCCTTCAT |
| | | TAGCTCTTTATCTTCTGGTTTCTTCCCCTGGGCAGACGAATGGATTCAAGAG |
| | | CCTGTGGCCCAGCAGCCCAGCACTCCAGGATCTCAGCACTTCAGCATCCC |
| | | AGTACCCTAGCATCTCAATACCCCAGCACCCCAGCACCATAGTATTCCAGC |
| | | ACCCCATTGTCCAAGCATCTCAGCACTCCAGCATCCCAGCACCCCAACACT |
| | | CCAGCAGCCCAGAATCTCAGCACCCTAGCACTGCAGCATCTCAGGACCCC |
| | | AGCACTTCAGCATCCCAGCACACTAGTACTCCAGCATCTCGGCACCCCAGC |
| | | ACCTAGGCATCCCAACACCCAGCACCCCAGCACTTAAGCATCCCACCACTA |
| | | CAGTATCTCAACACTCCAGCACCCCAGCACCATAGTGTTCCAGCACCCCAG |
| | | CATCCCAACACCCCAGCACTTAAGCATCCCAACACCTCGGCATCCCAACAC |
| | | CCCAGCACTGCAGCATCTCAGCACCTTAGCATCCCAGTGCCCTAGCATCTC |
| | | AATGCTCCAGCACACCAGTACTACAGTATTCCAGCACCCCAGCACTCCAGC |
| | | ATCTCAGCACTGCAGCACTGCAGCACTCCAGCATCCCAAAATCCCAGCATC |
| | | CCAACACCCCAGCAGACCAGCAGACCAGCATCTCAGCACCGCAGCATCCA |
| | | AGGACTATCCCAGCATCCCAGCAACCCAGCACCTCAGCATCCCAACACCC |
| | | CAGCATTTCAGCATGGCAACACCCCAGTACCCCAGCACTTCAGCACCCCAG |
| | | TATCCCAGCATCTCAGCGACCCAGTATCACAAAACCTCAGCATCCTAGCAC |
| | | CCCAGCACCCCAGCACCTTAGCACCTTAGCATCCCAGCATCTCAGCGCCTC |
| | | AGCATCTTGATATTCTGGCTGAGGTCAGCGTGGTGTATCTAGTCAGGGTCC |
| | | TAACTTTCACTTCGCAGGGAAATGCTGCTGGACTGGGTCTCATGTTGGGCT |
| | | GAAGCTCTCTAGACCCCTTGAAGACAGCATAAAAGAGCTTGGAGACGCTGG |
| | | GTGTCCCCCATGGAAGAGTTCACTCTCATCCTGCTTTGACAACAGCCTTCT |
| | | CTGGGGTCCCTCACGGGCCCCTCTTTCTTACTGCAAGTTTGTCTCTGAGAA |
| | | GACTGTGATGCAGAAGTCACTCAGCTGCCTGTGGCTCCTGAAGAGCTGAA |
| | | GGTGGAGGCCTGTAGGCCTCCCTATGAGAGGCGCAGAAAAAACCATGATT |
| | | GCTAGTGGGGAGGTGCTCCCTCTACAACCCACTCCATAATCTGCCCCCGC |
| | | CCAGCTCTGAGGCCAGCCCCAGGGGAAAATGCCAGATCCCCAGGGAGGT |
| | | GTGTGAGACCTCAGGGGCTCCCTCCTCCCTTACAGCAGGCTCAGGCCCCT |
| | | GGGGGCCTCAGGGCCAAGGTCTGTGGGTAAGCTACTATCTCTCACTTGTC |
| | | CTCTAGCCACAAAAGCCAGGGAGATCTGGCAATGGACATGAGGTTCTGAA |
| | | GAAGCACATATGACTGGCTTCCTAATGCGTGGTTGTTCAGTGATTCAATAAA |
| | | CACGCATGGGCCAGGCATGGGGAAATAGACAAACATGATCCCCAACCTCT |
| | | CCCAGAGTGAACTGGGAGGGAGGAGTGTTCATCCCTCAGGATTACACCAG |
| | | AGAAACAAACCAGCAGGAGATATATATGGTTTTGGGGGGTCAAGAAAGAGG |
| | | AAAAACCTGGCAAGGCAAGTCCAAAATCATAGGACAGGCTGTCAGGAAGG |
| | | GCAGCCTGGAACCTCTCAAGCAGGAGCTGATGCTGCAGTCCACAGGCAGA |
| | | ATTTCTTCTTCCTCGGGGAAATCTCAGCTTTGTTCTTAAGGCCTTTCAACTG |
| | | ATTGGCTGAGGTCTGCCCCTTCCCCCACATTCTCCAGGATAATCTTCCTTAC |
| | | TTAAAGTCAACTATTAATCACAGCTACAAAATCCCTTCACAGCTACACATAG |
| | | ATCAGTGTTTGATTGACGAACAGCCCCTACAGCCTAGCCAAGTTGACACAT |
| | | AAAACTAACCATCACAGGGGGACAAATGATGTAAACACATCAACAAATAAAA |
| | | CAGTAACAAGTTAAGGTCTATGGAAAAAACACAGAAGGGGCAGAGAGAAAG |
| | | AAAGCAAGAAGGAGAGTCCCAGTTTGCTAGGGCTTGTGGGAAGTGGGGAG |
| | | CAGTTCTCTTTAGCTAGGATATTTGGGAAAGGCATATCTGAAGGAGTGATAT |
| | | TTGAGCTTAGATTAAAAGATGGGAAGGAGCAAGCCATGCAAAGAGCTAGGA |
| | | TGTTCCAAGCAGAGACGGAACAGCAAGTGCAAATGTCAGGAGGAATAGAA |
| | | GGAGGCTGGTGGGTGGGGTCCAGTGAGCAAGAGGAGGGCAGGCAGGAGA |
| | | GGGGATGGGGAGGTGGGCAGGCCCAGACCACCCAGGGCCCTGGAGACTA |
| | | TCCTGATCCAACAAGGGAAGCCTTGAGTCACTTCAGTGTCCATGTGGAGAA |
| | | TGGACCTCAGACTGAATGAGGGAGGCAGTAAGGAGGGCCTCTACCTCCAG |
| | | GGCTTCGCCCTGTGGACTGCGCATAGACATCTCCAACTCAGAAAGTCTGAA |
| | | CCAAACTTTCCATAGTTCCCCCAAGTCTGGGCATCCTCCTACTCAGTGAAA |
| | | GGCAGCCATCACACCTCCCTGCCCTGCTCCCGGATGCCCCAAATCCTCTT |
| | | GGTCTCCAAGTCCAGAACCTGAGACTTGTCCTTGATGTTTGTCTTTCCCTCA |
| | | CCCTTTCTGTATTCTGGGAAGATGGGTTTTTTTCCCCCAGATGAATCTGTAA |
| | | AACTTCTGTGATCACAATAAAAATTCTGGCAGTATTATTTTCTGGAACATGAC |
| | | AAAGTGATTCAAAATTATTTATCTGGAAGACTACAAAACAAGAATAGCCAGG |
| | | AAATTTCTAAAAAGAAAGAAGAAGGAGGAGGAGAAAGAAGGAGGAGGAAAA |
| | | GGAGGAGAAGAAGAAAAGAAAAAGAACCAAGAAAGGGTTCTAGCTCTACCA |
| | | AATATTAAAACATATCATGAAGCTATTTAAAACAATATGGTTGTGGATACTGA |
| | | AAAAGATGTGAATAAAGTGGAAGGAAAATAAATAGAAATGCACATGGGGAT |
| | | TGAGACTGTGAAAAAGGCAGCATCTCACATCAGTGAGGGATGTTCAACACC |
| | | TGGTGTTGGGAAAACTGGCTAGTCATTTAAACCAAACAACTGGGTCCTCTA |
| | | CCTCACTCCTGACATTAAGATACATTTAGATGATTCAAAGAGTAAGACAGAA |
| | | AAAATAACACGTGAAAACACTATCAGAAAACAACGTGGGCCAGGTGTGGTG |
| | | GGTCACGCCTGTAATCCCAGCACTTTGGGAGGCCGAGGCAGACAGATCAC |
| | | CTGAGGTGGGGAGTTCAAGACCAGCCTGACCAACATGGTGAAATCCTGTCT |
| | | CTACTAAAAATACAAAATTAGCTGAGCGTGGTGGCGCATGCCTGTAATCCC |
| | | AGCTACTCAGGAGGCCGAGGCAGGAGAATCACTTGAACCTGGGAGGCAGA |
| | | GGTTGTGGTGAGCCGAGATCACGCCATTGCACTCCAGCCTGGGCAACAAG |
| | | AGTGAAAATCCATCTAAAAAAAAAAAAAAAAGCCAAGGTGGATATTTTTATA |
| | | GTATCAGGGTAGATCAAGCTTCTCCAATCATGACATGAAACCCAGAAACCA |
| | | TAAAAGAAAAGAATGATAAAATTGCCCACGTAAAGTAAAAAGCTTGCACACA |
| | | GAAAAACACCATACAGGTTACAAGATGAGCAGCAAAATCAGAGAAAAAACA |
| | | TTGCAATTCAGGACACACAGAGGCTATTGTTCCTAATATTTAAAAATAAAAG |
| | | TAGTGGATTGTCTACAAAAAGATGAAGACAAGAATTTCAGAAAACCAAATAC |
| | | TGCATGTTTTCACTTACAAGTGGAAGCTAAACACTGAGTACACGTGTACACA |
| | | AAGAATGGAACCATAGGCCAGGCACCGTGGCTCACGCCTGTAATCCCAGT |
| | | ACTTTGCGAGGCCGAAGCGGGCGGATCACCTGAGGTGAGGAGTTCGAGAC |
| | | CATCCTGGCCAACATGGTGAAACCCAGTCTCTACTAAAAATACAAAAATTAG |
| | | CCGGGCGTGGTGGTGGGTGCCTGTAATCCCAGCTACTCGGGAGGCTGCG |
| | | GCAGTAGAATCGCTTGAACCCTGGAGGTGGACCTTGCAGTGAGCCGAGAT |
| | | CGCACCACTGCACTCCAGCCTGGGCAACAGAGTGAGACTCCATCTCAAAAA |
| | | AAAAAAAAAGGAATAGAACAATAGACACTGGGGCCTACTTGAGGGAGGAG |
| | | GGTGAGGATCAAAAACCTGCCTATCAGGTACTATGCTTATTACCTGGGTGG |
| | | TGAAATAATCTGTACACCAAACCCCAGTGACATGCAATTTACCGATGTAACA |
| | | AACCTGCCCATGTACCCGCTGAACCTAAAATAAAAGTTGGAAAAAAATATAG |
| | | AAATTTTCTTTGTAATAGCCAAAAACTGCAAACAGCCCAGGTGTCTATTAGT |
| | | AGAATGCATAAACAAACTCGGGCATGTTCATACAATGTAAAACTACTCATCA |
| | | ATAAAAAGTGATACTTCTCAGCAATGAAAAGAAACTAGCTACTGATACCAGC |
| | | TACAACATGGATGGATTTCAAGTGCTTTATGATGAGAGCAAGAAGCCAGAC |
| | | ACAAAAGTGTCTATATATATATACAGTATATATACGTATATATACACATATATA |
| | | CAGTATATATATACATATACATGTATATATATACTGTATATATACTGTATATAT |
| | | ATACACAGTATATATATACATATATACAGTGTATATATACTGTGTATATATAC |
| | | ATGTATATATACTGTGTATATATACATGTATATATACTGTGTATATATACATGT |
| | | ATATATACTGTGTATATATACATGTATATATATGTATACTGTATATATACTGTA |
| | | TATATATATACACATATATACAGTATATATATACAGTATATACTGTATATATAC |
| | | AGTATATACGTGTATATATACATATATACAGTATATATGTAAATATACATATAT |
| | | ACAGTATATATGTAAATATACATATATACATGTATATATATACACTATATATAT |
| | | ACATATATAGTGTATATATACATATATACATGTATATATTTACTATATGATTCC |
| | | ATTTATATAAAGTGCCAAAACAGTCAAAAATAATCTATGTGGAAAAAATCAAC |
| | | AAAGGGATCCCCCGGGCTGCAGGAATTCGATGGCGCGCCGACGTCGCAT |
| | | GCAGTTAGGGATAACAGGGTAATACGACCATGGCATGTCCTCTAGACTCGA |
| | | GCGGCCGCAATAAAATATCTTTATTTTCATTACATCTGTGTGTTGGTTTTTTG |
| | | TGTGAATCGTAACTAACATACGCTCTCCATCAAAACAAAACGAAACAAAACA |
| | | AACTAGCAAAATAGGCTGTCCCCAGTGCAAGTGCAGGTGCCAGAACATTTC |
| | | TCTATCGAAGGATCTGCGATCGCTCCGGTGCCCGTCAGTGGGCAGAGCGC |
| | | ACATCGCCCACAGTCCCCGAGAAGTTGGGGGGAGGGGTCGGCAATTGAAC |
| | | CGGTGCCTAGAGAAGGTGGCGCGGGGTAAACTGGGAAAGTGATGTCGTGT |
| | | ACTGGCTCCGCCTTTTTCCCGAGGGTGGGGGAGAACCGTATATAAGTGCA |
| | | GTAGTCGCCGTGAACGTTCTTTTTCGCAACGGGTTTGCCGCCAGAACACAG |
| | | CTGAAGCTTCGAGGGGCTCGCATCTCTCCTTCACGCGCCCGCCGCCCTAC |
| | | CTGAGGCCGCCATCCACGCCGGTTGAGTCGCGTTCTGCCGCCTCCCGCCT |
| | | GTGGTGCCTCCTGAACTGCGTCCGCCGTCTAGGTAAGTTTAAAGCTCAGGT |
| | | CGAGACCGGGCCTTTGTCCGGCGCTCCCTTGGAGCCTACCTAGACTCAGC |
| | | CGGCTCTCCACGCTTTGCCTGACCCTGCTTGCTCAACTCTACGTCTTTGTTT |
| | | CGTTTTCTGTTCTGCGCCGTTACAGATCCAAGCTGTGACCGGCGCCTACGT |
| | | AAGTGATATCTACTAGATTTATCAAAAAGAGTGTTGACTTGTGAGCGCTCAC |
| | | AATTGATACTTAGATTCATCGAGAGGGACACGTCGACTACTAACCTTCTTCT |
| | | CTTTCCTACAGCTGAGATCACCGGCGAAGGAGGGCCACCATGGGTCACCA |
| | | GCAGTTGGTCATCTCTTGGTTTTCCCTGGTTTTTCTGGCATCTCCCCTCGTG |
| | | GCCATATGGGAACTGAAGAAAGATGTTTATGTCGTAGAATTGGATTGGTATC |
| | | CGGATGCCCCTGGAGAAATGGTGGTCCTCACCTGTGACACCCCTGAAGAA |
| | | GATGGTATCACCTGGACCTTGGACCAGAGCAGTGAGGTCTTAGGCTCTGG |
| | | CAAAACCCTGACCATCCAAGTCAAAGAGTTTGGAGATGCTGGCCAGTACAC |
| | | CTGTCACAAAGGAGGCGAGGTTCTAAGCCATTCGCTCCTGCTGCTTCACAA |
| | | AAAGGAAGATGGAATTTGGTCCACTGATATTTTAAAGGACCAGAAAGAACC |
| | | CAAAAATAAGACCTTTCTAAGATGCGAGGCCAAGAATTATTCTGGACGTTTC |
| | | ACCTGCTGGTGGCTGACGACAATCAGTACTGATTTGACATTCAGTGTCAAA |
| | | AGCAGCAGAGGCTCTTCTGACCCCCAAGGGGTGACGTGCGGAGCTGCTAC |
| | | ACTCTCTGCAGAGAGAGTCAGAGGGGACAACAAGGAGTATGAGTACTCAG |
| | | TGGAGTGCCAGGAGGACAGTGCCTGCCCAGCTGCTGAGGAGAGTCTGCC |
| | | CATTGAGGTCATGGTGGATGCCGTTCACAAGCTCAAGTATGAAAACTACAC |
| | | CAGCAGCTTCTTCATCAGGGACATCATCAAACCTGACCCACCCAAGAACTT |
| | | GCAGCTGAAGCCATTAAAGAATTCTCGGCAGGTGGAGGTCAGCTGGGAGT |
| | | ACCCTGACACCTGGAGTACTCCACATTCCTACTTCTCCCTGACATTCTGCGT |
| | | TCAGGTCCAGGGCAAGAGCAAGAGAGAAAAGAAAGATAGAGTCTTCACGG |
| | | ACAAGACCTCAGCCACGGTCATCTGCCGCAAAAATGCCAGCATTAGCGTGC |
| | | GGGCCCAGGACCGCTACTATAGCTCATCTTGGAGCGAATGGGCATCTGTG |
| | | CCCTGCAGTGTTCCTGGAGTAGGGGTACCTGGGGTGGGCGCCAGAAACCT |
| | | CCCCGTGGCCACTCCAGACCCAGGAATGTTCCCATGCCTTCACCACTCCCA |
| | | AAACCTGCTGAGGGCCGTCAGCAACATGCTCCAGAAGGCCAGACAAACTC |
| | | TAGAATTTTACCCTTGCACTTCTGAAGAGATTGATCATGAAGATATCACAAA |
| | | AGATAAAACCAGCACAGTGGAGGCCTGTTTACCATTGGAATTAACCAAGAA |
| | | TGAGAGTTGCCTAAATTCCAGAGAGACCTCTTTCATAACTAATGGGAGTTGC |
| | | CTGGCCTCCAGAAAGACCTCTTTTATGATGGCCCTGTGCCTTAGTAGTATTT |
| | | ATGAAGACTTGAAGATGTACCAGGTGGAGTTCAAGACCATGAATGCAAAGC |
| | | TGCTGATGGATCCTAAGAGGCAGATCTTTCTAGATCAAAACATGCTGGCAG |
| | | TTATTGATGAGCTGATGCAGGCCCTGAATTTCAACAGTGAGACTGTGCCAC |
| | | AAAAATCCTCCCTTGAAGAACCGGATTTTTATAAAACTAAAATCAAGCTCTG |
| | | CATACTTCTTCATGCTTTCAGAATTCGGGCAGTGACTATTGATAGAGTGATG |
| | | AGCTATCTGAATGCTTCCTAAAAAGCGAGGTCCCTCCAAACCGTTGTCATTT |
| | | TTATAAAACTTTGAAATGAGGAAACTTTGATAGGATGTGGATTAAGAACTAG |
| | | GGAGGGGCTAGCTCGACATGATAAGATACATTGATGAGTTTGGACAAACCA |
| | | CAACTAGAATGCAGTGAAAAAAATGCTTTATTTGTGAAATTTGTGATGCTATT |
| | | GCTTTATTTGTGAAATTTGTGATGCTATTGCTTTATTTGTAACCATTATAAGC |
| | | TGCAATAAACAAGTTAACAACAACAATTGCATTCATTTTATGTTTCAGGTTCA |
| | | GGGGGAGGTGTGGGAGGTTTTTTAAAGCAAGTAAAACCTCTACAAATGTGG |
| | | TAGATCCATTTATTAGCTAGGAGTTTCAGAAAAGGGGGCCTGAGTGGCCCC |
| | | TTTTTTCAACTTAATTAACCTGCAGGGCCTGAAATAACCTCTGAAAGAGGAA |
| | | CTTGGTTAGGTACCTTCTGAGGCTGAAAGAACCAGCTGTGGAATGTGTGTC |
| | | AGTTAGGGTGTGGAAAGTCCCCAGGCTCCCCAGCAGGCAGAAGTATGCAA |
| | | AGCATGCATCTCAATTAGTCAGCAACCAGGTGTGGAAAGTCCCCAGGCTCC |
| | | CCAGCAGGCAGAAGTATGCAAAGCATGCATCTCAATTAGTCAGCAACCATA |
| | | GTCCCACTAGTTTCATCACCACCGCCACCCCCCCGCCCCCCCGCCATCTG |
| | | AAAGGGTTCTAGGGGATTTGCAACCTCTCTCGTGTGTTTCTTCTTTCCGAGA |
| | | AGCGCCGCCACACGAGAAAGCTGGCCGCGAAAGTCGTGCTGGAATCACTT |
| | | CCAACGAAACCCCAGGCATAGATGGGAAAGGGTGAAGAACACGTTGTCAT |
| | | GGCTACCGTTTCCCCGGTCACGGAATAAACGCTCTCTAGGATCCGGAAGTA |
| | | GTTCCGCCGCGACCTCTCTAAAAGGATGGATGTGTTCTCTGCTTACATTCAT |
| | | TGGACGTTTTCCCTTAGAGGCCAAGGCCGCCCAGGCAAAGGGGCGGTCCC |
| | | ACGCGTGAGGGGCCCGCGGAGCCATTTGATTGGAGAAAAGCTGCAAACCC |
| | | TGACCAATCGGAAGGAGCCACGCTTCGGGCATCGGTCACCGCACCTGGAC |
| | | AGCTCCGATTGGTGGACTTCCGCCCCCCCTCACGAATCCTCATTGGGTGC |
| | | CGTGGGTGCGTGGTGCGGCGCGATTGGTGGGTTCATGTTTCCCGTCCCCC |
| | | GCCCGCGAGAAGTGGGGGTGAAAAGCGGCCCGACCTGCTTGGGGTGTAG |
| | | TGGGCGGACCGCGCGGCTGGAGGTGTGAGGATCCGAACCCAGGGGTGGG |
| | | GGGTGGAGGCGGCTCCTGCGATCGAAGGGGACTTGAGACTCACCGGTCG |
| | | CACGTCATGAATCTAGAACCATGGCTTCGTACCCCGGCCATCAGCACGCGT |
| | | CTGCGTTCGACCAGGCTGCGCGTTCTCGCGGCCATAGCAACCGACGTACG |
| | | GCGTTGCGCCCTCGCCGGCAGCAAGAAGCCACGGAAGTCCGCCCGGAGC |
| | | AGAAAATGCCCACGCTACTGCGGGTTTATATAGACGGTCCCCACGGGATG |
| | | GGGAAAACCACCACCACGCAACTGCTGGTGGCCCTGGGTTCGCGCGACGA |
| | | TATCGTCTACGTACCCGAGCCGATGACTTACTGGCGGGTGCTGGGGGCTT |
| | | CCGAGACAATCGCGAACATCTACACCACACAACACCGCCTTGACCAGGGT |
| | | GAGATATCGGCCGGGGACGCGGCGGTGGTAATGACAAGCGCCCAGATAA |
| | | CAATGGGCATGCCTTATGCCGTGACCGACGCCGTTCTGGCTCCTCATATCG |
| | | GGGGGGAGGCTGGGAGCTCACATGCCCCGCCCCCGGCCCTCACCCTCAT |
| | | CTTCGACCGCCATCCCATCGCCGCCCTCCTGTGCTACCCGGCCGCGCGAT |
| | | ACCTTATGGGCAGCATGACCCCCCAGGCCGTGCTGGCGTTCGTGGCCCTC |
| | | ATCCCGCCGACCTTGCCCGGCACAAACATCGTGTTGGGGGCCCTTCCGGA |
| | | GGACAGACACATCGACCGCCTGGCCAAACGCCAGCGCCCCGGCGAGCGG |
| | | CTTGACCTGGCTATGCTGGCCGCGATTCGCCGCGTTTACGGGCTGCTTGC |
| | | CAATACGGTGCGGTATCTGCAGGGCGGCGGGTCGTGGCGGGAGGATTGG |
| | | GGACAGCTTTCGGGGACGGCCGTGCCGCCCCAGGGTGCCGAGCCCCAGA |
| | | GCAACGCGGGCCCACGACCCCATATCGGGGACACGTTATTTACCCTGTTTC |
| | | GGGCCCCCGAGTTGCTGGCCCCCAACGGCGACCTGTACAACGTGTTTGCC |
| | | TGGGCCTTGGACGTCTTGGCCAAACGCCTCCGTCCCATGCACGTCTTTATC |
| | | CTGGATTACGACCAATCGCCCGCCGGCTGCCGGGACGCCCTGCTGCAACT |
| | | TACCTCCGGGATGATCCAGACCCACGTCACCACCCCAGGCTCCATACCGA |
| | | CGATCTGCGACCTGGCGCGCACGTTTGCCCGGGAGATGGGGGAGGCTAA |
| | | CTGAGTATACCCTAGGATTATCCCTAATACCTGCCACCCCACTCTTAATCAG |
| | | TGGTGGAAGAACGGTCTCAGAACTGTTTGTTTCAATTGGCCATTTAAGTTTA |
| | | GTAGTAAAAGACTGGTTAATGATAACAATGCATCGTAAAACCTTCAGAAGGA |
| | | AAGGAGAATGTTTTGTGGACCACTTTGGTTTTCTTTTTTGCGTGTGGCAGTT |
| | | TTAAGTTATTAGTTTTTAAAATCAGTACTTTTTAATGGAAACAACTTGACCAA |
| | | AAATTTGTCACAGAATTTTGAGACCCATTAAAAAAGTTAAATGAGAAACCTG |
| | | TGTGTTCCTTTGGTCAACACCGAGACATTTAGGTGAAAGACATCTAATTCTG |
| | | GTTTTACGAATCTGGAAACTTCTTGAAAATGTAATTCTTGAGTTAACACTTCT |
| | | GGGTGGAGAATAGGGTTGTTTTCCCCCCACATAATTGGAAGGGGAAGGAAT |
| | | ATCATTTAAAGCTATGGGAGGGTTTCTTTGATTACAACACTGGAGAGAAATG |
| | | CAGCATGTTGCTGATTGCCTGTCACTAAAACAGGCCAAAAACTGAGTCCTT |
| | | GGGTTGCATAGAAAGCTGCCTGCAGGCGTTACATAACTTACGGTAAATGGC |
| | | CCGCCTGGCTGACCGCCCAACGACCCCCGCCCATTGACGTCAATAATGAC |
| | | GTATGTTCCCATAGTAACGCCAATAGGGACTTTCCATTGACGTCAATGGGT |
| | | GGAGTATTTACGGTAAACTGCCCACTTGGCAGTACATCAAGTGTATCATATG |
| | | CCAAGTACGCCCCCTATTGACGTCAATGACGGTAAATGGCCCGCCTGGCAT |
| | | TATGCCCAGTACATGACCTTATGGGACTTTCCTACTTGGCAGTACATCTACG |
| | | TATTAGTCATCGCTATTACCATGATGATGCGGTTTTGGCAGTACATCAATGG |
| | | GCGTGGATAGCGGTTTGACTCACGGGGATTTCCAAGTCTCCACCCCATTGA |
| | | CGTCAATGGGAGTTTGTTTTGACTAGTTACCGGCGGAAACGGTCTCGGGTT |
| | | GAGAGGTCACCCGAGGGACAGGCAGCTGCTGAACCAATAGGACCGGCGC |
| | | ACAGGGCGGATGCTGCCCCTCATTGGCGGCCGTTGAGAGTGACCAAGAGC |
| | | CAATGAGTCAGCCCGGGGGGCGTAGCAGTGACGTAAGTTGCGGAGGAGG |
| | | CCGCTTCGAATCGGCAGCGGCCAGCTTGGTGGCATGGACCAATCAGCGTC |
| | | CTCCAACGAGGAGCGCCTTCGCCAATCGGAGGCCTCCACGACGGGGCTG |
| | | GGGGGAGGGTATATAAGCCGAGTCGGCGGCGGCGCGCTCCACACGGGCC |
| | | GAGACCACAGCGACGGGAGCGTCTGCCTCTGCGGGGCCGAGAGGTAAGC |
| | | GCCGCGGCCTGCCCTTTCCAGGCCAACTCGGAGCCCGTCTCGTGGCTCCG |
| | | CCTGATCGGGGGCTCCTGTCGCCCTCAGATCGGTCGGAACGCCGTCGCG |
| | | CTCCGGGACTACAAGCCTGTTGCTGGGCCCGGAGACTGCCGAAGGACCG |
| | | CTGAGCACTGTCCTCAGCGCCGGCACCATGGATTGGATCTGGCGGATCCT |
| | | GTTCCTTGTGGGAGCTGCCACAGGCGCCCATTCTGAAGTTCAGCTGGTTCA |
| | | GTCTGGCGCCGAAGTGAAGAAACCTGGCGCCTCTGTGAAGGTGTCCTGCA |
| | | AAGCTTCTGGCGGCACCTTCAGCAGCTACGCCATCTCTTGGGTTCGACAG |
| | | GCCCCTGGACAAGGCCTGGAATGGATGGGCAGAATCATCCCCATCCTGGG |
| | | AATCGCCAACTACGCCCAGAAATTCCAGGGCAGAGTGACCATCACCGCCG |
| | | ACAAGAGCACAAGCACCGCCTACATGGAACTGAGCAGCCTGAGAAGCGAG |
| | | GACACCGCCGTGTACTACTGTGCCAGAAGCGGCCACGGCTACAGCTACGG |
| | | CGCCTTTGATTATTGGGGCCAGGGCACCCTGGTCACCGTTTCTAGCGGAG |
| | | GCGGAGGTAGTGGTGGCGGAGGTTCAGGCGGCGGAGGATCTCAATCTGT |
| | | GCTGACACAGCCTCCAAGCGTGTCAGGTGCTCCTGGCCAGAGAGTGACAA |
| | | TCAGCTGTACAGGCAGCAGCAGCAACATCGGAGCCGGCTATGACGTGCAC |
| | | TGGTATCAGCAGCTGCCTGGCACAGCCCCTAAACTGCTGATCTACGGCAAC |
| | | AGCAACAGACCCAGCGGCGTGCCCGATAGATTTTCCGGCTCTAAGAGCGG |
| | | CACAAGCGCCAGCCTGGCTATTACTGGACTGCAGGCCGAGGACGAGGCC |
| | | GACTACTACTGTCAGAGCTACGACAGCAGCCTGTCCGGCAGCTACGTTGT |
| | | GTTTGGCGGCGGAACAAAGCTGACCGTGCTGGAAGCCAAGAGCTGCGACA |
| | | AGACCCACACCTGTCCTCCATGTCCTGCTCCAGAACTGCTCGGCGGACCTT |
| | | CCGTGTTCCTGTTTCCTCCAAAGCCTAAGGACACCCTGATGATCAGCAGAA |
| | | CCCCTGAAGTGACCTGCGTGGTGGTGGATGTGTCCCACGAGGACCCAGAA |
| | | GTGAAGTTCAATTGGTACGTGGACGGCGTGGAAGTGCACAACGCCAAGAC |
| | | CAAGCCTAGAGAGGAACAGTACAACAGCACCTACAGAGTGGTGTCCGTGC |
| | | TGACAGTGCTGCACCAGGATTGGCTGAACGGCAAAGAGTACAAGTGCAAG |
| | | GTGTCCAACAAGGCCCTGCCTGCTCCTATCGAGAAAACCATCAGCAAGGC |
| | | CAAGGGCCAGCCTAGGGAACCCCAGGTTTACACACTGCCACCTAGCAGGG |
| | | ACGAGCTGACCAAGAATCAGGTGTCCCTGACCTGCCTGGTCAAGGGCTTC |
| | | TACCCTTCCGATATCGCCGTGGAATGGGAGAGCAATGGCCAGCCAGAGAA |
| | | CAACTACAAGACAACCCCTCCTGTGCTGGACAGCGACGGCTCATTCTTCCT |
| | | GTACTCCAAGCTGACTGTGGACAAGAGCCGGTGGCAGCAGGGCAATGTGT |
| | | TCAGCTGTAGCGTGATGCACGAGGCCCTGCACAACCACTACACACAGAAG |
| | | TCCCTGTCTCTGAGCCCCGGAAAAGGTGGCGGTGGCTCTTACCCTTACGA |
| | | CGTGCCAGATTACGCCGGCTATCCCTACGATGTGCCTGACTATGCTGGCTA |
| | | CCCCTATGACGTCCCCGACTACGCTTAACTAGCTACGGAATTCCGGCTAGC |
| | | TGGCCAGACATGATAAGATACATTGATGAGTTTGGACAAACCACAACTAGA |
| | | ATGCAGTGAAAAAAATGCTTTATTTGTGAAATTTGTGATGCTATTGCTTTATT |
| | | TGTAACCATTATAAGCTGCAATAAACAAGTTAACAACAACAATTGCATTCATT |
| | | TTATGTTTCAGGTTCAGGGGGAGGTGTGGGAGGTTTTTTAAAGCAAGTAAA |
| | | ACCTCTACAAATGTGGTATGGAAATGTTAATTAACTAGCCATGACCAAAATC |
| | | CCTTAACGTGAGTTTTCGTTCCACTGAGCGTCAGACCCCGTAGAAAAGATC |
| | | AAAGGATCTTCTTGAGATCCTTTTTTTCTGCGCGTAATCTGCTGCTTGCAAA |
| | | CAAAAAAACCACCGCTACCAGCGGTGGTTTGTTTGCCGGATCAAGAGCTAC |
| | | CAACTCTTTTTCCGAAGGTAACTGGCTTCAGCAGAGCGCAGATACCAAATA |
| | | CTGTTCTTCTAGTGTAGCCGTAGTTAGGCCACCACTTCAAGAACTCTGTAG |
| | | CACCGCCTACATACCTCGCTCTGCTAATCCTGTTACCAGTGGCTGCTGCCA |
| | | GTGGCGATAAGTCGTGTCTTACCGGGTTGGACTCAAGACGATAGTTACCGG |
| | | ATAAGGCGCAGCGGTCGGGCTGAACGGGGGGTTCGTGCACACAGCCCAG |
| | | CTTGGAGCGAACGACCTACACCGAACTGAGATACCTACAGCGTGAGCTATG |
| | | AGAAAGCGCCACGCTTCCCGAAGGGAGAAAGGCGGACAGGTATCCGGTAA |
| | | GCGGCAGGGTCGGAACAGGAGAGCGCACGAGGGAGCTTCCAGGGGGAAA |
| | | CGCCTGGTATCTTTATAGTCCTGTCGGGTTTCGCCACCTCTGACTTGAGCG |
| | | TCGATTTTTGTGATGCTCGTCAGGGGGGCGGAGCCTATGGAAAAACGCCA |
| | | GCAACGCGGCCTTTTTACGGTTCCTGGCCTTTTGCTGGCCTTTTGCTCAGG |
| | | GTTCGAAATCGATAAGCTTGGATCCGGAGAGCTCCCAACGCGTCGGCTAG |
| | | CTAGTAGGGATAACAGGGTAATAAGCGTCGACGGCGCGCCCCTAGGGGCC |
| | | GGCCTTAATTAAATCAAGCTTATCGATACCGTCGAACCTCGAGGGGGGGCA |
| | | TCACTCCGCCCTAAAACCTACGTCACCCGCCCCGTTCCCACGCCCCGCGC |
| | | CACGTCACAAACTCCACCCCCTCATTATCATATTGGCTTCAATCCAAAATAA |
| | | GGTATATTATTGATGATGTTT |
| 51 | ICOSTAT | TAACATCATCAATTATACCTTCCATTTTGGATTGAAGCCAATATGATAATGAG |
| | | GGGGTGGAGTTTGTGACGTGGCGCGGGGCGTGGGAACGGGGCGGGTGA |
| | | CGTAGTAGTGTGGCGGAAGTGTGATGTTGCAAGTGTGGCGGAACACATGT |
| | | AAGCGACGGATGTGGCAAAAGTGACGTTTTTGGTGTGCGCCGGTGTACAC |
| | | AGGAAGTGACAATTTTCGCGCGGTTTTAGGCGGATGTTGTAGTAAATTTGG |
| | | GCGTAACCGAGTAAGATTTGGCCATTTTCGCGGGAAAACTGAATAAGAGGA |
| | | AGTGAAATCTGAATAATTTTGTGTTACTCATAGCGCGTAATATTTGTCTAGG |
| | | GCCGCGGGGACTTTGACCGTTTACGTGGAGACTCGCCCAGGTGTTTTTCTC |
| | | AGGTGTTTTCCGCGTACGTCGGCGGCTCGTGGCTCTTCCGGGAAAAGGAT |
| | | TCTCGGAAAGTGGTTCGAGTACGTCGGCGGCTCGTGGCTCTTCCGGGAAA |
| | | AGGATTCTCGGAAAGTGGTTCGAAGTACGTCGACCACAAACCCCGCCCAG |
| | | CGTCTTGTCATTGGCGTCGACGCTGTACGGGGTCAAAGTTGGCGTTTTATT |
| | | ATTATAGTCAGCTGACGTGTAGTGTATTTATACCCGGTGAGTTCCTCAAGAG |
| | | GCCACTCTTGAGTGCCAGCGAGTAGAGTTTTCTCCTCCGAGCCGCTCCGA |
| | | CACCGGGACTGAAAATGAGACATATTATCTGCCACGGAGGTGTTATTACCG |
| | | AAGAAATGGCCGCCAGTCTTTTGGACCAGCTGATCGAAGAGGTACTGGCT |
| | | GATAATCTTCCACCTCCTAGCCATTTTGAACCACCTACCCTTCACGAACTGT |
| | | ATGATTTAGACGTGACGGCCCCCGAAGATCCCAACGAGGAGGCGGTTTCG |
| | | CAGATTTTTCCCGACTCTGTAATGTTGGCGGTGCAGGAAGGGATTGACTTA |
| | | CTCACTTTTCCGCCGGCGCCCGGTTCTCCGGAGCCGCCTCACCTTTCCCG |
| | | GCAGCCCGAGCAGCCGGAGCAGAGAGCCTTGGGTCCGGTTTCTATGCCAA |
| | | ACCTTGTACCGGAGGTGATCGATCCACCCAGTGACGACGAGGATGAAGAG |
| | | GGTGAGGAGTTTGTGTTAGATTATGTGGAGCACCCCGGGCACGGTTGCAG |
| | | GTCTTGTCATTATCACCGGAGGAATACGGGGGACCCAGATATTATGTGTTC |
| | | GCTTTGCTATATGAGGACCTGTGGCATGTTTGTCTACAGTAAGTGAAAATTA |
| | | TGGGCAGTGGGTGATAGAGTGGTGGGTTTGGTGTGGTAATTTTTTTTTTAAT |
| | | TTTTACAGTTTTGTGGTTTAAAGAATTTTGTATTGTGATTTTTTTAAAAGGTCC |
| | | TGTGTCTGAACCTGAGCCTGAGCCCGAGCCAGAACCGGAGCCTGCAAGAC |
| | | CTACCCGCCGTCCTAAAATGGCGCCTGCTATCCTGAGACGCCCGACATCA |
| | | CCTGTGTCTAGAGAATGCAATAGTAGTACGGATAGCTGTGACTCCGGTCCT |
| | | TCTAACACACCTCCTGAGATACACCCGGTGGTCCCGCTGTGCCCCATTAAA |
| | | CCAGTTGCCGTGAGAGTTGGTGGGCGTCGCCAGGCTGTGGAATGTATCGA |
| | | GGACTTGCTTAACGAGCCTGGGCAACCTTTGGACTTGAGCTGTAAACGCCC |
| | | CAGGCCATAAGGTGTAAACCTGTGATTGCGTGTGTGGTTAACGCCTTTGTT |
| | | TGCTGAATGAGTTGATGTAAGTTTAATAAAGGGTGAGATAATGTTTAACTTG |
| | | CATGGCGTGTTAAATGGGGCGGGGCTTAAAGGGTATATAATGCGCCGTGG |
| | | GCTAATCTTGGTTACATCTGACCTCATGGAGGCTTGGGAGTGTTTGGAAGA |
| | | TTTTTCTGCTGTGCGTAACTTGCTGGAACAGAGCTCTAACAGTACCTCTTGG |
| | | TTTTGGAGGTTTCTGTGGGGCTCATCCCAGGCAAAGTTAGTCTGCAGAATT |
| | | AAGGAGGATTACAAGTGGGAATTTGAAGAGCTTTTGAAATCCTGTGGTGAG |
| | | CTGTTTGATTCTTTGAATCTGGGTCACCAGGCGCTTTTCCAAGAGAAGGTC |
| | | ATCAAGACTTTGGATTTTTCCACACCGGGGCGCGCTGCGGCTGCTGTTGCT |
| | | TTTTTGAGTTTTATAAAGGATAAATGGAGCGAAGAAACCCATCTGAGCGGG |
| | | GGGTACCTGCTGGATTTTCTGGCCATGCATCTGTGGAGAGCGGTTGTGAG |
| | | ACACAAGAATCGCCTGCTACTGTTGTCTTCCGTCCGCCCGGCGATAATACC |
| | | GACGGAGGAGCAGCAGCAGCAGCAGGAGGAAGCCAGGCGGCGGCGGCA |
| | | GGAGCAGAGCCCATGGAACCCGAGAGCCGGCCTGGACCCTCGGGAATGA |
| | | ATGTTGTACAGGTGGCTGAACTGTATCCAGAACTGAGACGCATTTTGACAA |
| | | TTACAGAGGATGGGCAGGGGCTAAAGGGGGTAAAGAGGGAGCGGGGGGC |
| | | TTGTGAGGCTACAGAGGAGGCTAGGAATCTAGCTTTTAGCTTAATGACCAG |
| | | ACACCGTCCTGAGTGTATTACTTTTCAACAGATCAAGGATAATTGCGCTAAT |
| | | GAGCTTGATCTGCTGGCGCAGAAGTATTCCATAGAGCAGCTGACCACTTAC |
| | | TGGCTGCAGCCAGGGGATGATTTTGAGGAGGCTATTAGGGTATATGCAAAG |
| | | GTGGCACTTAGGCCAGATTGCAAGTACAAGATCAGCAAACTTGTAAATATC |
| | | AGGAATTGTTGCTACATTTCTGGGAACGGGGCCGAGGTGGAGATAGATAC |
| | | GGAGGATAGGGTGGCCTTTAGATGTAGCATGATAAATATGTGGCCGGGGG |
| | | TGCTTGGCATGGACGGGGTGGTTATTATGAATGTAAGGTTTACTGGCCCCA |
| | | ATTTTAGCGGTACGGTTTTCCTGGCCAATACCAACCTTATCCTACACGGTGT |
| | | AAGCTTCTATGGGTTTAACAATACCTGTGTGGAAGCCTGGACCGATGTAAG |
| | | GGTTCGGGGCTGTGCCTTTTACTGCTGCTGGAAGGGGGTGGTGTGTCGCC |
| | | CCAAAAGCAGGGCTTCAATTAAGAAATGCCTCTTTGAAAGGTGTACCTTGG |
| | | GTATCCTGTCTGAGGGTAACTCCAGGGTGCGCCACAATGTGGCCTCCGAC |
| | | TGTGGTTGCTTCATGCTAGTGAAAAGCGTGGCTGTGATTAAGCATAACATG |
| | | GTATGTGGCAACTGCGAGGACAGGGCCTCTCAGATGCTGACCTGCTCGGA |
| | | CGGCAACTGTCACCTGCTGAAGACCATTCACGTAGCCAGCCACTCTCGCAA |
| | | GGCCTGGCCAGTGTTTGAGCATAACATACTGACCCGCTGTTCCTTGCATTT |
| | | GGGTAACAGGAGGGGGGTGTTCCTACCTTACCAATGCAATTTGAGTCACAC |
| | | TAAGATATTGCTTGAGCCCGAGAGCATGTCCAAGGTGAACCTGAACGGGGT |
| | | GTTTGACATGACCATGAAGATCTGGAAGGTGCTGAGGTACGATGAGACCC |
| | | GCACCAGGTGCAGACCCTGCGAGTGTGGCGGTAAACATATTAGGAACCAG |
| | | CCTGTGATGCTGGATGTGACCGAGGAGCTGAGGCCCGATCACTTGGTGCT |
| | | GGCCTGCACCCGCGCTGAGTTTGGCTCTAGCGATGAAGATACAGATTGAG |
| | | GTACTGAAATGTGTGGGCGTGGCTTAAGGGTGGGAAAGAATATATAAGGTG |
| | | GGGGTCTTATGTAGTTTGTATCTGTTTTGCAGCAGCCGCCGCCGCCATGA |
| | | GCACCAACTCGTTTGATGGAAGCATTGTGAGCTCATATTTGACAACGCGCA |
| | | TGCCCCCATGGGCCGGGGTGCGTCAGAATGTGATGGGCTCCAGCATTGAT |
| | | GGTCGCCCCGTCCTGCCCGCAAACTCTACTACCTTGACCTACGAGACCGT |
| | | GTCTGGAACGCCGTTGGAGACTGCAGCCTCCGCCGCCGCTTCAGCCGCTG |
| | | CAGCCACCGCCCGCGGGATTGTGACTGACTTTGCTTTCCTGAGCCCGCTT |
| | | GCAAGCAGTGCAGCTTCCCGTTCATCCGCCCGCGATGACAAGTTGACGGC |
| | | TCTTTTGGCACAATTGGATTCTTTGACCCGGGAACTTAATGTCGTTTCTCAG |
| | | CAGCTGTTGGATCTGCGCCAGCAGGTTTCTGCCCTGAAGGCTTCCTCCCCT |
| | | CCCAATGCGGTTTAAAACATAAATAAAAAACCAGACTCTGTTTGGATTTGGA |
| | | TCAAGCAAGTGTCTTGCTGTCTTTATTTAGGGGTTTTGCGCGCGCGGTAGG |
| | | CCCGGGACCAGCGGTCTCGGTCGTTGAGGGTCCTGTGTATTTTTTCCAGG |
| | | ACGTGGTAAAGGTGACTCTGGATGTTCAGATACATGGGCATAAGCCCGTCT |
| | | CTGGGGTGGAGGTAGCACCACTGCAGAGCTTCATGCTGCGGGGTGGTGTT |
| | | GTAGATGATCCAGTCGTAGCAGGAGCGCTGGGCGTGGTGCCTAAAAATGT |
| | | CTTTCAGTAGCAAGCTGATTGCCAGGGGCAGGCCCTTGGTGTAAGTGTTTA |
| | | CAAAGCGGTTAAGCTGGGATGGGTGCATACGTGGGGATATGAGATGCATC |
| | | TTGGACTGTATTTTTAGGTTGGCTATGTTCCCAGCCATATCCCTCCGGGGAT |
| | | TCATGTTGTGCAGAACCACCAGCACAGTGTATCCGGTGCACTTGGGAAATT |
| | | TGTCATGTAGCTTAGAAGGAAATGCGTGGAAGAACTTGGAGACGCCCTTGT |
| | | GACCTCCAAGATTTTCCATGCATTCGTCCATAATGATGGCAATGGGCCCAC |
| | | GGGCGGCGGCCTGGGCGAAGATATTTCTGGGATCACTAACGTCATAGTTG |
| | | TGTTCCAGGATGAGATCGTCATAGGCCATTTTTACAAAGCGCGGGCGGAG |
| | | GGTGCCAGACTGCGGTATAATGGTTCCATCCGGCCCAGGGGCGTAGTTAC |
| | | CCTCACAGATTTGCATTTCCCACGCTTTGAGTTCAGATGGGGGGATCATGT |
| | | CTACCTGCGGGGCGATGAAGAAAACGGTTTCCGGGGTAGGGGAGATCAGC |
| | | TGGGAAGAAAGCAGGTTCCTGAGCAGCTGCGACTTACCGCAGCCGGTGGG |
| | | CCCGTAAATCACACCTATTACCGGGTGCAACTGGTAGTTAAGAGAGCTGCA |
| | | GCTGCCGTCATCCCTGAGCAGGGGGGCCACTTCGTTAAGCATGTCCCTGA |
| | | CTCGCATGTTTTCCCTGACCAAATCCGCCAGAAGGCGCTCGCCGCCCAGC |
| | | GATAGCAGTTCTTGCAAGGAAGCAAAGTTTTTCAACGGTTTGAGACCGTCC |
| | | GCCGTAGGCATGCTTTTGAGCGTTTGACCAAGCAGTTCCAGGCGGTCCCA |
| | | CAGCTCGGTCACCTGCTCTACGGCATCTCGATCCAGCATATCTCCTCGTTT |
| | | CGCGGGTTGGGGCGGCTTTCGCTGTACGGCAGTAGTCGGTGCTCGTCCAG |
| | | ACGGGCCAGGGTCATGTCTTTCCACGGGCGCAGGGTCCTCGTCAGCGTAG |
| | | TCTGGGTCACGGTGAAGGGGTGCGCTCCGGGCTGCGCGCTGGCCAGGGT |
| | | GCGCTTGAGGCTGGTCCTGCTGGTGCTGAAGCGCTGCCGGTCTTCGCCCT |
| | | GCGCGTCGGCCAGGTAGCATTTGACCATGGTGTCATAGTCCAGCCCCTCC |
| | | GCGGCGTGGCCCTTGGCGCGCAGCTTGCCCTTGGAGGAGGCGCCGCACG |
| | | AGGGGCAGTGCAGACTTTTGAGGGCGTAGAGCTTGGGCGCGAGAAATACC |
| | | GATTCCGGGGAGTAGGCATCCGCGCCGCAGGCCCCGCAGACGGTCTCGC |
| | | ATTCCACGAGCCAGGTGAGCTCTGGCCGTTCGGGGTCAAAAACCAGGTTT |
| | | CCCCCATGCTTTTTGATGCGTTTCTTACCTCTGGTTTCCATGAGCCGGTGTC |
| | | CACGCTCGGTGACGAAAAGGCTGTCCGTGTCCCCGTATACAGACTNNNGT |
| | | TTTGAGAGGCCTGTCCTCGAGCGGTGTTCCGCGGTCCTCCTCGTATAGAAA |
| | | CTCGGACCACTCTGAGACAAAGGCTCGCGTCCAGGCCAGCACGAAGGAGG |
| | | CTAAGTGGGAGGGGTAGCGGTCGTTGTCCACTAGGGGGTCCACTCGCTCC |
| | | AGGGTGTGAAGACACATGTCGCCCTCTTCGGCATCAAGGAAGGTGATTGG |
| | | TTTGTAGGTGTAGGCCACGTGACCGGGTGTTCCTGAAGGGGGGCTATAAA |
| | | AGGGGGTGGGGGCGCGTTCGTCCTCACTCTCTTCCGCATCGCTGTCTGCG |
| | | AGGGCCAGCTGTTGGGGTGAGTACTCCCTCTGAAAAGCGGGCATGACTTC |
| | | TGCGCTAAGATTGTCAGTTTCCAAAAACGAGGAGGATTTGATATTCACCTG |
| | | GCCCGCGGTGATGCCTTTGAGGGTGGCCGCATCCATCTGGTCAGAAAAGA |
| | | CAATCTTTTTGTTGTCAAGCTTGGTGGCAAACGACCCGTAGAGGGCGTTGG |
| | | ACAGCAACTTGGCGATGGAGCGCAGGGTTTGGTTTTTGTCGCGATCGGCG |
| | | CGCTCCTTGGCCGCGATGTTTAGCTGCACGTATTCGCGCGCAACGCACCG |
| | | CCATTCGGGAAAGACGGTGGTGCGCTCGTCGGGCACCAGGTGCACGCGC |
| | | CAACCGCGGTTGTGCAGGGTGACAAGGTCAACGCTGGTGGCTACCTCTCC |
| | | GCGTAGGCGCTCGTTGGTCCAGCAGAGGCGGCCGCCCTTGCGCGAGCAG |
| | | AATGGCGGTAGGGGGTCTAGCTGCGTCTCGTCCGGGGGGTCTGCGTCCA |
| | | CGGTAAAGACCCCGGGCAGCAGGCGCGCGTCGAAGTAGTCTATCTTGCAT |
| | | CCTTGCAAGTCTAGCGCCTGCTGCCATGCGCGGGCGGCAAGCGCGCGCT |
| | | CGTATGGGTTGAGTGGGGGACCCCATGGCATGGGGTGGGTGAGCGCGGA |
| | | GGCGTACATGCCGCAAATGTCGTAAACGTAGAGGGGCTCTCTGAGTATTCC |
| | | AAGATATGTAGGGTAGCATCTTCCACCGCGGATGCTGGCGCGCACGTAAT |
| | | CGTATAGTTCGTGCGAGGGAGCGAGGAGGTCGGGACCGAGGTTGCTACG |
| | | GGCGGGCTGCTCTGCTCGGAAGACTATCTGCCTGAAGATGGCATGTGAGT |
| | | TGGATGATATGGTTGGACGCTGGAAGACGTTGAAGCTGGCGTCTGTGAGA |
| | | CCTACCGCGTCACGCACGAAGGAGGCGTAGGAGTCGCGCAGCTTGTTGAC |
| | | CAGCTCGGCGGTGACCTGCACGTCTAGGGCGCAGTAGTCCAGGGTTTCCT |
| | | TGATGATGTCATACTTATCCTGTCCCTTTTTTTTCCACAGCTCGCGGTTGAG |
| | | GACAAACTCTTCGCGGTCTTTCCAGTACTCTTGGATCGGAAACCCGTCGGC |
| | | CTCCGAACGGTAAGAGCCTAGCATGTAGAACTGGTTGACGGCCTGGTAGG |
| | | CGCAGCATCCCTTTTCTACGGGTAGCGCGTATGCCTGCGCGGCCTTCCGG |
| | | AGCGAGGTGTGGGTGAGCGCAAAGGTGTCCCTGACCATGACTTTGAGGTA |
| | | CTGGTATTTGAAGTCAGTGTCGTCGCATCCGCCCTGCTCCCAGAGCAAAAA |
| | | GTCCGTGCGCTTTTTGGAACGCGGATTTGGCAGGGCGAAGGTGACATCGT |
| | | TGAAGAGTATCTTTCCCGCGCGAGGCATAAAGTTGCGTGTGATGCGGAAG |
| | | GGTCCCGGCACCTCGGAACGGTTGTTAATTACCTGGGCGGCGAGCACGAT |
| | | CTCGTCAAAGCCGTTGATGTTGTGGCCCACAATGTAAAGTTCCAAGAAGCG |
| | | CGGGATGCCCTTGATGGAAGGCAATTTTTTAAGTTCCTCGTAGGTGAGCTC |
| | | TTCAGGGGAGCTGAGCCCGTGCTCTGAAAGGGCCCAGTCTGCAAGATGAG |
| | | GGTTGGAAGCGACGAATGAGCTCCACAGGTCACGGGCCATTAGCATTTGC |
| | | AGGTGGTCGCGAAAGGTCCTAAACTGGCGACCTATGGCCATTTTTTCTGGG |
| | | GTGATGCAGTAGAAGGTAAGCGGGTCTTGTTCCCAGCGGTCCCATCCAAG |
| | | GTTCGCGGCTAGGTCTCGCGCGGCAGTCACTAGAGGCTCATCTCCGCCGA |
| | | ACTTCATGACCAGCATGAAGGGCACGAGCTGCTTCCCAAAGGCCCCCATC |
| | | CAAGTATAGGTCTCTACATCGTAGGTGACAAAGAGACGCTCGGTGCGAGG |
| | | ATGCGAGCCGATCGGGAAGAACTGGATCTCCCGCCACCAATTGGAGGAGT |
| | | GGCTATTGATGTGGTGAAAGTAGAAGTCCCTGCGACGGGCCGAACACTCG |
| | | TGCTGGCTTTTGTAAAAACGTGCGCAGTACTGGCAGCGGTGCACGGGCTG |
| | | TACATCCTGCACGAGGTTGACCTGACGACCGCGCACAAGGAAGCAGAGTG |
| | | GGAATTTGAGCCCCTCGCCTGGCGGGTTTGGCTGGTGGTCTTCTACTTCG |
| | | GCTGCTTGTCCTTGACCGTCTGGCTGCTCGAGGGGAGTTACGGTGGATCG |
| | | GACCACCACGCCGCGCGAGCCCAAAGTCCAGATGTCCGCGCGCGGCGGT |
| | | CGGAGCTTGATGACAACATCGCGCAGATGGGAGCTGTCCATGGTCTGGAG |
| | | CTCCCGCGGCGTCAGGTCAGGCGGGAGCTCCTGCAGGTTTACCTCGCATA |
| | | GACGGGTCAGGGCGCGGGCTAGATCCAGGTGATACCTAATTTCCAGGGGC |
| | | TGGTTGGTGGCGGCGTCGATGGCTTGCAAGAGGCCGCATCCCCGCGGCG |
| | | CGACTACGGTACCGCGCGGCGGGCGGTGGGCCGCGGGGGTGTCCTTGGA |
| | | TGATGCATCTAAAAGCGGTGACGCGGGCGAGCCCCCGGAGGTAGGGGGG |
| | | GCTCCGGACCCGCCGGGAGAGGGGGCAGGGGCACGTCGGCGCCGCGCG |
| | | CGGGCAGGAGCTGGTGCTGCGCGCGTAGGTTGCTGGCGAACGCGACGAC |
| | | GCGGCGGTTGATCTCCTGAATCTGGCGCCTCTGCGTGAAGACGACGGGCC |
| | | CGGTGAGCTTGAGCCTGAAAGAGAGTTCGACAGAATCAATTTCGGTGTCGT |
| | | TGACGGCGGCCTGGCGCAAAATCTCCTGCACGTCTCCTGAGTTGTCTTGAT |
| | | AGGCGATCTCGGCCATGAACTGCTCGATCTCTTCCTCCTGGAGATCTCCGC |
| | | GTCCGGCTCGCTCCACGGTGGCGGCGAGGTCGTTGGAAATGCGGGCCAT |
| | | GAGCTGCGAGAAGGCGTTGAGGCCTCCCTCGTTCCAGACGCGGCTGTAGA |
| | | CCACGCCCCCTTCGGCATCGCGGGCGCGCATGACCACCTGCGCGAGATT |
| | | GAGCTCCACGTGCCGGGCGAAGACGGCGTAGTTTCGCAGGCGCTGAAAG |
| | | AGGTAGTTGAGGGTGGTGGCGGTGTGTTCTGCCACGAAGAAGTACATAAC |
| | | CCAGCGTCGCAACGTGGATTCGTTGATATCCCCCAAGGCCTCAAGGCGCT |
| | | CCATGGCCTCGTAGAAGTCCACGGCGAAGTTGAAAAACTGGGAGTTGCGC |
| | | GCCGACACGGTTAACTCCTCCTCCAGAAGACGGATGAGCTCGGCGACAGT |
| | | GTCGCGCACCTCGCGCTCAAAGGCTACAGGGGCCTCTTCTTCTTCTTCAAT |
| | | CTCCTCTTCCATAAGGGCCTCCCCTTCTTCTTCTTCTGGCGGCGGTGGGGG |
| | | AGGGGGGACACGGCGGCGACGACGGCGCACCGGGAGGCGGTCGACAAA |
| | | GCGCTCGATCATCTCCCCGCGGCGACGGCGCATGGTCTCGGTGACGGCG |
| | | CGGCCGTTCTCGCGGGGGCGCAGTTGGAAGACGCCGCCCGTCATGTCCC |
| | | GGTTATGGGTTGGCGGGGGGCTGCCATGCGGCAGGGATACGGCGCTAAC |
| | | GATGCATCTCAACAATTGTTGTGTAGGTACTCCGCCGCCGAGGGACCTGAG |
| | | CGAGTCCGCATCGACCGGATCGGAAAACCTCTCGAGAAAGGCGTCTAACC |
| | | AGTCACAGTCGCAAGGTAGGCTGAGCACCGTGGCGGGCGGCAGCGGGCG |
| | | GCGGTCGGGGTTGTTTCTGGCGGAGGTGCTGCTGATGATGTAATTAAAGTA |
| | | GGCGGTCTTGAGACGGCGGATGGTCGACAGAAGCACCATGTCCTTGGGTC |
| | | CGGCCTGCTGAATGCGCAGGCGGTCGGCCATGCCCCAGGCTTCGTTTTGA |
| | | CATCGGCGCAGGTCTTTGTAGTAGTCTTGCATGAGCCTTTCTACCGGCACT |
| | | TCTTCTTCTCCTTCCTCTTGTCCTGCATCTCTTGCATCTATCGCTGCGGCGG |
| | | CGGCGGAGTTTGGCCGTAGGTGGCGCCCTCTTCCTCCCATGCGTGTGACC |
| | | CCGAAGCCCCTCATCGGCTGAAGCAGGGCTAGGTCGGCGACAACGCGCT |
| | | CGGCTAATATGGCCTGCTGCACCTGCGTGAGGGTAGACTGGAAGTCATCC |
| | | ATGTCCACAAAGCGGTGGTATGCGCCCGTGTTGATGGTGTAAGTGCAGTTG |
| | | GCCATAACGGACCAGTTAACGGTCTGGTGACCCGGCTGCGAGAGCTCGGT |
| | | GTACCTGAGACGCGAGTAAGCCCTCGAGTCAAATACGTAGTCGTTGCAAGT |
| | | CCGCACCAGGTACTGGTATCCCACCAAAAAGTGCGGCGGCGGCTGGCGGT |
| | | AGAGGGGCCAGCGTAGGGTGGCCGGGGCTCCGGGGGCGAGATCTTCCAA |
| | | CATAAGGCGATGATATCCGTAGATGTACCTGGACATCCAGGTGATGCCGGC |
| | | GGCGGTGGTGGAGGCGCGCGGAAAGTCGCGGACGCGGTTCCAGATGTTG |
| | | CGCAGCGGCAAAAAGTGCTCCATGGTCGGGACGCTCTGGCCGGTCAGGC |
| | | GCGCGCAATCGTTGACGCTCTACCGTGCAAAAGGAGAGCCTGTAAGCGGG |
| | | CACTCTTCCGTGGTCTGGTGGATAAATTCGCAAGGGTATCATGGCGGACGA |
| | | CCGGGGTTCGAGCCCCGTATCCGGCCGTCCGCCGTGATCCATGCGGTTAC |
| | | CGCCCGCGTGTCGAACCCAGGTGTGCGACGTCAGACAACGGGGGAGTGC |
| | | TCCTTTTGGCTTCCTTCCAGGCGCGGCGGCTGCTGCGCTAGCTTTTTTGGC |
| | | CACTGGCCGCGCGCAGCGTAAGCGGTTAGGCTGGAAAGCGAAAGCATTAA |
| | | GTGGCTCGCTCCCTGTAGCCGGAGGGTTATTTTCCAAGGGTTGAGTCGCG |
| | | GGACCCCCGGTTCGAGTCTCGGACCGGCCGGACTGCGGCGAACGGGGGT |
| | | TTGCCTCCCCGTCATGCAAGACCCCGCTTGCAAATTCCTCCGGAAACAGGG |
| | | ACGAGCCCCTTTTTTGCTTTTCCCAGATGCATCCGGTGCTGCGGCAGATGC |
| | | GCCCCCCTCCTCAGCAGCGGCAAGAGCAAGAGCAGCGGCAGACATGCAG |
| | | GGCACCCTCCCCTCCTCCTACCGCGTCAGGAGGGGCGACATCCGCGGTTG |
| | | ACGCGGCAGCAGATGGTGATTACGAACCCCCGCGGCGCCGGGCCCGGCA |
| | | CTACCTGGACTTGGAGGAGGGCGAGGGCCTGGCGCGGCTAGGAGCGCCC |
| | | TCTCCTGAGCGGTACCCAAGGGTGCAGCTGAAGCGTGATACGCGTGAGGC |
| | | GTACGTGCCGCGGCAGAACCTGTTTCGCGACCGCGAGGGAGAGGAGCCC |
| | | GAGGAGATGCGGGATCGAAAGTTCCACGCAGGGCGCGAGCTGCGGCATG |
| | | GCCTGAATCGCGAGCGGTTGCTGCGCGAGGAGGACTTTGAGCCCGACGC |
| | | GCGAACCGGGATTAGTCCCGCGCGCGCACACGTGGCGGCCGCCGACCTG |
| | | GTAACCGCATACGAGCAGACGGTGAACCAGGAGATTAACTTTCAAAAAAGC |
| | | TTTAACAACCACGTGCGTACGCTTGTGGCGCGCGAGGAGGTGGCTATAGG |
| | | ACTGATGCATCTGTGGGACTTTGTAAGCGCGCTGGAGCAAAACCCAAATAG |
| | | CAAGCCGCTCATGGCGCAGCTGTTCCTTATAGTGCAGCACAGCAGGGACA |
| | | ACGAGGCATTCAGGGATGCGCTGCTAAACATAGTAGAGCCCGAGGGCCGC |
| | | TGGCTGCTCGATTTGATAAACATCCTGCAGAGCATAGTGGTGCAGGAGCGC |
| | | AGCTTGAGCCTGGCTGACAAGGTGGCCGCCATCAACTATTCCATGCTTAGC |
| | | CTGGGCAAGTTTTACGCCCGCAAGATATACCATACCCCTTACGTTCCCATA |
| | | GACAAGGAGGTAAAGATCGAGGGGTTCTACATGCGCATGGCGCTGAAGGT |
| | | GCTTACCTTGAGCGACGACCTGGGCGTTTATCGCAACGAGCGCATCCACA |
| | | AGGCCGTGAGCGTGAGCCGGCGGCGCGAGCTCAGCGACCGCGAGCTGAT |
| | | GCACAGCCTGCAAAGGGCCCTGGCTGGCACGGGCAGCGGCGATAGAGAG |
| | | GCCGAGTCCTACTTTGACGCGGGCGCTGACCTGCGCTGGGCCCCAAGCC |
| | | GACGCGCCCTGGAGGCAGCTGGGGCCGGACCTGGGCTGGCGGTGGCAC |
| | | CCGCGCGCGCTGGCAACGTCGGCGGCGTGGAGGAATATGACGAGGACGA |
| | | TGAGTACGAGCCAGAGGACGGCGAGTACTAAGCGGTGATGTTTCTGATCA |
| | | GATGATGCAAGACGCAACGGACCCGGCGGTGCGGGCGGCGCTGCAGAGC |
| | | CAGCCGTCCGGCCTTAACTCCACGGACGACTGGCGCCAGGTCATGGACCG |
| | | CATCATGTCGCTGACTGCGCGCAATCCTGACGCGTTCCGGCAGCAGCCGC |
| | | AGGCCAACCGGCTCTCCGCAATTCTGGAAGCGGTGGTCCCGGCGCGCGC |
| | | AAACCCCACGCACGAGAAGGTGCTGGCGATCGTAAACGCGCTGGCCGAAA |
| | | ACAGGGCCATCCGGCCCGACGAGGCCGGCCTGGTCTACGACGCGCTGCT |
| | | TCAGCGCGTGGCTCGTTACAACAGCGGCAACGTGCAGACCAACCTGGACC |
| | | GGCTGGTGGGGGATGTGCGCGAGGCCGTGGCGCAGCGTGAGCGCGCGC |
| | | AGCAGCAGGGCAACCTGGGCTCCATGGTTGCACTAAACGCCTTCCTGAGT |
| | | ACACAGCCCGCCAACGTGCCGCGGGGACAGGAGGACTACACCAACTTTGT |
| | | GAGCGCACTGCGGCTAATGGTGACTGAGACACCGCAAAGTGAGGTGTACC |
| | | AGTCTGGGCCAGACTATTTTTTCCAGACCAGTAGACAAGGCCTGCAGACCG |
| | | TAAACCTGAGCCAGGCTTTCAAAAACTTGCAGGGGCTGTGGGGGGTGCGG |
| | | GCTCCCACAGGCGACCGCGCGACCGTGTCTAGCTTGCTGACGCCCAACTC |
| | | GCGCCTGTTGCTGCTGCTAATAGCGCCCTTCACGGACAGTGGCAGCGTGT |
| | | CCCGGGACACATACCTAGGTCACTTGCTGACACTGTACCGCGAGGCCATA |
| | | GGTCAGGCGCATGTGGACGAGCATACTTTCCAGGAGATTACAAGTGTCAG |
| | | CCGCGCGCTGGGGCAGGAGGACACGGGCAGCCTGGAGGCAACCCTAAAC |
| | | TACCTGCTGACCAACCGGCGGCAGAAGATCCCCTCGTTGCACAGTTTAAAC |
| | | AGCGAGGAGGAGCGCATTTTGCGCTACGTGCAGCAGAGCGTGAGCCTTAA |
| | | CCTGATGCGCGACGGGGTAACGCCCAGCGTGGCGCTGGACATGACCGCG |
| | | CGCAACATGGAACCGGGCATGTATGCCTCAAACCGGCCGTTTATCAACCG |
| | | CCTAATGGACTACTTGCATCGCGCGGCCGCCGTGAACCCCGAGTATTTCAC |
| | | CAATGCCATCTTGAACCCGCACTGGCTACCGCCCCCTGGTTTCTACACCGG |
| | | GGGATTCGAGGTGCCCGAGGGTAACGATGGATTCCTCTGGGACGACATAG |
| | | ACGACAGCGTGTTTTCCCCGCAACCGCAGACCCTGCTAGAGTTGCAACAG |
| | | CGCGAGCAGGCAGAGGCGGCGCTGCGAAAGGAAAGCTTCCGCAGGCCAA |
| | | GCAGCTTGTCCGATCTAGGCGCTGCGGCCCCGCGGTCAGATGCTAGTAGC |
| | | CCATTTCCAAGCTTGATAGGGTCTCTTACCAGCACTCGCACCACCCGCCCG |
| | | CGCCTGCTGGGCGAGGAGGAGTACCTAAACAACTCGCTGCTGCAGCCGCA |
| | | GCGCGAAAAAAACCTGCCTCCGGCATTTCCCAACAACGGGATAGAGAGCC |
| | | TAGTGGACAAGATGAGTAGATGGAAGACGTACGCGCAGGAGCACAGGGAC |
| | | GTGCCAGGCCCGCGCCCGCCCACCCGTCGTCAAAGGCACGACCGTCAGC |
| | | GGGGTCTGGTGTGGGAGGACGATGACTCGGCAGACGACAGCAGCGTCCT |
| | | GGATTTGGGAGGGAGTGGCAACCCGTTTGCGCACCTTCGCCCCAGGCTGG |
| | | GGAGAATGTTTTAAAAAAAAAAAAGCATGATGCAAAATAAAAAACTCACCAA |
| | | GGCCATGGCACCGAGCGTTGGTTTTCTTGTATTCCCCTTAGTATGCGGCGC |
| | | GCGGCGATGTATGAGGAAGGTCCTCCTCCCTCCTACGAGAGTGTGGTGAG |
| | | CGCGGCGCCAGTGGCGGCGGCGCTGGGTTCTCCCTTCGATGCTCCCCTG |
| | | GACCCGCCGTTTGTGCCTCCGCGGTACCTGCGGCCTACCGGGGGGAGAA |
| | | ACAGCATCCGTTACTCTGAGTTGGCACCCCTATTCGACACCACCCGTGTGT |
| | | ACCTGGTGGACAACAAGTCAACGGATGTGGCATCCCTGAACTACCAGAAC |
| | | GACCACAGCAACTTTCTGACCACGGTCATTCAAAACAATGACTACAGCCCG |
| | | GGGGAGGCAAGCACACAGACCATCAATCTTGACGACCGGTCGCACTGGGG |
| | | CGGCGACCTGAAAACCATCCTGCATACCAACATGCCAAATGTGAACGAGTT |
| | | CATGTTTACCAATAAGTTTAAGGCGCGGGTGATGGTGTCGCGCTTGCCTAC |
| | | TAAGGACAATCAGGTGGAGCTGAAATACGAGTGGGTGGAGTTCACGCTGC |
| | | CCGAGGGCAACTACTCCGAGACCATGACCATAGACCTTATGAACAACGCGA |
| | | TCGTGGAGCACTACTTGAAAGTGGGCAGACAGAACGGGGTTCTGGAAAGC |
| | | GACATCGGGGTAAAGTTTGACACCCGCAACTTCAGACTGGGGTTTGACCCC |
| | | GTCACTGGTCTTGTCATGCCTGGGGTATATACAAACGAAGCCTTCCATCCA |
| | | GACATCATTTTGCTGCCAGGATGCGGGGTGGACTTCACCCACAGCCGCCT |
| | | GAGCAACTTGTTGGGCATCCGCAAGCGGCAACCCTTCCAGGAGGGCTTTA |
| | | GGATCACCTACGATGATCTGGAGGGTGGTAACATTCCCGCACTGTTGGATG |
| | | TGGACGCCTACCAGGCGAGCTTGAAAGATGACACCGAACAGGGCGGGGG |
| | | TGGCGCAGGCGGCAGCAACAGCAGTGGCAGCGGCGCGGAAGAGAACTCC |
| | | AACGCGGCAGCCGCGGCAATGCAGCCGGTGGAGGACATGAACGATCATG |
| | | CCATTCGCGGCGACACCTTTGCCACACGGGCTGAGGAGAAGCGCGCTGAG |
| | | GCCGAAGCAGCGGCCGAAGCTGCCGCCCCCGCTGCGCAACCCGAGGTCG |
| | | AGAAGCCTCAGAAGAAACCGGTGATCAAACCCCTGACAGAGGACAGCAAG |
| | | AAACGCAGTTACAACCTAATAAGCAATGACAGCACCTTCACCCAGTACCGC |
| | | AGCTGGTACCTTGCATACAACTACGGCGACCCTCAGACCGGAATCCGCTCA |
| | | TGGACCCTGCTTTGCACTCCTGACGTAACCTGCGGCTCGGAGCAGGTCTA |
| | | CTGGTCGTTGCCAGACATGATGCAAGACCCCGTGACCTTCCGCTCCACGC |
| | | GCCAGATCAGCAACTTTCCGGTGGTGGGCGCCGAGCTGTTGCCCGTGCAC |
| | | TCCAAGAGCTTCTACAACGACCAGGCCGTCTACTCCCAACTCATCCGCCAG |
| | | TTTACCTCTCTGACCCACGTGTTCAATCGCTTTCCCGAGAACCAGATTTTGG |
| | | CGCGCCCGCCAGCCCCCACCATCACCACCGTCAGTGAAAACGTTCCTGCT |
| | | CTCACAGATCACGGGACGCTACCGCTGCGCAACAGCATCGGAGGAGTCCA |
| | | GCGAGTGACCATTACTGACGCCAGACGCCGCACCTGCCCCTACGTTTACA |
| | | AGGCCCTGGGCATAGTCTCGCCGCGCGTCCTATCGAGCCGCACTTTTTGA |
| | | GCAAGCATGTCCATCCTTATATCGCCCAGCAATAACACAGGCTGGGGCCTG |
| | | CGCTTCCCAAGCAAGATGTTTGGCGGGGCCAAGAAGCGCTCCGACCAACA |
| | | CCCAGTGCGCGTGCGCGGGCACTACCGCGCGCCCTGGGGCGCGCACAAA |
| | | CGCGGCCGCACTGGGCGCACCACCGTCGATGACGCCATCGACGCGGTGG |
| | | TGGAGGAGGCGCGCAACTACACGCCCACGCCGCCACCAGTGTCCACAGT |
| | | GGACGCGGCCATTCAGACCGTGGTGCGCGGAGCCCGGCGCTATGCTAAA |
| | | ATGAAGAGACGGCGGAGGCGCGTAGCACGTCGCCACCGCCGCCGACCCG |
| | | GCACTGCCGCCCAACGCGCGGCGGCGGCCCTGCTTAACCGCGCACGTCG |
| | | CACCGGCCGACGGGCGGCCATGCGGGCCGCTCGAAGGCTGGCCGCGGG |
| | | TATTGTCACTGTGCCCCCCAGGTCCAGGCGACGAGCGGCCGCCGCAGCA |
| | | GCCGCGGCCATTAGTGCTATGACTCAGGGTCGCAGGGGCAACGTGTATTG |
| | | GGTGCGCGACTCGGTTAGCGGCCTGCGCGTGCCCGTGCGCACCCGCCCC |
| | | CCGCGCAACTAGATTGCAAGAAAAAACTACTTAGACTCGTACTGTTGTATGT |
| | | ATCCAGCGGCGGCGGCGCGCAACGAAGCTATGTCCAAGCGCAAAATCAAA |
| | | GAAGAGATGCTCCAGGTCATCGCGCCGGAGATCTATGGCCCCCCGAAGAA |
| | | GGAAGAGCAGGATTACAAGCCCCGAAAGCTAAAGCGGGTCAAAAAGAAAA |
| | | AGAAAGATGATGATGATGAACTTGACGACGAGGTGGAACTGCTGCACGCTA |
| | | CCGCGCCCAGGCGACGGGTACAGTGGAAAGGTCGACGCGTAAAACGTGTT |
| | | TTGCGACCCGGCACCACCGTAGTCTTTACGCCCGGTGAGCGCTCCACCCG |
| | | CACCTACAAGCGCGTGTATGATGAGGTGTACGGCGACGAGGACCTGCTTG |
| | | AGCAGGCCAACGAGCGCCTCGGGGAGTTTGCCTACGGAAAGCGGCATAAG |
| | | GACATGCTGGCGTTGCCGCTGGACGAGGGCAACCCAACACCTAGCCTAAA |
| | | GCCCGTAACACTGCAGCAGGTGCTGCCCGCGCTTGCACCGTCCGAAGAAA |
| | | AGCGCGGCCTAAAGCGCGAGTCTGGTGACTTGGCACCCACCGTGCAGCTG |
| | | ATGGTACCCAAGCGCCAGCGACTGGAAGATGTCTTGGAAAAAATGACCGT |
| | | GGAACCTGGGCTGGAGCCCGAGGTCCGCGTGCGGCCAATCAAGCAGGTG |
| | | GCGCCGGGACTGGGCGTGCAGACCGTGGACGTTCAGATACCCACTACCAG |
| | | TAGCACCAGTATTGCCACCGCCACAGAGGGCATGGAGACACAAACGTCCC |
| | | CGGTTGCCTCAGCGGTGGCGGATGCCGCGGTGCAGGCGGTCGCTGCGGC |
| | | CGCGTCCAAGACCTCTACGGAGGTGCAAACGGACCCGTGGATGTTTCGCG |
| | | TTTCAGCCCCCCGGCGCCCGCGCGGTTCGAGGAAGTACGGCGCCGCCAG |
| | | CGCGCTACTGCCCGAATATGCCCTACATCCTTCCATTGCGCCTACCCCCGG |
| | | CTATCGTGGCTACACCTACCGCCCCAGAAGACGAGCAACTACCCGACGCC |
| | | GAACCACCACTGGAACCCGCCGCCGCCGTCGCCGTCGCCAGCCCGTGCT |
| | | GGCCCCGATTTCCGTGCGCAGGGTGGCTCGCGAAGGAGGCAGGACCCTG |
| | | GTGCTGCCAACAGCGCGCTACCACCCCAGCATCGTTTAAAAGCCGGTCTTT |
| | | GTGGTTCTTGCAGATATGGCCCTCACCTGCCGCCTCCGTTTCCCGGTGCC |
| | | GGGATTCCGAGGAAGAATGCACCGTAGGAGGGGCATGGCCGGCCACGGC |
| | | CTGACGGGCGGCATGCGTCGTGCGCACCACCGGCGGCGGCGCGCGTCG |
| | | CACCGTCGCATGCGCGGCGGTATCCTGCCCCTCCTTATTCCACTGATCGC |
| | | CGCGGCGATTGGCGCCGTGCCCGGAATTGCATCCGTGGCCTTGCAGGCG |
| | | CAGAGACACTGATTAAAAACAAGTTGCATGTGGAAAAATCAAAATAAAAAGT |
| | | CTGGACTCTCACGCTCGCTTGGTCCTGTAACTATTTTGTAGAATGGAAGAC |
| | | ATCAACTTTGCGTCTCTGGCCCCGCGACACGGCTCGCGCCCGTTCATGGG |
| | | AAACTGGCAAGATATCGGCACCAGCAATATGAGCGGTGGCGCCTTCAGCT |
| | | GGGGCTCGCTGTGGAGCGGCATTAAAAATTTCGGTTCCACCGTTAAGAACT |
| | | ATGGCAGCAAGGCCTGGAACAGCAGCACAGGCCAGATGCTGAGGGATAAG |
| | | TTGAAAGAGCAAAATTTCCAACAAAAGGTGGTAGATGGCCTGGCCTCTGGC |
| | | ATTAGCGGGGTGGTGGACCTGGCCAACCAGGCAGTGCAAAATAAGATTAA |
| | | CAGTAAGCTTGATCCCCGCCCTCCCGTAGAGGAGCCTCCACCGGCCGTGG |
| | | AGACAGTGTCTCCAGAGGGGCGTGGCGAAAAGCGTCCGCGCCCCGACAG |
| | | GGAAGAAACTCTGGTGACGCAAATAGACGAGCCTCCCTCGTACGAGGAGG |
| | | CACTAAAGCAAGGCCTGCCCACCACCCGTCCCATCGCGCCCATGGCTACC |
| | | GGAGTGCTGGGCCAGCACACACCCGTAACGCTGGACCTGCCTCCCCCCG |
| | | CCGACACCCAGCAGAAACCTGTGCTGCCAGGCCCGACCGCCGTTGTTGTA |
| | | ACCCGTCCTAGCCGCGCGTCCCTGCGCCGCGCCGCCAGCGGTCCGCGAT |
| | | CGTTGCGGCCCGTAGCCAGTGGCAACTGGCAAAGCACACTGAACAGCATC |
| | | GTGGGTCTGGGGGTGCAATCCCTGAAGCGCCGACGATGCTTCTGAATAGC |
| | | TAACGTGTCGTATGTGTGTCATGTATGCGTCCATGTCGCCGCCAGAGGAGC |
| | | TGCTGAGCCGCCGCGCGCCCGCTTTCCAAGATGGCTACCCCTTCGATGAT |
| | | GCCGCAGTGGTCTTACATGCACATCTCGGGCCAGGACGCCTCGGAGTACC |
| | | TGAGCCCCGGGCTGGTGCAGTTTGCCCGCGCCACCGAGACGTACTTCAGC |
| | | CTGAATAACAAGTTTAGAAACCCCACGGTGGCGCCTACGCACGACGTGAC |
| | | CACAGACCGGTCCCAGCGTTTGACGCTGCGGTTCATCCCTGTGGACCGTG |
| | | AGGATACTGCGTACTCGTACAAGGCGCGGTTCACCCTAGCTGTGGGTGAT |
| | | AACCGTGTGCTGGACATGGCTTCCACGTACTTTGACATCCGCGGCGTGCT |
| | | GGACAGGGGCCCTACTTTTAAGCCCTACTCTGGCACTGCCTACAACGCCCT |
| | | GGCTCCCAAGGGTGCCCCAAATCCTTGCGAATGGGATGAAGCTGCTACTG |
| | | CTCTTGAAATAAACCTAGAAGAAGAGGACGATGACAACGAAGACGAAGTAG |
| | | ACGAGCAAGCTGAGCAGCAAAAAACTCACGTATTTGGGCAGGCGCCTTATT |
| | | CTGGTATAAATATTACAAAGGAGGGTATTCAAATAGGTGTCGAAGGTCAAAC |
| | | ACCTAAATATGCCGATAAAACATTTCAACCTGAACCTCAAATAGGAGAATCT |
| | | CAGTGGTACGAAACTGAAATTAATCATGCAGCTGGGAGAGTCCTTAAAAAG |
| | | ACTACCCCAATGAAACCATGTTACGGTTCATATGCAAAACCCACAAATGAAA |
| | | ATGGAGGGCAAGGCATTCTTGTAAAGCAACAAAATGGAAAGCTAGAAAGTC |
| | | AAGTGGAAATGCAATTTTTCTCAACTACTGAGGCGACCGCAGGCAATGGTG |
| | | ATAACTTGACTCCTAAAGTGGTATTGTACAGTGAAGATGTAGATATAGAAAC |
| | | CCCAGACACTCATATTTCTTACATGCCCACTATTAAGGAAGGTAACTCACGA |
| | | GAACTAATGGGCCAACAATCTATGCCCAACAGGCCTAATTACATTGCTTTTA |
| | | GGGACAATTTTATTGGTCTAATGTATTACAACAGCACGGGTAATATGGGTGT |
| | | TCTGGCGGGCCAAGCATCGCAGTTGAATGCTGTTGTAGATTTGCAAGACAG |
| | | AAACACAGAGCTTTCATACCAGCTTTTGCTTGATTCCATTGGTGATAGAACC |
| | | AGGTACTTTTCTATGTGGAATCAGGCTGTTGACAGCTATGATCCAGATGTTA |
| | | GAATTATTGAAAATCATGGAACTGAAGATGAACTTCCAAATTACTGCTTTCC |
| | | ACTGGGAGGTGTGATTAATACAGAGACTCTTACCAAGGTAAAACCTAAAAC |
| | | AGGTCAGGAAAATGGATGGGAAAAAGATGCTACAGAATTTTCAGATAAAAAT |
| | | GAAATAAGAGTTGGAAATAATTTTGCCATGGAAATCAATCTAAATGCCAACC |
| | | TGTGGAGAAATTTCCTGTACTCCAACATAGCGCTGTATTTGCCCGACAAGC |
| | | TAAAGTACAGTCCTTCCAACGTAAAAATTTCTGATAACCCAAACACCTACGA |
| | | CTACATGAACAAGCGAGTGGTGGCTCCCGGGTTAGTGGACTGCTACATTAA |
| | | CCTTGGAGCACGCTGGTCCCTTGACTATATGGACAACGTCAACCCATTTAA |
| | | CCACCACCGCAATGCTGGCCTGCGCTACCGCTCAATGTTGCTGGGCAATG |
| | | GTCGCTATGTGCCCTTCCACATCCAGGTGCCTCAGAAGTTCTTTGCCATTA |
| | | AAAACCTCCTTCTCCTGCCGGGCTCATACACCTACGAGTGGAACTTCAGGA |
| | | AGGATGTTAACATGGTTCTGCAGAGCTCCCTAGGAAATGACCTAAGGGTTG |
| | | ACGGAGCCAGCATTAAGTTTGATAGCATTTGCCTTTACGCCACCTTCTTCCC |
| | | CATGGCCCACAACACCGCCTCCACGCTTGAGGCCATGCTTAGAAACGACA |
| | | CCAACGACCAGTCCTTTAACGACTATCTCTCCGCCGCCAACATGCTCTACC |
| | | CTATACCCGCCAACGCTACCAACGTGCCCATATCCATCCCCTCCCGCAACT |
| | | GGGCGGCTTTCCGCGGCTGGGCCTTCACGCGCCTTAAGACTAAGGAAACC |
| | | CCATCACTGGGCTCGGGCTACGACCCTTATTACACCTACTCTGGCTCTATA |
| | | CCCTACCTAGATGGAACCTTTTACCTCAACCACACCTTTAAGAAGGTGGCC |
| | | ATTACCTTTGACTCTTCTGTCAGCTGGCCTGGCAATGACCGCCTGCTTACC |
| | | CCCAACGAGTTTGAAATTAAGCGCTCAGTTGACGGGGAGGGTTACAACGTT |
| | | GCCCAGTGTAACATGACCAAAGACTGGTTCCTGGTACAAATGCTAGCTAAC |
| | | TACAACATTGGCTACCAGGGCTTCTATATCCCAGAGAGCTACAAGGACCGC |
| | | ATGTACTCCTTCTTTAGAAACTTCCAGCCCATGAGCCGTCAGGTGGTGGAT |
| | | GATACTAAATACAAGGACTACCAACAGGTGGGCATCCTACACCAACACAAC |
| | | AACTCTGGATTTGTTGGCTACCTTGCCCCCACCATGCGCGAAGGACAGGC |
| | | CTACCCTGCTAACTTCCCCTATCCGCTTATAGGCAAGACCGCAGTTGACAG |
| | | CATTACCCAGAAAAAGTTTCTTTGCGATCGCACCCTTTGGCGCATCCCATTC |
| | | TCCAGTAACTTTATGTCCATGGGCGCACTCACAGACCTGGGCCAAAACCTT |
| | | CTCTACGCCAACTCCGCCCACGCGCTAGACATGACTTTTGAGGTGGATCCC |
| | | ATGGACGAGCCCACCCTTCTTTATGTTTTGTTTGAAGTCTTTGACGTGGTCC |
| | | GTGTGCACCGGCCGCACCGCGGCGTCATCGAAACCGTGTACCTGCGCAC |
| | | GCCCTTCTCGGCCGGCAACGCCACAACATAAAGAAGCAAGCAACATCAACA |
| | | ACAGCTGCCGCCATGGGCTCCAGTGAGCAGGAACTGAAAGCCATTGTCAA |
| | | AGATCTTGGTTGTGGGCCATATTTTTTGGGCACCTATGACAAGCGCTTTCCA |
| | | GGCTTTGTTTCTCCACACAAGCTCGCCTGCGCCATAGTCAATACGGCCGGT |
| | | CGCGAGACTGGGGGCGTACACTGGATGGCCTTTGCCTGGAACCCGCACTC |
| | | AAAAACATGCTACCTCTTTGAGCCCTTTGGCTTTTCTGACCAGCGACTCAAG |
| | | CAGGTTTACCAGTTTGAGTACGAGTCACTCCTGCGCCGTAGCGCCATTGCT |
| | | TCTTCCCCCGACCGCTGTATAACGCTGGAAAAGTCCACCCAAAGCGTACAG |
| | | GGGCCCAACTCGGCCGCCTGTGGACTATTCTGCTGCATGTTTCTCCACGC |
| | | CTTTGCCAACTGGCCCCAAACTCCCATGGATCACAACCCCACCATGAACCT |
| | | TATTACCGGGGTACCCAACTCCATGCTCAACAGTCCCCAGGTACAGCCCAC |
| | | CCTGCGTCGCAACCAGGAACAGCTCTACAGCTTCCTGGAGCGCCACTCGC |
| | | CCTACTTCCGCAGCCACAGTGCGCAGATTAGGAGCGCCACTTCTTTTTGTC |
| | | ACTTGAAAAACATGTAAAAATAATGTACTAGAGACACTTTCAATAAAGGCAA |
| | | ATGCTTTTATTTGTACACTCTCGGGTGATTATTTACCCCCACCCTTGCCGTC |
| | | TGCGCCGTTTAAAAATCAAAGGGGTTCTGCCGCGCATCGCTATGCGCCACT |
| | | GGCAGGGACACGTTGCGATACTGGTGTTTAGTGCTCCACTTAAACTCAGGC |
| | | ACAACCATCCGCGGCAGCTCGGTGAAGTTTTCACTCCACAGGCTGCGCAC |
| | | CATCACCAACGCGTTTAGCAGGTCGGGCGCCGATATCTTGAAGTCGCAGTT |
| | | GGGGCCTCCGCCCTGCGCGCGCGAGTTGCGATACACAGGGTTGCAGCAC |
| | | TGGAACACTATCAGCGCCGGGTGGTGCACGCTGGCCAGCACGCTCTTGTC |
| | | GGAGATCAGATCCGCGTCCAGGTCCTCCGCGTTGCTCAGGGCGAACGGAG |
| | | TCAACTTTGGTAGCTGCCTTCCCAAAAAGGGCGCGTGCCCAGGCTTTGAGT |
| | | TGCACTCGCACCGTAGTGGCATCAAAAGGTGACCGTGCCCGGTCTGGGCG |
| | | TTAGGATACAGCGCCTGCATAAAAGCCTTGATCTGCTTAAAAGCCACCTGA |
| | | GCCTTTGCGCCTTCAGAGAAGAACATGCCGCAAGACTTGCCGGAAAACTGA |
| | | TTGGCCGGACAGGCCGCGTCGTGCACGCAGCACCTTGCGTCGGTGTTGGA |
| | | GATCTGCACCACATTTCGGCCCCACCGGTTCTTCACGATCTTGGCCTTGCT |
| | | AGACTGCTCCTTCAGCGCGCGCTGCCCGTTTTCGCTCGTCACATCCATTTC |
| | | AATCACGTGCTCCTTATTTATCATAATGCTTCCGTGTAGACACTTAAGCTCG |
| | | CCTTCGATCTCAGCGCAGCGGTGCAGCCACAACGCGCAGCCCGTGGGCTC |
| | | GTGATGCTTGTAGGTCACCTCTGCAAACGACTGCAGGTACGCCTGCAGGA |
| | | ATCGCCCCATCATCGTCACAAAGGTCTTGTTGCTGGTGAAGGTCAGCTGCA |
| | | ACCCGCGGTGCTCCTCGTTCAGCCAGGTCTTGCATACGGCCGCCAGAGCT |
| | | TCCACTTGGTCAGGCAGTAGTTTGAAGTTCGCCTTTAGATCGTTATCCACGT |
| | | GGTACTTGTCCATCAGCGCGCGCGCAGCCTCCATGCCCTTCTCCCACGCA |
| | | GACACGATCGGCACACTCAGCGGGTTCATCACCGTAATTTCACTTTCCGCT |
| | | TCGCTGGGCTCTTCCTCTTCCTCTTGCGTCCGCATACCACGCGCCACTGGG |
| | | TCGTCTTCATTCAGCCGCCGCACTGTGCGCTTACCTCCTTTGCCATGCTTG |
| | | ATTAGCACCGGTGGGTTGCTGAAACCCACCATTTGTAGCGCCACATCTTCT |
| | | CTTTCTTCCTCGCTGTCCACGATTACCTCTGGTGATGGCGGGCGCTCGGGC |
| | | TTGGGAGAAGGGCGCTTCTTTTTCTTCTTGGGCGCAATGGCCAAATCCGCC |
| | | GCCGAGGTCGATGGCCGCGGGCTGGGTGTGCGCGGCACCAGCGCGTCTT |
| | | GTGATGAGTCTTCCTCGTCCTCGGACTCGATACGCCGCCTCATCCGCTTTT |
| | | TTGGGGGCGCCCGGGGAGGCGGCGGCGACGGGGACGGGGACGACACGT |
| | | CCTCCATGGTTGGGGGACGTCGCGCCGCACCGCGTCCGCGCTCGGGGGT |
| | | GGTTTCGCGCTGCTCCTCTTCCCGACTGGCCATTTCCTTCTCCTATAGGCA |
| | | GAAAAAGATCATGGAGTCAGTCGAGAAGAAGGACAGCCTAACCGCCCCCT |
| | | CTGAGTTCGCCACCACCGCCTCCACCGATGCCGCCAACGCGCCTACCACC |
| | | TTCCCCGTCGAGGCACCCCCGCTTGAGGAGGAGGAAGTGATTATCGAGCA |
| | | GGACCCAGGTTTTGTAAGCGAAGACGACGAGGACCGCTCAGTACCAACAG |
| | | AGGATAAAAAGCAAGACCAGGACAACGCAGAGGCAAACGAGGAACAAGTC |
| | | GGGCGGGGGGACGAAAGGCATGGCGACTACCTAGATGTGGGAGACGACG |
| | | TGCTGTTGAAGCATCTGCAGCGCCAGTGCGCCATTATCTGCGACGCGTTG |
| | | CAAGAGCGCAGCGATGTGCCCCTCGCCATAGCGGATGTCAGCCTTGCCTA |
| | | CGAACGCCACCTATTCTCACCGCGCGTACCCCCCAAACGCCAAGAAAACG |
| | | GCACATGCGAGCCCAACCCGCGCCTCAACTTCTACCCCGTATTTGCCGTG |
| | | CCAGAGGTGCTTGCCACCTATCACATCTTTTTCCAAAACTGCAAGATACCCC |
| | | TATCCTGCCGTGCCAACCGCAGCCGAGCGGACAAGCAGCTGGCCTTGCGG |
| | | CAGGGCGCTGTCATACCTGATATCGCCTCGCTCAACGAAGTGCCAAAAATC |
| | | TTTGAGGGTCTTGGACGCGACGAGAAGCGCGCGGCAAACGCTCTGCAACA |
| | | GGAAAACAGCGAAAATGAAAGTCACTCTGGAGTGTTGGTGGAACTCGAGG |
| | | GTGACAACGCGCGCCTAGCCGTACTAAAACGCAGCATCGAGGTCACCCAC |
| | | TTTGCCTACCCGGCACTTAACCTACCCCCCAAGGTCATGAGCACAGTCATG |
| | | AGTGAGCTGATCGTGCGCCGTGCGCAGCCCCTGGAGAGGGATGCAAATTT |
| | | GCAAGAACAAACAGAGGAGGGCCTACCCGCAGTTGGCGACGAGCAGCTAG |
| | | CGCGCTGGCTTCAAACGCGCGAGCCTGCCGACTTGGAGGAGCGACGCAA |
| | | ACTAATGATGGCCGCAGTGCTCGTTACCGTGGAGCTTGAGTGCATGCAGC |
| | | GGTTCTTTGCTGACCCGGAGATGCAGCGCAAGCTAGAGGAAACATTGCACT |
| | | ACACCTTTCGACAGGGCTACGTACGCCAGGCCTGCAAGATCTCCAACGTG |
| | | GAGCTCTGCAACCTGGTCTCCTACCTTGGAATTTTGCACGAAAACCGCCTT |
| | | GGGCAAAACGTGCTTCATTCCACGCTCAAGGGCGAGGCGCGCCGCGACTA |
| | | CGTCCGCGACTGCGTTTACTTATTTCTATGCTACACCTGGCAGACGGCCAT |
| | | GGGCGTTTGGCAGCAGTGCTTGGAGGAGTGCAACCTCAAGGAGCTGCAGA |
| | | AACTGCTAAAGCAAAACTTGAAGGACCTATGGACGGCCTTCAACGAGCGCT |
| | | CCGTGGCCGCGCACCTGGCGGACATCATTTTCCCCGAACGCCTGCTTAAA |
| | | ACCCTGCAACAGGGTCTGCCAGACTTCACCAGTCAAAGCATGTTGCAGAAC |
| | | TTTAGGAACTTTATCCTAGAGCGCTCAGGAATCTTGCCCGCCACCTGCTGT |
| | | GCACTTCCTAGCGACTTTGTGCCCATTAAGTACCGCGAATGCCCTCCGCCG |
| | | CTTTGGGGCCACTGCTACCTTCTGCAGCTAGCCAACTACCTTGCCTACCAC |
| | | TCTGACATAATGGAAGACGTGAGCGGTGACGGTCTACTGGAGTGTCACTGT |
| | | CGCTGCAACCTATGCACCCCGCACCGCTCCCTGGTTTGCAATTCGCAGCT |
| | | GCTTAACGAAAGTCAAATTATCGGTACCTTTGAGCTGCAGGGTCCCTCGCC |
| | | TGACGAAAAGTCCGCGGCTCCGGGGTTGAAACTCACTCCGGGGCTGTGGA |
| | | CGTCGGCTTACCTTCGCAAATTTGTACCTGAGGACTACCACGCCCACGAGA |
| | | TTAGGTTCTACGAAGACCAATCCCGCCCGCCAAATGCGGAGCTTACCGCCT |
| | | GCGTCATTACCCAGGGCCACATTCTTGGCCAATTGCAAGCCATCAACAAAG |
| | | CCCGCCAAGAGTTTCTGCTACGAAAGGGACGGGGGGTTTACTTGGACCCC |
| | | CAGTCCGGCGAGGAGCTCAACCCAATCCCCCCGCCGCCGCAGCCCTATCA |
| | | GCAGCAGCCGCGGGCCCTTGCTTCCCAGGATGGCACCCAAAAAGAAGCTG |
| | | CAGCTGCCGCCGCCACCCACGGACGAGGAGGAATACTGGGACAGTCAGG |
| | | CAGAGGAGGTTTTGGACGAGGAGGAGGAGGACATGATGGAAGACTGGGA |
| | | GAGCCTAGACGAGGAAGCTTCCGAGGTCGAAGAGGTGTCAGACGAAACAC |
| | | CGTCACCCTCGGTCGCATTCCCCTCGCCGGCGCCCCAGAAATCGGCAACC |
| | | GGTTCCAGCATGGCTACAACCTCCGCTCCTCAGGCGCCGCCGGCACTGCC |
| | | CGTTCGCCGACCCAACCGTAGATGGGACACCACTGGAACCAGGGCCGGTA |
| | | AGTCCAAGCAGCCGCCGCCGTTAGCCCAAGAGCAACAACAGCGCCAAGGC |
| | | TACCGCTCATGGCGCGGGCACAAGAACGCCATAGTTGCTTGCTTGCAAGA |
| | | CTGTGGGGGCAACATCTCCTTCGCCCGCCGCTTTCTTCTCTACCATCACGG |
| | | CGTGGCCTTCCCCCGTAACATCCTGCATTACTACCGTCATCTCTACAGCCC |
| | | ATACTGCACCGGCGGCAGCGGCAGCGGCAGCAACAGCAGCGGCCACACA |
| | | GAAGCAAAGGCGACCGGATAGCAAGACTCTGACAAAGCCCAAGAAATCCA |
| | | CAGCGGCGGCAGCAGCAGGAGGAGGAGCGCTGCGTCTGGCGCCCAACGA |
| | | ACCCGTATCGACCCGCGAGCTTAGAAACAGGATTTTTCCCACTCTGTATGC |
| | | TATATTTCAACAGAGCAGGGGCCAAGAACAAGAGCTGAAAATAAAAAACAG |
| | | GTCTCTGCGATCCCTCACCCGCAGCTGCCTGTATCACAAAAGCGAAGATCA |
| | | GCTTCGGCGCACGCTGGAAGACGCGGAGGCTCTCTTCAGTAAATACTGCG |
| | | CGCTGACTCTTAAGGACTAGTTTCGCGCCCTTTCTCAAATTTAAGCGCGAAA |
| | | ACTACGTCATCTCCAGCGGCCACACCCGGCGCCAGCACCTGTCGTCAGCG |
| | | CCATTATGAGCAAGGAAATTCCCACGCCCTACATGTGGAGTTACCAGCCAC |
| | | AAATGGGACTTGCGGCTGGAGCTGCCCAAGACTACTCAACCCGAATAAACT |
| | | ACATGAGCGCGGGACCCCACATGATATCCCGGGTCAACGGAATCCGCGCC |
| | | CACCGAAACCGAATTCTCTTGGAACAGGCGGCTATTACCACCACACCTCGT |
| | | AATAACCTTAATCCCCGTAGTTGGCCCGCTGCCCTGGTGTACCAGGAAAGT |
| | | CCCGCTCCCACCACTGTGGTACTTCCCAGAGACGCCCAGGCCGAAGTTCA |
| | | GATGACTAACTCAGGGGCGCAGCTTGCGGGCGGCTTTCGTCACAGGGTGC |
| | | GGTCGCCCGGGCAGGGTATAACTCACCTGACAATCAGAGGGCGAGGTATT |
| | | CAGCTCAACGACGAGTCGGTGAGCTCCTCGCTTGGTCTCCGTCCGGACGG |
| | | GACATTTCAGATCGGCGGCGCCGGCCGCTCTTCATTCACGCCTCGTCAGG |
| | | CAATCCTAACTCTGCAGACCTCGTCCTCTGAGCCGCGCTCTGGAGGCATTG |
| | | GAACTCTGCAATTTATTGAGGAGTTTGTGCCATCGGTCTACTTTAACCCCTT |
| | | CTCGGGACCTCCCGGCCACTATCCGGATCAATTTATTCCTAACTTTGACGC |
| | | GGTAAAGGACTCGGCGGATGGCTACGACTGAATGTTAAGTGGAGAGGCAG |
| | | AGCAACTGCGCCTGAAACACCTGGTCCACTGTCGCCGCCACAAGTGCTTT |
| | | GCCCGCGACTCCGGTGAGTTTTGCTACTTTGAATTGCCCGAGGATCATATC |
| | | GAGGGCCCGGCGCACGGCGTCCGGCTTACCGCCCAGGGAGAGCTTGCCC |
| | | GTAGCCTGATTCGGGAGTTTACCCAGCGCCCCCTGCTAGTTGAGCGGGAC |
| | | AGGGGACCCTGTGTTCTCACTGTGATTTGCAACTGTCCTAACCCTGGATTA |
| | | CATCAAGATCTTTGTTGCCATCTCTGTGCTGAGTATAATAAATACAGAAATTA |
| | | AAATATACTGGGGCTCCTATCGCCATCCTGTAAACGCCACCGTCTTCACCC |
| | | GCCCAAGCAAACCAAGGCGAACCTTACCTGGTACTTTTAACATCTCTCCCT |
| | | CTGTGATTTACAACAGTTTCAACCCAGACGGAGTGAGTCTACGAGAGAACC |
| | | TCTCCGAGCTCAGCTACTCCATCAGAAAAAACACCACCCTCCTTACCTGCC |
| | | GGGAACGTACGACCTAGGGATAACAGGGTAATAAGCAATTGACTCTATGTG |
| | | GGATATGCTCCAGCGCTACAACCTTGAAGTCAGGCTTCCTGGATGTCAGCA |
| | | TCTGACTTTGGCCAGCACCTGTCCCGCGGATTTGTTCCAGTCCAACTACAG |
| | | CGACCCACCCTAACAGAGATGACCAACACAACCAACGCGGCCGCCGCTAC |
| | | CGGACTTACATCTACCACAAATACACCCCAAGTTTCTGCCTTTGTCAATAAC |
| | | TGGGATAACTTGGGCATGTGGTGGTTCTCCATAGCGCTTATGTTTGTATGC |
| | | CTTATTATTATGTGGCTCATCTGCTGCCTAAAGCGCAAACGCGCCCGACCA |
| | | CCCATCTATAGTCCCATCATTGTGCTACACCCAAACAATGATGGAATCCATA |
| | | GATTGGACGGACTGAAACACATGTTCTTTTCTCTTACAGTATGATTAAATGA |
| | | GACATGATTCCTCGAGTTTTTATATTACTGACCCTTGTTGCGCTTTTTTGTGC |
| | | GTGCTCCACATTGGCTGCGGTTTCTCACATCGAAGTAGACTGCATTCCAGC |
| | | CTTCACAGTCTATTTGCTTTACGGATTTGTCACCCTCACGCTCATCTGCAGC |
| | | CTCATCACTGTGGTCATCGCCTTTATCCAGTGCATTGACTGGGTCTGTGTG |
| | | CGCTTTGCATATCTCAGACACCATCCCCAGTACAGGGACAGGACTATAGCT |
| | | GAGCTTCTTAGAATTCTTTAATTATGAAATTTACTGTGACTTTTCTGCTGATT |
| | | ATTTGCACCCTATCTGCGTTTTGTTCCCCGACCTCCAAGCCTCAAAGACATA |
| | | TATCATGCAGATTCACTCGTATATGGAATATTCCAAGTTGCTACAATGAAAA |
| | | AAGCGATCTTTCCGAAGCCTGGTTATATGCAATCATCTCTGTTATGGTGTTC |
| | | TGCAGTACCATCTTAGCCCTAGCTATATATCCCTACCTTGACATTGGCTGGA |
| | | AACGAATAGATGCCATGAACCACCCAACTTTCCCCGCGCCCGCTATGCTTC |
| | | CACTGCAACAAGTTGTTGCCGGCGGCTTTGTCCCAGCCAATCAGCCTCGC |
| | | CCCACTTCTCCCACCCCCACTGAAATCAGCTACTTTAATCTAACAGGAGGA |
| | | GATGACTGACACCCTAGATCTAGAAATGGACGGAATTATTACAGAGCAGCG |
| | | CCTGCTAGAAAGACGCAGGGCAGCGGCCGAGCAACAGCGCATGAATCAAG |
| | | AGCTCCAAGACATGGTTAACTTGCACCAGTGCAAAAGGGGTATCTTTTGTC |
| | | TGGTAAAGCAGGCCAAAGTCACCTACGACAGTAATACCACCGGACACCGC |
| | | CTTAGCTACAAGTTGCCAACCAAGCGTCAGAAATTGGTGGTCATGGTGGGA |
| | | GAAAAGCCCATTACCATAACTCAGCACTCGGTAGAAACCGAAGGCTGCATT |
| | | CACTCACCTTGTCAAGGACCTGAGGATCTCTGCACCCTTATTAAGACCCTG |
| | | TGCGGTCTCAAAGATCTTATTCCCTTTAACTAATAAAAAAAAATAATAAAGCA |
| | | TCACTTACTTAAAATCAGTTAGCAAATTTCTGTCCAGTTTATTCAGCAGCACC |
| | | TCCTTGCCCTCCTCCCAGCTCTGGTATTGCAGCTTCCTCCTGGCTGCAAAC |
| | | TTTCTCCACAATCTAAATGGAATGTCAGTTTCCTCCTGTTCCTGTCCATCCG |
| | | CACCCACTATCTTCATGTTGTTGCAGATGAAGCGCGCAAGACCGTCTGAAG |
| | | ATACCTTCAACCCCGTGTATCCATATGACACGGAAACCGGTCCTCCAACTG |
| | | TGCCTTTTCTTACTCCTCCCTTTGTATCCCCCAATGGGTTTCAAGAGAGTCC |
| | | CCCTGGGGTACTCTCTTTGCGCCTATCCGAACCTCTAGTTACCTCCAATGG |
| | | CATGCTTGCGCTCAAAATGGGCAACGGCCTCTCTCTGGACGAGGCCGGCA |
| | | ACCTTACCTCCCAAAATGTAACCACTGTGAGCCCACCTCTCAAAAAAACCAA |
| | | GTCAAACATAAACCTGGAAATATCTGCACCCCTCACAGTTACCTCAGAAGC |
| | | CCTAACTGTGGCTGCCGCCGCACCTCTAATGGTCGCGGGCAACACACTCA |
| | | CCATGCAATCACAGGCCCCGCTAACCGTGCACGACTCCAAACTTAGCATTG |
| | | CCACCCAAGGACCCCTCACAGTGTCAGAAGGAAAGCTAGCCCTGCAAACA |
| | | TCAGGCCCCCTCACCACCACCGATAGCAGTACCCTTACTATCACTGCCTCA |
| | | CCCCCTCTAACTACTGCCACTGGTAGCTTGGGCATTGACTTGAAAGAGCCC |
| | | ATTTATACACAAAATGGAAAACTAGGACTAAAGTACGGGGCTCCTTTGCATG |
| | | TAACAGACGACCTAAACACTTTGACCGTAGCAACTGGTCCAGGTGTGACTA |
| | | TTAATAATACTTCCTTGCAAACTAAAGTTACTGGAGCCTTGGGTTTTGATTCA |
| | | CAAGGCAATATGCAACTTAATGTAGCAGGAGGACTAAGGATTGATTCTCAA |
| | | AACAGACGCCTTATACTTGATGTTAGTTATCCGTTTGATGCTCAAAACCAAC |
| | | TAAATCTAAGACTAGGACAGGGCCCTCTTTTTATAAACTCAGCCCACAACTT |
| | | GGATATTAACTACAACAAAGGCCTTTACTTGTTTACAGCTTCAAACAATTCC |
| | | AAAAAGCTTGAGGTTAACCTAAGCACTGCCAAGGGGTTGATGTTTGACGCT |
| | | ACAGCCATAGCCATTAATGCAGGAGATGGGCTTGAATTTGGTTCACCTAAT |
| | | GCACCAAACACAAATCCCCTCAAAACAAAAATTGGCCATGGCCTAGAATTT |
| | | GATTCAAACAAGGCTATGGTTCCTAAACTAGGAACTGGCCTTAGTTTTGACA |
| | | GCACAGGTGCCATTACAGTAGGAAACAAAAATAATGATAAGCTAACCCTAT |
| | | GGACAGGTCCAAAACCAGAAGCCAACTGCATAATTGAATACGGGAAACAAA |
| | | ACCCAGATAGCAAACTAACTTTAATCCTTGTAAAAAATGGAGGAATTGTTAA |
| | | TGGATATGTAACGCTAATGGGAGCCTCAGACTACGTTAACACCTTATTTAAA |
| | | AACAAAAATGTCTCCATTAATGTAGAACTATACTTTGATGCCACTGGTCATAT |
| | | ATTACCAGACTCATCTTCTCTTAAAACAGATCTAGAACTAAAATACAAGCAAA |
| | | CCGCTGACTTTAGTGCAAGAGGTTTTATGCCAAGTACTACAGCGTATCCATT |
| | | TGTCCTTCCTAATGCGGGAACACATAATGAAAATTATATTTTTGGTCAATGC |
| | | TACTACAAAGCAAGCGATGGTGCCCTTTTTCCGTTGGAAGTTACTGTTATGC |
| | | TTAATAAACGCCTGCCAGATAGTCGCACATCCTATGTTATGACTTTTTTATG |
| | | GTCCTTGAATGCTGGTCTAGCTCCAGAAACTACTCAGGCAACCCTCATAAC |
| | | CTCCCCATTTACCTTTTCCTATATTAGAGAAGATGACTAATAAACTCTAAAGA |
| | | ATCGTTTGTGTTATGTTTCAACGTGTTTATTTTTCAATTGCAGAAAATTTCAA |
| | | GTCATTTTTCATTCAGTAGTATAGCCCCACCACCACATAGCTTATACAGATC |
| | | ACCGTACCTTAATCAAACTCACAGAACCCTAGTATTCAACCTGCCACCTCCC |
| | | TCCCAACACACAGAGTACACAGTCCTTTCTCCCCGGCTGGCCTTAAAAAGC |
| | | ATCATATCATGGGTAACAGACATATTCTTAGGTGTTATATTCCACACGGTTT |
| | | CCTGTCGAGCCAAACGCTCATCAAGTGATATTAATAAACTCCCCGGGCAGC |
| | | TCACTTAAGTTCATGTCGCTGTCCAGCTGCTGAGCCACAGGCTGCTGTCCA |
| | | ACTTGCGGTTGCTTAACGGGCGGCGAAGGAGAAGTCCACGCCTACATGGG |
| | | GGGAGAGTCATAATCGTGCATCAGGATAGGGCGGTGGTGCTGCAGCAGCG |
| | | CGCGAATAAACTGCTGCCGCCGCCGCTCCGTCCTGCAGGAATACAACATG |
| | | GCAGTGGTCTCCTCAGCGATGATTCGCACCGCCCGCAGCATAAGGCGCTT |
| | | GTCCTCCGGGCACAGCAGCGCACCCTGATCTCACTTAAATCAGCACAGTAA |
| | | CTGCAGCACAGCACCACAATATTGTTCAAAATCCCACAGTGCAAGGCGCTG |
| | | TATCCAAAGCTCATGGCGGGGACCACAGAACCCACGTGGCCATCATACCA |
| | | CAAGCGCAGGTAGATTAAGTGGCGACCCCTCATAAACACGCTGGACATAAA |
| | | CATTACCTCTTTTGGCATGTTGTAATTCACCACCTCCCGGTACCATATAAAC |
| | | CTCTGATTAAACATGGCGCCATCCACCACCATCCTAAACCAGCTGGCCAAA |
| | | ACCTGCCCCGCCGGGNTATACACTGCAGGGAACCGGGACTTGGACAATGA |
| | | CAAGTGGGAGAGCCCAGGACTCGTAACCATGGATCATCATGCTCGTCATGA |
| | | TATCAATGTTGGCACAACACAGGCACACGTGCATACACTTCCTCAGGATTA |
| | | CAAGCTCCTCCCGCGTTAGAACCATATCCCAGGGAACAACCCATTCCTGAA |
| | | TCAGCGTAAATCCCACACTGCAGGGAAGACCTCGCACGTAACTCACGTTGT |
| | | GCATTGTCAAAGTGTTACATTCGGGCAGCAGCGGATGATCCTCCAGTATGG |
| | | TAGCGCGGGTTTCTGTCTCAAAAGGAGGTAGACGATCCCTACTGTACGGAG |
| | | TGCGCCGAGACAACCGAGATCGTGTTGGTCGTAGTGTCATGCCAAATGGA |
| | | ACGCCGGACGTAGTCATATTTCCTGAAGCAAAACCAGGTGCGGGCGTGAC |
| | | AAACAGATCTGCGTCTCCGGTCTCGCCGCTTAGATCGCTCTGTGTAGTAGT |
| | | TGTAGTATATCCACTCTCTCAAAGCATCCAGGCGCCCCCTGGCTTCGGGTT |
| | | CTATGTAAACTCCTTCATGCGCCGCTGCCCTGATAACATCCACCACCGCAG |
| | | AATAAGCCACACCCAGCCAACCTACACATTCGTTCTGCGAGTCACACACGG |
| | | GAGGAGCGGGAAGAGCTGGAAGAACCATGTTTTTTTTTTTATTCCAAAAGAT |
| | | TATCCAAAACCTCAAAATGAAGATCTATTAAGTGAACGCGCTCCCCTCCGGT |
| | | GGCGTGGTCAAACTCTACAGCCAAAGAACAGATAATGGCATTTGTAAGATG |
| | | TTGCACAATGGCTTCCAAAAGGCAAACGGCCCTCACGTCCAAGTGGACGTA |
| | | AAGGCTAAACCCTTCAGGGTGAATCTCCTCTATAAACATTCCAGCACCTTCA |
| | | ACCATGCCCAAATAATTCTCATCTCGCCACCTTCTCAATATATCTCTAAGCA |
| | | AATCCCGAATATTTAAGTCCGGGCCATTGTAAAAAATTTGGCTCCAGAGCG |
| | | CCCTCCACCTTCAGCCTCAAGCAGCGAATCATGATTGCAAAAATTCAGGTT |
| | | CCTCACAGACCTGTATAAGATTCAAAAGCGGAACATTAACAAAAATACCGC |
| | | GATCCCGTAGGTCCCTTCGCAGGGCCAGCTGAACATAATCGTGCAGGTCT |
| | | GCACGGACCAGCGCGGCCACTTCCCCGCCAGGAACCATGACAAAAGAACC |
| | | CACACTGATTATGACACGCATACTCGGAGCTATGCTAACCAGCGTAGCCCC |
| | | GATGTAAGCTTGTTGCATGGGCGGCGATATAAAATGCAAGGTGCTGCTCAA |
| | | AAAATCAGGCAAAGCCTCGCGCAAAAAAGAAAGCACATCGTAGTCATGCTC |
| | | ATGCAGATAAAGGCAGGTAAGCTCCGGAACCACCACAGAAAAAGACACCAT |
| | | TTTTCTCTCAAACATGTCTGCGGGTTTCTGCATAAACACAAAATAAAATAACA |
| | | AAAAAACATTTAAACATTAGAAGCCTGTCTTACAACAGGAAAAACAACCCTT |
| | | ATAAGCATAAGACGGACTACGGCCATGCCGGCGTGACCGTAAAAAAACTG |
| | | GTCACCGTGATTAAAAAGCACCACCGACAGCTCCTCGGTCAGTCCGGAGT |
| | | CATAATGTAAGACTCGGTAAACACATCAGGTTGATTCACATCGGTCAGTGTT |
| | | AAAAAGCGACCGAAATAGCCNGGGGGAATACAATACCCGCAGGCGTAGAG |
| | | ACAACATTACAGCCCCCATAGGAGGTATAACAAAATTAATAGGAGAGAAAAA |
| | | CACATAAACACCTGAAAAACCCTCCTGCCTAGGCAAAATAGCACCCTCCCG |
| | | CTCCAGAACAACATACAGCGCTTCCACAGCGGCAGCCATAACAGTCAGCCT |
| | | TACCAGTAAAAAAGAAAACCTATTAAAAAAACACCACTCGACACGGCACCA |
| | | GCTCAATCAGTCACAGTGTAAAAAAGGGCCAAGTGCAGAGCGAGTATATAT |
| | | AGGACTAAAAAATGACGGTAACGGTTAAAGTCCACAAAAAACACCCAGAAA |
| | | ACCGCACGCGAACCTACGCCCAGAAACGAAAGCCAAAAAACCCACAACTTC |
| | | CTCAAATCGTCACTTCCGTTTTCCCACGTTACGTCACTTCCCATTTTAAGAA |
| | | AACTACAATTCCCAACACATACAAGTTACTCCGCCCTAAAACCTACGTCACC |
| | | CGCCCCGTTCCCACGCCCCGCGCCACGTCACAAACTCCACCCCCTCATTA |
| | | TCATATTGGCTTCAATCCAAAATAAGGTATATTATTGATGATGTTAATTAACA |
| | | TGCATGGATCCTCGTCTCGACGATGCCCTTGAGAGCCTTCAACCCAGTCAG |
| | | CTCCTTCCGGTGGGCGCGGGGCATGACTATCGTCGCCGCACTTATGACTG |
| | | TCTTCTTTATCATGCAACTCGTAGGACAGGTGCCGGCAGCGCTCTGGGTCA |
| | | TTTTCGGCGAGGACCGCTTTCGCTGGAGCGCGACGATGATCGGCCTGTCG |
| | | CTTGCGGTATTCGGAATCTTGCACGCCCTCGCTCAAGCCTTCGTCACTGGT |
| | | CCCGCCACCAAACGTTTCGGCGAGAAGCAGGCCATTATCGCCGGCATGGC |
| | | GGCCGACGCGCTGGGCTACGTCTTGCTGGCGTTCGCGACGCGAGGCTGG |
| | | ATGGCCTTCCCCATTATGATTCTTCTCGCTTCCGGCGGCATCGGGATGCCC |
| | | GCGTTGCAGGCCATGCTGTCCAGGCAGGTAGATGACGACCATCAGGGACA |
| | | GCTTCAAGGATCGCTCGCGGCTCTTACCAGCCTAACTTCGATCACTGGACC |
| | | GCTGATCGTCACGGCGATTTATGCCGCCTCGGCGAGCACATGGAACGGGT |
| | | TGGCATGGATTGTAGGCGCCGCCCTATACCTTGTCTGCCTCCCCGCGTTG |
| | | CGTCGCGGTGCATGGAGCCGGGCCACCTCGACCTGAATGGAAGCCGGCG |
| | | GCACCTCGCTAACGGATTCACCACTCCAAGAATTGGAGCCAATCAATTCTT |
| | | GCGGAGAACTGTGAATGCGCAAACCAACCCTTGGCAGAACATATCCATCGC |
| | | GTCCGCCATCTCCAGCAGCCGCACGCGGCGCATCTCGGGCAGCGTTGGG |
| | | TCCTGGCCACGGGTGCGCATGATCGTGCTCCTGTCGTTGAGGACCCGGCT |
| | | AGGCTGGCGGGGTTGCCTTACTGGTTAGCAGAATGAATCACCGATACGCG |
| | | AGCGAACGTGAAGCGACTGCTGCTGCAAAACGTCTGCGACCTGAGCAACA |
| | | ACATGAATGGTCTTCGGTTTCCGTGTTTCGTAAAGTCTGGAAACGCGGAAG |
| | | TCAGCGCCCTGCACCATTATGTTCCGGATCTGCATCGCAGGATGCTGCTGG |
| | | CTACCCTGTGGAACACCTACATCTGTATTAACGAAGCGCTGGCATTGACCC |
| | | TGAGTGATTTTTCTCTGGTCCCGCCGCATCCATACCGCCAGTTGTTTACCCT |
| | | CACAACGTTCCAGTAACCGGGCATGTTCATCATCAGTAACCCGTATCGTGA |
| | | GCATCCTCTCTCGTTTCATCGGTATCATTACCCCCATGAACAGAAATTCCCC |
| | | CTTACACGGAGGCATCAAGTGACCAAACAGGAAAAAACCGCCCTTAACATG |
| | | GCCCGCTTTATCAGAAGCCAGACATTAACGCTTCTGGAGAAACTCAACGAG |
| | | CTGGACGCGGATGAACAGGCAGACATCTGTGAATCGCTTCACGACCACGC |
| | | TGATGAGCTTTACCGCAGCTGCCTCGCGCGTTTCGGTGATGACGGTGAAAA |
| | | CCTCTGACACATGCAGCTCCCGGAGACGGTCACAGCTTGTCTGTAAGCGG |
| | | ATGCCGGGAGCAGACAAGCCCGTCAGGGCGCGTCAGCGGGTGTTGGCGG |
| | | GTGTCGGGGCGCAGCCATGACCCAGTCACGTAGCGATAGCGGAGTGTATA |
| | | CTGGCTTAACTATGCGGCATCAGAGCAGATTGTACTGAGAGTGCACCATAT |
| | | GCGGTGTGAAATACCGCACAGATGCGTAAGGAGAAAATACCGCATCAGGC |
| | | GCTCTTCCGCTTCCTCGCTCACTGACTCGCTGCGCTCGGTCGTTCGGCTGC |
| | | GGCGAGCGGTATCAGCTCACTCAAAGGCGGTAATACGGTTATCCACAGAAT |
| | | CAGGGGATAACGCAGGAAAGAACATGTGAGCAAAAGGCCAGCAAAAGGCC |
| | | AGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACC |
| | | CGACAGGACTATAAAGATACCAGGCGTTTCCCCCTGGAAGCTCCCTCGTGC |
| | | GCTCTCCTGTTCCGACCCTGCCGCTTACCGGATACCTGTCCGCCTTTCTCC |
| | | CTTCGGGAAGCGTGGCGCTTTCTCAATGCTCACGCTGTAGGTATCTCAGTT |
| | | CGGTGTAGGTCGTTCGCTCCAAGCTGGGCTGTGTGCACGAACCCCCCGTT |
| | | CAGCCCGACCGCTGCGCCTTATCCGGTAACTATCGTCTTGAGTCCAACCCG |
| | | GTAAGACACGACTTATCGCCACTGGCAGCAGCCACTGGTAACAGGATTAGC |
| | | AGAGCGAGGTATGTAGGCGGTGCTACAGAGTTCTTGAAGTGGTGGCCTAA |
| | | CTACGGCTACACTAGAAGGACAGTATTTGGTATCTGCGCTCTGCTGAAGCC |
| | | AGTTACCTTCGGAAAAAGAGTTGGTAGCTCTTGATCCGGCAAACAAACCAC |
| | | CGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCA |
| | | GTGGAACGAAAACTCACGTTAAGGGATTTTGGTCATGAGATTATCAAAAAG |
| | | GATCTTCACCTAGATCCTTTTAAATTAAAAATGAAGTTTTAAATCAATCTAAA |
| | | GTATATATGAGTAAACTTGGTCTGACAGTTACCAATGCTTAATCAGTGAGGC |
| | | ACCTATCTCAGCGATCTGTCTATTTCGTTCATCCATAGTTGCCTGACTCCCC |
| | | GTCGTGTAGATAACTACGATACGGGAGGGCTTACCATCTGGCCCCAGTGCT |
| | | GCAATGATACCGCGAGACCCACGCTCACCGGCTCCAGATTTATCAGCAATA |
| | | AACCAGCCAGCCGGAAGGGCCGAGCGCAGAAGTGGTCCTGCAACTTTATC |
| | | CGCCTCCATCCAGTCTATTAATTGTTGCCGGGAAGCTAGAGTAAGTAGTTC |
| | | GCCAGTTAATAGTTTGCGCAACGTTGGTTGNNNNNNAAAAAGGATCTTCAC |
| | | CTAGATCCTTTTCACGTAGAAAGCCAGTCCGCAGAAACGGTGCTGACCCCG |
| | | GATGAATGTCAGCTACTGGGCTATCTGGACAAGGGAAAACGCAAGCGCAA |
| | | AGAGAAAGCAGGTAGCTTGCAGTGGGCTTACATGGCGATAGCTAGACTGG |
| | | GCGGTTTTATGGACAGCAAGCGAACCGGAATTGCCAGCTGGGGCGCCCTC |
| | | TGGTAAGGTTGGGAAGCCCTGCAAAGTAAACTGGATGGCTTTCTCGCCGC |
| | | CAAGGATCTGATGGCGCAGGGGATCAAGCTCTGATCAAGAGACAGGATGA |
| | | GGATCGTTTCGCATGATTGAACAAGATGGATTGCACGCAGGTTCTCCGGCC |
| | | GCTTGGGTGGAGAGGCTATTCGGCTATGACTGGGCACAACAGACAATCGG |
| | | CTGCTCTGATGCCGCCGTGTTCCGGCTGTCAGCGCAGGGGCGCCCGGTTC |
| | | TTTTTGTCAAGACCGACCTGTCCGGTGCCCTGAATGAACTGCAAGACGAGG |
| | | CAGCGCGGCTATCGTGGCTGGCCACGACGGGCGTTCCTTGCGCAGCTGT |
| | | GCTCGACGTTGTCACTGAAGCGGGAAGGGACTGGCTGCTATTGGGCGAAG |
| | | TGCCGGGGCAGGATCTCCTGTCATCTCACCTTGCTCCTGCCGAGAAAGTAT |
| | | CCATCATGGCTGATGCAATGCGGCGGCTGCATACGCTTGATCCGGCTACC |
| | | TGCCCATTCGACCACCAAGCGAAACATCGCATCGAGCGAGCACGTACTCG |
| | | GATGGAAGCCGGTCTTGTCGATCAGGATGATCTGGACGAAGAGCATCAGG |
| | | GGCTCGCGCCAGCCGAACTGTTCGCCAGGCTCAAGGCGAGCATGCCCGA |
| | | CGGCGAGGATCTCGTCGTGACCCATGGCGATGCCTGCTTGCCGAATATCA |
| | | TGGTGGAAAATGGCCGCTTTTCTGGATTCATCGACTGTGGCCGGCTGGGT |
| | | GTGGCGGACCGCTATCAGGACATAGCGTTGGCTACCCGTGATATTGCTGA |
| | | AGAGCTTGGCGGCGAATGGGCTGACCGCTTCCTCGTGCTTTACGGTATCG |
| | | CCGCTCCCGATTCGCAGCGCATCGCCTTCTATCGCCTTCTTGACGAGTTCT |
| | | TCTGAATTTTGTTAAAATTTTTGTTAAATCAGCTCATTTTTTAACCAATAGGC |
| | | CGAAATCGGCAACATCCCTTATAAATCAAAAGAATAGACCGCGATAGGGTT |
| | | GAGTGTTGTTCCAGTTTGGAACAAGAGTCCACTATTAAAGAACGTGGACTC |
| | | CAACGTCAAAGGGCGAAAAACCGTCTATCAGGGCGATGGCCCACTACGTG |
| | | AACCATCACCCAAATCAAGTTTTTTGCGGTCGAGGTGCCGTAAAGCTCTAA |
| | | ATCGGAACCCTAAAGGGAGCCCCCGATTTAGAGCTTGACGGGGAAAGCCG |
| | | GCGAACGTGGCGAGAAAGGAAGGGAAGAAAGCGAAAGGAGCGGGCGCTA |
| | | GGGCGCTGGCAAGTGTAGCGGTCACGCTGCGCGTAACCACCACACCCGC |
| | | GCGCTTAATGCGCCGCTACAGGGCGCGTCCATTCGCCATTCAGGATCGAA |
| | | TTAATTCTTAAT |
| 52 | Amino acids | LTCHEACF |
| | 121-128 of Ad | |
| | E1A protein | |
| 53 | STAT1 binding | TTCCGGGAA |
| | site (1) | |
| 54 | STAT1 binding | TTCTCGGAA |
| | site (2) | |
| 55 | Ad5/3Ad2E1AΔ | TAACATCATCAATAATATACCTTATTTTGGATTGAAGCCAATATGATAATGAG |
| | 24 | GGGGTGGAGTTTGTGACGTGGCGCGGGGCGTGGGAACGGGGCGGGTGA |
| | | CGTAGTAGTGTGGCGGAAGTGTGATGTTGCAAGTGTGGCGGAACACATGT |
| | | AAGCGACGGATGTGGCAAAAGTGACGTTTTTGGTGTGCGCCGGTGTACAC |
| | | AGGAAGTGACAATTTTCGCGCGGTTTTAGGCGGATGTTGTAGTAAATTTGG |
| | | GCGTAACCGAGTAAGATTTGGCCATTTTCGCGGGAAAACTGAATAAGAGGA |
| | | AGTGAAATCTGAATAATTTTGTGTTACTCATAGCGCGTAATATTTGTCTAGG |
| | | GCCGCGGGGACTTTGACCGTTTACGTGGAGACTCGCCCAGGTGTTTTTCTC |
| | | AGGTGTTTTCCGCGTTCCGGGTCAAAGTTGGCGTTTTATTATTATAGTCAGC |
| | | TGACGTGTAGTGTATTTATACCCGGTGAGTTCCTCAAGAGGCCACTCTTGA |
| | | GTGCCAGCGAGTAGAGTTTTCTCCTCCGAGCCGCTCCGACACCGGGACTG |
| | | AAAATGAGACATATTATCTGCCACGGAGGTGTTATTACCGAAGAAATGGCC |
| | | GCCAGTCTTTTGGACCAGCTGATCGAAGAGGTACTGGCTGATAATCTTCCA |
| | | CCTCCTAGCCATTTTGAACCACCTACCCTTCACGAACTGTATGATTTAGACG |
| | | TGACGGCCCCCGAAGATCCCAACGAGGAGGCGGTTTCGCAGATTTTTCCC |
| | | GAGTCTGTAATGTTGGCGGTGCAGGAAGGGATTGACTTATTCACTTTTCCG |
| | | CCGGCGCCCGGTTCTCCGGAGCCGCCTCACCTTTCCCGGCAGCCCGAGC |
| | | AGCCGGAGCAGAGAGCCTTGGGTCCGGTTTCTATGCCAAACCTTGTGCCG |
| | | GAGGTGATCGATCCACCCAGTGACGACGAGGATGAAGAGGGTGAGGAGTT |
| | | TGTGTTAGATTATGTGGAGCACCCCGGGCACGGTTGCAGGTCTTGTCATTA |
| | | TCACCGGAGGAATACGGGGGACCCAGATATTATGTGTTCGCTTTGCTATAT |
| | | GAGGACCTGTGGCATGTTTGTCTACAGTAAGTGAAAATTATGGGCAGTCGG |
| | | TGATAGAGTGGTGGGTTTGGTGTGGTAATTTTTTTTTAATTTTTACAGTTTTG |
| | | TGGTTTAAAGAATTTTGTATTGTGATTTTTTAAAAGGTCCTGTGTCTGAACCT |
| | | GAGCCTGAGCCCGAGCCAGAACCGGAGCCTGCAAGACCTACCCGGCGTC |
| | | CTAAATTGGTGCCTGCTATCCTGAGACGCCCGACATCACCTGTGTCTAGAG |
| | | AATGCAATAGTAGTACGGATAGCTGTGACTCCGGTCCTTCTAACACACCTC |
| | | CTGAGATACACCCGGTGGTCCCGCTGTGCCCCATTAAACCAGTTGCCGTG |
| | | AGAGTTGGTGGGCGTCGCCAGGCTGTGGAATGTATCGAGGACTTGCTTAA |
| | | CGAGTCTGGGCAACCTTTGGACTTGAGCTGTAAACGCCCCAGGCCATAAG |
| | | GTGTAAACCTGTGATTGCGTGTGTGGTTAACGCCTTTGTTTGCTGAATGAGT |
| | | TGATGTAAGTTTAATAAAGGGTGAGATAATGTTTAACTTGCATGGCGTGTTA |
| | | AATGGGGCGGGGCTTAAAGGGTATATAATGCGCCGTGGGCTAATCTTGGTT |
| | | ACATCTGACCTCATGGAGGCTTGGGAGTGTTTGGAAGATTTTTCTGCTGTG |
| | | CGTAACTTGCTGGAACAGAGCTCTAACAGTACCTCTTGGTTTTGGAGGTTT |
| | | CTGTGGGGCTCATCCCAGGCAAAGTTAGTCTGCAGAATTAAGGAGGATTAC |
| | | AAGTGGGAATTTGAAGAGCTTTTGAAATCCTGTGGTGAGCTGTTTGATTCTT |
| | | TGAATCTGGGTCACCAGGCGCTTTTCCAAGAGAAGGTCATCAAGACTTTGG |
| | | ATTTTTCCACACCGGGGCGCGCTGCGGCTGCTGTTGCTTTTTTGAGTTTTAT |
| | | AAAGGATAAATGGAGCGAAGAAACCCATCTGAGCGGGGGGTACCTGCTGG |
| | | ATTTTCTGGCCATGCATCTGTGGAGAGCGGTTGTGAGACACAAGAATCGCC |
| | | TGCTACTGTTGTCTTCCGTCCGCCCGGCGATAATACCGACGGAGGAGCAG |
| | | CAGCAGCAGCAGGAGGAAGCCAGGCGGCGGCGGCAGGAGCAGAGCCCAT |
| | | GGAACCCGAGAGCCGGCCTGGACCCTCGGGAATGAATGTTGTTCAGGTGG |
| | | CTGAACTGTATCCAGAACTGAGACGCATTTTGACAATTACAGAGGATGGGC |
| | | AGGGGCTAAAGGGGGTAAAGAGGGAGCGGGGGGCTTGTGAGGCTACAGA |
| | | GGAGGCTAGGAATCTAGCTTTTAGCTTAATGACCAGACACCGTCCTGAGTG |
| | | TATTACTTTTCAACAGATCAAGGATAATTGCGCTAATGAGCTTGATCTGCTG |
| | | GCGCAGAAGTATTCCATAGAGCAGCTGACCACTTACTGGCTGCAGCCAGG |
| | | GGATGATTTTGAGGAGGCTATTAGGGTATATGCAAAGGTGGCACTTAGGCC |
| | | AGATTGCAAGTACAAGATCAGCAAACTTGTAAATATCAGGAATTGTTGCTAC |
| | | ATTTCTGGGAACGGGGCCGAGGTGGAGATAGATACGGAGGATAGGGTGGC |
| | | CTTTAGATGTAGCATGATAAATATGTGGCCGGGGGTGCTTGGCATGGACGG |
| | | GGTGGTTATTATGAATGTAAGGTTTACTGGCCCCAATTTTAGCGGTACGGTT |
| | | TTCCTGGCCAATACCAACCTTATCCTACACGGTGTAAGCTTCTATGGGTTTA |
| | | ACAATACCTGTGTGGAAGCCTGGACCGATGTAAGGGTTCGGGGCTGTGCC |
| | | TTTTACTGCTGCTGGAAGGGGGTGGTGTGTCGCCCCAAAAGCAGGGCTTC |
| | | AATTAAGAAATGCCTCTTTGAAAGGTGTACCTTGGGTATCCTGTCTGAGGGT |
| | | AACTCCAGGGTGCGCCACAATGTGGCCTCCGACTGTGGTTGCTTCATGCTA |
| | | GTGAAAAGCGTGGCTGTGATTAAGCATAACATGGTATGTGGCAACTGCGAG |
| | | GACAGGGCCTCTCAGATGCTGACCTGCTCGGACGGCAACTGTCACCTTCT |
| | | GAAGACCATTCACGTAGCCAGCCACTCTCGCAAGGCCTGGCCAGTGTTTG |
| | | AGCATAACATACTGACCCGCTGTTCCTTGCATTTGGGTAACAGGAGGGGGG |
| | | TGTTCCTACCTTACCAATGCAATTTGAGTCACACTAAGATATTGCTTGAGCC |
| | | CGAGAGCATGTCCAAGGTGAACCTGAACGGGGTGTTTGACATGACCATGA |
| | | AGATCTGGAAGGTGCTGAGGTACGATGAGACCCGCACCAGGTGCAGACCC |
| | | TGCGAGTGTGGCGGTAAACATATTAGGAACCAGCCTGTGATGCTGGATGTG |
| | | ACCGAGGAGCTGAGGCCCGATCACTTGGTGCTGGCCTGCACCCGCGCTGA |
| | | GTTTGGCTCTAGCGATGAAGATACAGATTGAGGTACTGAAATGTGTGGGCG |
| | | TGGCTTAAGGGTGGGAAAGAATATATAAGGTGGGGGTCTTATGTAGTTTTG |
| | | TATCTGTTTTGCAGCAGCCGCCGCCGCCATGAGCACCAACTCGTTTGATGG |
| | | AAGCATTGTGAGCTCATATTTGACAACGCGCATGCCCCCATGGGCCGGGG |
| | | TGCGTCAGAATGTGATGGGCTCCAGCATTGATGGTCGCCCCGTCCTGCCC |
| | | GCAAACTCTACTACCTTGACCTACGAGACCGTGTCTGGAACGCCGTTGGAG |
| | | ACTGCAGCCTCCGCCGCCGCTTCAGCCGCTGCAGCCACCGCCCGCGGGA |
| | | TTGTGACTGACTTTGCTTTCCTGAGCCCGCTTGCAAGCAGTGCAGCTTCCC |
| | | GTTCATCCGCCCGCGATGACAAGTTGACGGCTCTTTTGGCACAATTGGATT |
| | | CTTTGACCCGGGAACTTAATGTCGTTTCTCAGCAGCTGTTGGATCTGCGCC |
| | | AGCAGGTTTCTGCCCTGAAGGCTTCCTCCCCTCCCAATGCGGTTTAAAACA |
| | | TAAATAAAAAACCAGACTCTGTTTGGATTTGGATCAAGCAAGTGTCTTGCTG |
| | | TCTTTATTTAGGGGTTTTGCGCGCGCGGTAGGCCCGGGACCAGCGGTCTC |
| | | GGTCGTTGAGGGTCCTGTGTATTTTTTCCAGGACGTGGTAAAGGTGACTCT |
| | | GGATGTTCAGATACATGGGCATAAGCCCGTCTCTGGGGTGGAGGTAGCAC |
| | | CACTGCAGAGCTTCATGCTGCGGGGTGGTGTTGTAGATGATCCAGTCGTA |
| | | GCAGGAGCGCTGGGCGTGGTGCCTAAAAATGTCTTTCAGTAGCAAGCTGA |
| | | TTGCCAGGGGCAGGCCCTTGGTGTAAGTGTTTACAAAGCGGTTAAGCTGG |
| | | GATGGGTGCATACGTGGGGATATGAGATGCATCTTGGACTGTATTTTTAGG |
| | | TTGGCTATGTTCCCAGCCATATCCCTCCGGGGATTCATGTTGTGCAGAACC |
| | | ACCAGCACAGTGTATCCGGTGCACTTGGGAAATTTGTCATGTAGCTTAGAA |
| | | GGAAATGCGTGGAAGAACTTGGAGACGCCCTTGTGACCTCCAAGATTTTCC |
| | | ATGCATTCGTCCATAATGATGGCAATGGGCCCACGGGCGGCGGCCTGGGC |
| | | GAAGATATTTCTGGGATCACTAACGTCATAGTTGTGTTCCAGGATGAGATC |
| | | GTCATAGGCCATTTTTACAAAGCGCGGGCGGAGGGTGCCAGACTGCGGTA |
| | | TAATGGTTCCATCCGGCCCAGGGGCGTAGTTACCCTCACAGATTTGCATTT |
| | | CCCACGCTTTGAGTTCAGATGGGGGGATCATGTCTACCTGCGGGGCGATG |
| | | AAGAAAACGGTTTCCGGGGTAGGGGAGATCAGCTGGGAAGAAAGCAGGTT |
| | | CCTGAGCAGCTGCGACTTACCGCAGCCGGTGGGCCCGTAAATCACACCTA |
| | | TTACCGGGTGCAACTGGTAGTTAAGAGAGCTGCAGCTGCCGTCATCCCTGA |
| | | GCAGGGGGGCCACTTCGTTAAGCATGTCCCTGACTCGCATGTTTTCCCTGA |
| | | CCAAATCCGCCAGAAGGCGCTCGCCGCCCAGCGATAGCAGTTCTTGCAAG |
| | | GAAGCAAAGTTTTTCAACGGTTTGAGACCGTCCGCCGTAGGCATGCTTTTG |
| | | AGCGTTTGACCAAGCAGTTCCAGGCGGTCCCACAGCTCGGTTACCTGCTCT |
| | | ACGGCATCTCGATCCAGCATATCTCCTCGTTTCGCGGGTTGGGGCGGCTTT |
| | | CGCTGTACGGCAGTAGTCGGTGCTCGTCCAGACGGGCCAGGGTCATGTCT |
| | | TTCCACGGGCGCAGGGTCCTCGTCAGCGTAGTCTGGGTCACGGTGAAGGG |
| | | GTGCGCTCCGGGCTGCGCGCTGGCCAGGGTGCGCTTGAGGCTGGTCCTG |
| | | CTGGTGCTGAAGCGCTGCCGGTCTTCGCCCTGCGCGTCGGCCAGGTAGCA |
| | | TTTGACCATGGTGTCATAGTCCAGCCCCTCCGCGGCGTGGCCCTTGGCGC |
| | | GCAGCTTGCCCTTGGAGGAGGCGCCGCACGAGGGGCAGTGCAGACTTTT |
| | | GAGGGCGTAGAGCTTGGGCGCGAGAAATACCGATTCCGGGGAGTAGGCAT |
| | | CCGCGCCGCAGGCCCCGCAGACGGTCTCGCATTCCACGAGCCAGGTGAG |
| | | CTCTGGCCGTTCGGGGTCAAAAACCAGGTTTCCCCCATGCTTTTTGATGCG |
| | | TTTCTTACCTCTGGTTTCCATGAGCCGGTGTCCACGCTCGGTGACGAAAAG |
| | | GCTGTCCGTGTCCCCGTATACAGACTTGAGAGGCCTGTCCTCGAGCGGTG |
| | | TTCCGCGGTCCTCCTCGTATAGAAACTCGGACCACTCTGAGACAAAGGCTC |
| | | GCGTCCAGGCCAGCACGAAGGAGGCTAAGTGGGAGGGGTAGCGGTCGTT |
| | | GTCCACTAGGGGGTCCACTCGCTCCAGGGTGTGAAGACACATGTCGCCCT |
| | | CTTCGGCATCAAGGAAGGTGATTGGTTTGTAGGTGTAGGCCACGTGACCG |
| | | GGTGTTCCTGAAGGGGGGCTATAAAAGGGGGTGGGGGCGCGTTCGTCCT |
| | | CACTCTCTTCCGCATCGCTGTCTGCGAGGGCCAGCTGTTGGGGTGAGTAC |
| | | TCCCTCTGAAAAGCGGGCATGACTTCTGCGCTAAGATTGTCAGTTTCCAAA |
| | | AACGAGGAGGATTTGATATTCACCTGGCCCGCGGTGATGCCTTTGAGGGT |
| | | GGCCGCATCCATCTGGTCAGAAAAGACAATCTTTTTGTTGTCAAGCTTGGT |
| | | GGCAAACGACCCGTAGAGGGCGTTGGACAGCAACTTGGCGATGGAGCGC |
| | | AGGGTTTGGTTTTTGTCGCGATCGGCGCGCTCCTTGGCCGCGATGTTTAGC |
| | | TGCACGTATTCGCGCGCAACGCACCGCCATTCGGGAAAGACGGTGGTGCG |
| | | CTCGTCGGGCACCAGGTGCACGCGCCAACCGCGGTTGTGCAGGGTGACA |
| | | AGGTCAACGCTGGTGGCTACCTCTCCGCGTAGGCGCTCGTTGGTCCAGCA |
| | | GAGGCGGCCGCCCTTGCGCGAGCAGAATGGCGGTAGGGGGTCTAGCTGC |
| | | GTCTCGTCCGGGGGGTCTGCGTCCACGGTAAAGACCCCGGGCAGCAGGC |
| | | GCGCGTCGAAGTAGTCTATCTTGCATCCTTGCAAGTCTAGCGCCTGCTGCC |
| | | ATGCGCGGGCGGCAAGCGCGCGCTCGTATGGGTTGAGTGGGGGACCCCA |
| | | TGGCATGGGGTGGGTGAGCGCGGAGGCGTACATGCCGCAAATGTCGTAAA |
| | | CGTAGAGGGGCTCTCTGAGTATTCCAAGATATGTAGGGTAGCATCTTCCAC |
| | | CGCGGATGCTGGCGCGCACGTAATCGTATAGTTCGTGCGAGGGAGCGAG |
| | | GAGGTCGGGACCGAGGTTGCTACGGGCGGGCTGCTCTGCTCGGAAGACT |
| | | ATCTGCCTGAAGATGGCATGTGAGTTGGATGATATGGTTGGACGCTGGAAG |
| | | ACGTTGAAGCTGGCGTCTGTGAGACCTACCGCGTCACGCACGAAGGAGGC |
| | | GTAGGAGTCGCGCAGCTTGTTGACCAGCTCGGCGGTGACCTGCACGTCTA |
| | | GGGCGCAGTAGTCCAGGGTTTCCTTGATGATGTCATACTTATCCTGTCCCT |
| | | TTTTTTTCCACAGCTCGCGGTTGAGGACAAACTCTTCGCGGTCTTTCCAGTA |
| | | CTCTTGGATCGGAAACCCGTCGGCCTCCGAACGGTAAGAGCCTAGCATGT |
| | | AGAACTGGTTGACGGCCTGGTAGGCGCAGCATCCCTTTTCTACGGGTAGC |
| | | GCGTATGCCTGCGCGGCCTTCCGGAGCGAGGTGTGGGTGAGCGCAAAGG |
| | | TGTCCCTGACCATGACTTTGAGGTACTGGTATTTGAAGTCAGTGTCGTCGC |
| | | ATCCGCCCTGCTCCCAGAGCAAAAAGTCCGTGCGCTTTTTGGAACGCGGAT |
| | | TTGGCAGGGCGAAGGTGACATCGTTGAAGAGTATCTTTCCCGCGCGAGGC |
| | | ATAAAGTTGCGTGTGATGCGGAAGGGTCCCGGCACCTCGGAACGGTTGTT |
| | | AATTACCTGGGCGGCGAGCACGATCTCGTCAAAGCCGTTGATGTTGTGGC |
| | | CCACAATGTAAAGTTCCAAGAAGCGCGGGATGCCCTTGATGGAAGGCAATT |
| | | TTTTAAGTTCCTCGTAGGTGAGCTCTTCAGGGGAGCTGAGCCCGTGCTCTG |
| | | AAAGGGCCCAGTCTGCAAGATGAGGGTTGGAAGCGACGAATGAGCTCCAC |
| | | AGGTCACGGGCCATTAGCATTTGCAGGTGGTCGCGAAAGGTCCTAAACTG |
| | | GCGACCTATGGCCATTTTTTCTGGGGTGATGCAGTAGAAGGTAAGCGGGTC |
| | | TTGTTCCCAGCGGTCCCATCCAAGGTTCGCGGCTAGGTCTCGCGCGGCAG |
| | | TCACTAGAGGCTCATCTCCGCCGAACTTCATGACCAGCATGAAGGGCACGA |
| | | GCTGCTTCCCAAAGGCCCCCATCCAAGTATAGGTCTCTACATCGTAGGTGA |
| | | CAAAGAGACGCTCGGTGCGAGGATGCGAGCCGATCGGGAAGAACTGGATC |
| | | TCCCGCCACCAATTGGAGGAGTGGCTATTGATGTGGTGAAAGTAGAAGTCC |
| | | CTGCGACGGGCCGAACACTCGTGCTGGCTTTTGTAAAAACGTGCGCAGTA |
| | | CTGGCAGCGGTGCACGGGCTGTACATCCTGCACGAGGTTGACCTGACGAC |
| | | CGCGCACAAGGAAGCAGAGTGGGAATTTGAGCCCCTCGCCTGGCGGGTTT |
| | | GGCTGGTGGTCTTCTACTTCGGCTGCTTGTCCTTGACCGTCTGGCTGCTCG |
| | | AGGGGAGTTACGGTGGATCGGACCACCACGCCGCGCGAGCCCAAAGTCC |
| | | AGATGTCCGCGCGCGGCGGTCGGAGCTTGATGACAACATCGCGCAGATGG |
| | | GAGCTGTCCATGGTCTGGAGCTCCCGCGGCGTCAGGTCAGGCGGGAGCT |
| | | CCTGCAGGTTTACCTCGCATAGACGGGTCAGGGCGCGGGCTAGATCCAGG |
| | | TGATACCTAATTTCCAGGGGCTGGTTGGTGGCGGCGTCGATGGCTTGCAA |
| | | GAGGCCGCATCCCCGCGGCGCGACTACGGTACCGCGCGGCGGGCGGTG |
| | | GGCCGCGGGGGTGTCCTTGGATGATGCATCTAAAAGCGGTGACGCGGGC |
| | | GAGCCCCCGGAGGTAGGGGGGGCTCCGGACCCGCCGGGAGAGGGGGCA |
| | | GGGGCACGTCGGCGCCGCGCGCGGGCAGGAGCTGGTGCTGCGCGCGTA |
| | | GGTTGCTGGCGAACGCGACGACGCGGCGGTTGATCTCCTGAATCTGGCGC |
| | | CTCTGCGTGAAGACGACGGGCCCGGTGAGCTTGAGCCTGAAAGAGAGTTC |
| | | GACAGAATCAATTTCGGTGTCGTTGACGGCGGCCTGGCGCAAAATCTCCTG |
| | | CACGTCTCCTGAGTTGTCTTGATAGGCGATCTCGGCCATGAACTGCTCGAT |
| | | CTCTTCCTCCTGGAGATCTCCGCGTCCGGCTCGCTCCACGGTGGCGGCGA |
| | | GGTCGTTGGAAATGCGGGCCATGAGCTGCGAGAAGGCGTTGAGGCCTCCC |
| | | TCGTTCCAGACGCGGCTGTAGACCACGCCCCCTTCGGCATCGCGGGCGC |
| | | GCATGACCACCTGCGCGAGATTGAGCTCCACGTGCCGGGCGAAGACGGC |
| | | GTAGTTTCGCAGGCGCTGAAAGAGGTAGTTGAGGGTGGTGGCGGTGTGTT |
| | | CTGCCACGAAGAAGTACATAACCCAGCGTCGCAACGTGGATTCGTTGATAT |
| | | CCCCCAAGGCCTCAAGGCGCTCCATGGCCTCGTAGAAGTCCACGGCGAAG |
| | | TTGAAAAACTGGGAGTTGCGCGCCGACACGGTTAACTCCTCCTCCAGAAGA |
| | | CGGATGAGCTCGGCGACAGTGTCGCGCACCTCGCGCTCAAAGGCTACAGG |
| | | GGCCTCTTCTTCTTCTTCAATCTCCTCTTCCATAAGGGCCTCCCCTTCTTCT |
| | | TCTTCTGGCGGCGGTGGGGGAGGGGGGACACGGCGGCGACGACGGCGC |
| | | ACCGGGAGGCGGTCGACAAAGCGCTCGATCATCTCCCCGCGGCGACGGC |
| | | GCATGGTCTCGGTGACGGCGCGGCCGTTCTCGCGGGGGCGCAGTTGGAA |
| | | GACGCCGCCCGTCATGTCCCGGTTATGGGTTGGCGGGGGGCTGCCATGC |
| | | GGCAGGGATACGGCGCTAACGATGCATCTCAACAATTGTTGTGTAGGTACT |
| | | CCGCCGCCGAGGGACCTGAGCGAGTCCGCATCGACCGGATCGGAAAACC |
| | | TCTCGAGAAAGGCGTCTAACCAGTCACAGTCGCAAGGTAGGCTGAGCACC |
| | | GTGGCGGGCGGCAGCGGGCGGCGGTCGGGGTTGTTTCTGGCGGAGGTG |
| | | CTGCTGATGATGTAATTAAAGTAGGCGGTCTTGAGACGGCGGATGGTCGAC |
| | | AGAAGCACCATGTCCTTGGGTCCGGCCTGCTGAATGCGCAGGCGGTCGGC |
| | | CATGCCCCAGGCTTCGTTTTGACATCGGCGCAGGTCTTTGTAGTAGTCTTG |
| | | CATGAGCCTTTCTACCGGCACTTCTTCTTCTCCTTCCTCTTGTCCTGCATCT |
| | | CTTGCATCTATCGCTGCGGCGGCGGCGGAGTTTGGCCGTAGGTGGCGCC |
| | | CTCTTCCTCCCATGCGTGTGACCCCGAAGCCCCTCATCGGCTGAAGCAGG |
| | | GCTAGGTCGGCGACAACGCGCTCGGCTAATATGGCCTGCTGCACCTGCGT |
| | | GAGGGTAGACTGGAAGTCATCCATGTCCACAAAGCGGTGGTATGCGCCCG |
| | | TGTTGATGGTGTAAGTGCAGTTGGCCATAACGGACCAGTTAACGGTCTGGT |
| | | GACCCGGCTGCGAGAGCTCGGTGTACCTGAGACGCGAGTAAGCCCTCGA |
| | | GTCAAATACGTAGTCGTTGCAAGTCCGCACCAGGTACTGGTATCCCACCAA |
| | | AAAGTGCGGCGGCGGCTGGCGGTAGAGGGGCCAGCGTAGGGTGGCCGG |
| | | GGCTCCGGGGGCGAGATCTTCCAACATAAGGCGATGATATCCGTAGATGT |
| | | ACCTGGACATCCAGGTGATGCCGGCGGCGGTGGTGGAGGCGCGCGGAAA |
| | | GTCGCGGACGCGGTTCCAGATGTTGCGCAGCGGCAAAAAGTGCTCCATGG |
| | | TCGGGACGCTCTGGCCGGTCAGGCGCGCGCAATCGTTGACGCTCTAGACC |
| | | GTGCAAAAGGAGAGCCTGTAAGCGGGCACTCTTCCGTGGTCTGGTGGATA |
| | | AATTCGCAAGGGTATCATGGCGGACGACCGGGGTTCGAGCCCCGTATCCG |
| | | GCCGTCCGCCGTGATCCATGCGGTTACCGCCCGCGTGTCGAACCCAGGTG |
| | | TGCGACGTCAGACAACGGGGGAGTGCTCCTTTTGGCTTCCTTCCAGGCGC |
| | | GGCGGCTGCTGCGCTAGCTTTTTTGGCCACTGGCCGCGCGCAGCGTAAGC |
| | | GGTTAGGCTGGAAAGCGAAAGCATTAAGTGGCTCGCTCCCTGTAGCCGGA |
| | | GGGTTATTTTCCAAGGGTTGAGTCGCGGGACCCCCGGTTCGAGTCTCGGA |
| | | CCGGCCGGACTGCGGCGAACGGGGGTTTGCCTCCCCGTCATGCAAGACC |
| | | CCGCTTGCAAATTCCTCCGGAAACAGGGACGAGCCCCTTTTTTGCTTTTCC |
| | | CAGATGCATCCGGTGCTGCGGCAGATGCGCCCCCCTCCTCAGCAGCGGCA |
| | | AGAGCAAGAGCAGCGGCAGACATGCAGGGCACCCTCCCCTCCTCCTACCG |
| | | CGTCAGGAGGGGCGACATCCGCGGTTGACGCGGCAGCAGATGGTGATTA |
| | | CGAACCCCCGCGGCGCCGGGCCCGGCACTACCTGGACTTGGAGGAGGGC |
| | | GAGGGCCTGGCGCGGCTAGGAGCGCCCTCTCCTGAGCGGTACCCAAGGG |
| | | TGCAGCTGAAGCGTGATACGCGTGAGGCGTACGTGCCGCGGCAGAACCTG |
| | | TTTCGCGACCGCGAGGGAGAGGAGCCCGAGGAGATGCGGGATCGAAAGT |
| | | TCCACGCAGGGCGCGAGCTGCGGCATGGCCTGAATCGCGAGCGGTTGCT |
| | | GCGCGAGGAGGACTTTGAGCCCGACGCGCGAACCGGGATTAGTCCCGCG |
| | | CGCGCACACGTGGCGGCCGCCGACCTGGTAACCGCATACGAGCAGACGG |
| | | TGAACCAGGAGATTAACTTTCAAAAAAGCTTTAACAACCACGTGCGTACGCT |
| | | TGTGGCGCGCGAGGAGGTGGCTATAGGACTGATGCATCTGTGGGACTTTG |
| | | TAAGCGCGCTGGAGCAAAACCCAAATAGCAAGCCGCTCATGGCGCAGCTG |
| | | TTCCTTATAGTGCAGCACAGCAGGGACAACGAGGCATTCAGGGATGCGCT |
| | | GCTAAACATAGTAGAGCCCGAGGGCCGCTGGCTGCTCGATTTGATAAACAT |
| | | CCTGCAGAGCATAGTGGTGCAGGAGCGCAGCTTGAGCCTGGCTGACAAGG |
| | | TGGCCGCCATCAACTATTCCATGCTTAGCCTGGGCAAGTTTTACGCCCGCA |
| | | AGATATACCATACCCCTTACGTTCCCATAGACAAGGAGGTAAAGATCGAGG |
| | | GGTTCTACATGCGCATGGCGCTGAAGGTGCTTACCTTGAGCGACGACCTG |
| | | GGCGTTTATCGCAACGAGCGCATCCACAAGGCCGTGAGCGTGAGCCGGC |
| | | GGCGCGAGCTCAGCGACCGCGAGCTGATGCACAGCCTGCAAAGGGCCCT |
| | | GGCTGGCACGGGCAGCGGCGATAGAGAGGCCGAGTCCTACTTTGACGCG |
| | | GGCGCTGACCTGCGCTGGGCCCCAAGCCGACGCGCCCTGGAGGCAGCTG |
| | | GGGCCGGACCTGGGCTGGCGGTGGCACCCGCGCGCGCTGGCAACGTCG |
| | | GCGGCGTGGAGGAATATGACGAGGACGATGAGTACGAGCCAGAGGACGG |
| | | CGAGTACTAAGCGGTGATGTTTCTGATCAGATGATGCAAGACGCAACGGAC |
| | | CCGGCGGTGCGGGCGGCGCTGCAGAGCCAGCCGTCCGGCCTTAACTCCA |
| | | CGGACGACTGGCGCCAGGTCATGGACCGCATCATGTCGCTGACTGCGCGC |
| | | AATCCTGACGCGTTCCGGCAGCAGCCGCAGGCCAACCGGCTCTCCGCAAT |
| | | TCTGGAAGCGGTGGTCCCGGCGCGCGCAAACCCCACGCACGAGAAGGTG |
| | | CTGGCGATCGTAAACGCGCTGGCCGAAAACAGGGCCATCCGGCCCGACG |
| | | AGGCCGGCCTGGTCTACGACGCGCTGCTTCAGCGCGTGGCTCGTTACAAC |
| | | AGCGGCAACGTGCAGACCAACCTGGACCGGCTGGTGGGGGATGTGCGCG |
| | | AGGCCGTGGCGCAGCGTGAGCGCGCGCAGCAGCAGGGCAACCTGGGCTC |
| | | CATGGTTGCACTAAACGCCTTCCTGAGTACACAGCCCGCCAACGTGCCGC |
| | | GGGGACAGGAGGACTACACCAACTTTGTGAGCGCACTGCGGCTAATGGTG |
| | | ACTGAGACACCGCAAAGTGAGGTGTACCAGTCTGGGCCAGACTATTTTTTC |
| | | CAGACCAGTAGACAAGGCCTGCAGACCGTAAACCTGAGCCAGGCTTTCAA |
| | | AAACTTGCAGGGGCTGTGGGGGGTGCGGGCTCCCACAGGCGACCGCGCG |
| | | ACCGTGTCTAGCTTGCTGACGCCCAACTCGCGCCTGTTGCTGCTGCTAATA |
| | | GCGCCCTTCACGGACAGTGGCAGCGTGTCCCGGGACACATACCTAGGTCA |
| | | CTTGCTGACACTGTACCGCGAGGCCATAGGTCAGGCGCATGTGGACGAGC |
| | | ATACTTTCCAGGAGATTACAAGTGTCAGCCGCGCGCTGGGGCAGGAGGAC |
| | | ACGGGCAGCCTGGAGGCAACCCTAAACTACCTGCTGACCAACCGGCGGCA |
| | | GAAGATCCCCTCGTTGCACAGTTTAAACAGCGAGGAGGAGCGCATTTTGCG |
| | | CTACGTGCAGCAGAGCGTGAGCCTTAACCTGATGCGCGACGGGGTAACGC |
| | | CCAGCGTGGCGCTGGACATGACCGCGCGCAACATGGAACCGGGCATGTAT |
| | | GCCTCAAACCGGCCGTTTATCAACCGCCTAATGGACTACTTGCATCGCGCG |
| | | GCCGCCGTGAACCCCGAGTATTTCACCAATGCCATCTTGAACCCGCACTGG |
| | | CTACCGCCCCCTGGTTTCTACACCGGGGGATTCGAGGTGCCCGAGGGTAA |
| | | CGATGGATTCCTCTGGGACGACATAGACGACAGCGTGTTTTCCCCGCAACC |
| | | GCAGACCCTGCTAGAGTTGCAACAGCGCGAGCAGGCAGAGGCGGCGCTG |
| | | CGAAAGGAAAGCTTCCGCAGGCCAAGCAGCTTGTCCGATCTAGGCGCTGC |
| | | GGCCCCGCGGTCAGATGCTAGTAGCCCATTTCCAAGCTTGATAGGGTCTCT |
| | | TACCAGCACTCGCACCACCCGCCCGCGCCTGCTGGGCGAGGAGGAGTAC |
| | | CTAAACAACTCGCTGCTGCAGCCGCAGCGCGAAAAAAACCTGCCTCCGGC |
| | | ATTTCCCAACAACGGGATAGAGAGCCTAGTGGACAAGATGAGTAGATGGAA |
| | | GACGTACGCGCAGGAGCACAGGGACGTGCCAGGCCCGCGCCCGCCCACC |
| | | CGTCGTCAAAGGCACGACCGTCAGCGGGGTCTGGTGTGGGAGGACGATG |
| | | ACTCGGCAGACGACAGCAGCGTCCTGGATTTGGGAGGGAGTGGCAACCC |
| | | GTTTGCGCACCTTCGCCCCAGGCTGGGGAGAATGTTTTAAAAAAAAAAAAG |
| | | CATGATGCAAAATAAAAAACTCACCAAGGCCATGGCACCGAGCGTTGGTTT |
| | | TCTTGTATTCCCCTTAGTATGCGGCGCGCGGCGATGTATGAGGAAGGTCCT |
| | | CCTCCCTCCTACGAGAGTGTGGTGAGCGCGGCGCCAGTGGCGGCGGCGC |
| | | TGGGTTCTCCCTTCGATGCTCCCCTGGACCCGCCGTTTGTGCCTCCGCGG |
| | | TACCTGCGGCCTACCGGGGGGAGAAACAGCATCCGTTACTCTGAGTTGGC |
| | | ACCCCTATTCGACACCACCCGTGTGTACCTGGTGGACAACAAGTCAACGGA |
| | | TGTGGCATCCCTGAACTACCAGAACGACCACAGCAACTTTCTGACCACGGT |
| | | CATTCAAAACAATGACTACAGCCCGGGGGAGGCAAGCACACAGACCATCA |
| | | ATCTTGACGACCGGTCGCACTGGGGCGGCGACCTGAAAACCATCCTGCAT |
| | | ACCAACATGCCAAATGTGAACGAGTTCATGTTTACCAATAAGTTTAAGGCGC |
| | | GGGTGATGGTGTCGCGCTTGCCTACTAAGGACAATCAGGTGGAGCTGAAA |
| | | TACGAGTGGGTGGAGTTCACGCTGCCCGAGGGCAACTACTCCGAGACCAT |
| | | GACCATAGACCTTATGAACAACGCGATCGTGGAGCACTACTTGAAAGTGGG |
| | | CAGACAGAACGGGGTTCTGGAAAGCGACATCGGGGTAAAGTTTGACACCC |
| | | GCAACTTCAGACTGGGGTTTGACCCCGTCACTGGTCTTGTCATGCCTGGG |
| | | GTATATACAAACGAAGCCTTCCATCCAGACATCATTTTGCTGCCAGGATGC |
| | | GGGGTGGACTTCACCCACAGCCGCCTGAGCAACTTGTTGGGCATCCGCAA |
| | | GCGGCAACCCTTCCAGGAGGGCTTTAGGATCACCTACGATGATCTGGAGG |
| | | GTGGTAACATTCCCGCACTGTTGGATGTGGACGCCTACCAGGCGAGCTTG |
| | | AAAGATGACACCGAACAGGGCGGGGGTGGCGCAGGCGGCAGCAACAGCA |
| | | GTGGCAGCGGCGCGGAAGAGAACTCCAACGCGGCAGCCGCGGCAATGCA |
| | | GCCGGTGGAGGACATGAACGATCATGCCATTCGCGGCGACACCTTTGCCA |
| | | CACGGGCTGAGGAGAAGCGCGCTGAGGCCGAAGCAGCGGCCGAAGCTGC |
| | | CGCCCCCGCTGCGCAACCCGAGGTCGAGAAGCCTCAGAAGAAACCGGTG |
| | | ATCAAACCCCTGACAGAGGACAGCAAGAAACGCAGTTACAACCTAATAAGC |
| | | AATGACAGCACCTTCACCCAGTACCGCAGCTGGTACCTTGCATACAACTAC |
| | | GGCGACCCTCAGACCGGAATCCGCTCATGGACCCTGCTTTGCACTCCTGA |
| | | CGTAACCTGCGGCTCGGAGCAGGTCTACTGGTCGTTGCCAGACATGATGC |
| | | AAGACCCCGTGACCTTCCGCTCCACGCGCCAGATCAGCAACTTTCCGGTG |
| | | GTGGGCGCCGAGCTGTTGCCCGTGCACTCCAAGAGCTTCTACAACGACCA |
| | | GGCCGTCTACTCCCAACTCATCCGCCAGTTTACCTCTCTGACCCACGTGTT |
| | | CAATCGCTTTCCCGAGAACCAGATTTTGGCGCGCCCGCCAGCCCCCACCA |
| | | TCACCACCGTCAGTGAAAACGTTCCTGCTCTCACAGATCACGGGACGCTAC |
| | | CGCTGCGCAACAGCATCGGAGGAGTCCAGCGAGTGACCATTACTGACGCC |
| | | AGACGCCGCACCTGCCCCTACGTTTACAAGGCCCTGGGCATAGTCTCGCC |
| | | GCGCGTCCTATCGAGCCGCACTTTTTGAGCAAGCATGTCCATCCTTATATC |
| | | GCCCAGCAATAACACAGGCTGGGGCCTGCGCTTCCCAAGCAAGATGTTTG |
| | | GCGGGGCCAAGAAGCGCTCCGACCAACACCCAGTGCGCGTGCGCGGGCA |
| | | CTACCGCGCGCCCTGGGGCGCGCACAAACGCGGCCGCACTGGGCGCACC |
| | | ACCGTCGATGACGCCATCGACGCGGTGGTGGAGGAGGCGCGCAACTACA |
| | | CGCCCACGCCGCCACCAGTGTCCACAGTGGACGCGGCCATTCAGACCGTG |
| | | GTGCGCGGAGCCCGGCGCTATGCTAAAATGAAGAGACGGCGGAGGCGCG |
| | | TAGCACGTCGCCACCGCCGCCGACCCGGCACTGCCGCCCAACGCGCGGC |
| | | GGCGGCCCTGCTTAACCGCGCACGTCGCACCGGCCGACGGGCGGCCATG |
| | | CGGGCCGCTCGAAGGCTGGCCGCGGGTATTGTCACTGTGCCCCCCAGGT |
| | | CCAGGCGACGAGCGGCCGCCGCAGCAGCCGCGGCCATTAGTGCTATGAC |
| | | TCAGGGTCGCAGGGGCAACGTGTATTGGGTGCGCGACTCGGTTAGCGGC |
| | | CTGCGCGTGCCCGTGCGCACCCGCCCCCCGCGCAACTAGATTGCAAGAAA |
| | | AAACTACTTAGACTCGTACTGTTGTATGTATCCAGCGGCGGCGGCGCGCAA |
| | | CGAAGCTATGTCCAAGCGCAAAATCAAAGAAGAGATGCTCCAGGTCATCGC |
| | | GCCGGAGATCTATGGCCCCCCGAAGAAGGAAGAGCAGGATTACAAGCCCC |
| | | GAAAGCTAAAGCGGGTCAAAAAGAAAAAGAAAGATGATGATGATGAACTTG |
| | | ACGACGAGGTGGAACTGCTGCACGCTACCGCGCCCAGGCGACGGGTACA |
| | | GTGGAAAGGTCGACGCGTAAAACGTGTTTTGCGACCCGGCACCACCGTAG |
| | | TCTTTACGCCCGGTGAGCGCTCCACCCGCACCTACAAGCGCGTGTATGAT |
| | | GAGGTGTACGGCGACGAGGACCTGCTTGAGCAGGCCAACGAGCGCCTCG |
| | | GGGAGTTTGCCTACGGAAAGCGGCATAAGGACATGCTGGCGTTGCCGCTG |
| | | GACGAGGGCAACCCAACACCTAGCCTAAAGCCCGTAACACTGCAGCAGGT |
| | | GCTGCCCGCGCTTGCACCGTCCGAAGAAAAGCGCGGCCTAAAGCGCGAGT |
| | | CTGGTGACTTGGCACCCACCGTGCAGCTGATGGTACCCAAGCGCCAGCGA |
| | | CTGGAAGATGTCTTGGAAAAAATGACCGTGGAACCTGGGCTGGAGCCCGA |
| | | GGTCCGCGTGCGGCCAATCAAGCAGGTGGCGCCGGGACTGGGCGTGCAG |
| | | ACCGTGGACGTTCAGATACCCACTACCAGTAGCACCAGTATTGCCACCGCC |
| | | ACAGAGGGCATGGAGACACAAACGTCCCCGGTTGCCTCAGCGGTGGCGG |
| | | ATGCCGCGGTGCAGGCGGTCGCTGCGGCCGCGTCCAAGACCTCTACGGA |
| | | GGTGCAAACGGACCCGTGGATGTTTCGCGTTTCAGCCCCCCGGCGCCCGC |
| | | GCGGTTCGAGGAAGTACGGCGCCGCCAGCGCGCTACTGCCCGAATATGC |
| | | CCTACATCCTTCCATTGCGCCTACCCCCGGCTATCGTGGCTACACCTACCG |
| | | CCCCAGAAGACGAGCAACTACCCGACGCCGAACCACCACTGGAACCCGCC |
| | | GCCGCCGTCGCCGTCGCCAGCCCGTGCTGGCCCCGATTTCCGTGCGCAG |
| | | GGTGGCTCGCGAAGGAGGCAGGACCCTGGTGCTGCCAACAGCGCGCTAC |
| | | CACCCCAGCATCGTTTAAAAGCCGGTCTTTGTGGTTCTTGCAGATATGGCC |
| | | CTCACCTGCCGCCTCCGTTTCCCGGTGCCGGGATTCCGAGGAAGAATGCA |
| | | CCGTAGGAGGGGCATGGCCGGCCACGGCCTGACGGGCGGCATGCGTCGT |
| | | GCGCACCACCGGCGGCGGCGCGCGTCGCACCGTCGCATGCGCGGCGGT |
| | | ATCCTGCCCCTCCTTATTCCACTGATCGCCGCGGCGATTGGCGCCGTGCC |
| | | CGGAATTGCATCCGTGGCCTTGCAGGCGCAGAGACACTGATTAAAAACAAG |
| | | TTGCATGTGGAAAAATCAAAATAAAAAGTCTGGACTCTCACGCTCGCTTGGT |
| | | CCTGTAACTATTTTGTAGAATGGAAGACATCAACTTTGCGTCTCTGGCCCCG |
| | | CGACACGGCTCGCGCCCGTTCATGGGAAACTGGCAAGATATCGGCACCAG |
| | | CAATATGAGCGGTGGCGCCTTCAGCTGGGGCTCGCTGTGGAGCGGCATTA |
| | | AAAATTTCGGTTCCACCGTTAAGAACTATGGCAGCAAGGCCTGGAACAGCA |
| | | GCACAGGCCAGATGCTGAGGGATAAGTTGAAAGAGCAAAATTTCCAACAAA |
| | | AGGTGGTAGATGGCCTGGCCTCTGGCATTAGCGGGGTGGTGGACCTGGC |
| | | CAACCAGGCAGTGCAAAATAAGATTAACAGTAAGCTTGATCCCCGCCCTCC |
| | | CGTAGAGGAGCCTCCACCGGCCGTGGAGACAGTGTCTCCAGAGGGGCGT |
| | | GGCGAAAAGCGTCCGCGCCCCGACAGGGAAGAAACTCTGGTGACGCAAAT |
| | | AGACGAGCCTCCCTCGTACGAGGAGGCACTAAAGCAAGGCCTGCCCACCA |
| | | CCCGTCCCATCGCGCCCATGGCTACCGGAGTGCTGGGCCAGCACACACCC |
| | | GTAACGCTGGACCTGCCTCCCCCCGCCGACACCCAGCAGAAACCTGTGCT |
| | | GCCAGGCCCGACCGCCGTTGTTGTAACCCGTCCTAGCCGCGCGTCCCTGC |
| | | GCCGCGCCGCCAGCGGTCCGCGATCGTTGCGGCCCGTAGCCAGTGGCAA |
| | | CTGGCAAAGCACACTGAACAGCATCGTGGGTCTGGGGGTGCAATCCCTGA |
| | | AGCGCCGACGATGCTTCTGAATAGCTAACGTGTCGTATGTGTGTCATGTAT |
| | | GCGTCCATGTCGCCGCCAGAGGAGCTGCTGAGCCGCCGCGCGCCCGCTT |
| | | TCCAAGATGGCTACCCCTTCGATGATGCCGCAGTGGTCTTACATGCACATC |
| | | TCGGGCCAGGACGCCTCGGAGTACCTGAGCCCCGGGCTGGTGCAGTTTG |
| | | CCCGCGCCACCGAGACGTACTTCAGCCTGAATAACAAGTTTAGAAACCCCA |
| | | CGGTGGCGCCTACGCACGACGTGACCACAGACCGGTCCCAGCGTTTGACG |
| | | CTGCGGTTCATCCCTGTGGACCGTGAGGATACTGCGTACTCGTACAAGGC |
| | | GCGGTTCACCCTAGCTGTGGGTGATAACCGTGTGCTGGACATGGCTTCCA |
| | | CGTACTTTGACATCCGCGGCGTGCTGGACAGGGGCCCTACTTTTAAGCCCT |
| | | ACTCTGGCACTGCCTACAACGCCCTGGCTCCCAAGGGTGCCCCAAATCCTT |
| | | GCGAATGGGATGAAGCTGCTACTGCTCTTGAAATAAACCTAGAAGAAGAGG |
| | | ACGATGACAACGAAGACGAAGTAGACGAGCAAGCTGAGCAGCAAAAAACT |
| | | CACGTATTTGGGCAGGCGCCTTATTCTGGTATAAATATTACAAAGGAGGGT |
| | | ATTCAAATAGGTGTCGAAGGTCAAACACCTAAATATGCCGATAAAACATTTC |
| | | AACCTGAACCTCAAATAGGAGAATCTCAGTGGTACGAAACTGAAATTAATCA |
| | | TGCAGCTGGGAGAGTCCTTAAAAAGACTACCCCAATGAAACCATGTTACGG |
| | | TTCATATGCAAAACCCACAAATGAAAATGGAGGGCAAGGCATTCTTGTAAA |
| | | GCAACAAAATGGAAAGCTAGAAAGTCAAGTGGAAATGCAATTTTTCTCAACT |
| | | ACTGAGGCGACCGCAGGCAATGGTGATAACTTGACTCCTAAAGTGGTATTG |
| | | TACAGTGAAGATGTAGATATAGAAACCCCAGACACTCATATTTCTTACATGC |
| | | CCACTATTAAGGAAGGTAACTCACGAGAACTAATGGGCCAACAATCTATGC |
| | | CCAACAGGCCTAATTACATTGCTTTTAGGGACAATTTTATTGGTCTAATGTAT |
| | | TACAACAGCACGGGTAATATGGGTGTTCTGGCGGGCCAAGCATCGCAGTT |
| | | GAATGCTGTTGTAGATTTGCAAGACAGAAACACAGAGCTTTCATACCAGCTT |
| | | TTGCTTGATTCCATTGGTGATAGAACCAGGTACTTTTCTATGTGGAATCAGG |
| | | CTGTTGACAGCTATGATCCAGATGTTAGAATTATTGAAAATCATGGAACTGA |
| | | AGATGAACTTCCAAATTACTGCTTTCCACTGGGAGGTGTGATTAATACAGAG |
| | | ACTCTTACCAAGGTAAAACCTAAAACAGGTCAGGAAAATGGATGGGAAAAA |
| | | GATGCTACAGAATTTTCAGATAAAAATGAAATAAGAGTTGGAAATAATTTTG |
| | | CCATGGAAATCAATCTAAATGCCAACCTGTGGAGAAATTTCCTGTACTCCAA |
| | | CATAGCGCTGTATTTGCCCGACAAGCTAAAGTACAGTCCTTCCAACGTAAA |
| | | AATTTCTGATAACCCAAACACCTACGACTACATGAACAAGCGAGTGGTGGC |
| | | TCCCGGGTTAGTGGACTGCTACATTAACCTTGGAGCACGCTGGTCCCTTGA |
| | | CTATATGGACAACGTCAACCCATTTAACCACCACCGCAATGCTGGCCTGCG |
| | | CTACCGCTCAATGTTGCTGGGCAATGGTCGCTATGTGCCCTTCCACATCCA |
| | | GGTGCCTCAGAAGTTCTTTGCCATTAAAAACCTCCTTCTCCTGCCGGGCTC |
| | | ATACACCTACGAGTGGAACTTCAGGAAGGATGTTAACATGGTTCTGCAGAG |
| | | CTCCCTAGGAAATGACCTAAGGGTTGACGGAGCCAGCATTAAGTTTGATAG |
| | | CATTTGCCTTTACGCCACCTTCTTCCCCATGGCCCACAACACCGCCTCCAC |
| | | GCTTGAGGCCATGCTTAGAAACGACACCAACGACCAGTCCTTTAACGACTA |
| | | TCTCTCCGCCGCCAACATGCTCTACCCTATACCCGCCAACGCTACCAACGT |
| | | GCCCATATCCATCCCCTCCCGCAACTGGGCGGCTTTCCGCGGCTGGGCCT |
| | | TCACGCGCCTTAAGACTAAGGAAACCCCATCACTGGGCTCGGGCTACGAC |
| | | CCTTATTACACCTACTCTGGCTCTATACCCTACCTAGATGGAACCTTTTACC |
| | | TCAACCACACCTTTAAGAAGGTGGCCATTACCTTTGACTCTTCTGTCAGCTG |
| | | GCCTGGCAATGACCGCCTGCTTACCCCCAACGAGTTTGAAATTAAGCGCTC |
| | | AGTTGACGGGGAGGGTTACAACGTTGCCCAGTGTAACATGACCAAAGACT |
| | | GGTTCCTGGTACAAATGCTAGCTAACTACAACATTGGCTACCAGGGCTTCT |
| | | ATATCCCAGAGAGCTACAAGGACCGCATGTACTCCTTCTTTAGAAACTTCCA |
| | | GCCCATGAGCCGTCAGGTGGTGGATGATACTAAATACAAGGACTACCAACA |
| | | GGTGGGCATCCTACACCAACACAACAACTCTGGATTTGTTGGCTACCTTGC |
| | | CCCCACCATGCGCGAAGGACAGGCCTACCCTGCTAACTTCCCCTATCCGC |
| | | TTATAGGCAAGACCGCAGTTGACAGCATTACCCAGAAAAAGTTTCTTTGCG |
| | | ATCGCACCCTTTGGCGCATCCCATTCTCCAGTAACTTTATGTCCATGGGCG |
| | | CACTCACAGACCTGGGCCAAAACCTTCTCTACGCCAACTCCGCCCACGCG |
| | | CTAGACATGACTTTTGAGGTGGATCCCATGGACGAGCCCACCCTTCTTTAT |
| | | GTTTTGTTTGAAGTCTTTGACGTGGTCCGTGTGCACCGGCCGCACCGCGG |
| | | CGTCATCGAAACCGTGTACCTGCGCACGCCCTTCTCGGCCGGCAACGCCA |
| | | CAACATAAAGAAGCAAGCAACATCAACAACAGCTGCCGCCATGGGCTCCAG |
| | | TGAGCAGGAACTGAAAGCCATTGTCAAAGATCTTGGTTGTGGGCCATATTT |
| | | TTTGGGCACCTATGACAAGCGCTTTCCAGGCTTTGTTTCTCCACACAAGCT |
| | | CGCCTGCGCCATAGTCAATACGGCCGGTCGCGAGACTGGGGGCGTACACT |
| | | GGATGGCCTTTGCCTGGAACCCGCACTCAAAAACATGCTACCTCTTTGAGC |
| | | CCTTTGGCTTTTCTGACCAGCGACTCAAGCAGGTTTACCAGTTTGAGTACG |
| | | AGTCACTCCTGCGCCGTAGCGCCATTGCTTCTTCCCCCGACCGCTGTATAA |
| | | CGCTGGAAAAGTCCACCCAAAGCGTACAGGGGCCCAACTCGGCCGCCTGT |
| | | GGACTATTCTGCTGCATGTTTCTCCACGCCTTTGCCAACTGGCCCCAAACT |
| | | CCCATGGATCACAACCCCACCATGAACCTTATTACCGGGGTACCCAACTCC |
| | | ATGCTCAACAGTCCCCAGGTACAGCCCACCCTGCGTCGCAACCAGGAACA |
| | | GCTCTACAGCTTCCTGGAGCGCCACTCGCCCTACTTCCGCAGCCACAGTG |
| | | CGCAGATTAGGAGCGCCACTTCTTTTTGTCACTTGAAAAACATGTAAAAATA |
| | | ATGTACTAGAGACACTTTCAATAAAGGCAAATGCTTTTATTTGTACACTCTC |
| | | GGGTGATTATTTACCCCCACCCTTGCCGTCTGCGCCGTTTAAAAATCAAAG |
| | | GGGTTCTGCCGCGCATCGCTATGCGCCACTGGCAGGGACACGTTGCGATA |
| | | CTGGTGTTTAGTGCTCCACTTAAACTCAGGCACAACCATCCGCGGCAGCTC |
| | | GGTGAAGTTTTCACTCCACAGGCTGCGCACCATCACCAACGCGTTTAGCAG |
| | | GTCGGGCGCCGATATCTTGAAGTCGCAGTTGGGGCCTCCGCCCTGCGCGC |
| | | GCGAGTTGCGATACACAGGGTTGCAGCACTGGAACACTATCAGCGCCGGG |
| | | TGGTGCACGCTGGCCAGCACGCTCTTGTCGGAGATCAGATCCGCGTCCAG |
| | | GTCCTCCGCGTTGCTCAGGGCGAACGGAGTCAACTTTGGTAGCTGCCTTC |
| | | CCAAAAAGGGCGCGTGCCCAGGCTTTGAGTTGCACTCGCACCGTAGTGGC |
| | | ATCAAAAGGTGACCGTGCCCGGTCTGGGCGTTAGGATACAGCGCCTGCAT |
| | | AAAAGCCTTGATCTGCTTAAAAGCCACCTGAGCCTTTGCGCCTTCAGAGAA |
| | | GAACATGCCGCAAGACTTGCCGGAAAACTGATTGGCCGGACAGGCCGCGT |
| | | CGTGCACGCAGCACCTTGCGTCGGTGTTGGAGATCTGCACCACATTTCGG |
| | | CCCCACCGGTTCTTCACGATCTTGGCCTTGCTAGACTGCTCCTTCAGCGCG |
| | | CGCTGCCCGTTTTCGCTCGTCACATCCATTTCAATCACGTGCTCCTTATTTA |
| | | TCATAATGCTTCCGTGTAGACACTTAAGCTCGCCTTCGATCTCAGCGCAGC |
| | | GGTGCAGCCACAACGCGCAGCCCGTGGGCTCGTGATGCTTGTAGGTCACC |
| | | TCTGCAAACGACTGCAGGTACGCCTGCAGGAATCGCCCCATCATCGTCACA |
| | | AAGGTCTTGTTGCTGGTGAAGGTCAGCTGCAACCCGCGGTGCTCCTCGTT |
| | | CAGCCAGGTCTTGCATACGGCCGCCAGAGCTTCCACTTGGTCAGGCAGTA |
| | | GTTTGAAGTTCGCCTTTAGATCGTTATCCACGTGGTACTTGTCCATCAGCGC |
| | | GCGCGCAGCCTCCATGCCCTTCTCCCACGCAGACACGATCGGCACACTCA |
| | | GCGGGTTCATCACCGTAATTTCACTTTCCGCTTCGCTGGGCTCTTCCTCTTC |
| | | CTCTTGCGTCCGCATACCACGCGCCACTGGGTCGTCTTCATTCAGCCGCC |
| | | GCACTGTGCGCTTACCTCCTTTGCCATGCTTGATTAGCACCGGTGGGTTGC |
| | | TGAAACCCACCATTTGTAGCGCCACATCTTCTCTTTCTTCCTCGCTGTCCAC |
| | | GATTACCTCTGGTGATGGCGGGCGCTCGGGCTTGGGAGAAGGGCGCTTCT |
| | | TTTTCTTCTTGGGCGCAATGGCCAAATCCGCCGCCGAGGTCGATGGCCGC |
| | | GGGCTGGGTGTGCGCGGCACCAGCGCGTCTTGTGATGAGTCTTCCTCGTC |
| | | CTCGGACTCGATACGCCGCCTCATCCGCTTTTTTGGGGGCGCCCGGGGAG |
| | | GCGGCGGCGACGGGGACGGGGACGACACGTCCTCCATGGTTGGGGGAC |
| | | GTCGCGCCGCACCGCGTCCGCGCTCGGGGGTGGTTTCGCGCTGCTCCTC |
| | | TTCCCGACTGGCCATTTCCTTCTCCTATAGGCAGAAAAAGATCATGGAGTC |
| | | AGTCGAGAAGAAGGACAGCCTAACCGCCCCCTCTGAGTTCGCCACCACCG |
| | | CCTCCACCGATGCCGCCAACGCGCCTACCACCTTCCCCGTCGAGGCACCC |
| | | CCGCTTGAGGAGGAGGAAGTGATTATCGAGCAGGACCCAGGTTTTGTAAG |
| | | CGAAGACGACGAGGACCGCTCAGTACCAACAGAGGATAAAAAGCAAGACC |
| | | AGGACAACGCAGAGGCAAACGAGGAACAAGTCGGGCGGGGGGACGAAAG |
| | | GCATGGCGACTACCTAGATGTGGGAGACGACGTGCTGTTGAAGCATCTGC |
| | | AGCGCCAGTGCGCCATTATCTGCGACGCGTTGCAAGAGCGCAGCGATGTG |
| | | CCCCTCGCCATAGCGGATGTCAGCCTTGCCTACGAACGCCACCTATTCTCA |
| | | CCGCGCGTACCCCCCAAACGCCAAGAAAACGGCACATGCGAGCCCAACCC |
| | | GCGCCTCAACTTCTACCCCGTATTTGCCGTGCCAGAGGTGCTTGCCACCTA |
| | | TCACATCTTTTTCCAAAACTGCAAGATACCCCTATCCTGCCGTGCCAACCGC |
| | | AGCCGAGCGGACAAGCAGCTGGCCTTGCGGCAGGGCGCTGTCATACCTG |
| | | ATATCGCCTCGCTCAACGAAGTGCCAAAAATCTTTGAGGGTCTTGGACGCG |
| | | ACGAGAAGCGCGCGGCAAACGCTCTGCAACAGGAAAACAGCGAAAATGAA |
| | | AGTCACTCTGGAGTGTTGGTGGAACTCGAGGGTGACAACGCGCGCCTAGC |
| | | CGTACTAAAACGCAGCATCGAGGTCACCCACTTTGCCTACCCGGCACTTAA |
| | | CCTACCCCCCAAGGTCATGAGCACAGTCATGAGTGAGCTGATCGTGCGCC |
| | | GTGCGCAGCCCCTGGAGAGGGATGCAAATTTGCAAGAACAAACAGAGGAG |
| | | GGCCTACCCGCAGTTGGCGACGAGCAGCTAGCGCGCTGGCTTCAAACGC |
| | | GCGAGCCTGCCGACTTGGAGGAGCGACGCAAACTAATGATGGCCGCAGTG |
| | | CTCGTTACCGTGGAGCTTGAGTGCATGCAGCGGTTCTTTGCTGACCCGGA |
| | | GATGCAGCGCAAGCTAGAGGAAACATTGCACTACACCTTTCGACAGGGCTA |
| | | CGTACGCCAGGCCTGCAAGATCTCCAACGTGGAGCTCTGCAACCTGGTCT |
| | | CCTACCTTGGAATTTTGCACGAAAACCGCCTTGGGCAAAACGTGCTTCATT |
| | | CCACGCTCAAGGGCGAGGCGCGCCGCGACTACGTCCGCGACTGCGTTTA |
| | | CTTATTTCTATGCTACACCTGGCAGACGGCCATGGGCGTTTGGCAGCAGTG |
| | | CTTGGAGGAGTGCAACCTCAAGGAGCTGCAGAAACTGCTAAAGCAAAACTT |
| | | GAAGGACCTATGGACGGCCTTCAACGAGCGCTCCGTGGCCGCGCACCTG |
| | | GCGGACATCATTTTCCCCGAACGCCTGCTTAAAACCCTGCAACAGGGTCTG |
| | | CCAGACTTCACCAGTCAAAGCATGTTGCAGAACTTTAGGAACTTTATCCTAG |
| | | AGCGCTCAGGAATCTTGCCCGCCACCTGCTGTGCACTTCCTAGCGACTTTG |
| | | TGCCCATTAAGTACCGCGAATGCCCTCCGCCGCTTTGGGGCCACTGCTAC |
| | | CTTCTGCAGCTAGCCAACTACCTTGCCTACCACTCTGACATAATGGAAGAC |
| | | GTGAGCGGTGACGGTCTACTGGAGTGTCACTGTCGCTGCAACCTATGCAC |
| | | CCCGCACCGCTCCCTGGTTTGCAATTCGCAGCTGCTTAACGAAAGTCAAAT |
| | | TATCGGTACCTTTGAGCTGCAGGGTCCCTCGCCTGACGAAAAGTCCGCGG |
| | | CTCCGGGGTTGAAACTCACTCCGGGGCTGTGGACGTCGGCTTACCTTCGC |
| | | AAATTTGTACCTGAGGACTACCACGCCCACGAGATTAGGTTCTACGAAGAC |
| | | CAATCCCGCCCGCCAAATGCGGAGCTTACCGCCTGCGTCATTACCCAGGG |
| | | CCACATTCTTGGCCAATTGCAAGCCATCAACAAAGCCCGCCAAGAGTTTCT |
| | | GCTACGAAAGGGACGGGGGGTTTACTTGGACCCCCAGTCCGGCGAGGAG |
| | | CTCAACCCAATCCCCCCGCCGCCGCAGCCCTATCAGCAGCAGCCGCGGG |
| | | CCCTTGCTTCCCAGGATGGCACCCAAAAAGAAGCTGCAGCTGCCGCCGCC |
| | | ACCCACGGACGAGGAGGAATACTGGGACAGTCAGGCAGAGGAGGTTTTGG |
| | | ACGAGGAGGAGGAGGACATGATGGAAGACTGGGAGAGCCTAGACGAGGA |
| | | AGCTTCCGAGGTCGAAGAGGTGTCAGACGAAACACCGTCACCCTCGGTCG |
| | | CATTCCCCTCGCCGGCGCCCCAGAAATCGGCAACCGGTTCCAGCATGGCT |
| | | ACAACCTCCGCTCCTCAGGCGCCGCCGGCACTGCCCGTTCGCCGACCCAA |
| | | CCGTAGATGGGACACCACTGGAACCAGGGCCGGTAAGTCCAAGCAGCCGC |
| | | CGCCGTTAGCCCAAGAGCAACAACAGCGCCAAGGCTACCGCTCATGGCGC |
| | | GGGCACAAGAACGCCATAGTTGCTTGCTTGCAAGACTGTGGGGGCAACAT |
| | | CTCCTTCGCCCGCCGCTTTCTTCTCTACCATCACGGCGTGGCCTTCCCCCG |
| | | TAACATCCTGCATTACTACCGTCATCTCTACAGCCCATACTGCACCGGCGG |
| | | CAGCGGCAGCGGCAGCAACAGCAGCGGCCACACAGAAGCAAAGGCGACC |
| | | GGATAGCAAGACTCTGACAAAGCCCAAGAAATCCACAGCGGCGGCAGCAG |
| | | CAGGAGGAGGAGCGCTGCGTCTGGCGCCCAACGAACCCGTATCGACCCG |
| | | CGAGCTTAGAAACAGGATTTTTCCCACTCTGTATGCTATATTTCAACAGAGC |
| | | AGGGGCCAAGAACAAGAGCTGAAAATAAAAAACAGGTCTCTGCGATCCCTC |
| | | ACCCGCAGCTGCCTGTATCACAAAAGCGAAGATCAGCTTCGGCGCACGCT |
| | | GGAAGACGCGGAGGCTCTCTTCAGTAAATACTGCGCGCTGACTCTTAAGGA |
| | | CTAGTTTCGCGCCCTTTCTCAAATTTAAGCGCGAAAACTACGTCATCTCCAG |
| | | CGGCCACACCCGGCGCCAGCACCTGTCGTCAGCGCCATTATGAGCAAGGA |
| | | AATTCCCACGCCCTACATGTGGAGTTACCAGCCACAAATGGGACTTGCGGC |
| | | TGGAGCTGCCCAAGACTACTCAACCCGAATAAACTACATGAGCGCGGGAC |
| | | CCCACATGATATCCCGGGTCAACGGAATCCGCGCCCACCGAAACCGAATT |
| | | CTCTTGGAACAGGCGGCTATTACCACCACACCTCGTAATAACCTTAATCCC |
| | | CGTAGTTGGCCCGCTGCCCTGGTGTACCAGGAAAGTCCCGCTCCCACCAC |
| | | TGTGGTACTTCCCAGAGACGCCCAGGCCGAAGTTCAGATGACTAACTCAG |
| | | GGGCGCAGCTTGCGGGCGGCTTTCGTCACAGGGTGCGGTCGCCCGGGCA |
| | | GGGTATAACTCACCTGACAATCAGAGGGCGAGGTATTCAGCTCAACGACGA |
| | | GTCGGTGAGCTCCTCGCTTGGTCTCCGTCCGGACGGGACATTTCAGATCG |
| | | GCGGCGCCGGCCGCTCTTCATTCACGCCTCGTCAGGCAATCCTAACTCTG |
| | | CAGACCTCGTCCTCTGAGCCGCGCTCTGGAGGCATTGGAACTCTGCAATTT |
| | | ATTGAGGAGTTTGTGCCATCGGTCTACTTTAACCCCTTCTCGGGACCTCCC |
| | | GGCCACTATCCGGATCAATTTATTCCTAACTTTGACGCGGTAAAGGACTCG |
| | | GCGGATGGCTACGACTGAATGTTAAGTGGAGAGGCAGAGCAACTGCGCCT |
| | | GAAACACCTGGTCCACTGTCGCCGCCACAAGTGCTTTGCCCGCGACTCCG |
| | | GTGAGTTTTGCTACTTTGAATTGCCCGAGGATCATATCGAGGGCCCGGCGC |
| | | ACGGCGTCCGGCTTACCGCCCAGGGAGAGCTTGCCCGTAGCCTGATTCGG |
| | | GAGTTTACCCAGCGCCCCCTGCTAGTTGAGCGGGACAGGGGACCCTGTGT |
| | | TCTCACTGTGATTTGCAACTGTCCTAACCCTGGATTACATCAAGATCTTTGT |
| | | TGCCATCTCTGTGCTGAGTATAATAAATACAGAAATTAAAATATACTGGGGC |
| | | TCCTATCGCCATCCTGTAAACGCCACCGTCTTCACCCGCCCAAGCAAACCA |
| | | AGGCGAACCTTACCTGGTACTTTTAACATCTCTCCCTCTGTGATTTACAACA |
| | | GTTTCAACCCAGACGGAGTGAGTCTACGAGAGAACCTCTCCGAGCTCAGCT |
| | | ACTCCATCAGAAAAAACACCACCCTCCTTACCTGCCGGGAACGTACGACCT |
| | | AGGGATAACAGGGTAATAAGCAATTGACTCTATGTGGGATATGCTCCAGCG |
| | | CTACAACCTTGAAGTCAGGCTTCCTGGATGTCAGCATCTGACTTTGGCCAG |
| | | CACCTGTCCCGCGGATTTGTTCCAGTCCAACTACAGCGACCCACCCTAACA |
| | | GAGATGACCAACACAACCAACGCGGCCGCCGCTACCGGACTTACATCTAC |
| | | CACAAATACACCCCAAGTTTCTGCCTTTGTCAATAACTGGGATAACTTGGGC |
| | | ATGTGGTGGTTCTCCATAGCGCTTATGTTTGTATGCCTTATTATTATGTGGC |
| | | TCATCTGCTGCCTAAAGCGCAAACGCGCCCGACCACCCATCTATAGTCCCA |
| | | TCATTGTGCTACACCCAAACAATGATGGAATCCATAGATTGGACGGACTGA |
| | | AACACATGTTCTTTTCTCTTACAGTATGATTAAATGAGACATGATTCCTCGAG |
| | | TTTTTATATTACTGACCCTTGTTGCGCTTTTTTGTGCGTGCTCCACATTGGCT |
| | | GCGGTTTCTCACATCGAAGTAGACTGCATTCCAGCCTTCACAGTCTATTTGC |
| | | TTTACGGATTTGTCACCCTCACGCTCATCTGCAGCCTCATCACTGTGGTCAT |
| | | CGCCTTTATCCAGTGCATTGACTGGGTCTGTGTGCGCTTTGCATATCTCAG |
| | | ACACCATCCCCAGTACAGGGACAGGACTATAGCTGAGCTTCTTAGAATTCT |
| | | TTAATTATGAAATTTACTGTGACTTTTCTGCTGATTATTTGCACCCTATCTGC |
| | | GTTTTGTTCCCCGACCTCCAAGCCTCAAAGACATATATCATGCAGATTCACT |
| | | CGTATATGGAATATTCCAAGTTGCTACAATGAAAAAAGCGATCTTTCCGAAG |
| | | CCTGGTTATATGCAATCATCTCTGTTATGGTGTTCTGCAGTACCATCTTAGC |
| | | CCTAGCTATATATCCCTACCTTGACATTGGCTGGAAACGAATAGATGCCATG |
| | | AACCACCCAACTTTCCCCGCGCCCGCTATGCTTCCACTGCAACAAGTTGTT |
| | | GCCGGCGGCTTTGTCCCAGCCAATCAGCCTCGCCCCACTTCTCCCACCCC |
| | | CACTGAAATCAGCTACTTTAATCTAACAGGAGGAGATGACTGACACCCTAG |
| | | ATCTAGAAATGGACGGAATTATTACAGAGCAGCGCCTGCTAGAAAGACGCA |
| | | GGGCAGCGGCCGAGCAACAGCGCATGAATCAAGAGCTCCAAGACATGGTT |
| | | AACTTGCACCAGTGCAAAAGGGGTATCTTTTGTCTGGTAAAGCAGGCCAAA |
| | | GTCACCTACGACAGTAATACCACCGGACACCGCCTTAGCTACAAGTTGCCA |
| | | ACCAAGCGTCAGAAATTGGTGGTCATGGTGGGAGAAAAGCCCATTACCATA |
| | | ACTCAGCACTCGGTAGAAACCGAAGGCTGCATTCACTCACCTTGTCAAGGA |
| | | CCTGAGGATCTCTGCACCCTTATTAAGACCCTGTGCGGTCTCAAAGATCTT |
| | | ATTCCCTTTAACTAATAAAAAAAAATAATAAAGCATCACTTACTTAAAATCAG |
| | | TTAGCAAATTTCTGTCCAGTTTATTCAGCAGCACCTCCTTGCCCTCCTCCCA |
| | | GCTCTGGTATTGCAGCTTCCTCCTGGCTGCAAACTTTCTCCACAATCTAAAT |
| | | GGAATGTCAGTTTCCTCCTGTTCCTGTCCATCCGCACCCACTATCTTCATGT |
| | | TGTTGCAGATGAAGCGCGCAAGACCGTCTGAAGATACCTTCAACCCCGTGT |
| | | ATCCATATGACACGGAAACCGGTCCTCCAACTGTGCCTTTTCTTACTCCTCC |
| | | CTTTGTATCCCCCAATGGGTTTCAAGAGAGTCCCCCTGGGGTACTCTCTTT |
| | | GCGCCTATCCGAACCTCTAGTTACCTCCAATGGCATGCTTGCGCTCAAAAT |
| | | GGGCAACGGCCTCTCTCTGGACGAGGCCGGCAACCTTACCTCCCAAAATG |
| | | TAACCACTGTGAGCCCACCTCTCAAAAAAACCAAGTCAAACATAAACCTGG |
| | | AAATATCTGCACCCCTCACAGTTACCTCAGAAGCCCTAACTGTGGCTGCCG |
| | | CCGCACCTCTAATGGTCGCGGGCAACACACTCACCATGCAATCACAGGCC |
| | | CCGCTAACCGTGCACGACTCCAAACTTAGCATTGCCACCCAAGGACCCCTC |
| | | ACAGTGTCAGAAGGAAAGCTAGCCCTGCAAACATCAGGCCCCCTCACCAC |
| | | CACCGATAGCAGTACCCTTACTATCACTGCCTCACCCCCTCTAACTACTGC |
| | | CACTGGTAGCTTGGGCATTGACTTGAAAGAGCCCATTTATACACAAAATGG |
| | | AAAACTAGGACTAAAGTACGGGGCTCCTTTGCATGTAACAGACGACCTAAA |
| | | CACTTTGACCGTAGCAACTGGTCCAGGTGTGACTATTAATAATACTTCCTTG |
| | | CAAACTAAAGTTACTGGAGCCTTGGGTTTTGATTCACAAGGCAATATGCAAC |
| | | TTAATGTAGCAGGAGGACTAAGGATTGATTCTCAAAACAGACGCCTTATACT |
| | | TGATGTTAGTTATCCGTTTGATGCTCAAAACCAACTAAATCTAAGACTAGGA |
| | | CAGGGCCCTCTTTTTATAAACTCAGCCCACAACTTGGATATTAACTACAACA |
| | | AAGGCCTTTACTTGTTTACAGCTTCAAACAATTCCAAAAAGCTTGAGGTTAA |
| | | CCTAAGCACTGCCAAGGGGTTGATGTTTGACGCTACAGCCATAGCCATTAA |
| | | TGCAGGAGATGGGCTTGAATTTGGTTCACCTAATGCACCAAACACAAATCC |
| | | CCTCAAAACAAAAATTGGCCATGGCCTAGAATTTGATTCAAACAAGGCTATG |
| | | GTTCCTAAACTAGGAACTGGCCTTAGTTTTGACAGCACAGGTGCCATTACA |
| | | GTAGGAAACAAAAATAATGATAAGCTAACCCTATGGACAGGTCCAAAACCA |
| | | GAAGCCAACTGCATAATTGAATACGGGAAACAAAACCCAGATAGCAAACTA |
| | | ACTTTAATCCTTGTAAAAAATGGAGGAATTGTTAATGGATATGTAACGCTAAT |
| | | GGGAGCCTCAGACTACGTTAACACCTTATTTAAAAACAAAAATGTCTCCATT |
| | | AATGTAGAACTATACTTTGATGCCACTGGTCATATATTACCAGACTCATCTT |
| | | CTCTTAAAACAGATCTAGAACTAAAATACAAGCAAACCGCTGACTTTAGTGC |
| | | AAGAGGTTTTATGCCAAGTACTACAGCGTATCCATTTGTCCTTCCTAATGCG |
| | | GGAACACATAATGAAAATTATATTTTTGGTCAATGCTACTACAAAGCAAGCG |
| | | ATGGTGCCCTTTTTCCGTTGGAAGTTACTGTTATGCTTAATAAACGCCTGCC |
| | | AGATAGTCGCACATCCTATGTTATGACTTTTTTATGGTCCTTGAATGCTGGT |
| | | CTAGCTCCAGAAACTACTCAGGCAACCCTCATAACCTCCCCATTTACCTTTT |
| | | CCTATATTAGAGAAGATGACTAATAAACTCTAAAGAATCGTTTGTGTTATGTT |
| | | TCAACGTGTTTATTTTTCAATTGCAGAAAATTTCAAGTCATTTTCATTCAGT |
| | | AGTATAGCCCCACCACCACATAGCTTATACAGATCACCGTACCTTAATCAAA |
| | | CTCACAGAACCCTAGTATTCAACCTGCCACCTCCCTCCCAACACACAGAGT |
| | | ACACAGTCCTTTCTCCCCGGCTGGCCTTAAAAAGCATCATATCATGGGTAA |
| | | CAGACATATTCTTAGGTGTTATATTCCACACGGTTTCCTGTCGAGCCAAACG |
| | | CTCATCAGTGATATTAATAAACTCCCCGGGCAGCTCACTTAAGTTCATGTCG |
| | | CTGTCCAGCTGCTGAGCCACAGGCTGCTGTCCAACTTGCGGTTGCTTAACG |
| | | GGCGGCGAAGGAGAAGTCCACGCCTACATGGGGGTAGAGTCATAATCGTG |
| | | CATCAGGATAGGGCGGTGGTGCTGCAGCAGCGCGCGAATAAACTGCTGCC |
| | | GCCGCCGCTCCGTCCTGCAGGAATACAACATGGCAGTGGTCTCCTCAGCG |
| | | ATGATTCGCACCGCCCGCAGCATAAGGCGCCTTGTCCTCCGGGCACAGCA |
| | | GCGCACCCTGATCTCACTTAAATCAGCACAGTAACTGCAGCACAGCACCAC |
| | | AATATTGTTCAAAATCCCACAGTGCAAGGCGCTGTATCCAAAGCTCATGGC |
| | | GGGGACCACAGAACCCACGTGGCCATCATACCACAAGCGCAGGTAGATTA |
| | | AGTGGCGACCCCTCATAAACACGCTGGACATAAACATTACCTCTTTTGGCA |
| | | TGTTGTAATTCACCACCTCCCGGTACCATATAAACCTCTGATTAAACATGGC |
| | | GCCATCCACCACCATCCTAAACCAGCTGGCCAAAACCTGCCCGCCGGCTA |
| | | TACACTGCAGGGAACCGGGACTGGAACAATGACAGTGGAGAGCCCAGGAC |
| | | TCGTAACCATGGATCATCATGCTCGTCATGATATCAATGTTGGCACAACACA |
| | | GGCACACGTGCATACACTTCCTCAGGATTACAAGCTCCTCCCGCGTTAGAA |
| | | CCATATCCCAGGGAACAACCCATTCCTGAATCAGCGTAAATCCCACACTGC |
| | | AGGGAAGACCTCGCACGTAACTCACGTTGTGCATTGTCAAAGTGTTACATT |
| | | CGGGCAGCAGCGGATGATCCTCCAGTATGGTAGCGCGGGTTTCTGTCTCA |
| | | AAAGGAGGTAGACGATCCCTACTGTACGGAGTGCGCCGAGACAACCGAGA |
| | | TCGTGTTGGTCGTAGTGTCATGCCAAATGGAACGCCGGACGTAGTCATATT |
| | | TCCTGAAGCAAAACCAGGTGCGGGCGTGACAAACAGATCTGCGTCTCCGG |
| | | TCTCGCCGCTTAGATCGCTCTGTGTAGTAGTTGTAGTATATCCACTCTCTCA |
| | | AAGCATCCAGGCGCCCCCTGGCTTCGGGTTCTATGTAAACTCCTTCATGCG |
| | | CCGCTGCCCTGATAACATCCACCACCGCAGAATAAGCCACACCCAGCCAA |
| | | CCTACACATTCGTTCTGCGAGTCACACACGGGAGGAGCGGGAAGAGCTGG |
| | | AAGAACCATGTTTTTTTTTTTATTCCAAAAGATTATCCAAAACCTCAAAATGA |
| | | AGATCTATTAAGTGAACGCGCTCCCCTCCGGTGGCGTGGTCAAACTCTACA |
| | | GCCAAAGAACAGATAATGGCATTTGTAAGATGTTGCACAATGGCTTCCAAAA |
| | | GGCAAACGGCCCTCACGTCCAAGTGGACGTAAAGGCTAAACCCTTCAGGG |
| | | TGAATCTCCTCTATAAACATTCCAGCACCTTCAACCATGCCCAAATAATTCT |
| | | CATCTCGCCACCTTCTCAATATATCTCTAAGCAAATCCCGAATATTAAGTCC |
| | | GGCCATTGTAAAAATCTGCTCCAGAGCGCCCTCCACCTTCAGCCTCAAGCA |
| | | GCGAATCATGATTGCAAAAATTCAGGTTCCTCACAGACCTGTATAAGATTCA |
| | | AAAGCGGAACATTAACAAAAATACCGCGATCCCGTAGGTCCCTTCGCAGGG |
| | | CCAGCTGAACATAATCGTGCAGGTCTGCACGGACCAGCGCGGCCACTTCC |
| | | CCGCCAGGAACCTTGACAAAAGAACCCACACTGATTATGACACGCATACTC |
| | | GGAGCTATGCTAACCAGCGTAGCCCCGATGTAAGCTTTGTTGCATGGGCG |
| | | GCGATATAAAATGCAAGGTGCTGCTCAAAAAATCAGGCAAAGCCTCGCGCA |
| | | AAAAAGAAAGCACATCGTAGTCATGCTCATGCAGATAAAGGCAGGTAAGCT |
| | | CCGGAACCACCACAGAAAAAGACACCATTTTTCTCTCAAACATGTCTGCGG |
| | | GTTTCTGCATAAACACAAAATAAAATAACAAAAAAACATTTAAACATTAGAAG |
| | | CCTGTCTTACAACAGGAAAAACAACCCTTATAAGCATAAGACGGACTACGG |
| | | CCATGCCGGCGTGACCGTAAAAAAACTGGTCACCGTGATTAAAAAGCACCA |
| | | CCGACAGCTCCTCGGTCATGTCCGGAGTCATAATGTAAGACTCGGTAAACA |
| | | CATCAGGTTGATTCATCGGTCAGTGCTAAAAAGCGACCGAAATAGCCCGGG |
| | | GGAATACATACCCGCAGGCGTAGAGACAACATTACAGCCCCCATAGGAGG |
| | | TATAACAAAATTAATAGGAGAGAAAAACACATAAACACCTGAAAAACCCTCC |
| | | TGCCTAGGCAAAATAGCACCCTCCCGCTCCAGAACAACATACAGCGCTTCA |
| | | CAGCGGCAGCCTAACAGTCAGCCTTACCAGTAAAAAAGAAAACCTATTAAA |
| | | AAAACACCACTCGACACGGCACCAGCTCAATCAGTCACAGTGTAAAAAAGG |
| | | GCCAAGTGCAGAGCGAGTATATATAGGACTAAAAAATGACGTAACGGTTAA |
| | | AGTCCACAAAAAACACCCAGAAAACCGCACGCGAACCTACGCCCAGAAAC |
| | | GAAAGCCAAAAAACCCACAACTTCCTCAAATCGTCACTTCCGTTTTCCCACG |
| | | TTACGTAACTTCCCATTTTAAGAAAACTACAATTCCCAACACATACAAGTTAC |
| | | TCCGCCCTAAAACCTACGTCACCCGCCCCGTTCCCACGCCCCGCGCCACG |
| | | TCACAAACTCCACCCCCTCATTATCATATTGGCTTCAATCCAAAATAAGGTA |
| | | TATTATTGATGATGTTAAT |
| 56 | HER2(A3)- | MTRAMDWIWRILFLVGAATGAHSEVQLVQSGTEVKKPGASVRVSCKSSGYTF |
| | CD28TM, ICD- | TSYYIHWVRQAPGQGLEWMAIINPGNGDTNYAQRFQGRVTMTRDTSTSTVYM |
| | CD3Z CAR | ELRSLRSDDTAVYFCAREIASYSGSYYDYWGQGTLVTVSSGGGGSGGGGSG |
| | | GGGSQAVVLQEPSLSVSPGGTVTLTCGLSSGSVSTGHYASWYQQTPGQAPR |
| | | TLFYNTNTRSSGVPDRFSGSIVGNKAALTITGAQADDESDYYCVLYVGDGIWVF |
| | | GGGTKLTVLEPKSCDKTHTCPTRFWVLVVVGGVLACYSLLVTVAFIIFWVRSKR |
| | | SRLLHSDYMNMTPRRPGPTRKHYQPYAPPRDFAAYRSRVKFSRSADAPAYQ |
| | | QGQNQLYNELNLGRREEYDVLDKRRGRDPEMGGKPQRRKNPQEGLYNELQK |
| | | DKMAEAYSEIGMKGERRRGKGHDGLYQGLSTATKDTYDALHMQALPPR |
| 57 | HER2(A3) LC- | GLSSGSVSTGHYAS |
| | CDR1 | |
| 58 | HER2(A3) LC- | NTNTRSS |
| | CDR2 | |
| 59 | HER2(A3) LC- | VLYVGDGIWV |
| | CDR3 | |
| 60 | HER2(A3) HC- | SYYIHWVRQA |
| | CDR1 | |
| 61 | HER2(A3) HC- | IINPGNGDTNYAQRFQG |
| | CDR2 | |
| 62 | HER2(A3) HC- | EIASYSGSYYDY |
| | CDR3 | |
| 63 | HER2(A3) VL | QAVVLQEPSLSVSPGGTVTLTCGLSSGSVSTGHYASWYQQTPGQAPRTLFYN |
| | | TNTRSSGVPDRFSGSIVGNKAALTITGAQADDESDYYCVLYVGDGIWVFGGGT |
| | | KLTVL |
| 64 | HER2(A3) VH | EVQLVQSGTEVKKPGASVRVSCKSSGYTFTSYYIHWVRQAPGQGLEWMAIINP |
| | | GNGDTNYAQRFQGRVTMTRDTSTSTVYMELRSLRSDDTAVYFCAREIASYSG |
| | | SYYDYWGQGTLVTVSS |

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence in disclosed the reverse complement thereof is also expressly contemplated.

### BRIEF DESCRIPTION OF THE FIGURES

Embodiments and studies illustrating the principles of the disclosure will now be discussed with reference to the accompanying figures.

FIGS. 1A and 1B. FIG. 1A shows schematic representations of examples of HER2-specific CAR constructs. FIG. 1B shows a schematic of an example of a protocol for transducing T cells to produce HER2-specific CAR-T.

FIG. 2. Graphs showing expression of the HER2-CARs, CCR7, CD45RO and PD-1 on T cells transduced with the indicated HER2-CAR constructs, as determined by flow cytometry.

FIG. 3. Graphs showing expression of HER2-CAR, CCR7, CD45RO, PD-1, LAG-3 and TIM-3 on CD4 and CD8 T cells following transduction with anti-HER2 clone E4 CAR construct, as determined by flow cytometry.

FIGS. 4A and 4B. FIG. 4A is a bar chart showing in vitro cell killing of MDA cells (which do not express HER2 at the cell surface; negative control), MDA-HER2 cells (which express HER2 at the cell surface; positive control), FaDu and SCC47 cells by anti-HER2 clone C5, E4 and F1 CAR-T cells (or non-transduced (NT) cells), as determined by ⁵¹Cr release assay. FIG. 4B shows graphs indicating expression of HER2 on MDA-HER2 cells, FaDu and SCC47 cells but not on MDA cells, as determined by flow cytometry.

FIG. 5. Bar chart showing in vitro cell killing of FaDu and SCC47 cells genetically modified to express firefly luciferase (ffLuc) by anti-HER2 clone C5, E4 and F1 CAR-T cells (or non-transduced (NT) cells), as determined by ffLuc activity assay. Data are presented as mean±SD (n=4). *P<0.001.

FIG. 6. FIG. 6 shows a schematic representation of the sequences of an example of an ICOSTAT oncolytic adenovirus construct.

FIGS. 7A to 7F. Graphs showing the ability of ICOSTAT oncolytic adenovirus to kill A549 cells (FIGS. 7A and 7F), FaDu cells (FIG. 7B), SCC47 cells (FIG. 7C), WI-38 cells (FIG. 7D) and ARPE-19 cells (FIG. 7E) following infection with the indicated concentration of viral particles (Vp), as determined by MTS viability assay. Helper-dependent adenovirus (HDAd) is included as a control condition.

FIGS. 8A and 8B. Bar charts showing ability of ICOSTAT oncolytic adenovirus to replicate and act as helper for replication of helper-dependent adenovirus (HDAd), as determined by copy number analysis by quantitative real-time PCR. The virus designated “Onc5/3AdicoSTAT” is ICOSTAT. “+HD” indicates co-infection of ICOSTAT with HDAd.

FIGS. 9A and 9B. Graphs showing the replication of ICOSTAT oncolytic adenovirus in FaDu cells (FIG. 9A) and SCC47 cells (FIG. 9B), in the presence or absence of 10 ng/ml IFNγ in the cell culture media.

FIGS. 10A to 10D. FIG. 10A is a schematic representation of the HDAd/L-12_TK_PDL1 construct. FIG. 10B is a bar chart showing production of IL-12p70 by cells transfected with the indicated helper-dependent adenovirus (HDAd) constructs. FIG. 10C is a photograph of a western blot showing production of anti-PD-L1 minibody by cells transfected with the HDAd constructs. FIG. 10D is a photograph of a wells demonstrating HSV thymidine kinase production by cells transfected with the HDAd constructs.

FIG. 11. Graph showing ELISA analysis of PD-L1 minibody avidity to recombinant human PD-L1, using serially diluted cell culture media of A549 cells which had been transfected with plasmid encoding GFP (pGFP; negative control), plasmid encoding the anti-PD-L1 minibody described in Tanoue et al. supra, (pPDL1 mini Tanoue) or plasmid encoding the anti-PD-L1 minibody encoded by HDAd/L-12_TK_PD-L1 (pPDL1 mini). Serially diluted anti-human PD-L1 antibody was used as a positive control (PDL1 IgG).

FIGS. 12A and 12B. Schematic representations of the sequences of (FIG. 12A) an example of an Onc5/2E1A24 oncolytic adenovirus construct, and (FIG. 12B) a plasmid encoding an Onc5/2E1A24 oncolytic adenovirus construct.

FIGS. 13A to 13D. Graphs showing the ability of Onc5/3Ad2E1A oncolytic adenovirus to kill FaDu cells (FIG. 13A), SCC47 cells (FIG. 13B), WI-38 cells (FIG. 13C) and ARPE-19 cells (FIG. 13D) following infection with the indicated concentration of viral particles (Vp), as determined by MTS viability assay. Helper-dependent adenovirus (HDAd) is included as a control condition.

FIG. 14. Graph showing numbers of HER2-specific CAR T cells following the indicated number of days of in vitro cell culture after transaction with the indicated CAR constructs.

FIGS. 15A to 15C. Images and graph showing the results of in vivo analysis of the anticancer activity of adoptively-transferred luciferase-expressing T cells in an orthotopic FaDu cell-derived model of squamous cell head and neck carcinoma. FIGS. 15A and 15B show the number and location of luciferase-expressing non-transduced T cells (NT), and cells expressing luciferase-expressing T cells expressing C5, F1 or A3 HER2-specific CARs within mice at the indicated number of days after infusion of the cells. FIG. 15C shows the percentage of surviving subjects in the different treatment groups at the indicated number of days after infusion of the cells. A negative control condition wherein mice were not administered with T cells is also shown (-).

FIGS. 16A to 16C. Images and graphs showing the results of in vivo analysis of adoptively-transferred T cells in NSG mice. FIG. 16A shows the number and location of luciferase-expressing non-transduced T cells (NT), and cells expressing luciferase-expressing T cells expressing C5, F1 or A3 HER2-specific CARs within mice at the indicated number of days after infusion of the cells. FIG. 16B shows measurements for total flux (in photons per second; p/s) of ventral surface for mice of the different groups at the indicated number of days after infusion of the cells. FIG. 16C shows the weights of mice in the different treatment groups at the indicated number of days after infusion of the cells, expressed as a percentage of body weight at day 0.

FIGS. 17A to 17C. Scatterplots and histograms showing the results of characterisation by flow cytometry of F1 HER2-specific CAR T cells used in experiments for in vivo analysis of the anti-cancer activity of the combination of CAdtrio and adoptively-transferred T cells. FIG. 17A shows the percentages of CD4+ T cells and CD8+ T cells within the F1.CAR-T population. FIG. 17B shows the percentage cells expressing HER2 CAR at the cell surface. FIG. 17C shows the percentages of cells within the F1.CAR-T population expressing CCR7 and/or CD45RO.

FIGS. 18A to 18D. Images and graphs showing the results of in vivo analysis of the anti-cancer activity of the combination of CAdtrio and adoptively-transferred T cells, in an orthotopic FaDu cell-derived model of squamous cell head and neck carcinoma. FIG. 18A shows the number and location of luciferase-expressing non-transduced T cells (NT), and cells expressing luciferase-expressing T cells expressing F1 HER2-specific CAR within mice at the indicated number of days after infusion of the cells Top right figure (Y-axis is labelled as Total Flux) is “Days post-injection of CAR T-cells”. Bottom 2 figures are “Days post-injection of CAdtrio. FIG. 18B shows measurements for total flux (in photons per second; p/s) of ventral surface for mice of the different groups at the indicated number of days after administration of CAdtrio. FIG. 18C shows the weights of mice in the different treatment groups at the indicated number of days after administration of CAdtrio, expressed as a percentage of body weight at day 0. FIG. 18D shows the percentage of surviving subjects in the different treatment groups at the indicated number of days after administration of CAdtrio. A negative control condition wherein mice were not administered with CAdtrio or T cells is also shown (-).

FIGS. 19A to 19C. Images and graphs showing the results of in vivo analysis of the anti-cancer activity of the combination of different ratios of Onc5/3Ad2E1Δ24:HDAd/L-12_TK_PD-L1 and adoptively-transferred HER2-specific CAR T cells, in an orthotopic FaDu cell-derived model of squamous cell head and neck carcinoma. FIG. 19A shows the number and location of luciferase-expressing FaDu cells within mice at the indicated number of days after administration of CAdtrio. FIG. 19B shows measurements for total flux (in photons per second; p/s) of ventral surface for mice of the different groups at the indicated number of days after administration of CAdtrio. FIG. 19C shows the weights of mice in the different treatment groups at the indicated number of days after administration of CAdtrio, expressed as a percentage of body weight at day 0.

FIGS. 20A to 20D. Bar charts and graphs showing the results of in vivo analysis of the combination of Onc5/3Ad2E1Δ24 and HDAd/L-12_TK_PD-L1 and ganciclovir (GCV), in an ectopic FaDu cell-derived model of squamous cell head and neck carcinoma. FIGS. 20A and 20B show the GAPDH-normalised copy number of (FIG. 20A) Onc5/3Ad2E1Δ24 and (FIG. 20B) HDAd/L-12_TK_PD-L1 in tumors of mice administered with the combination of Onc5/3Ad2E1Δ24 and HDAd/L-12_TK_PD-L1 (CAdtrio) at 22 days post infection, with or without GCV treatment. FIG. 20C shows tumor volume in mm3 of mice administered with the combination of Onc5/3Ad2E1Δ24 and HDAd/L-12_TK_PD-L1 (CAdtrio) at the indicated number of days post-injection of CAdtrio, with or without GCV treatment. FIG. 20D shows IL-12 levels detected by ELISA analysis of blood samples obtained at the indicated number of days post-injection of CAdtrio, with or without GCV treatment.

FIGS. 21A to 21C. Bar chart and images showing the results of analysis of transgene expression in cancer cell lines infected with different HDAd viruses, cultured in the presence or absence of ganciclovir (GCV). FIG. 21A shows the level of IL-12 in cell culture supernatant as determined by ELISA. FIG. 21B shows anti-PD-L1 minibody detected in cell culture supernatant by western blot. FIG. 21C shows viable cells detected by Crystal Violet staining at the end of the experiment.

FIGS. 22A and 22B. Scatterplots showing the results of characterisation by flow cytometry of Adenovirus-specific T cells (AdVSTs) used in experiments of Example 9. FIG. 22A shows the percentages of CD4+ T cells and CD8+ T cells within the AdVST population. FIG. 22B shows the percentages of cells within the AdVST population expressing CCR7 and/or CD45RO.

FIGS. 23A to 23C. Scatterplots and histograms showing the results of characterisation by flow cytometry F1.CAR-transduced AdVSTs used in experiments of Example 9. FIG. 23A shows the percentages of CD4+ T cells and CD8+ T cells within the transduced population. FIG. 23B shows the percentage cells expressing HER2 CAR at the cell surface. FIG. 23C shows the percentages of cells within the F1.CAR-AdVST population expressing CCR7 and/or CD45RO.

FIGS. 24A to 24D. Images and graphs showing the results of in vivo analysis of the anti-cancer activity of Adenovirus-specific T cells (AdVSTs), F1.CAR-transduced AdVSTs, the combination of F1.CAR-transduced AdVSTs with Onc5/3Ad2E1Δ24, and the combination of F1.CAR-transduced AdVSTs with Onc5/3Ad2E1Δ24+HDAd/L-12_TK_PD-L1 (“CAdtrio”). FIG. 24A shows the number and location of luciferase-expressing FaDu cells within mice at the indicated number of days after administration of CAdtrio. FIG. 24B shows measurements for total flux (in photons per second; p/s) of ventral surface for mice of the different groups at the indicated number of days after administration of CAdtrio. FIG. 24C shows the weights of mice in the different treatment groups at the indicated number of days after administration of CAdtrio, expressed as a percentage of body weight at day 0. FIG. 24D shows the percentage of surviving subjects in the different treatment groups at the indicated number of days after administration of CAdtrio.

FIGS. 25A to 25D. Graphs showing the results of in vivo analysis of the anti-cancer activity of CAdtrio alone, HER2.CAR T cells alone, and the combination of CAdtrio and HER2.CAR T cells in xenograph models using PC-3, SiHa, FaDu and SCC 47 cells. Tumour volume and percentage survival are shown for each cell line. Data are presented as means+/−SD (n=5-10). *P<0.01, **P=0.002 and ***P<0.001 for PC-3, *P=0.008, **P<0.001 and ***P=0.003 for SiHa, *P<0.001 for FaDu, *P<0.001 for SCC47.

FIGS. 26A to 26C. Graphs showing the results of in vivo analysis of the anti-cancer activity of CAdtrio alone, HER2.CAR T cells alone, and the combination of CAdtrio and HER2.CAR T cells in an orthotopic (metastatic) model of HNSCC. (A) Total flux of tongue implanted FaDu cells. (B) Percentage survival of NSG mice. (C) Body weight. Data are presented as means+/−SD (n=6-8). Total flux: *P=0.017, **P<0.005. Survival: *P=0.009, **P=0.006, ***P<0.003.

FIGS. 27A to 27C. CAdVEC induces host immune stimulation. (A) FaDu tumour growth in humanised mice. Day 0=day of CAdVEC treatment. (B) Human cytokine levels in serum. Data are presented as means±SD (n=5). (C) Serum levels of human anti-Ad IgG measured by ELISA-based assay. *P<0.05.

FIGS. 28A to 28C. Combination treatment controls tumour growth and extends median survival in subcutaneous xenograft immunodeficient mice. (A) FaDu tumour growth in NSG mice. Day 0=day of CAdVEC treatment. (B) Body weight of NSG mice. Percent changes compared to day 0 were calculated. (C) Survival curve after single or combination treatment.

FIGS. 29A to 29D. Combination treatment controls primary and metastatic tumour growth and extends median survival in orthotopic xenograft immunodeficient mice. (A and B) FaDu tumour growth in NSG mice. Day 0=day of CAdVEC treatment. (C) Survival curve after single or combination treatment. (D) Body weight of NSG mice. Percent changes compared to day 0 were calculated.

### NUMBERED STATEMENTS OF DISCLOSURE

Following numbered paragraphs (paras) describe particular aspects and embodiments of the present disclosure:

1. A method of treating a cancer, comprising administering to a subject:
(i) an oncolytic virus;
(ii) a virus comprising nucleic acid encoding an immunomodulatory factor; and
(iii) at least one cell comprising a chimeric antigen receptor (CAR) specific for a cancer cell antigen.

2. The method of para 1, wherein the oncolytic virus is an oncolytic adenovirus (OncAd).

3. The method of para 1 or para 2, wherein the oncolytic virus is derived from adenovirus 5 (Ad5).

4. The method of any one of paras 1 to 3, wherein the oncolytic virus encodes an E1A protein which displays reduced binding to Rb protein as compared to E1A protein encoded by Ad5.

5. The method of any one of paras 1 to 4, wherein the oncolytic virus encodes an E1A protein lacking the amino acid sequence LTCHEACF (SEQ ID NO:52).

6. The method of any one of paras 1 to 5, wherein the oncolytic virus encodes an E1A protein comprising, or consisting of, the amino acid sequence SEQ ID NO:34.

7. The method of any one of paras 1 to 6, wherein the oncolytic virus comprises nucleic acid having one or more binding sites for one or more transcription factors.

8. The method of any one of paras 1 to 7, wherein the oncolytic virus comprises nucleic acid having one or more binding sites for STAT1.

9. The method of any one of paras 1 to 8, wherein the virus comprising nucleic acid encoding an immunomodulatory factor is a helper-dependent adenovirus (HDAd).

10. The method of any one of paras 1 to 9, wherein the immunomodulatory factor is selected from: an agonist of an effector immune response or antagonist of an immunoregulatory response.

11. The method of any one of paras 1 to 10, wherein the virus comprising nucleic acid encoding an immunomodulatory factor comprises nucleic acid encoding IL-12 and/or antagonist anti-PD-L1 antibody.

12. The method of any one of paras 1 to 11, wherein the virus comprising nucleic acid encoding an immunomodulatory factor comprises nucleic acid encoding a thymidine kinase.

13. The method of any one of paras 1 to 12, wherein the at least one cell comprising a CAR specific for a cancer cell antigen is a T cell.

14. The method of any one of paras 1 to 13, wherein the CAR comprises an antigen binding domain capable of specific binding to HER2.

15. The method of any one of paras 1 to 14, wherein the CAR comprises an antigen binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;
and a VH domain comprising:
   HC-CRD1: SEQ ID NO:21;
   HC-CRD2: SEQ ID NO:22;
   HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31.

16. The method of any one of paras 1 to 15, wherein the CAR comprises an antigen binding domain comprising:
a VL comprising, or consisting of or consisting essentially of, an amino acid sequence having at least 75% sequence identity to SEQ ID NO:16 and a VH comprising, or consisting of or consisting essentially of, an amino acid sequence having at least 75% sequence identity to SEQ ID NO:17;

or
a VL comprising, or consisting of or consisting essentially of, an amino acid sequence having at least 75% sequence identity to SEQ ID NO:24 and a VH comprising, or consisting of or consisting essentially of, an amino acid sequence having at least 75% sequence identity to SEQ ID NO:25;

or
a VL comprising, or consisting of or consisting essentially of, an amino acid sequence having at least 75% sequence identity to SEQ ID NO:32 and a VH comprising, or consisting of or consisting essentially of, an amino acid sequence having at least 75% sequence identity to SEQ ID NO:33.

17. The method of any one of paras 1 to 16, wherein the method additionally comprises:
(a) isolating at least one cell from a subject;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject.

18. The method of any one of paras 1 to 17, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

19. An oncolytic adenovirus (OncAd) encoding an E1A protein comprising, or consisting of, the amino acid sequence SEQ ID NO:34.

20. An oncolytic adenovirus (OncAd) comprising nucleic acid having one or more binding sites for STAT1.

21. The OncAd according to para 20, wherein the OncAd comprises a nucleic acid sequence having at least 60% sequence identity to SEQ ID NO:51 or an equivalent sequence as a result of codon degeneracy.

22. A helper-dependent adenovirus (HDAd) comprising nucleic acid encoding IL-12 and/or antagonist anti-PD-L1 antibody.

23. The HDAd according to para 22, wherein the HDAd additionally comprises nucleic acid encoding a thymidine kinase.

24. A chimeric antigen receptor (CAR) comprising an antigen binding domain comprising:
a VL domain comprising:
LC-CRD1: SEQ ID NO:10;
LC-CRD2: SEQ ID NO:11;
LC-CRD3: SEQ ID NO:12;
and a VH domain comprising:
HC-CRD1: SEQ ID NO:13;
HC-CRD2: SEQ ID NO:14;
HC-CRD3: SEQ ID NO:15;

or
a VL domain comprising:
LC-CRD1: SEQ ID NO:18;
LC-CRD2: SEQ ID NO:19;
LC-CRD3: SEQ ID NO:20;
and a VH domain comprising:
HC-CRD1: SEQ ID NO:21;
HC-CRD2: SEQ ID NO:22;
HC-CRD3: SEQ ID NO:23;

or
a VL domain comprising:
LC-CRD1: SEQ ID NO:26;
LC-CRD2: SEQ ID NO:27;
LC-CRD3: SEQ ID NO:28;
and a VH domain comprising:
HC-CRD1: SEQ ID NO:29;
HC-CRD2: SEQ ID NO:30;
HC-CRD3: SEQ ID NO:31.

25. The CAR according to para 24, wherein the CAR comprises an antigen binding domain comprising:
a VL comprising, or consisting of, an amino acid sequence having at least 75% sequence identity to SEQ ID NO:16 and a VH comprising, or consisting of or consisting essentially of, an amino acid sequence having at least 75% sequence identity to SEQ ID NO:17;

or
a VL comprising, or consisting of or consisting essentially of, an amino acid sequence having at least 75% sequence identity to SEQ ID NO:24 and a VH comprising, or consisting of or consisting essentially of, an amino acid sequence having at least 75% sequence identity to SEQ ID NO:25;

or
a VL comprising, or consisting of, an amino acid sequence having at least 75% sequence identity to SEQ ID NO:32 and a VH comprising, or consisting of or consisting essentially of, an amino acid sequence having at least 75% sequence identity to SEQ ID NO:33.

26. A nucleic acid, optionally isolated or man-made, encoding the oncolytic adenovirus (OncAd) according to any one of paras 19 to 21, the helper-dependent adenovirus (HDAd) according to para 22 or para 23, or the chimeric antigen receptor (CAR) according to para 24 or para 25.

27. A cell comprising the oncolytic adenovirus (OncAd) according to any one of paras 19 to 21, the helper-dependent adenovirus (HDAd) according to para 22 or para 23, the chimeric antigen receptor (CAR) according to para 24 or para 25, or the nucleic acid according to para 26, optionally wherein the cell is man-made and not found in nature.

28. A pharmaceutical composition comprising the oncolytic adenovirus (OncAd) according to any one of paras 19 to 21, the helper-dependent adenovirus (HDAd) according to para 22 or para 23, the chimeric antigen receptor (CAR) according to para 24 or para 25, the nucleic acid according to para 26 or the cell according to para 27 and a pharmaceutically acceptable carrier, diluent, excipient or adjuvant.

29. A method of treating cancer comprising administering to a subject the oncolytic adenovirus (OncAd) according to any one of paras 19 to 21, the helper-dependent adenovirus (HDAd) according to para 22 or para 23, the chimeric antigen receptor (CAR) according to para 24 or para 25, the nucleic acid according to para 26, the cell according to para 27 or the pharmaceutical composition according to para 28.

30. The oncolytic adenovirus (OncAd) according to any one of paras 19 to 21, the helper-dependent adenovirus (HDAd) according to para 22 or para 23, the chimeric antigen receptor (CAR) according to para 24 or para 25, the nucleic acid according to para 26, the cell according to para 27 or the pharmaceutical composition according to para 28 for use in a method of treating a cancer.

31. Use of the oncolytic adenovirus (OncAd) according to any one of paras 19 to 21, the helper-dependent adenovirus (HDAd) according to para 22 or para 23, the chimeric antigen receptor (CAR) according to para 24 or para 25, the nucleic acid according to para 26, the cell according to para 27 or the pharmaceutical composition according to para 28 in the manufacture of a medicament for treating a cancer.

32. The method, the use or the use according to any one of paras 29 to 31, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

33. A kit of parts comprising a predetermined quantity of the oncolytic adenovirus (OncAd) according to any one of paras 19 to 21, the helper-dependent adenovirus (HDAd) according to para 22 or para 23, the chimeric antigen receptor (CAR) according to para 24 or para 25, the nucleic acid according to para 26, the cell according to para 27 or the pharmaceutical composition according to para 28.

1A. A method of treating a cancer, comprising administering to a subject:
(i) an oncolytic virus;
(ii) a virus comprising nucleic acid encoding an immunomodulatory factor; and
(iii) at least one cell comprising a chimeric antigen receptor (CAR) specific for a cancer cell antigen.

2A. A combination of (i) an oncolytic virus, (ii) a virus comprising nucleic acid encoding an immunomodulatory factor, and (iii) at least one cell comprising a chimeric antigen receptor (CAR) specific for a cancer cell antigen, for use in a method of treating a cancer.

3A. Use of (i) an oncolytic virus, (ii) a virus comprising nucleic acid encoding an immunomodulatory factor, and (iii) at least one cell comprising a chimeric antigen receptor (CAR) specific for a cancer cell antigen, in the manufacture of a medicament for use in a method of treating a cancer.

4A. A method of treating a cancer, comprising administering to a subject:
(i) an oncolytic virus; and
(ii) at least one cell comprising a chimeric antigen receptor (CAR) specific for a cancer cell antigen.

5A. A combination of (i) an oncolytic virus, and (ii) at least one cell comprising a chimeric antigen receptor (CAR) specific for a cancer cell antigen, for use in a method of treating a cancer.

6A. Use of (i) an oncolytic virus, and (ii) at least one cell comprising a chimeric antigen receptor (CAR) specific for a cancer cell antigen, in the manufacture of a medicament for use in a method of treating a cancer.

7A. The method according to para 1A or para 4A, the combination according to para 2A or para 5A, or the use according to para 3A or para 6A, wherein the cell comprising a CAR is specific for the oncolytic virus.

8A. A method of treating a cancer, comprising administering to a subject:
(i) an oncolytic virus; and
(ii) at least one immune cell specific for the oncolytic virus.

9A. A combination of (i) an oncolytic virus, and (ii) at least one immune cell specific for the oncolytic virus, for use in a method of treating a cancer.

10A. Use of (i) an oncolytic virus, and (ii) at least one immune cell specific for the oncolytic virus, in the manufacture of a medicament for use in a method of treating a cancer.

11A. The method, combination or use according to any one of paras 1A to 10A, wherein the oncolytic virus is an oncolytic adenovirus (OncAd).

12A. The method, combination or use according to any one of paras 1A to 11A, wherein the oncolytic virus is derived from adenovirus 5 (Ad5).

13A. The method, combination or use according to any one of paras 1A to 12A, wherein the oncolytic virus encodes an E1A protein which displays reduced binding to Rb protein as compared to E1A protein encoded by Ad5.

14A. The method, combination or use according to any one of paras 1A to 13A, wherein the oncolytic virus encodes an E1A protein lacking the amino acid sequence LTCHEACF (SEQ ID NO:52).

15A. The method, combination or use according to any one of paras 1A to 14A, wherein the oncolytic virus encodes an E1A protein comprising, or consisting of, the amino acid sequence SEQ ID NO:34.

16A. The method, combination or use according to any one of paras 1A to 15A, wherein the oncolytic virus comprises nucleic acid having one or more binding sites for one or more transcription factors.

17A. The method, combination or use according to any one of paras 1A to 16A, wherein the oncolytic virus comprises nucleic acid having one or more binding sites for STAT1.

18A. The method, combination or use according to any one of paras 1A to 7A, or 11A to 17A, wherein the at least one cell comprising a CAR specific for a cancer cell antigen is a T cell.

19A. The method, combination or use according to any one of paras 1A to 7A, or 11A to 18A, wherein the CAR comprises an antigen binding domain capable of specific binding to HER2.

20A. The method, combination or use according to any one of paras 1A to 7A, or 11A to 19A, wherein the CAR comprises an antigen-binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;
and a VH domain comprising:
   HC-CRD1: SEQ ID NO:21;
   HC-CRD2: SEQ ID NO:22;
   HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

21A. The method, combination or use according to any one of paras 1A to 7A, or 11A to 20A, wherein the CAR comprises an antigen binding domain comprising:
a VL comprising, or consisting of, an amino acid sequence having at least 75% sequence identity to SEQ ID NO:16 and a VH comprising, or consisting of, an amino acid sequence having at least 75% sequence identity to SEQ ID NO:17;

or
a VL comprising, or consisting of, an amino acid sequence having at least 75% sequence identity to SEQ ID NO:24 and a VH comprising, or consisting of, an amino acid sequence having at least 75% sequence identity to SEQ ID NO:25;

or
a VL comprising, or consisting of, an amino acid sequence having at least 75% sequence identity to SEQ ID NO:32 and a VH comprising, or consisting of, an amino acid sequence having at least 75% sequence identity to SEQ ID NO:33;

or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

22A. The method, combination or use according to any one of paras 1A to 3A, or 11A to 21A, wherein the virus comprising nucleic acid encoding an immunomodulatory factor is a helper-dependent adenovirus (HDAd).

23A. The method, combination or use according to any one of paras 1A to 3A, or 11A to 22A, wherein the immunomodulatory factor is selected from: an agonist of an effector immune response or antagonist of an immunoregulatory response.

24A. The method, combination or use according to any one of paras 1A to 3A, or 11A to 23A, wherein the virus comprising nucleic acid encoding an immunomodulatory factor comprises nucleic acid encoding IL-12 and/or antagonist anti-PD-L1 antibody.

25A. The method, combination or use according to any one of paras 1A to 3A, or 11A to 24A, wherein the virus comprising nucleic acid encoding an immunomodulatory factor comprises nucleic acid encoding an enzyme capable of catalysing conversion of a non-toxic factor to a cytotoxic form.

26A. The method, combination or use according to para 25A, wherein the enzyme is selected from: thymidine kinase, cytosine deaminase, nitroreductase, cytochrome P450, carboxypeptidase G2, purine nucleoside phosphorylase, horseradish peroxidase and carboxylesterase.

27A. The method, combination or use according to any one of paras 1A to 26A, wherein the method of treating a cancer comprises:
(a) isolating at least one cell from a subject;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject.

28A. The method, combination or use according to any one of paras 1A to 27A, wherein the method of treating a cancer comprises:
(a) isolating immune cells from a subject;
(b) generating or expanding a population of immune cells specific for an oncolytic virus by a method comprising: stimulating the immune cells by culture in the presence of antigen presenting cells (APCs) presenting a peptide of the oncolytic virus, and;
(c) administering at least one immune cell specific for the oncolytic virus to a subject.

29A. The method, combination or use according to any one of paras 1A to 28A, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

30A. An oncolytic adenovirus (OncAd) encoding an E1A protein comprising, or consisting of, the amino acid sequence SEQ ID NO:34.

31A. An oncolytic adenovirus (OncAd) comprising nucleic acid having one or more binding sites for STAT1.

32A. An oncolytic adenovirus (OncAd) comprising a nucleic acid sequence having at least 60% sequence identity to SEQ ID NO:51 or an equivalent sequence as a result of codon degeneracy.

33A. An oncolytic adenovirus (OncAd) comprising a nucleic acid sequence having at least 60% sequence identity to SEQ ID NO:55 or an equivalent sequence as a result of codon degeneracy.

34A. A helper-dependent adenovirus (HDAd) comprising nucleic acid encoding IL-12 and/or antagonist anti-PD-L1 antibody.

35A. The HDAd according to para 34A, wherein the HDAd additionally comprises nucleic acid encoding an enzyme capable of catalysing conversion of a non-toxic factor to a cytotoxic form.

36A. The HDAd according to para 34A or para 35A, wherein the enzyme is selected from:
thymidine kinase, cytosine deaminase, nitroreductase, cytochrome P450, carboxypeptidase G2, purine nucleoside phosphorylase, horseradish peroxidase and carboxylesterase.

37A. A chimeric antigen receptor (CAR) comprising an antigen binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;
and a VH domain comprising:
   HC-CRD1: SEQ ID NO:21;
   HC-CRD2: SEQ ID NO:22;
   HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

38A. The CAR according to para 37A, wherein the CAR comprises an antigen binding domain comprising:
a VL comprising, or consisting of, an amino acid sequence having at least 75% sequence identity to SEQ ID NO:16 and a VH comprising, or consisting of, an amino acid sequence having at least 75% sequence identity to SEQ ID NO:17;

or
a VL comprising, or consisting of, an amino acid sequence having at least 75% sequence identity to SEQ ID NO:24 and a VH comprising, or consisting of, an amino acid sequence having at least 75% sequence identity to SEQ ID NO:25;

or
a VL comprising, or consisting of, an amino acid sequence having at least 75% sequence identity to SEQ ID NO:32 and a VH comprising, or consisting of, an amino acid sequence having at least 75% sequence identity to SEQ ID NO:33

or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

39A. A nucleic acid, or a plurality of nucleic acids, optionally isolated or man-made, encoding the oncolytic adenovirus (OncAd) according to any one of para 30A to 33A, the helper-dependent adenovirus (HDAd) according to any one of paras 34A to 36A, or the chimeric antigen receptor (CAR) according to para 37A or para 38A.

40A. A cell comprising the oncolytic adenovirus (OncAd) according to any one of para 30A to 33A, the helper-dependent adenovirus (HDAd) according to any one of paras 34A to 36A, the chimeric antigen receptor (CAR) according to para 37A or para 38A, or the nucleic acid or plurality of nucleic acids according to para 39A.

41A. A pharmaceutical composition comprising the oncolytic adenovirus (OncAd) according to any one of paras 30A to 33A, the helper-dependent adenovirus (HDAd) according to any one of paras 34A to 36A, the chimeric antigen receptor (CAR) according to para 37A or para 38A, the nucleic acid or plurality of nucleic acids according to para 39A, or the cell according to para 40A, and a pharmaceutically acceptable carrier, diluent, excipient or adjuvant.

42A. A method of treating cancer comprising administering to a subject the oncolytic adenovirus (OncAd) according to any one of paras 30A to 33A, the helper-dependent adenovirus (HDAd) according to any one of paras 34A to 36A, the chimeric antigen receptor (CAR) according to para 37A or para 38A, the nucleic acid or plurality of nucleic acids according to para 39A, the cell according to para 40A, or the pharmaceutical composition according to para 41A.

43A. The oncolytic adenovirus (OncAd) according to any one of paras 30A to 33A, the helper-dependent adenovirus (HDAd) according to any one of paras 34A to 36A, the chimeric antigen receptor (CAR) according to para 37A or para 38A, the nucleic acid or plurality of nucleic acids according to para 39A, the cell according to para 40A, or the pharmaceutical composition according to para 41A, for use in a method of treating a cancer.

44A. Use of the oncolytic adenovirus (OncAd) according to any one of paras 30A to 33A, the helper-dependent adenovirus (HDAd) according to any one of paras 34A to 36A, the chimeric antigen receptor (CAR) according to para 37A or para 38A, the nucleic acid or plurality of nucleic acids according to para 39A, the cell according to para 40A, or the pharmaceutical composition according to para 41A, in the manufacture of a medicament for treating a cancer.

45A. The method according to para 42A, the OncAd, HDAd, CAR, nucleic acid or plurality of nucleic acids, cell, or pharmaceutical composition for use according to para 43A, or the use according to para 44A, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

46A. A kit of parts comprising a predetermined quantity of the oncolytic adenovirus (OncAd) according to any one of paras 30A to 33A, the helper-dependent adenovirus (HDAd) according to any one of paras 34A to 36A, the chimeric antigen receptor (CAR) according to para 37A or para 38A, the nucleic acid or plurality of nucleic acids according to para 39A, the cell according to para 40A, or the pharmaceutical composition according to para 41A.

1B. A method of treating a cancer, comprising administering to a subject:
(i) an oncolytic virus; and
(ii) at least one cell comprising a chimeric antigen receptor (CAR) specific for a cancer cell antigen.

2B. A combination of (i) an oncolytic virus, and (ii) at least one cell comprising a chimeric antigen receptor (CAR) specific for a cancer cell antigen, for use in a method of treating a cancer.

3B. Use of (i) an oncolytic virus, and (ii) at least one cell comprising a chimeric antigen receptor (CAR) specific for a cancer cell antigen, in the manufacture of a medicament for use in a method of treating a cancer.

4B. The method according to para 1B, the combination according to para 2B, or the use according to para 3B, wherein the cell comprising a CAR is specific for the oncolytic virus.

5B. A method of treating a cancer, comprising administering to a subject:
(i) an oncolytic virus; and
(ii) at least one immune cell specific for the oncolytic virus.

6B. A combination of (i) an oncolytic virus, and (ii) at least one immune cell specific for the oncolytic virus, for use in a method of treating a cancer.

7B. Use of (i) an oncolytic virus, and (ii) at least one immune cell specific for the oncolytic virus, in the manufacture of a medicament for use in a method of treating a cancer.

8B. The method, combination or use according to any one of paras 1B to 7B, wherein the oncolytic virus is an oncolytic adenovirus (OncAd).

9B. The method, combination or use according to any one of paras 1B to 8B, wherein the oncolytic virus is derived from adenovirus 5 (Ad5).

10B. The method, combination or use according to any one of paras 1B to 9B, wherein the oncolytic virus encodes an E1A protein which displays reduced binding to Rb protein as compared to E1A protein encoded by Ad5.

11B. The method, combination or use according to any one of paras 1B to 10B, wherein the oncolytic virus encodes an E1A protein lacking the amino acid sequence LTCHEACF (SEQ ID NO:52).

12B. The method, combination or use according to any one of paras 1B to 11B, wherein the oncolytic virus encodes an E1A protein comprising, or consisting of, the amino acid sequence SEQ ID NO:34.

13B. The method, combination or use according to any one of paras 1B to 12B, wherein the oncolytic virus comprises nucleic acid having one or more binding sites for one or more transcription factors.

14B. The method, combination or use according to any one of paras 1B to 13B, wherein the oncolytic virus comprises nucleic acid having one or more binding sites for STAT1.

15B. The method, combination or use according to any one of paras 1B to 4B, or 8B to 14B, wherein the at least one cell comprising a CAR specific for a cancer cell antigen is a T cell.

16B. The method, combination or use according to any one of paras 1B to 4B, or 8B to 15B, wherein the CAR comprises an antigen binding domain capable of specific binding to HER2.

17B. The method, combination or use according to any one of paras 1B to 4B, or 8B to 16B, wherein the CAR comprises an antigen-binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;
and a VH domain comprising:
   HC-CRD1: SEQ ID NO:21;
   HC-CRD2: SEQ ID NO:22;
   HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

18B. The method, combination or use according to any one of paras 1B to 4B, or 8B to 17B, wherein the CAR comprises an antigen binding domain comprising:
a VL comprising, or consisting of, an amino acid sequence having at least 75% sequence identity to SEQ ID NO:16 and a VH comprising, or consisting of, an amino acid sequence having at least 75% sequence identity to SEQ ID NO:17;

or
a VL comprising, or consisting of, an amino acid sequence having at least 75% sequence identity to SEQ ID NO:24 and a VH comprising, or consisting of, an amino acid sequence having at least 75% sequence identity to SEQ ID NO:25;

or
a VL comprising, or consisting of, an amino acid sequence having at least 75% sequence identity to SEQ ID NO:32 and a VH comprising, or consisting of, an amino acid sequence having at least 75% sequence identity to SEQ ID NO:33;

or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

19B. The method, combination or use according to any one of paras 1B to 18B wherein the method, combination or use further comprises a virus comprising nucleic acid encoding an immunomodulatory factor.

20B. The method, combination or use according to para 19B wherein the virus comprising nucleic acid encoding an immunomodulatory factor is a helper-dependent adenovirus (HDAd).

21B. The method, combination or use according to para 19B or 20B, wherein the immunomodulatory factor is selected from: an agonist of an effector immune response or antagonist of an immunoregulatory response.

22B. The method, combination or use according to any one of paras 19B to 21B, wherein the virus comprising nucleic acid encoding an immunomodulatory factor comprises nucleic acid encoding IL-12 and/or antagonist anti-PD-L1 antibody.

23B. The method, combination or use according to any one of paras 19B to 22B, wherein the virus comprising nucleic acid encoding an immunomodulatory factor comprises nucleic acid encoding an enzyme capable of catalysing conversion of a non-toxic factor to a cytotoxic form.

24B. The method, combination or use according to para 23B, wherein the enzyme is selected from: thymidine kinase, cytosine deaminase, nitroreductase, cytochrome P450, carboxypeptidase G2, purine nucleoside phosphorylase, horseradish peroxidase and carboxylesterase.

25B. The method, combination or use according to any one of paras 1B to 24B, wherein the method of treating a cancer comprises:
(a) isolating at least one cell from a subject;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject.

26B. The method, combination or use according to any one of paras 1B to 25B, wherein the method of treating a cancer comprises:
(a) isolating immune cells from a subject;
(b) generating or expanding a population of immune cells specific for an oncolytic virus by a method comprising: stimulating the immune cells by culture in the presence of antigen presenting cells (APCs) presenting a peptide of the oncolytic virus, and;
(c) administering at least one immune cell specific for the oncolytic virus to a subject.

27B. The method, combination or use according to any one of paras 1B to 26B, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

28B. An oncolytic adenovirus (OncAd) encoding an E1A protein comprising, or consisting of, the amino acid sequence SEQ ID NO:34.

29B. An oncolytic adenovirus (OncAd) comprising nucleic acid having one or more binding sites for STAT1.

30B. An oncolytic adenovirus (OncAd) comprising a nucleic acid sequence having at least 60% sequence identity to SEQ ID NO:51 or an equivalent sequence as a result of codon degeneracy.

31B. An oncolytic adenovirus (OncAd) comprising a nucleic acid sequence having at least 60% sequence identity to SEQ ID NO:55 or an equivalent sequence as a result of codon degeneracy.

32B. A helper-dependent adenovirus (HDAd) comprising nucleic acid encoding IL-12 and/or antagonist anti-PD-L1 antibody.

33B. The HDAd according to para 32B, wherein the HDAd additionally comprises nucleic acid encoding an enzyme capable of catalysing conversion of a non-toxic factor to a cytotoxic form.

34B. The HDAd according to para 32B or para 33B, wherein the enzyme is selected from: thymidine kinase, cytosine deaminase, nitroreductase, cytochrome P450, carboxypeptidase G2, purine nucleoside phosphorylase, horseradish peroxidase and carboxylesterase.

35B. A chimeric antigen receptor (CAR) comprising an antigen binding domain comprising:
a VL domain comprising:
   LC-CRD1: SEQ ID NO:10;
   LC-CRD2: SEQ ID NO:11;
   LC-CRD3: SEQ ID NO:12;
and a VH domain comprising:
   HC-CRD1: SEQ ID NO:13;
   HC-CRD2: SEQ ID NO:14;
   HC-CRD3: SEQ ID NO:15;

or
a VL domain comprising:
   LC-CRD1: SEQ ID NO:18;
   LC-CRD2: SEQ ID NO:19;
   LC-CRD3: SEQ ID NO:20;
and a VH domain comprising:
   HC-CRD1: SEQ ID NO:21;
   HC-CRD2: SEQ ID NO:22;
   HC-CRD3: SEQ ID NO:23;

or
a VL domain comprising:
   LC-CRD1: SEQ ID NO:26;
   LC-CRD2: SEQ ID NO:27;
   LC-CRD3: SEQ ID NO:28;
and a VH domain comprising:
   HC-CRD1: SEQ ID NO:29;
   HC-CRD2: SEQ ID NO:30;
   HC-CRD3: SEQ ID NO:31;

or
a VL domain comprising:
   LC-CRD1: SEQ ID NO:57;
   LC-CRD2: SEQ ID NO:58;
   LC-CRD3: SEQ ID NO:59;
and a VH domain comprising:
   HC-CRD1: SEQ ID NO:60;
   HC-CRD2: SEQ ID NO:61;
   HC-CRD3: SEQ ID NO:62.

36B. The CAR according to para 35B, wherein the CAR comprises an antigen binding domain comprising:
a VL comprising, or consisting of, an amino acid sequence having at least 75% sequence identity to SEQ ID NO:16 and a VH comprising, or consisting of, an amino acid sequence having at least 75% sequence identity to SEQ ID NO:17;

or
a VL comprising, or consisting of, an amino acid sequence having at least 75% sequence identity to SEQ ID NO:24 and a VH comprising, or consisting of, an amino acid sequence having at least 75% sequence identity to SEQ ID NO:25;

or
a VL comprising, or consisting of, an amino acid sequence having at least 75% sequence identity to SEQ ID NO:32 and a VH comprising, or consisting of, an amino acid sequence having at least 75% sequence identity to SEQ ID NO:33

or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

37B. A nucleic acid, or a plurality of nucleic acids, optionally isolated or man-made, encoding the oncolytic adenovirus (OncAd) according to any one of paras 28B to 31B, the helper-dependent adenovirus (HDAd) according to any one of paras 32B to 24B, or the chimeric antigen receptor (CAR) according to para 35B or para 36B.

38B. A cell comprising the oncolytic adenovirus (OncAd) according to any one of paras 28B to 31B, the helper-dependent adenovirus (HDAd) according to any one of paras 32B to 34B, the chimeric antigen receptor (CAR) according to para 35B or para 36B, or the nucleic acid or plurality of nucleic acids according to para 37B.

39B. A pharmaceutical composition comprising the oncolytic adenovirus (OncAd) according to any one of paras 28B to 31B, the helper-dependent adenovirus (HDAd) according to any one of paras 32B to 34B, the chimeric antigen receptor (CAR) according to para 35B or para 36B, the nucleic acid or plurality of nucleic acids according to para 37B, or the cell according to para 38B, and a pharmaceutically acceptable carrier, diluent, excipient or adjuvant.

40B. A method of treating cancer comprising administering to a subject the oncolytic adenovirus (OncAd) according to any one of paras 28B to 31B, the helper-dependent adenovirus (HDAd) according to any one of paras 32B to 34B, the chimeric antigen receptor (CAR) according to para 35B or para 36B, the nucleic acid or plurality of nucleic acids according to para 37B, the cell according to para 38B, or the pharmaceutical composition according to para 39B.

41B. The oncolytic adenovirus (OncAd) according to any one of paraS 28B to 31B, the helper-dependent adenovirus (HDAd) according to any one of paraS 32B to 34B, the chimeric antigen receptor (CAR) according to para 35B or para 36B, the nucleic acid or plurality of nucleic acids according to para 37B, the cell according to para 38B, or the pharmaceutical composition according to para 39B, for use in a method of treating a cancer.

42B. Use of the oncolytic adenovirus (OncAd) according to any one of paras 28B to 31B, the helper-dependent adenovirus (HDAd) according to any one of paras 32B to 34B, the chimeric antigen receptor (CAR) according to para 35B or para 36B, the nucleic acid or plurality of nucleic acids according to para 37B, the cell according to para 38B, or the pharmaceutical composition according to para 39B, in the manufacture of a medicament for treating a cancer.

43B. The method according to para 40B, the OncAd, HDAd, CAR, nucleic acid or plurality of nucleic acids, cell, or pharmaceutical composition for use according to para 41B, or the use according to para 42B, wherein the cancer is selected from head and neck cancer, nasopharyngeal carcinoma (NPC), cervical carcinoma (CC), oropharyngeal carcinoma (OPC), gastric carcinoma (GC), hepatocellular carcinoma (HCC) and lung cancer.

44B. A kit of parts comprising a predetermined quantity of the oncolytic adenovirus (OncAd) according to any one of paras 28B to 31B, the helper-dependent adenovirus (HDAd) according to any one of paras 32B to 34B, the chimeric antigen receptor (CAR) according to para 35B or para 36B, the nucleic acid or plurality of nucleic acids according to para 37B, the cell according to para 38B, or the pharmaceutical composition according to para 39B.

### EXAMPLES

In the following Examples, the inventors describe the generation functional characterisation of novel HER-2 specific CARs and CAR-T cells, oncolytic adenoviruses and helper-dependent adenovirus.

### Example 1: HER2-Specific CAR-T Cells

1.1 Generation of HER2-Specific CAR Constructs and CAR-T Cells

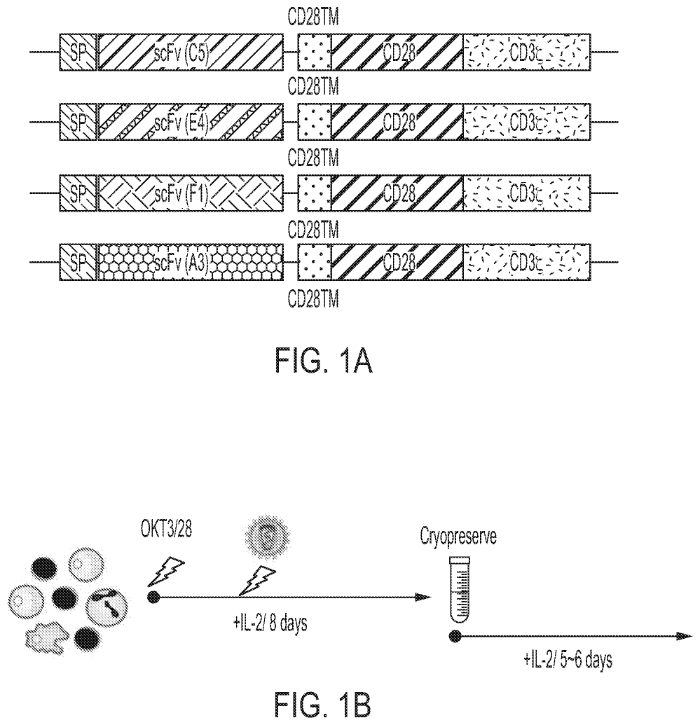

HER2-binding CAR constructs were prepared. Briefly, DNA encoding scFv (i.e. VL domain and VH domain joined by a linker sequence) for the anti-HER2 antibody clone C5, E4, F1 or A3 was cloned into a CAR construct backbone comprising a 5′ signal peptide (SP), and CD28 transmembrane (TM) and intracellular domain sequence, with a 3′ CD3ζ intracellular domain sequence. The three HER2-binding CAR constructs are represented schematically in FIG. 1A.

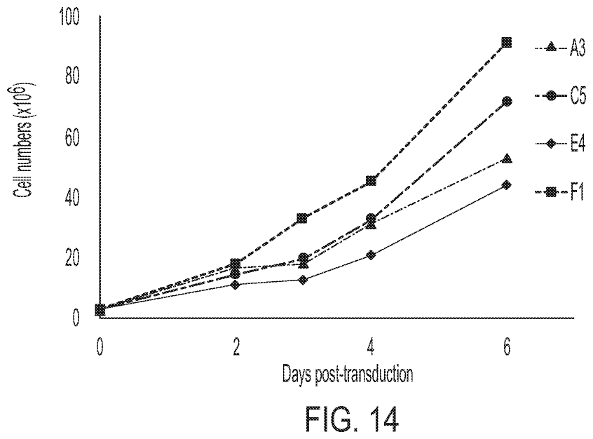

HER2 specific CAR-T cells were generated as represented graphically in FIG. 1B. Briefly, human PBMCs were isolated from blood samples by with Ficoll density gradient centrifugation. Cells were treated by stimulation with anti-CD3(OKT3)/anti-CD28 in the presence of IL-2 to promote T cell activation and proliferation, and the cells were transduced with retrovirus encoding the HER2 CAR constructs. T-cells were expanded by culture in the presence of 100 IU/mL recombinant human IL-2, and were frozen at 6 days post-transduction. The HER2-specific CAR construct-transduced T cells were readily expanded by culture in vitro (see e.g. FIG. 14).

T-cells were thawed and expanded in the presence of 100 IU/mL of recombinant human IL-2 for 5 days and used for in vitro/in vivo experiments and phenotypic analysis.

1.2 Characterisation of the HER2-Specific CAR-T Cells

1.2.1 Expression of Surface Markers and HER2 CARs

T cells transduced with HER2 CAR construct encoding scFv for anti-HER2 antibody clone E4 were characterised by flow cytometry for expression of different cell surface molecules. Expanded HER2 specific CAR T-cells were stained with fluorescently-labelled monoclonal antibodies for 30 minutes at 4° C. Discrimination of live/dead cells was achieved by including 7AAD in stainings (BD Pharmingen). Stained cells were analyzed using a Gallios flow cytometer and Kaluza software (BD Bioscience), according to manufacturer's instructions.

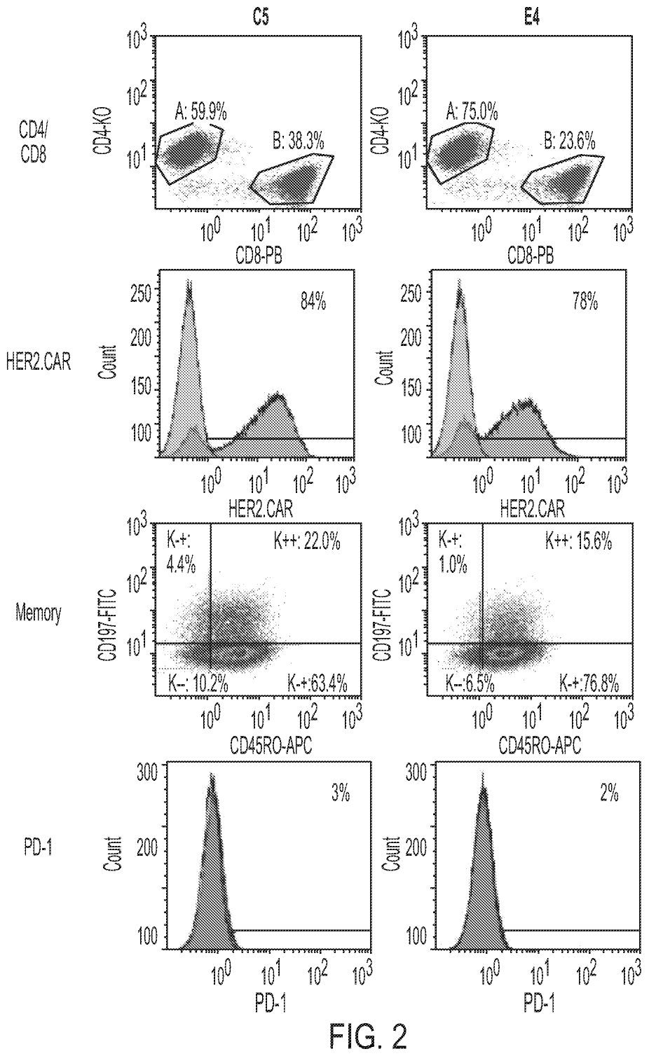

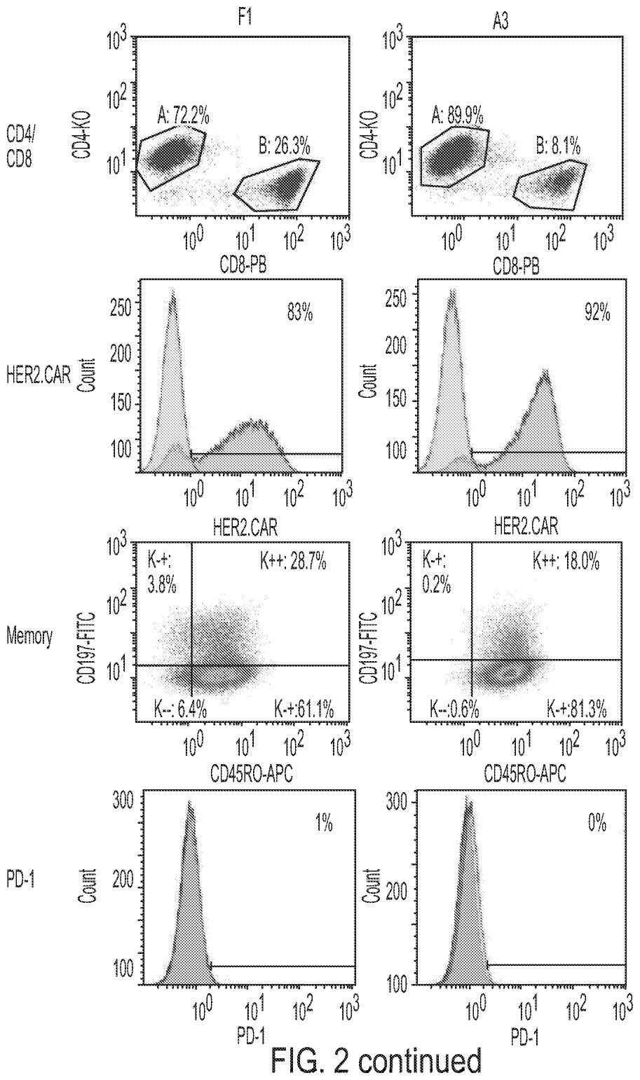

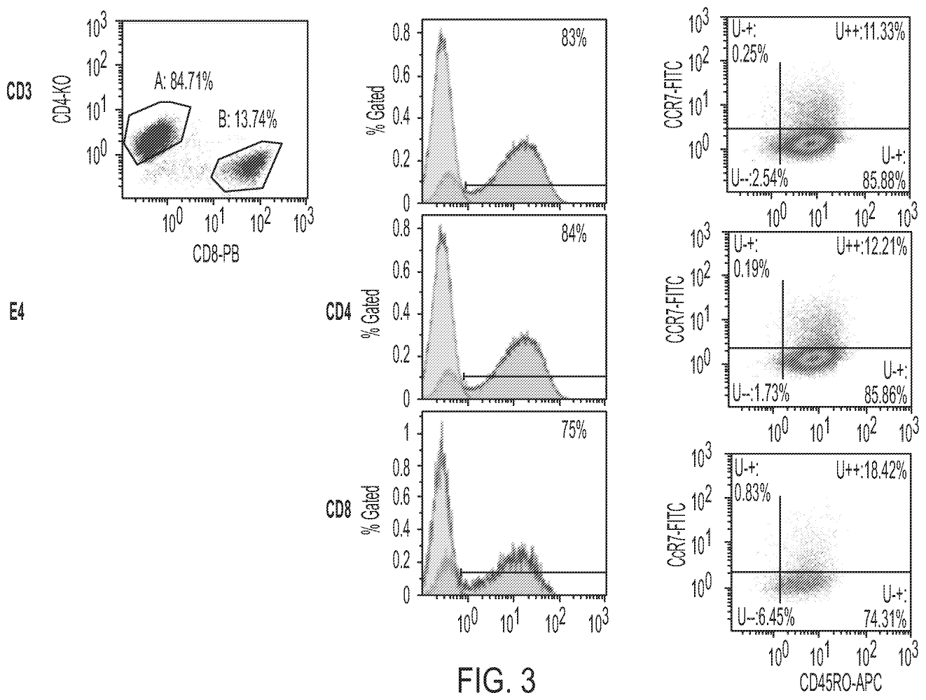

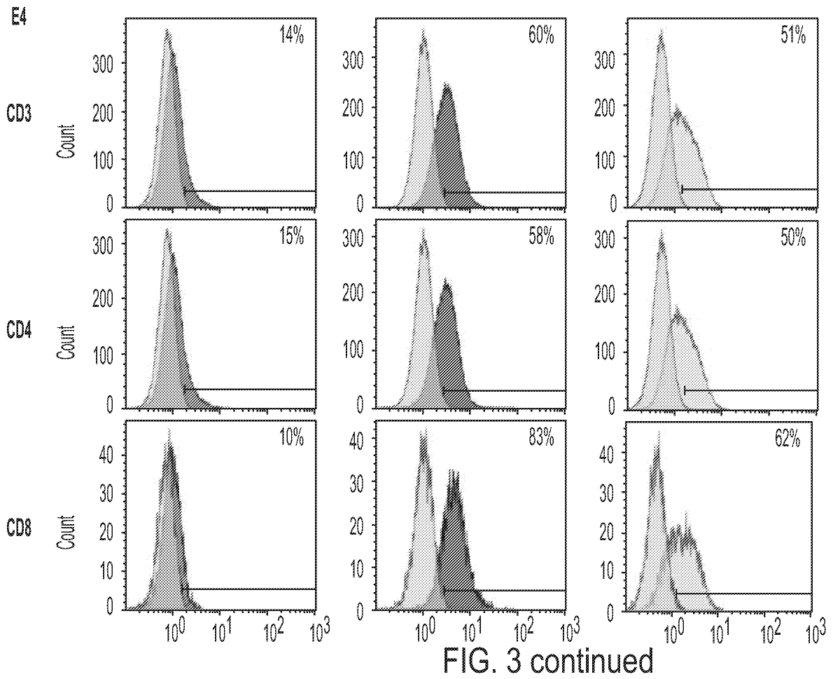

The results are shown in FIGS. 2 and 3. Strong surface expression of the HER2-CARs was detected on the transduced cells (FIG. 2).

FIG. 3 shows the results of characterisation of T cells transduced with HER2(E4)-CAR. CD3+ cells, CD4+ cells and CD8+ cells expressing HER2(E4)-CAR were shown to have increased expression of PD-1, LAG-3 and TIM-3, and to have reduced level of expression of CCR7 as compared to non-transduced cells (FIG. 3).

1.2.2 Cell Killing Activity

The HER2-CAR-T cells were analysed for their ability to kill HER2 expressing cancer cells in vitro in cell killing assays.

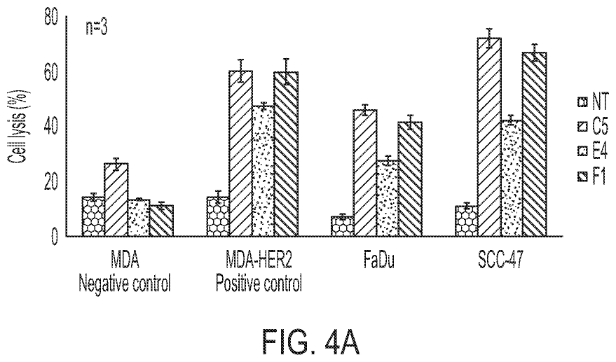

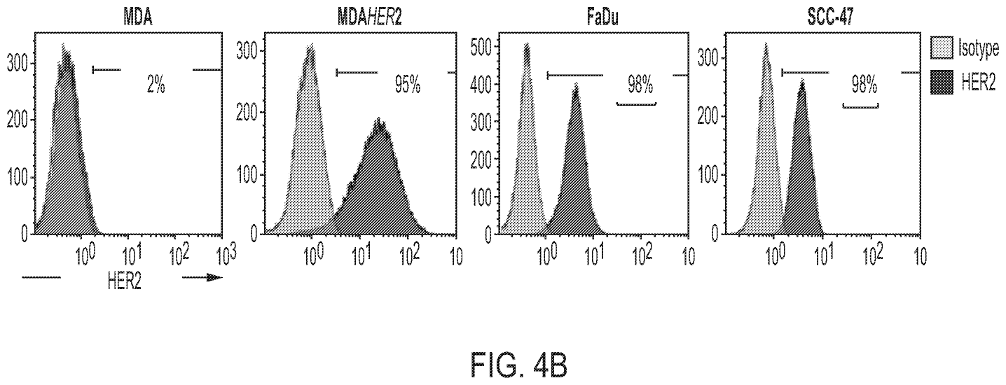

In a first experiment, cells of the HER2 negative MDA cell line (negative control), MDA cells stably expressing HER2 (MDA-HER2; positive control), pharynx squamous cell carcinoma cell line FaDu or the head and neck squamous carcinoma cell line SCC47 cells were labelled with Chromium-51 (⁵¹Cr) and co-cultured with non-transduced T-cells (NT) or the HER2-CAR-T cells expressing the indicated CARs at an effector:target cell ratio of 20:1 for 4 hours. After centrifugation, ⁵¹Cr levels in the cell culture media were counted using a liquid scintillation counter. The results are shown in FIG. 4A; the HER2-CAR-T cells were shown to kill HER2-expressing cancer cells. Similar results were obtained when the experiments were performed using an effector:target cell ratio of 10:1.

Expression of HER2 on MDA-HER2, FaDu and SCC47 was confirmed by flow cytometry. Briefly, the cells were stained with fluorescently-labelled monoclonal anti-HER2 antibody or isotype control antibody for 30 minutes at 4° C. Discrimination of live/dead cells was achieved by including 7AAD in stainings (BD Pharmingen). Stained cells were analyzed using a Gallios flow cytometer and Kaluza software (BD Bioscience), according to manufacturer's instructions. The results are shown in FIG. 4B; MDA cells were confirmed not to express HER2, whilst MDA-HER2, FaDu and SCC47 express HER2.

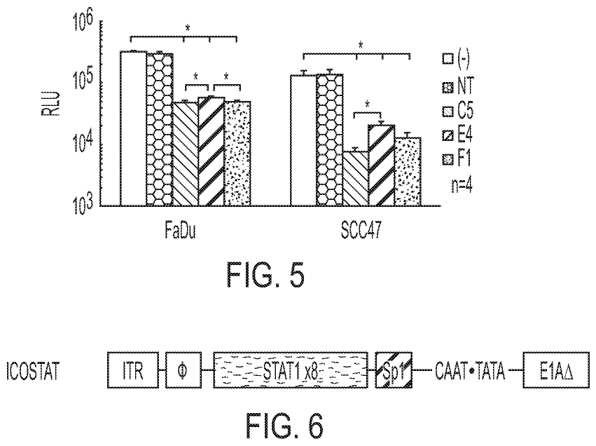

In a separate experiment, FaDu and SCC47 cells genetically modified to express firefly luciferase (ffLuc) were seeded in wells of 24-well plates, and co-cultured with HER2(C5)-CAR-T cells, HER2(E4)-CAR-T cells, or HER2(F1)-CAR-T cells at an effector:target cell ratio of 1:5 for 3 days, and ffLuc activity was measured using a plate reader (Life Technologies). The results are shown in FIG. 5; the HER2-CAR-T cells were shown to kill HER2-expressing cancer cells, as evidenced by a reduction in ffLuc activity (relative light units, RLU). Similar results were obtained when the experiment was performed using an effector:target cell ratio of 1:20.

### Example 2: OncAd Constructs

2.1 Generation of OncAd Constructs

Novel constructs encoding oncolytic adenovirus are prepared using recombinant DNA techniques. In particular embodiments, an OncAd is produced upon modification of a known virus. For example, a region encoding E1A protein from adenovirus 5, such as one lacking the sequence LTCHEACF (SEQ ID NO:52) involved in binding the Rb protein, is replaced with sequence encoding E1A protein from adenovirus 2, similarly lacking the sequence LTCHEACF (SEQ ID NO:52).

ICOSTAT shown in FIG. 6 was produced from ICOVIR15 disclosed e.g. in Rojas et al. 2010 Mol Ther 18 1960-1971. Briefly, the region of ICOVIR15 encoding eight copies of a binding site for the transcription factor E2F was replaced with a region encoding eight tandem copies of a binding site for the transcription factor STAT1. The sequence of ICOSTAT is shown in SEQ ID NO:51.

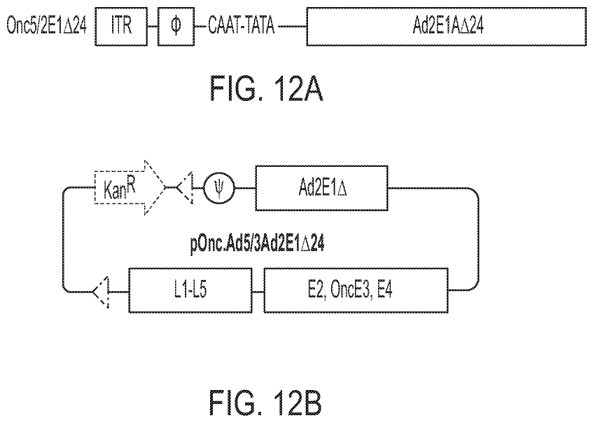

Onc5/3Ad2E1Δ24 (also referred to herein as “Onc5/2E1A24”) shown in SEQ ID NO:55 and represented schematically in FIG. 12 was also prepared by using recombinant DNA techniques. Onc5/3Ad2E1Δ24 has a similar structure as Onc5A24 disclosed e.g. in Fueyo et al. 2000 Oncogene 19:2-12 (hereby incorporated by reference in its entirety; Onc5A24 is also referred to in Fueyo et al. as “A24”), but differs in that Onc5/3Ad2E1Δ24 encodes E1A protein from adenovirus type 2 (Ad2) lacking the sequence LTCHEACF (SEQ ID NO:52), rather than E1A protein from adenovirus type 5 (Ad5) lacking the sequence LTCHEACF (SEQ ID NO:52).

2.2 Cell Killing Activity

The ability of an oncolytic adenovirus of choice or ICOSTAT as generated in Example 2.1 to kill cancer cells may be analysed for example by MTS assay. Briefly, cells of the human alveolar basal epithelial adenocarcinoma cell line A549 cells, FaDu cells, SCC47 cells, or non-cancerous WI-38 human lung fibroblasts or ARPE-19 human retinal pigmented epithelial cells were seeded in wells of 96-well plates and infected with different amounts of a helper-dependent, non-replicating adenovirus (HDAd; as a negative control), an oncolytic adenovirus of choice (e.g. Onc5/3Ad2E1Δ24 described in Example 2.1), or ICOSTAT described in Example 2.1 above.

Cells may be cultured for 4 days, for example, and then MTS reagents (Promega) may be added to each well, with cells being incubated at 37° C. for 2 hours. Live cells may be analyzed by measuring the absorbance at 490 nm with a plate reader. Readings may be normalized using the readings for untreated cells of each type (i.e. untreated cells =100% cell viability), and wells lacking cells would be considered 0%.

In particular embodiments, the oncolytic virus of choice is able to kill cancer cells in a dose-dependent manner. The oncolytic virus of choice also exhibits a lower level of cell killing of non-cancerous cells, such as WI-38 and ARPE-19 cells as compared to the level of killing by the virus of cancerous cells, in specific embodiments.

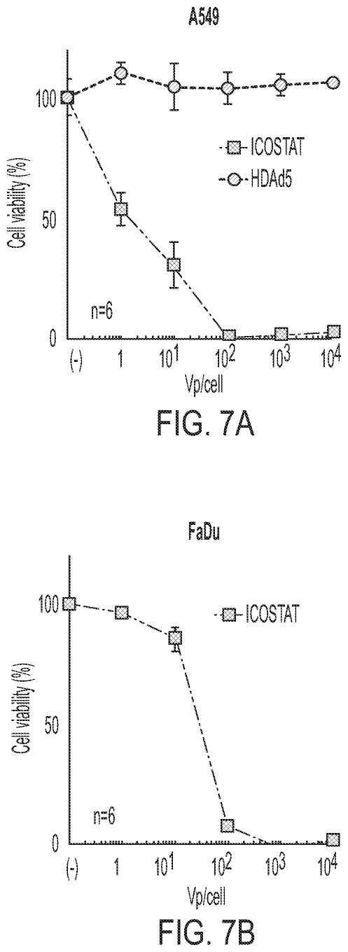

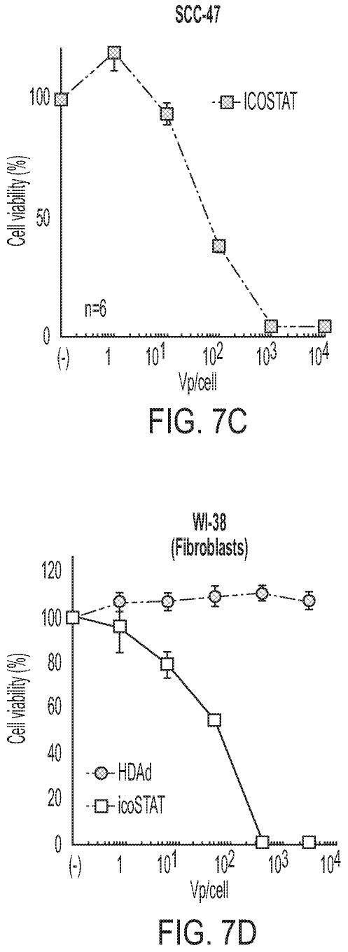

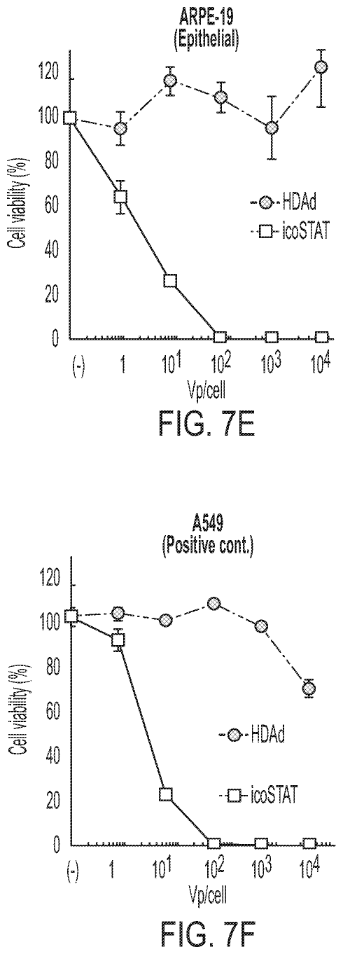

FIGS. 7A to 7F show that ICOSTAT is able to kill cancer cells (i.e. A549, FaDu and SCC47 cells) in a dose-dependent manner (FIGS. 7A to 7C and 7F), and exhibits a lower level of cell killing of non-cancerous cells WI-38 and ARPE-19 cells as compared to the level of killing of the cancerous cells (FIGS. 7D and 7E).

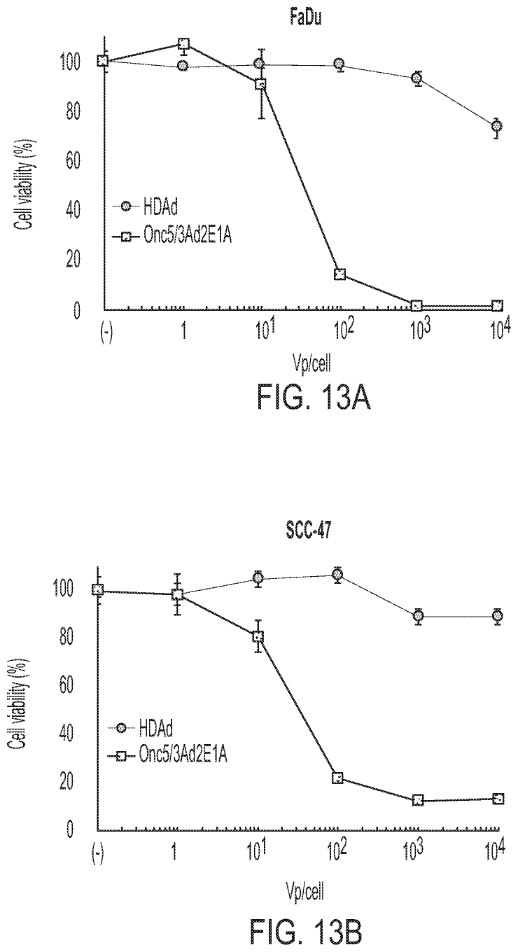

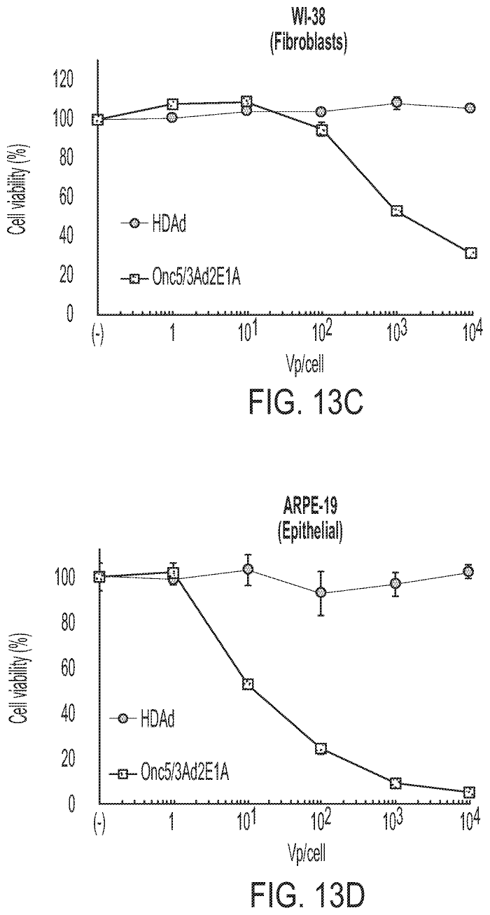

FIGS. 13A to 13D show that Onc5/3Ad2E1Δ24 is able to kill cancer cells (i.e. FaDu and SCC47 cells) in a dose-dependent manner (FIGS. 13A and 13B), and exhibits a lower level of cell killing of non-cancerous WI-38 and ARPE-19 cells as compared to the level of killing of the cancerous cells (FIGS. 13C and 13D).

2.3 Ability to Help Helper-Dependent Adenovirus (HDAd)

The ability of an oncolytic adenovirus of choice or ICOSTAT as generated in Example 2.1 to assist replication of a helper-dependent adenovirus (HDAd) may be analysed by co-infecting cancer cells with the OncoAd and HDAd, and determining virus copy number. Briefly, FaDu or SCC47 cells are plated in 24-well plates and infected with 10 viral particles per cell of HDAd alone, or OncAd+HDAd (at an OncAd:HDAd ratio of 1:10). Cells are harvested at 48 hours post-infection, DNA is extracted and both HDAd and Onc.Ad vector copies are analyzed by quantitative real-time PCR (10 min at 95° C. and then 45 cycles of 10 s at 95° C., 15 s at 60° C., and 30 s at 72° C.) using a Bio-Rad iQ5 real-time PCR detection system (Bio-Rad), and Applied Biosystems SYBR green PCR master mix (Life Technologies). Copy number is normalized using copy number detected for GAPDH.

In particular embodiments, the oncolytic virus of choice is able to replicate itself and the HDAd sufficiently.

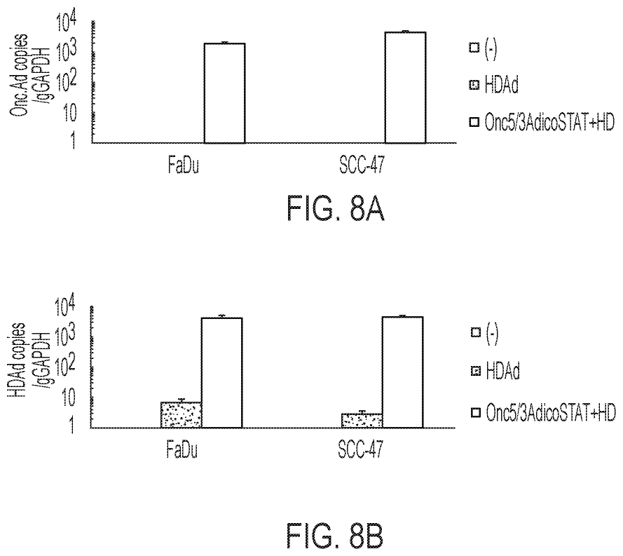

FIGS. 8A and 8B show that ICOSTAT (designated “Onc5/3AdicoSTAT” in the figures) was found to be able to replicate itself (FIG. 8A) and the HDAd (FIG. 8B).

2.4 Effect of IFNγ on Replication of ICOSTAT in Cancer Cells

The effect of IFNγ treatment on replication of ICOSTAT OncAd was analysed. Briefly, FaDu and SCC47 cells are plated in 24-well plates, and the cells are infected with 10 vp/cell of the oncolytic virus of choice or icoSTAT 3 hours post-infection cell culture medium is replaced with medium containing, or not containing, 10 ng/mL recombinant IFNγ at 3 hours post-infection, and cell culture media are replaced with fresh media with/without 10 ng/mL recombinant IFNγ again at 24 and 48 hours post-infection. Cells are harvested at 3, 24, 48 and 72 hours post-infection, DNA is extracted from the cells, viral copy numbers are analysed by quantitative real-time PCR and normalized using copy number detected for GAPDH.

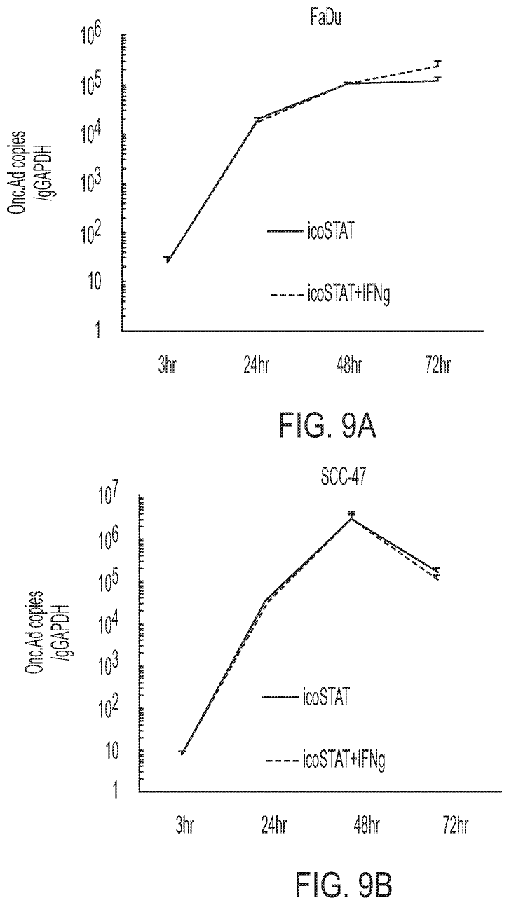

FIGS. 9A and 9B show that ICOSTAT was able to replicate in FaDu cells and SCC47 cells, in the presence or absence of IFNγ.

### Example 3: Helper-Dependent Ad (HDAd) Constructs

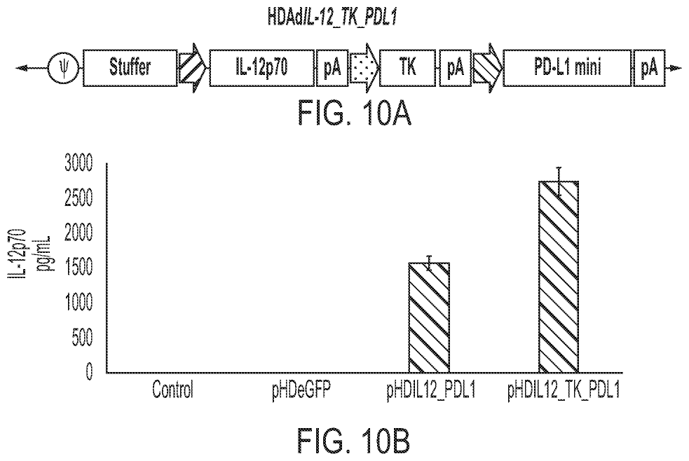

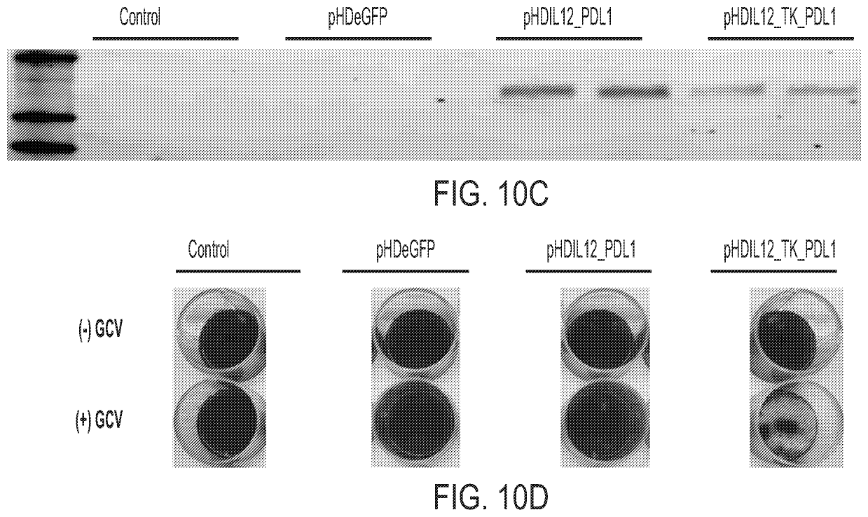

3.1 HDAd Constructs and Production A novel construct encoding a helper-dependent adenovirus was prepared using recombinant DNA techniques. The coding sequence of the resulting construct designated HDAd/L-12_TK_PD-L1 is represented schematically in FIG. 10A. HDAdIL-12_TK_PD-L1 contains sequence encoding expression cassettes for (i) human IL-12p70 (sequence encoding alpha and beta chains), (ii) HSV-1 thymidine kinase, and (iii) an anti-PD-L1 minibody (comprising the CDRs of anti-PD-L1 clone H12_gl described e.g. in WO 2016111645 A1) including a HA tag. The three coding sequences each have their own polyA signal sequences.

The HDAd HDA28E4EGFP construct containing an EGFP transgene driven by the CMV promoter (HDAdeGFP) was produced as described in Farzad et al. Oncolytics 2014 1: 14008.

The HDAd “HDIL12_PDL1” contains sequence encoding human IL-12p70 protein and anti-PD-L1 minibody derived from YW243.55.S70 (atezolizumab). The anti-PD-L1 minibody of this construct consists of scFv for YW243.55.S70 fused with a hinge, CH2 and CH3 regions of human IgG1 and a C-terminal HA tag (as described e.g. in Tanoue et al. Cancer Res. (2017) 77(8):2040-2051).

3.2 Expression of Encoded Proteins

Cancer cells were transfected with plasmid HDAd vectors, and medium samples were collected to analyze IL-12p70 and anti-PD-L1 minibody levels in the cell culture media of the transfected cells at 48 hours post-transfection.

IL-12p70 levels in media were measured using the BD cytokine multiplex bead array system (BD Biosciences), according to manufacturer's instructions. The results are shown in FIG. 10B. Cells transfected with the HDAd/L-12_TK_PD-L1 construct were found to produce higher levels of IL-12p70 than cells transfected with the HDIL-12_PD-L1 construct.

Secretion of anti-PD-L1 minibodies into the cell culture medium was detected by western blot analysis, using an anti-HA antibody (to detect the HA-tagged minibodies). FIG. 10C shows that cells transfected with the HDAd/L-12_TK_PD-L1 construct secreted the anti-PD-L1 minibody into the cell culture medium.

In another experiment, cells were transfected with the different constructs and at 8 hours post-transfection the cell culture media was replaced with medium containing 10 ng/ml Ganciclovir (GCV). Cell culture medium was then replaced with medium containing 10 ng/ml every 24 hours, and after 7 days, the wells were stained with Crystal Violet solution to reveal viable cells.

The results are shown in FIG. 10D, and confirm that cells transfected with the HDAd/L-12 TK_PD-L1 construct express thymidine kinase.

In further experiments A549, FaDu or SCC47 cells (n=4 wells per condition) were infected in vitro with HDAd/L-12_TK_PD-L1, HDAd_PD-L1 (see e.g. Tanoue et al., supra), or a control HDAd encoding eGFP (see Farzad et al., supra). The cells were either cultured for 48 hours in the absence of ganciclovir, or medium was changed at 8 hours post-infection and every 24 hours thereafter with medium containing 10 ng/ml ganciclovir.

Secretion of IL-12 into the cell culture supernatant was analysed by ELISA, and secretion of anti-PD-L1 minibody was analysed by western blot using an anti-HA antibody (the anti-PD-L1 minibody comprises a C-terminal HA-tag). At the end of the experiment wells were stained with Crystal Violet solution to reveal viable cells.

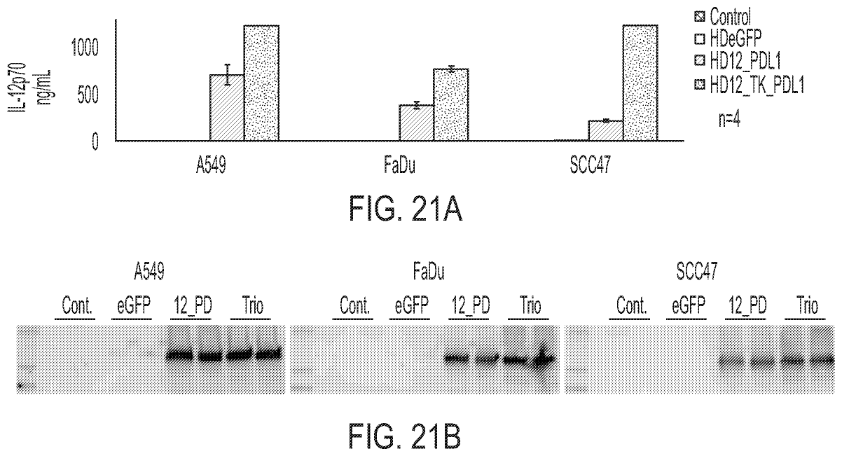

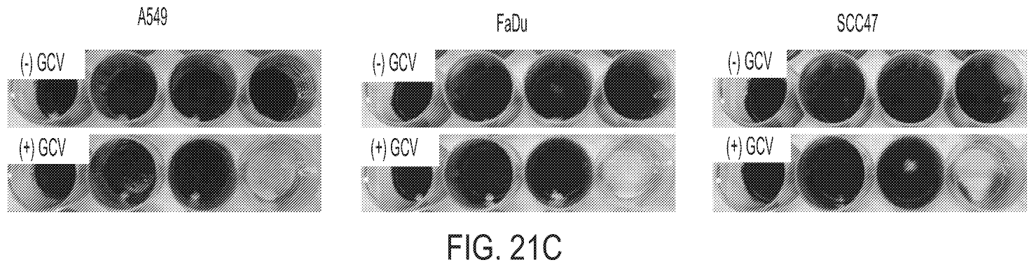

The results are shown in FIGS. 21A to 21C, and confirmed expression of the transgenes encoded by the HDAds in the different cancer cell lines analysed.

3.3 Confirmation of Anti-PD-L1 Minibody Binding to PD-L1

The ability of the anti-PD-L1 minibody encoded by HDAd/L-12_TK_PD-L1 to bind to PD-L1 was analysed by ELISA.

Briefly, Immulon 2 high binding 96-well plates (VWR) were coated with 500 ng/well of recombinant human PD-L1 (BioVision). After blocking plate with PBS-T containing 3% BSA, serially diluted cell culture media of A549 cells which had been transfected with plasmid encoding GFP (pGFP; negative control), plasmid encoding the anti-PD-L1 minibody described in Tanoue et al. supra, (pPDL1 mini Tanoue) or plasmid encoding the anti-PD-L1 minibody encoded by HDAd/L-12 TK_PD-L1 (pPDL1 mini) were added and incubated at 4° C. for 24 hours. Serially diluted anti-human PD-L1 antibody starting from 10 μg/well (BioLegend) was used as a positive control (PDL1 IgG). After washing plate with PBS-T, HRP-labeled anti-human IgG (for PD-L1 mini and PDL1 mini Tanoue) or HRP-labeled anti-mouse IgG (BioRad; for PD-L1 IgG and Iso IgG) were added for detection, and incubated at room temperature for 1 hour. The plate was then developed, and absorbance at 450 nm was measured using Tecan reader (TECAN).

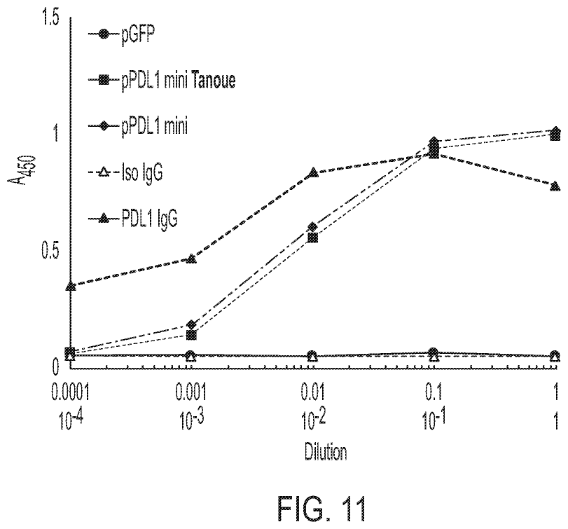

The results are shown in FIG. 11. The anti-PD-L1 minibody comprising the CDRs of anti-PD-L1 antibody clone H12 was found to bind to human PD-L1 in a dose-dependent fashion, with comparable (or greater) avidity as compared to the avidity of binding by anti-PD-L1 minibody described in Tanoue et al. supra.

### Example 4: Analysis of Treatment of Cancer In Vivo

The anticancer effect of treatment with the combination of (1) an oncolytic virus of choice+HDAd/L-12_TK_PD-L1+HER2-CAR-T and (2) ICOSTAT+HDAd/L-12_TK_PD-L1+HER2-CAR-T is demonstrated in vivo in mouse xenograft tumour models.

In a first experiment, 1×10⁶ FaDu cells are injected subcutaneously in PBS into NSG male mice. After 12 days, 1×10⁸ viral particles (1) oncolytic virus and HDAd/L-12_TK_PD-L1 or (2) ICOSTAT+HDAd/L-12_TK_PD-L1 are injected intratumorally at an OncAd:HDAd ratio of 1:20.

In a second experiment, 0.5×10⁶ FaDu cells are injected orthotopically into NSG male mice. After 6 days, 1×10⁸ viral particles (1) oncolytic virus and HDAd/L-12_TK_PD-L1 or (2) ICOSTAT+HDAd/L-12_TK_PD-L1 are injected intratumorally at an OncAd:HDAd ratio of 1:20.

In both experiments, 3 days after administration of the viral particles, 1×10⁶ HER2-CAR T cells are administered intravenously.

In both experiments, control conditions are included as follows:

| Condition | OncAd | HDAd | CAR T |
|---|---|---|---|
| 1 (test condition) | Of choice | HDAdIL-12_TK_PD-L1 | HER2 CAR-T |
| 2 (test condition) | ICOSTAT | HDAdIL-12_TK_PD-L1 | HER2 CAR-T |
| 3 | — | HDAdIL-12_TK_PD-L1 | HER2 CAR-T |
| 4 | Of choice | — | HER2 CAR-T |
| 5 | ICOSTAT | — | HER2 CAR-T |
| 6 | Of choice | HDAdIL-12_TK_PD-L1 | — |
| 7 | ICOSTAT | HDAdIL-12_TK_PD-L1 | — |
| 8 | Of choice | — | — |
| 9 | ICOSTAT | — | — |
| 10 | — | HDAdIL-12_TK_PD-L1 | — |
| 11 | — | — | HER2 CAR-T |

Tumor size is monitored and tumour volumes are calculated using the formula: Width²×Length×0.5.

The use of the combination of oncolytic virus, HDAdIL-12_TK_PD-L1 and HER2 CAR-T (test condition 1) is found to have an improved antitumour effect as compared to the use of any of the agents alone (conditions 8, 10 or 11), or compared to the use of two of the three agents (conditions 3, 4 and 6).

Similarly, the use of the combination of ICOSTAT, HDAdIL-12_TK_PD-L1 and HER2 CAR-T (test condition 2) is found to have an improved antitumour effect as compared to the use of any of the agents alone (conditions 9, 10 or 11), or compared to the use of two of the three agents (conditions 3, 5 and 7).

Similar results are observed when xenograft tumours are established using SCC47 cells and A549 cells.

### Example 5: Analysis of the Anti-Cancer Activity of the HER2-Specific CAR-T Cells In Vivo

The anti-cancer activity of the HER2-specific CAR-T cells (see Example 1 above) was investigated in vivo in a FaDu cell-derived xenograft model of squamous cell head and neck cancer.

Briefly, 0.5×10⁶ FaDu cells were injected orthotopically into NSG male mice. After 9 days, mice were injected via the tail vein with 1×10⁶ T cells genetically modified to express firefly luciferase, which had not been transduced with a HER2-CAR construct, or with 1×10⁶ firefly luciferase-expressing T cells which had been transduced with the C5, F1 or A3 CAR constructs. A control condition was included in the experiment in which mice were not injected with T cells at day 9.

Luciferase activity (and thus number and distribution of the administered T cells), body weight, survival of the mice was monitored overtime. Luciferase activity was monitored by intraperitoneal injection of D-Luciferin (1.5 mg per mouse), and imaging of the mice 10 min later using an IVIS imager (Xenogen).

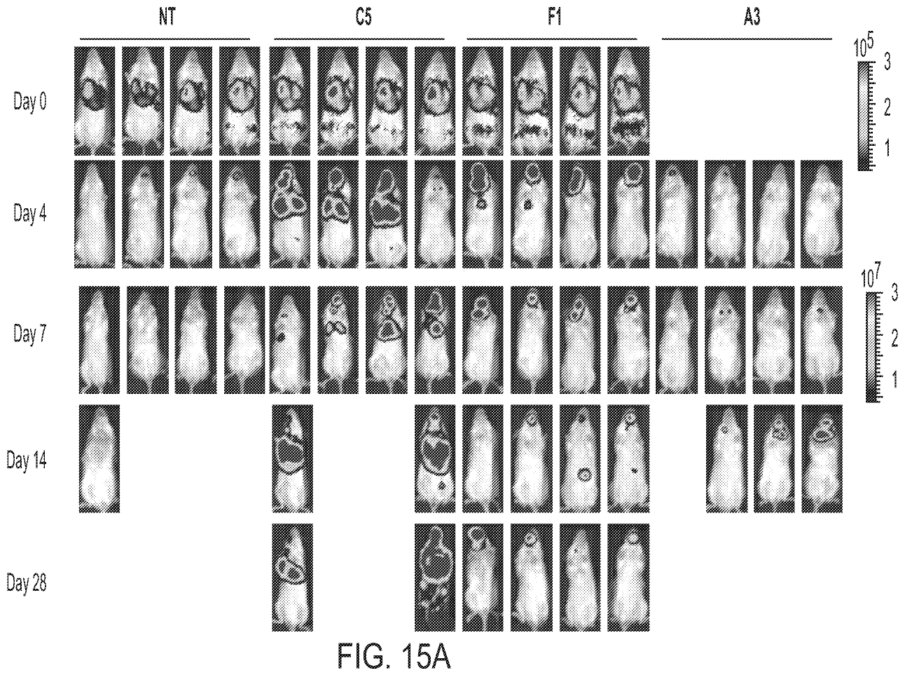

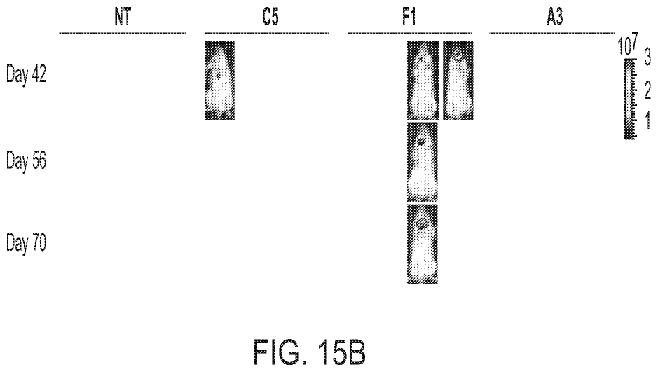

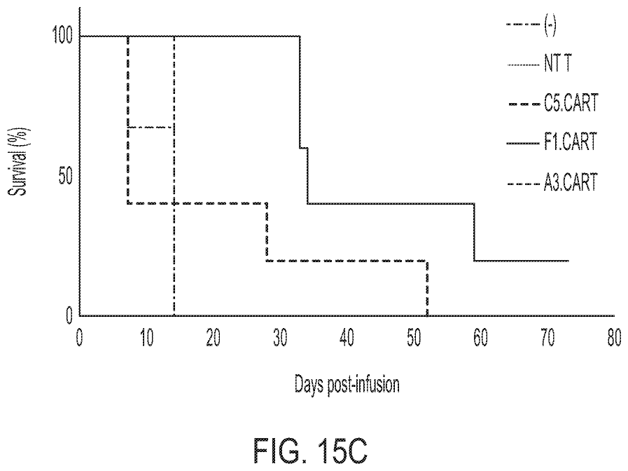

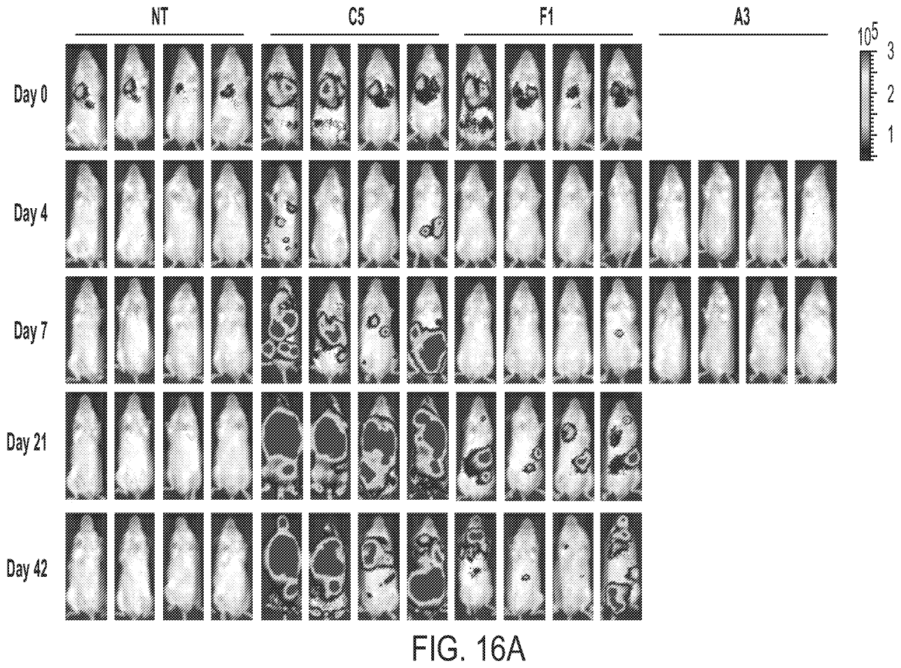

FIGS. 15A and 15B show the images acquired on days 0, 4, 7, 14, 28, 42, 56 and 70 following injection of the luciferase-expressing T cells (i.e. the non-transduced T cells or HER2-specific CAR-T cells) (days refer to days after ffLuc T cell injection). The systemically infused T cells were shown to migrate to the site of the orthotopic tumors. The T cells which had not been modified to express HER2-specific CARs were undetectable after 7 days. By contrast, the HER2-specific CAR-T cells persisted and remained detectable throughout the experiment.

FIG. 15C shows percentage survival of mice subjected to the different treatments over the course of the experiment. Administration of HER2-specific CAR-T cells was found to increase survival.

In a separate experiment NOD scid gamma (NSG) mice were injected via the tail vein with 1×10⁶ firefly luciferase-expressing T cells which had not been transduced with a HER2-CAR construct, or with 1×10⁶ firefly luciferase-expressing T cells which had been transduced with the C5, F1 or A3 CAR construct. Luciferase activity was monitored as described above, and body weight of the mice was also monitored overtime.

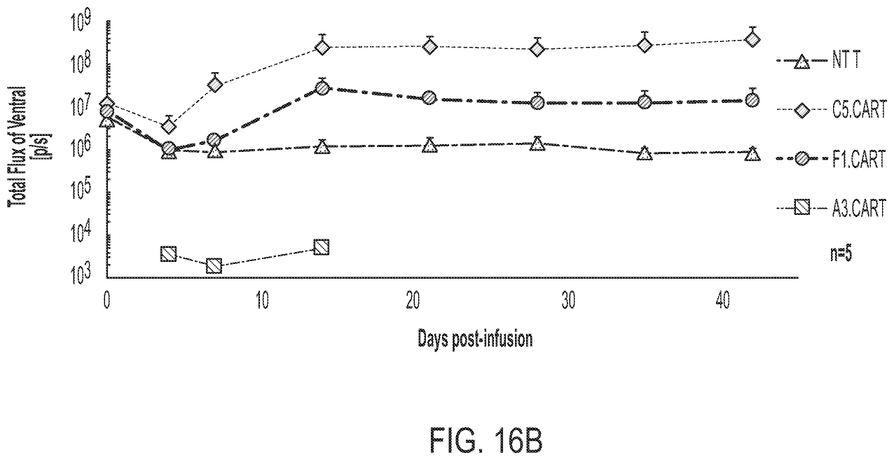

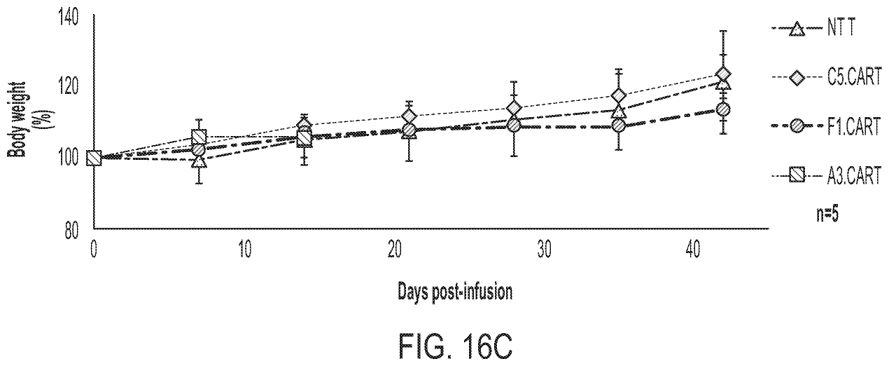

The results of the experiment are shown in FIGS. 16A to 16C. The C5 CAR-T cells were found to expand non-specifically in NSG mice (FIG. 16A). No significant weight loss was observed in NSG mice administered with the HER2-specific CAR-T cells (FIG. 16C).

### Example 6: Analysis of the Anti-Cancer Activity of the Combination of Oncolytic Virus, HDAd Virus and HER2-Specific CAR-T Cells In Vivo

The anti-cancer activity of a combination of oncolytic virus, HdAd and HER-specific CAR-T cell therapy was investigated in vivo in a FaDu cell-derived xenograft model of squamous cell head and neck cancer.

Briefly, 0.5×10⁶ FaDu cells were injected orthotopically into NSG male mice. After 6 days, one group of mice was then injected intratumorally with a combination of Onc5/3Ad2E1Δ24 (described in Example 2.1) and HDAd/L-12_TK_PD-L1 described in Example 3.1 (this combination of OncAd and HdAd is referred to herein as “CAdtrio”). A total of 1×10⁷ viral particles were administered, at a 1:10 ratio of Onc5/3Ad2E1Δ24:HDAd/L-12_TK_PD-L1.

Three days later, mice were injected via the tail vein with 1×10⁶ T cells engineered to express firefly luciferase, which had been transduced with the HER2-specific CAR construct corresponding to clone F1. A control group of mice which had not been administered with CAdtrio was injected via the tail vein with 1×10⁶ firefly luciferase-expressing T cells which had not been transduced with a HER2-CAR construct, and a further control group of mice was not administered with CAdtrio nor injected with T cells. Luciferase activity, body weight and survival of the mice was monitored over time.

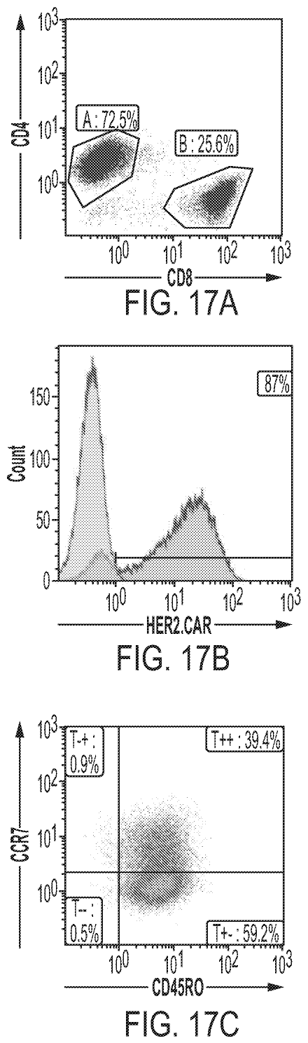

Prior to their use in the experiment the F1.CART cells were characterised flow cytometry, and the results are shown in FIGS. 17A to 17C. The cells were found to comprise 72.5% CD4+ cells and CD8+ cells. 87% of the cells were determined to express HER2 CAR at the cell surface. 39% of the cells were CCDR7+CD45RO+, and 59.2% of the cells were CCR7-CD45RO+.

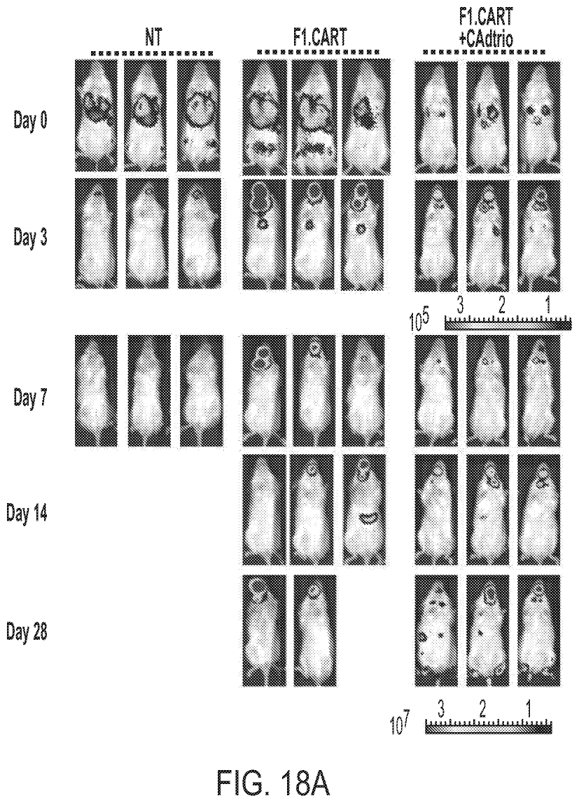

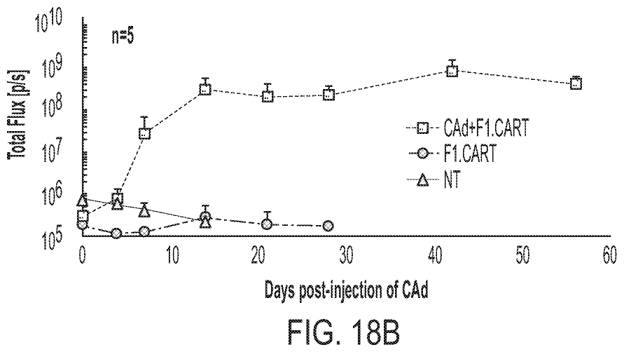

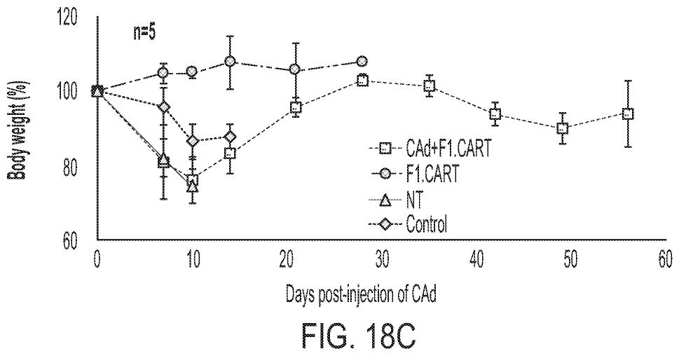

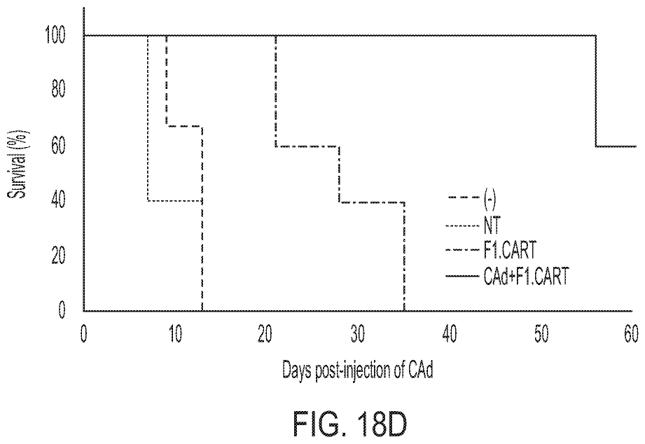

The results of the experiments analysing the therapeutic efficacy of the combination of oncolytic virus, HDAd virus and HER2-specific CAR-T cells to treat cancer in vivo are shown in FIGS. 18A to 18D. The combination of Onc5/3Ad2E1Δ24, HDAd/L-12 TK_PD-L1 and F1.CART was found to improve survival over treatment with F1.CART cells alone.

In further experiments two different ratios of Onc5/3Ad2E1Δ24 to HDAd/L-12_TK_PD-L1 were investigated.

Briefly, 0.5×10⁶ FaDu cells modified to express firefly luciferase were injected orthotopically into NSG male mice. After 6 days, mice were injected intratumorally with:
(i) 1×10⁷ viral particles of CAdtrio, at a ratio of Onc5/3Ad2E1Δ24:HDAd/L-12_TK_PD-L1 of 1:10;
(ii) 1×10⁷ viral particles of CAdtrio, at a ratio of Onc5/3Ad2E1Δ24:HDAd/L-12_TK_PD-L1 of 1:20;
(iii) 1×10⁸ viral particles of CAdtrio, at a ratio of Onc5/3Ad2E1Δ24:HDAd/L-12_TK_PD-L1 of 1:10; or
(iv) 1×10⁸ viral particles of CAdtrio, at a ratio of Onc5/3Ad2E1Δ24:HDAd/L-12_TK_PD-L1 of 1:20.

Three days later, mice were injected via the tail vein with 1×10⁶ T cells which had been transduced with the F1 CAR construct (not expressing firefly luciferase). The cancer was monitored overtime by analysis of luciferase activity as described above, and the body weight of the mice was also monitored.

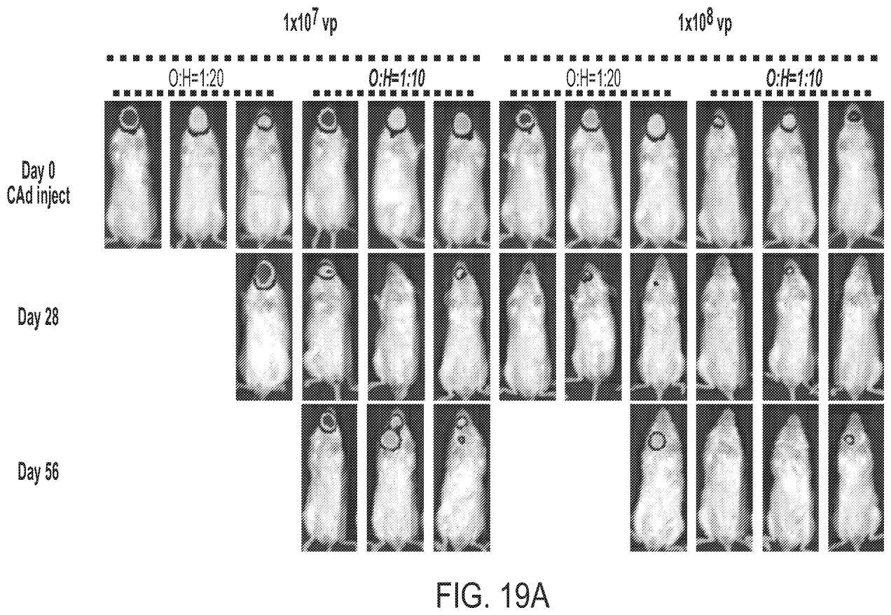

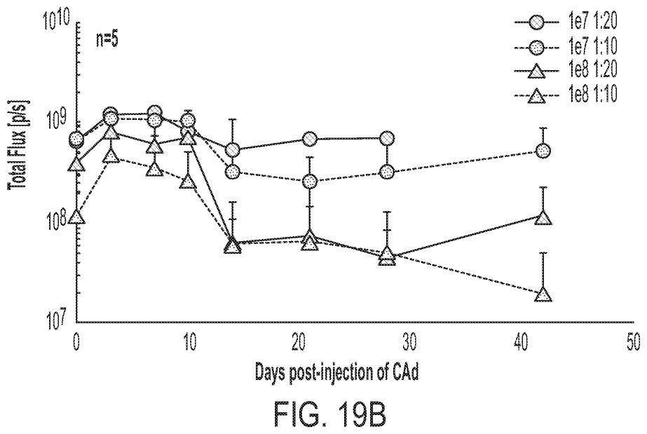

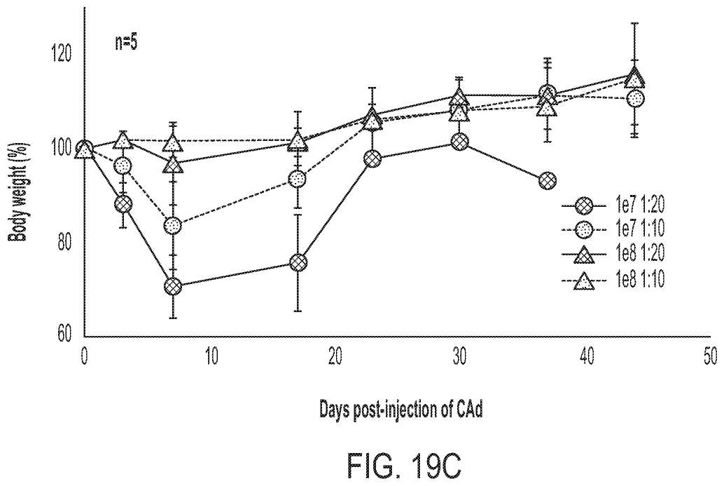

The results of the experiments are shown in FIGS. 19A to 19C. Mice administered with a 1:10 ratio of Onc5/3Ad2E1Δ24:HDAd/L-12_TK_PD-L1 generally had fewer luciferase-expressing FaDu cells than those administered with a 1:20 ratio of Onc5/3Ad2E1Δ24:HDAd/L-12 TK_PD-L1, and mice administered with 1×10⁸ viral particles of CAdtrio generally had fewer luciferase-expressing FaDu cells than those administered with 1×10⁷ viral particles of CAdtrio (FIG. 19B).

### Example 7: Analysis of the Anti-Cancer Activity of the Combination of Oncolytic Virus, HDAd Virus and Ganciclovir (GCV) In Vivo

The anti-cancer activity of a combination of oncolytic virus and HdAd (encoding thymidine kinase) (I,e, CAdtrio) in conjunction with ganciclovir (GCV) was investigated in vivo in a FaDu cell-derived xenograft model of squamous cell head and neck cancer.

Ectopic FaDu tumors were established by subcutaneous injection of FaDu cells into the flanks of mice. The mice were subsequently injected intratumorally with 1×10⁸ viral particles of CAdtrio, at a ratio of Onc5/3Ad2E1Δ24:HDAd/L-12_TK_PD-L1 of 1:10. One group of mice (n=5) was then injected intraperitoneally on days 2, 3, 4, 5, 7, 10, 14, 17 and 21 days after CAdtrio injection with 10 mg/kg of ganciclovir.

Blood samples were collected from the mice on days 2, 7, 14 and 21 and analysed by ELISA for IL-12 expression. Tumor volumes were monitored throughout the experiment. At day 22 Onc.Ad and HDAd vector copy numbers were determined in DNA extracted from the tumors by quantitative real-time PCR analysis, and normalised using the copy number detected for GAPDH.

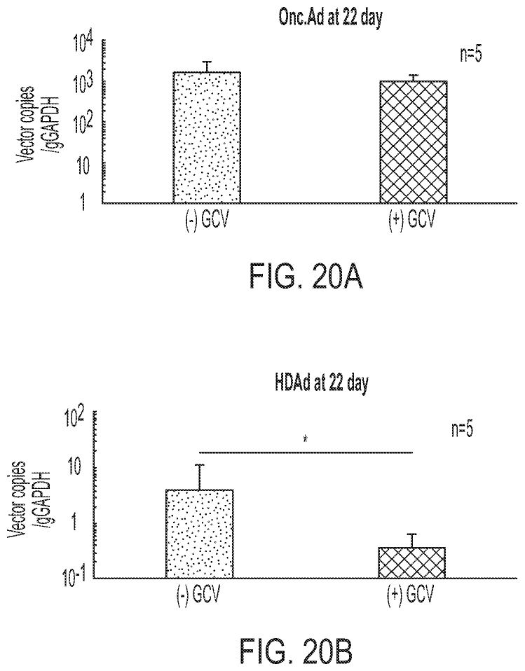

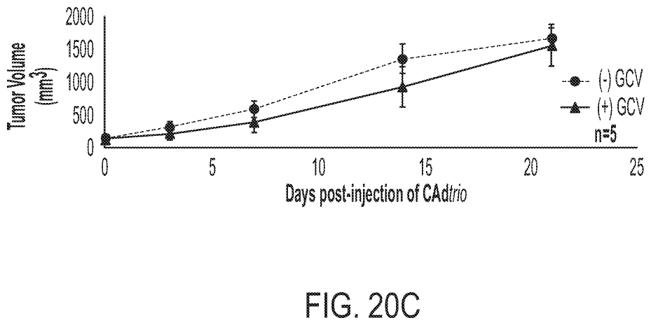

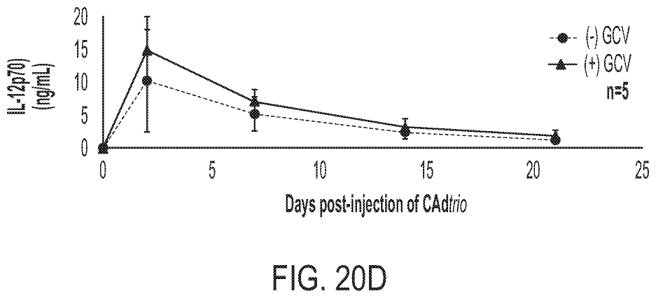

The results of the experiments are shown in FIGS. 20A to 20D. Ganciclovir (GCV) treatment did not significantly influence Onc.Ad vector copy number at day 22 (FIG. 20A), but significantly decreased HDAd vector copy number (FIG. 20B). GCV treatment was also found to improve tumor control (FIG. 20C), but did not significantly influence the levels of IL-12 in the blood (FIG. 20D).

### Example 8: Generation of Oncolytic Virus-Specific T Cells and HER-Specific CAR-Expressing Oncolytic Virus-Specific T Cells

8.1 Generation and Characterisation of Oncolytic Virus-Specific T Cells

Adenovirus-specific T cells (AdVSTs) and activated T cells (ATCs) were prepared as follows.

Anti-CD3 (clone OKT3) and anti-CD28 agonist antibodies were coated onto wells of tissue culture plates by addition of 0.5 ml of 1:1000 dilution of 1 mg/ml antibodies, and incubation for 2-4 hr at 37° C., or at 4° C. overnight.

PBMCs were isolated from blood samples obtained from healthy donors according to the standard Ficoll-Paque method.

ATCs:

1×10⁶ PBMCs (in 2 ml of cell culture medium) were stimulated by culture on the anti-CD3/CD28 agonist antibody-coated plates in CTL cell culture medium (containing 50% Advanced RPMI, 50% Click's medium, 10% FBS, 1% GlutaMax, 1% Pen/Strep) supplemented with 10 ng/ml IL-7 and 5 ng/ml IL-15. The cells were maintained at 37° C. in a 5% CO₂ atmosphere. The next day, 1 ml of the cell culture medium was replaced with fresh CTL medium containing 20 ng/ml IL-7 and 10 ng/ml IL-15.

ATCs were maintained in culture, and subsequently harvested and used in experiments or cryopreserved between days 5-7.

AdVSTs:

1×10⁶ PBMCs (in 2 ml of cell culture medium) were stimulated by culture on the anti-CD3/CD28 agonist antibody-coated plates in CTL cell culture medium supplemented with 10 ng/ml IL-7 and 100 ng/ml IL-15.

20 μl of a 200-fold dilution of Adenovirus-specific Hexon Pepmix (JPT Cat #PM-HAdV3) or Penton PepMix (JPT Cat #PM-HAdV5) was added to the wells. The cells were maintained at 37° C. in a 5% CO₂ atmosphere. After 48 hours cells were fed with CTL medium, with added IL-7 and IL-15 to a final concentration of 10 ng/ml IL-7 and 100 ng/ml IL-15.

8.2 Generation of CAR-Expressing, Oncolytic Virus-Specific T Cells

On day 3, AdVSTs were resuspended at a concentration of 0.125×10⁶ cells/ml in CTL cell culture medium containing 10 ng/ml IL-7 and 100 ng/ml IL-15.

Retronectin coated plates were prepared by incubation of RetroNectin (Clontech) diluted 1:100 in PBS for 2-4 hr at 37° C., or at 4° C. overnight. The wells were washed with CTL medium, 1 ml of retroviral supernatant of HER2-specific CAR retrovirus was added to wells, and plates were centrifuged at 2000g for 1.5 hr. At the end of the centrifugation step retroviral supernatant was aspirated, and 2 ml of AdVST suspension (i.e. 0.25×10⁶ cells) was added to wells of the plate. Plates were centrifuged at 400g for 5 min, and incubated at 37° C. in a 5% CO₂ atmosphere.

After 48 hrs (i.e. on day 6) the cell culture medium was aspirated and replaced with CTL cell culture medium containing 10 ng/ml IL-7 and 100 ng/ml IL-15.

On day 9 cells were harvested and used in experiments or cryopreserved, or subjected to a second stimulation to expand CAR-expressing AdVSTs (see Example 8.3).

8.3 Expansion of AdVSTs and CAR-AdVSTs

AdVSTs and CAR-expressing AdVSTs were expanded by further stimulations as desired, as follows.

Pepmix-pulsed autologous ATCs were used as APCs, and K562cs cells (see e.g. Ngo et al., J Immunother. (2014) 37(4):193-203) were used as costimulatory cells. The final ratio of AdVSTs or CAR-AdVSTs:ATCs:K562cs cells in the stimulation cultures was 1:1:3-5.

AdVSTs or CAR-AdVSTs were resuspended to a concentration of 0.2×10⁶ cells/ml in CTL medium.

1×10⁶ ATCs were incubated with 10 μl of 200-fold dilution of Adenovirus-specific Hexon Pepmix (JPT Cat #PM-HAdV3) or Penton PepMix (JPT Cat #PM-HAdV5) at 37° C. for 30 min. The ATCs were subsequently irradiated at 30 Gy and harvested. 3-5×10⁶ K562cs cells were irradiated at 100 Gy.

The ATCs and K562cs cells were then mixed in a total volume of 5 ml CTL medium, and 20 ng/ml IL-7 and 200 ng/ml IL-15 was added, 1 ml of this mixture was added to wells of a 24 well plate, and 1 ml of AdVST suspension or CAR-AdVST suspension was added to the wells.

Cells were maintained at 37° C. in a 5% CO₂ atmosphere. After 3-4 days cell culture medium was added as necessary, and after 6-7 days cells the expanded AdVSTs or CAR-AdVSTs were harvested for use in experiments.

### Example 9: Analysis of the Anti-Cancer Activity of Combinations of Oncolytic Virus, HDAd, Oncolytic Virus-Specific T Cells and CAR-Expressing Oncolytic Virus-Specific T Cells In Vivo

The anti-cancer activity of different combinations of oncolytic virus, HDAd, oncolytic virus-specific T cells and CAR-expressing oncolytic virus-specific T cells was investigated in vivo in a FaDu cell-derived xenograft model of squamous cell head and neck cancer.

Briefly, 0.5×10⁶ FaDu cells engineered to express firefly luciferase were injected orthotopically into NSG male mice. After 6 days groups of mice were injected intratumorally with:
(i) 1×10⁷ viral particles of CAdtrio, at a ratio of Onc5/3Ad2E1Δ24:HDAd/L-12_TK_PD-L1 of 1:10; or
(ii) 1×10⁷ viral particles of Onc5/3Ad2E1Δ24.

Three days later, mice were injected via the tail vein with:
(a) 1×10⁶ AdVSTs, or
(b) 1×10⁶ AdVSTs transduced with anti-HER2 CAR clone F1 (prepared as described in Example 8).

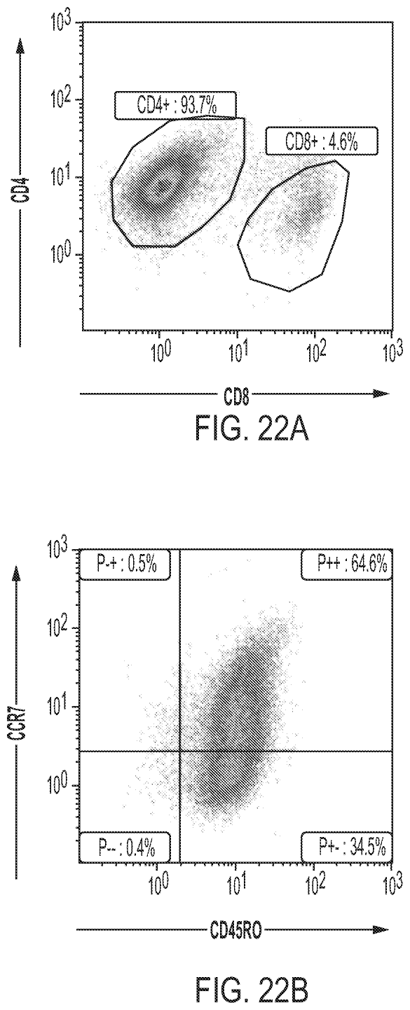

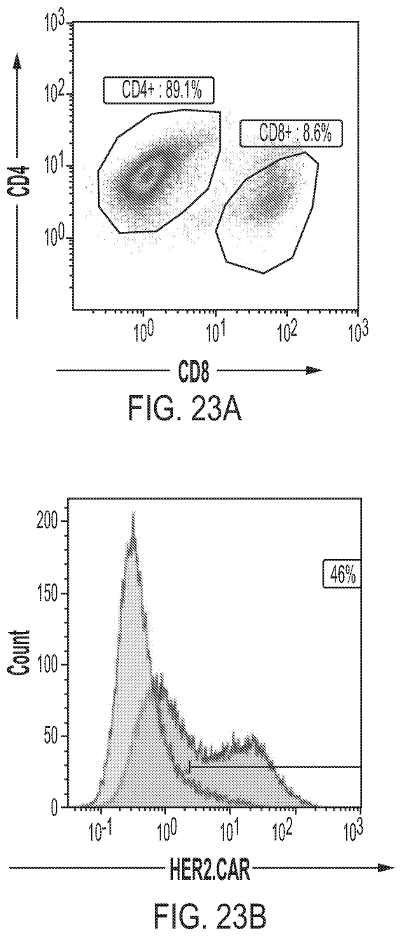

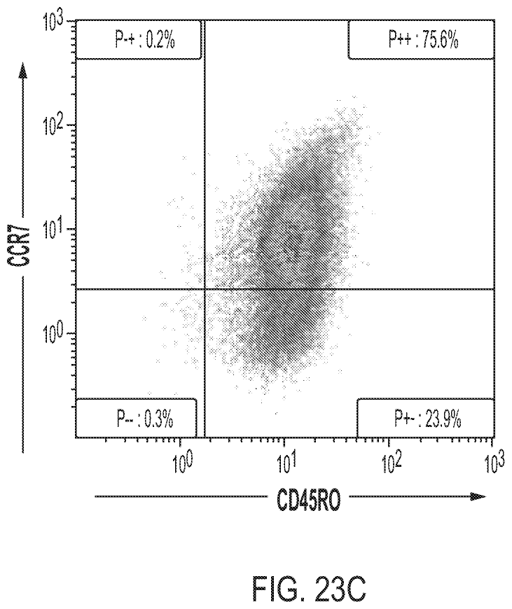

Prior to their use in the experiment the AdVSTs and F1.CAR-AdVSTs were characterised by flow cytometry, and the results of the analysis are shown in FIGS. 22A and 22B, and FIGS. 23A to 23C.

The cancer was monitored over time by analysis of luciferase activity as described above, and the body weight of the mice was also monitored.

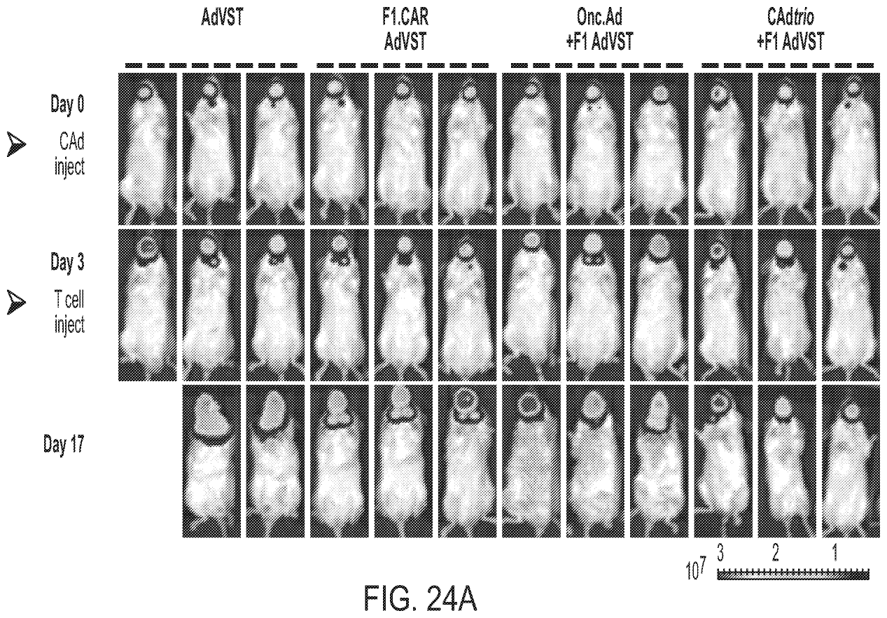

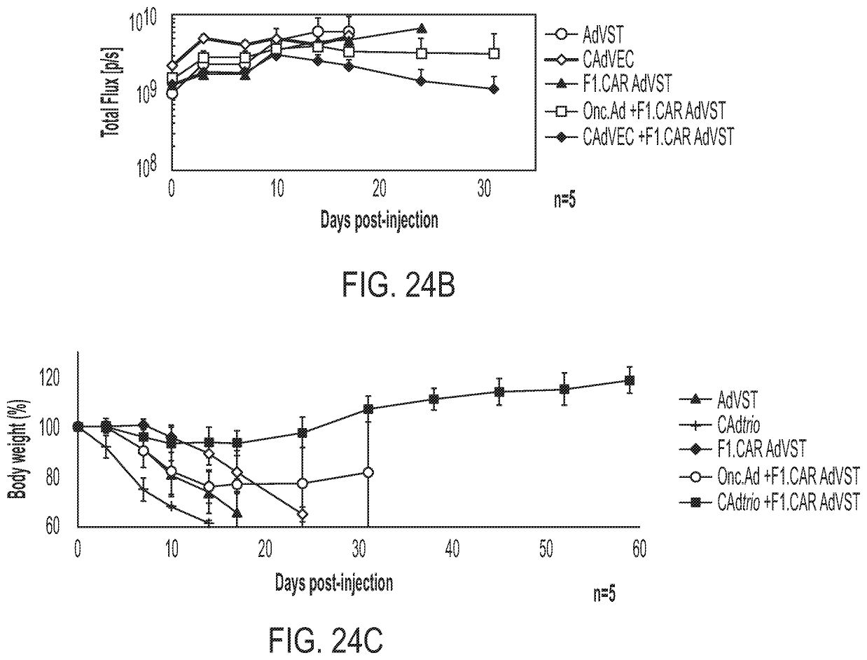

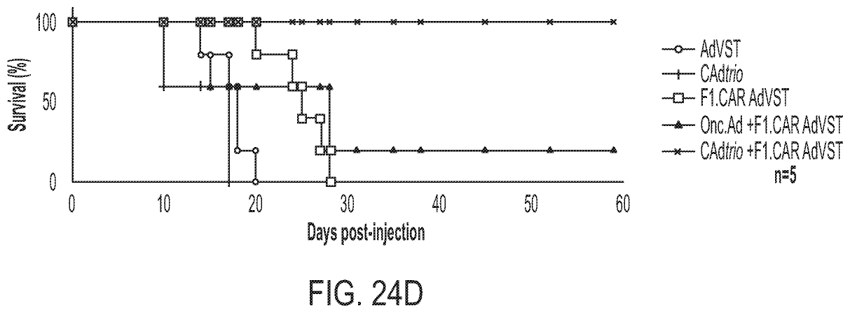

The results of the experiments are shown in FIGS. 24A to 24D. The greatest levels of tumor control and survival were observed in mice treated with a combination of CAdtrio+HER2-specific CAR-expressing AdVSTs (i.e. treatment group (i)(b)).

### Example 10: Analysis of the Anti-Cancer Activity of the Combination of Oncolytic Virus, HDAd Virus and HER2-Specific CAR-T Cells in In Vivo Mouse Models

Tumour cells derived from HNSCC (SCC 47), prostate cancer (PC-3), cervical cancer (SiHa) and nasopharyngeal SSC (FaDu) were implanted into the right flanks of NSG mice. Animals were treated with control, CAdtrio (Onc5/3Ad2E1Δ24:HDAd/L-12_TK_PD-L1), HER2 specific CAR T cells, or a combination of CAdtrio and HER2.CAR T cells.

The mice received an intratumoural injection of 1×10⁸ viral particles of CAdtrio at a ratio of 1:20 Onc5/3Ad2E1Δ24:HDAd/L-12_TK_PD-L1. Three days later, the groups receiving CAR T cell therapy received an intravenous, systemic, administration via tail vein injection of a total of 1×10⁶ HER2.CAR T cells. Tumour volumes were measured at different time points. The end point was established at a tumour volume of >1500 mm³.

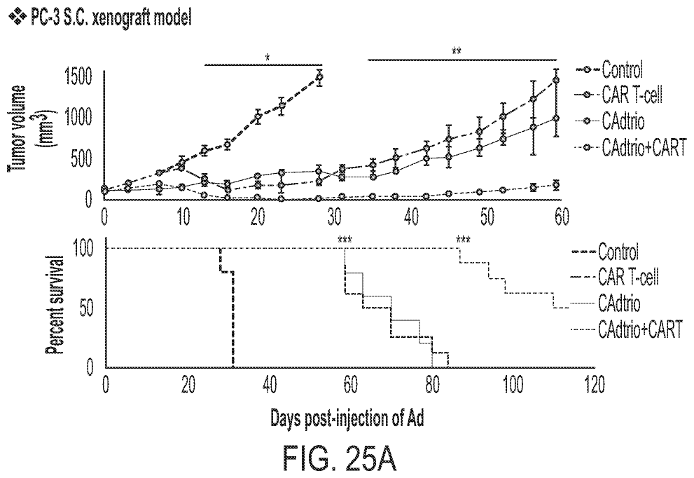

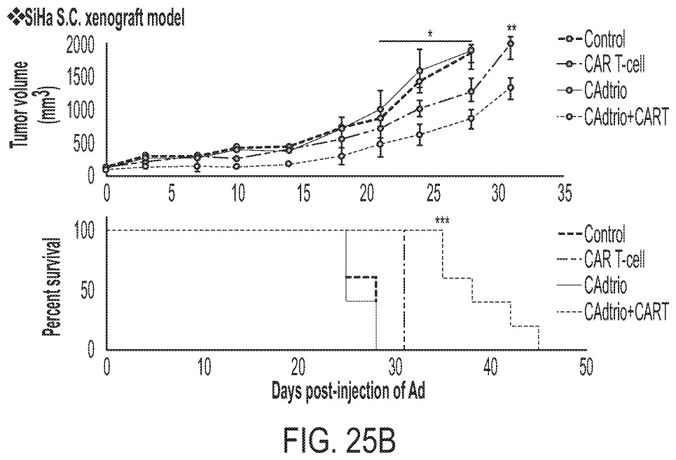

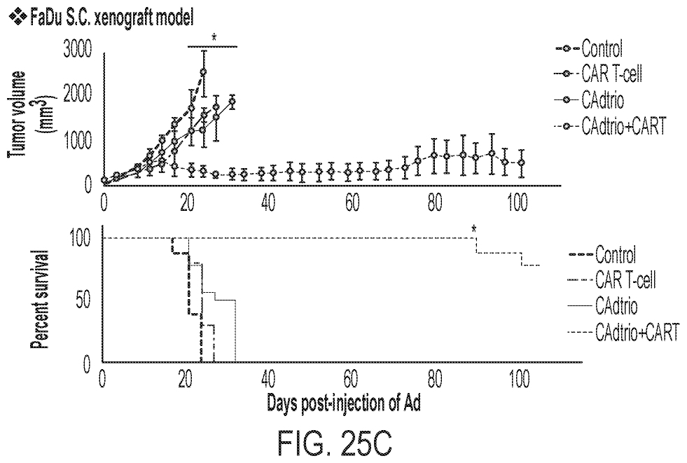

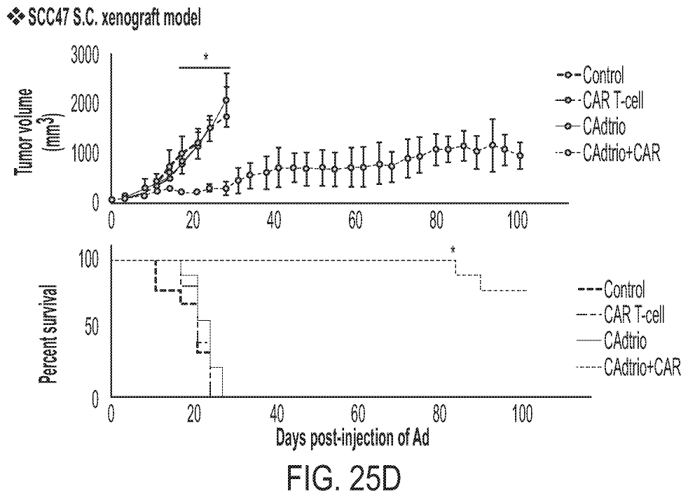

The results are shown in FIGS. 25A to 25D. Combination treatment of CAdtrio and HER2.CAR T-cells significantly improved anti-tumour effects compared to single agents and prolonged animal survival in different xenograft models.

The same treatment was assessed in a metastatic (orthotopic) tumour model of HNSCC. FaDu cells labelled with firefly luciferase were transplanted into the tongues of NSG mice. 1×10⁸ viral particles of CAdtrio were injected into the tongue. Three days later, 1×10⁶ HER2.CAR T cells were administered systemically by injection. Bioluminescence of FaDu cells, indicating cancer cell distribution, and body weight were monitored at different time points. The end point was established as animal body weight <80%.

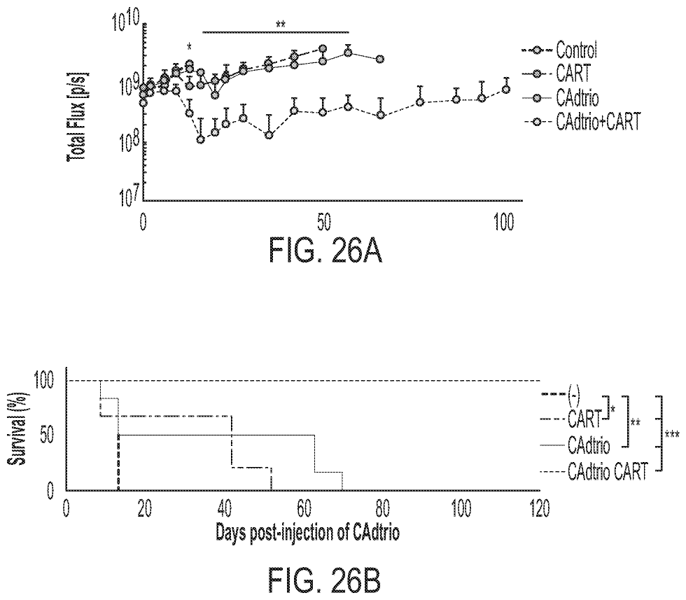

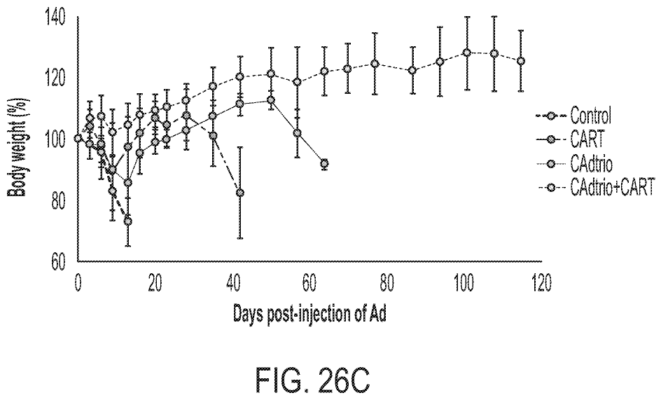

The results are shown in FIGS. 26A to 26C. Combination treatment of CAdtrio and HER2.CAR T-cells was effective in reducing tumor volume, decreasing incidence of metastasis and significantly improving survival in comparison to individual therapies, without loss of body weight. Combination treatment extended median survival from 13 days (untreated group) to more than 100 days. These data indicate that combination treatment eradicates both bulky primary tumors (through oncolysis) and distant metastases (through CAR T cells). Additionally, combination treatment showed durable anti-tumor effects with 1-2 log lower dosages of both CAdtrio and CAR T-cells than previous preclinical studies in immunodeficient models using either Onc.Ad or CAR T-cells alone.

### Example 11: Analysis of Anti-Tumour Efficacy and Host Immune Stimulation by CAdVEC in Humanised Mice

In the following examples, CAdtrio (oncolytic virus and helper-dependent adenovirus combination described herein) is referred to as ‘CAdVEC’. To address how the combination of local oncolysis-dependent inflammation, IL-12, and PD-L1 blocking mini-antibody produced by CAdVEC affects the human immune system, humanized mouse models were generated that reconstitute human innate and adaptive immune cells.

Transgenic NSG mice expressing human SCF, IL-3, and GM-CSF (NSGSGM3) were reconstituted with human innate and adaptive immune cells after hepatic transplantation of human cord blood-derived CD34+ cells. Once the presence in the blood of human CD45⁺ cells was confirmed, human FaDu cells were transplanted into the hind flank. Mice received a single intratumoural injection of TBG (buffer control), 2.5×10⁶ viral particles of CAdVEC, or 2.5×10⁹ viral particles of CAdVEC. A dose of 2.5×10⁶ VP dose/mouse corresponds to a human dose of 1×10¹⁰ VP/patient. A dose of 2.5×10⁹ VP dose/mouse corresponds to 1-log higher than a human dose of 1×10¹² VP/patient.

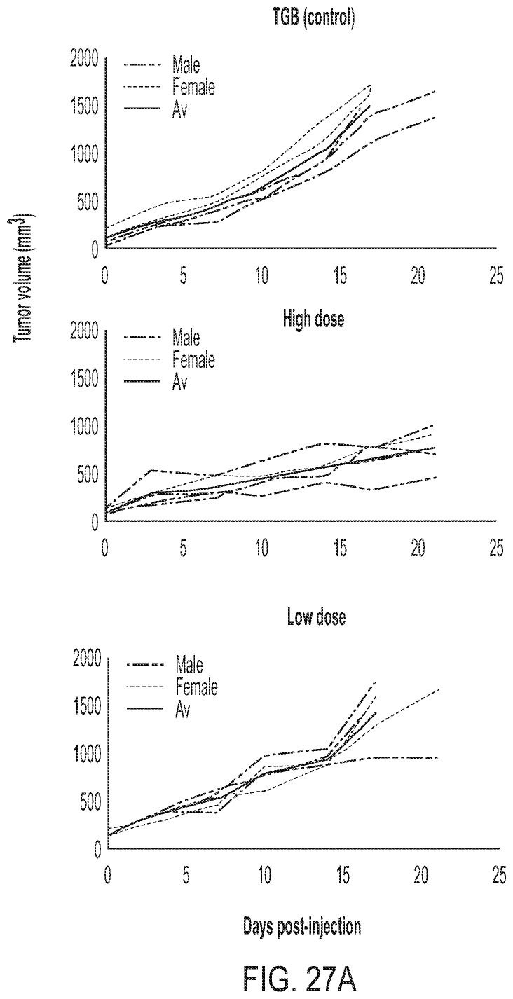

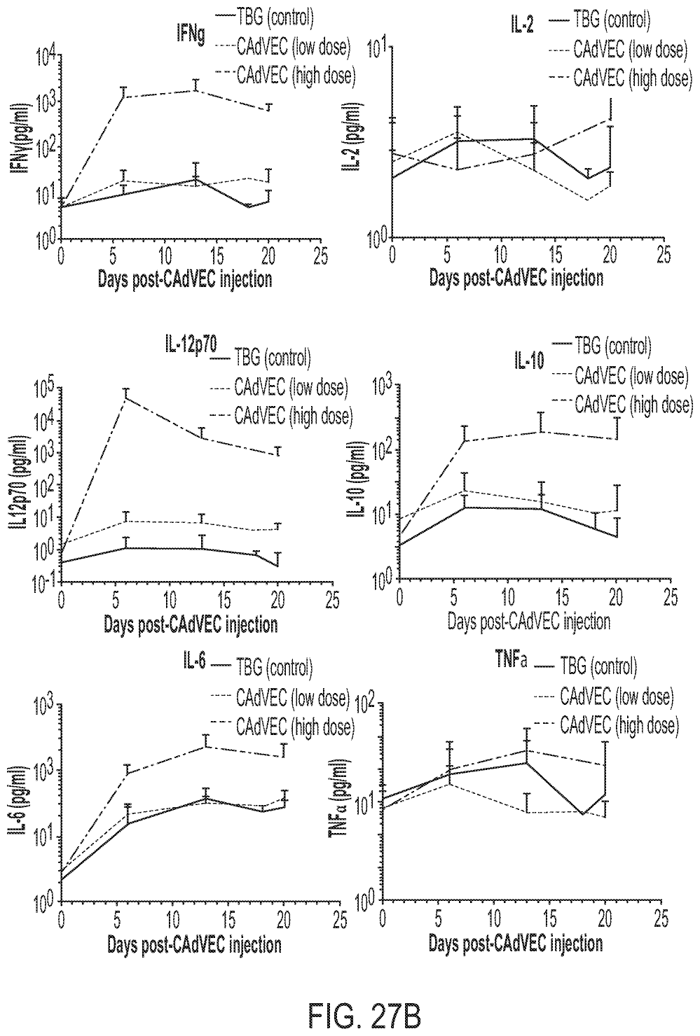

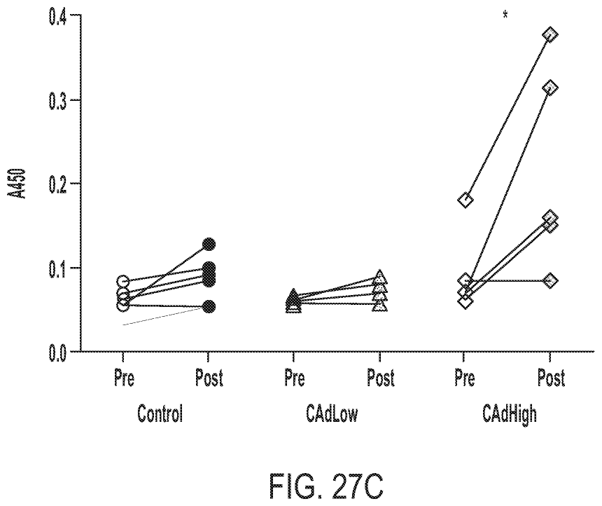

The results are shown in FIGS. 27A to 27C. Mice treated with CAdVEC showed dose-dependent local tumour control (27A), serum cytokine elevation (27B) and circulating human IgG antibodies to the injected adenoviral vectors (27C). These host immune responses, e.g. Th1 cytokine expression and anti-Ad IgG development, induced by intratumoural CAdVEC treatment may contribute to local tumour control in mice treated with CAdVEC.

### Example 12: CAdVEC and HER2.CAR T Cell Treatment in Subcutaneous Xenograft Immunodeficient Mice

CAdVEC was studied for its ability to enhance the anti-tumour activity of adoptively-transferred HER2.CAR T cells in vivo.

Monocytes infiltrate many solid tumour types (e.g., lung adenocarcinoma, pancreatic adenocarcinoma, and renal cell carcinoma). To mimic the human tumour microenvironment in subcutaneous xenograft mouse models, human cancer cells were co-transplanted with monocytes that were partially HLA-matched to tumour cells. CAR T cells were generated from the same donor and the anti-tumour effects of combination treatment were studied.

Eight-week old female NSG mice were subcutaneously transplanted with ffLuc-labeled human FaDu cells mixed with partially HLA-matched monocytes. Mice were split into four treatment groups:

1. TGB buffer control (day 0)

2. intratumoral injection of 1×10⁷ viral particles CAdVEC (Onc.Ad:HDAd=1:1) (day 0)

3. systemic administration of 1×10⁶ HER2.CAR T cells (day 3)

4. intratumoral injection of 1×10⁷ viral particles CAdVEC (Onc.Ad:HDAd=1:1) (day 0) followed by systemic administration of 1×10⁶ HER2.CAR T cells (day 3)

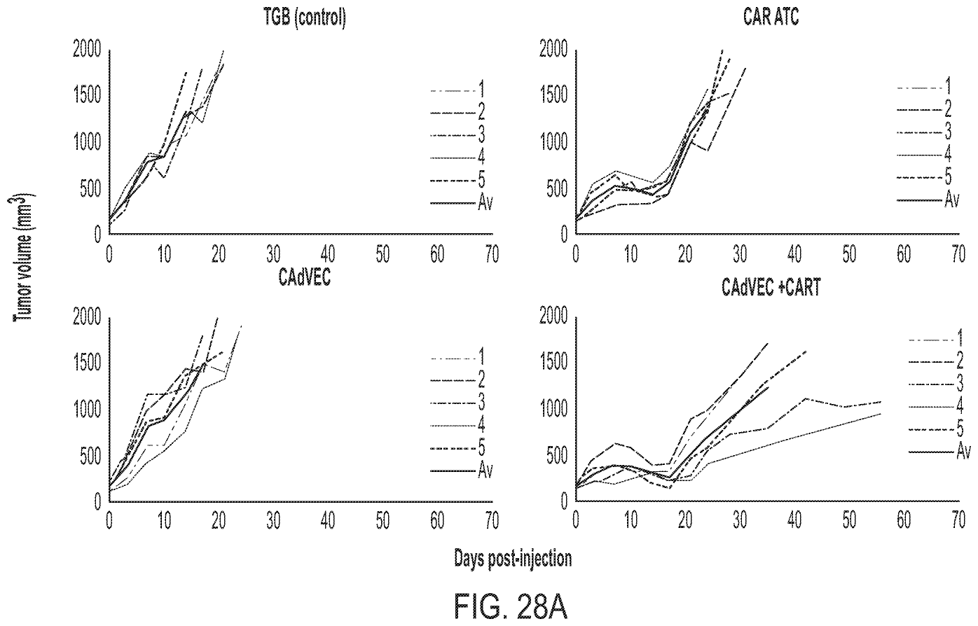

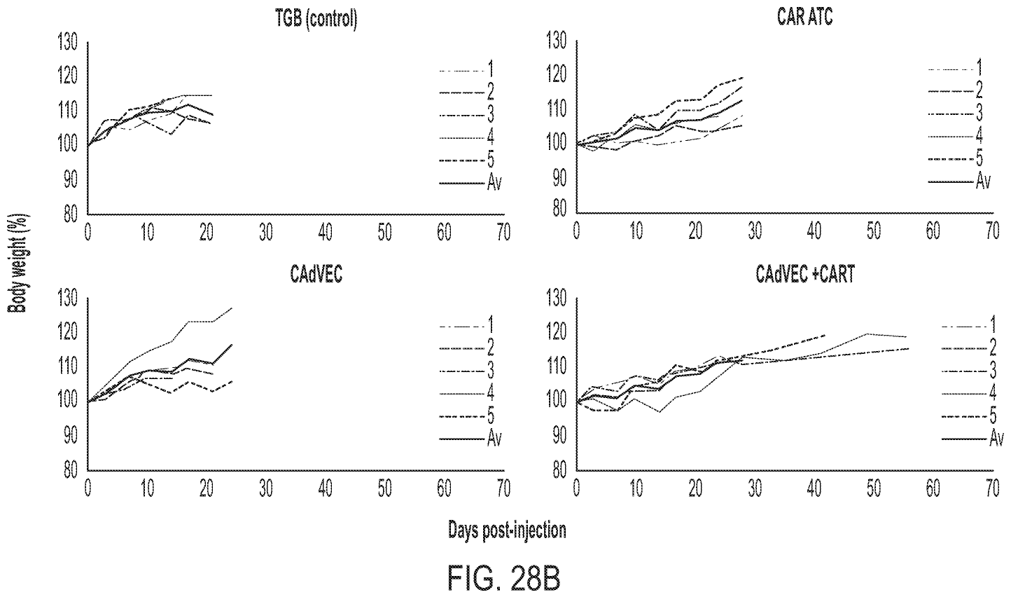

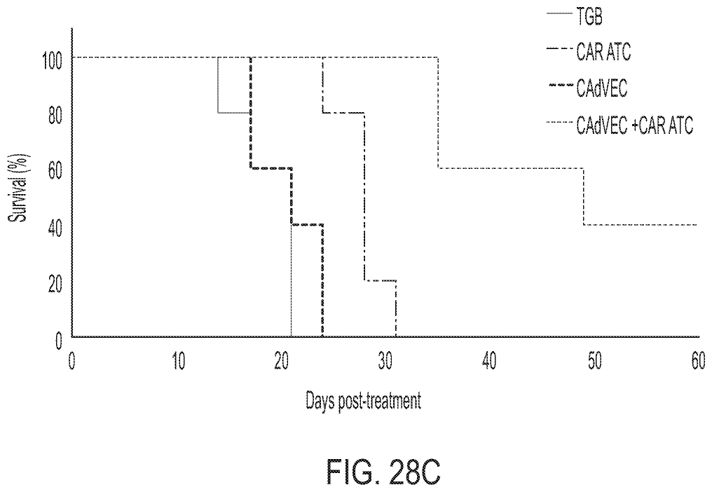

The results are shown in FIGS. 28A to 28C. Combinatorial treatment with CAdVEC and HER2.CAR T-cells controlled tumor growth (28A) and significantly extended median survival (28C) compared to mice treated with single agents in this animal model (Median survival: Control 21 days, CAdVEC 21 days, HER2.CAR T-cell 28 days, CAdVEC+CAR T-cell 49 days).

Thus, local CAdVEC treatment augments the anti-tumour effects of the CAR T cells.

### Example 13: CAdVEC and HER2.CAR T Cell Treatment in Orthotopic Xenograft Immunodeficient Mice

An orthotopic mouse model was generated to reflect patient outcomes as accurately as possible. Monocytes were co-transplanted with cancer cells and the anti-tumour efficacy of the combination treatment was studied. The model was used to study the efficacy of the combination treatment against both primary and metastatic tumour growth.

Nine-week old female NSG mice were orthotopically transplanted with ffLuc-labeled human FaDu cells and partially HLA-matched monocytes. Upon confirming local lymph node metastasis with IVIS imaging (Xenogen), the mice were divided into 4 groups and treated with one of:

1. TGB buffer control (day 0)

2. intratumoral injection of 2.5×10⁶ viral particles CAdVEC (Onc.Ad:HDAd=1:1) (day 0)

3. systemic administration of 1×10⁶ HER2.CAR T cells (day 3)

4. intratumoral injection of 2.5×10⁶ viral particles CAdVEC (Onc.Ad:HDAd=1:1) (day 0) followed by systemic administration of 1×10⁶ HER2.CAR T cells (day 3)

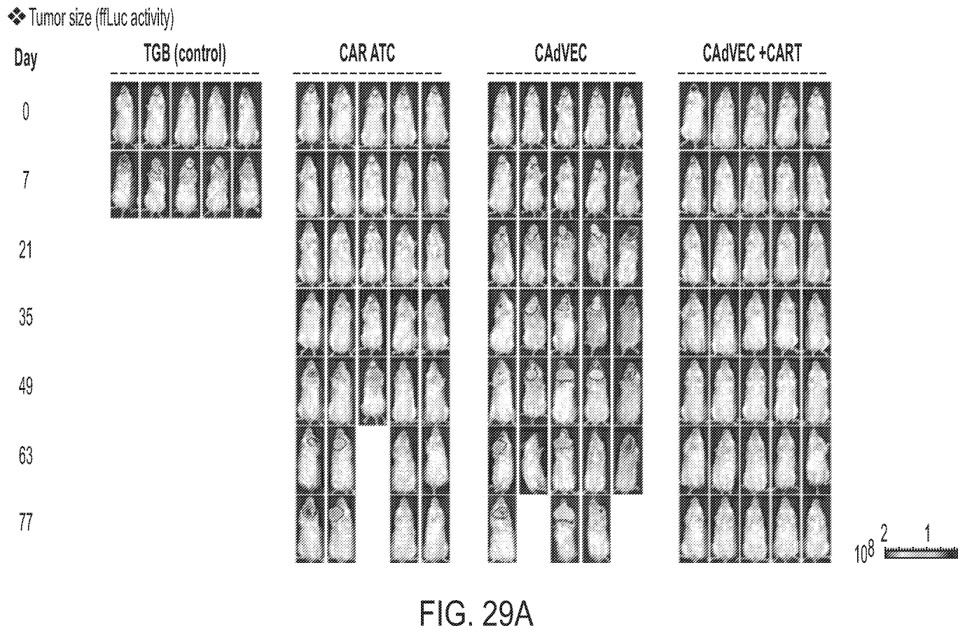

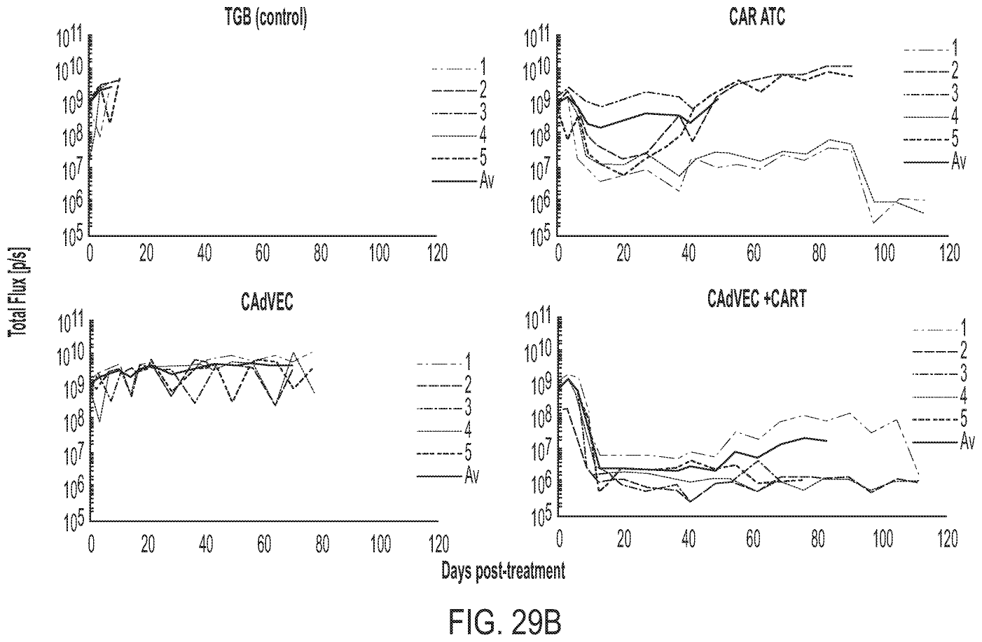

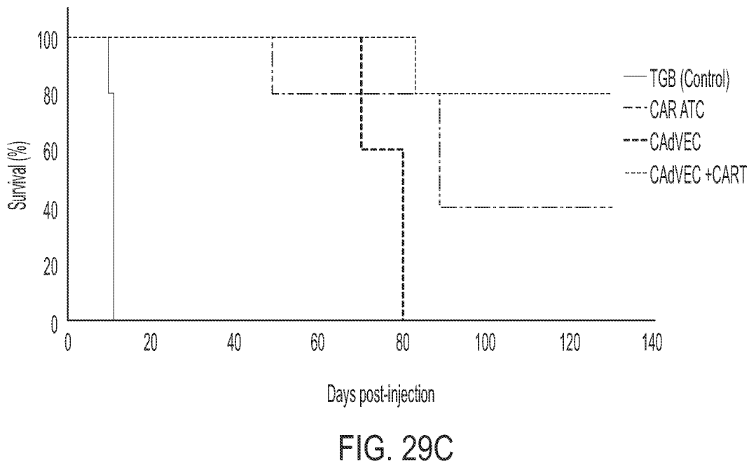

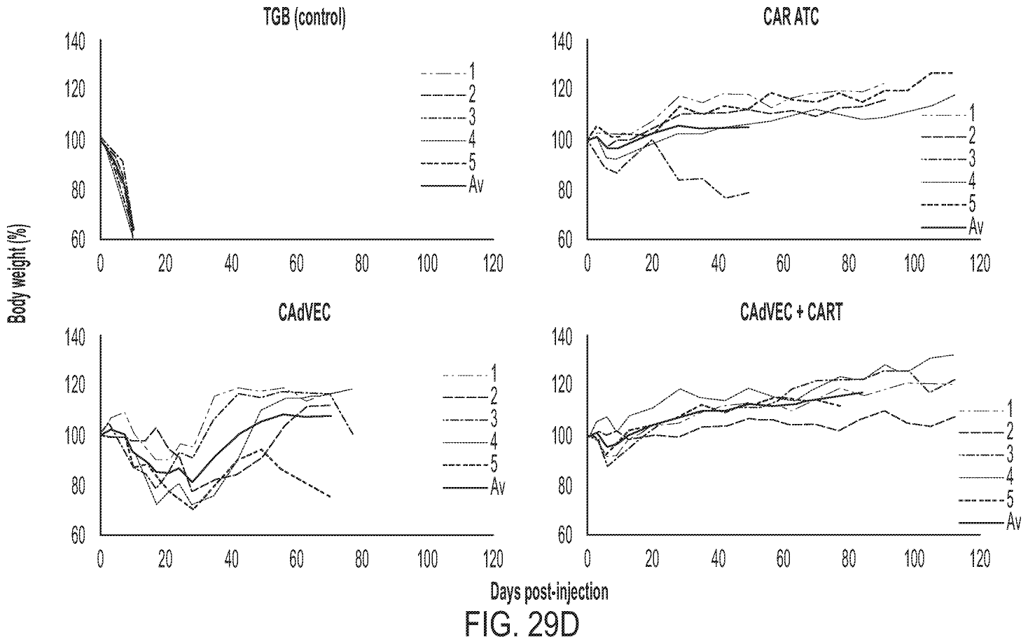

The results are shown in FIGS. 29A to 29D. Compared to mice receiving single treatments, mice treated with both CAdVEC and HER2.CAR T cells showed significantly reduced tumour signals (ffLuc activity; 29A and 29B) at both primary and metastatic tumour sites at 14 days post-CAdVEC injection. Dual treatment also extended median survival from 13 days (untreated group) to more than 130 days (29C), and these mice showed control at both primary and metastatic sites. In contrast to mice treated with single agents (either CAdVEC or HER2.CAR T-cell), mice treated with combination treatment histologically showed no detectable tumors in tongue (primary), lymph node and lung (metastases) tissue at 22 days post-treatment of CAdVEC. Mice receiving the combination treatment displayed minimal and transient weight loss due to local inflammation in the oropharynx at the site of primary tumour, but regained weight within 2 weeks post-treatment as the tumour became controlled by the treatment (29D).

Since combination but not single treatment provided long-term control over both primary and metastatic tumours in orthotopic xenograft mice, it was concluded that each treatment overcomes the inherent limitations of the other (CAdVEC: limited anti-tumour effect to metastasized tumors; CART: limited anti-tumour effect to bulky tumour and tumours can escape detection by downregulating the target antigen).

### Example 14: Treatment of Advanced HER2-Positive Solid Tumors Using Binary Oncolytic Adenovirus in Combination with HER2-Specific CAR-T

14.1 Brief Summary

This study involves patients with a type of cancer called HER2 (Human Epidermal Growth Factor Receptor 2) positive cancer. Because there is no standard treatment for this cancer at this time or because the currently used treatments do not work fully, this study asks patients to volunteer to take part in a research study investigating the safety and efficacy of using special immune cells called HER2 chimeric antigen receptor-modified Adenovirus-specific cytotoxic T lymphocytes (HER2-AdVST), in combination with intra-tumor injection of CAdVEC, an oncolytic adenovirus that is designed to help the immune system including HER2-AdVST react to the tumor.

The body has different ways of fighting infection and disease. No single way seems perfect for fighting cancers. This research study combines two different ways of fighting cancer: oncolytic adenovirus (CAdVEC) and T cells (HER2-AdVST).

CAdVEC is an oncolytic adenovirus that has been modified to include additional immune system stimulators. Specifically, genes that stimulate the immune system have been added to the oncolytic adenovirus. Oncolytic adenoviruses are viruses that are made to target and kill cancer cells without killing normal cells. Other oncolytic adenoviruses have been used in patients before however CAdVEC has not. In this study, CAdVEC will be injected into participants tumor at one tumor site which is most easiest to reach. Once the oncolytic adenovirus infects the cancer cells, the genes will be expressed, resulting in activation of the immune response so it can attack and kill cancer cells. However, because this approach may have limited effects on the other tumor sites that have not received the oncolytic virus injection, patients will also receive specific T cells following the intratumor CAdVEC injection. These T cells are special infection-fighting blood cells that can kill cells infected with viruses and tumor cells. Investigators have found from previous research that investigators can put a new gene into T cells and make them specifically recognize and kill cancer cells expressing HER2. In the past patients have signed a consent form that allowed us to put on their T cells the HER2 chimeric receptor with viral antigen receptor on their T cells. Investigators want to see if these cells can survive in a patient's blood and affect the tumor. Although HER2 chimeric antigen receptor modified cytotoxic T lymphocytes have been used in patients before, HER2-AdVST has not.

14.2 Detailed Description

Treatment with CAdVEC:
On the first day of participants' treatment, participants receive an intratumor injection of CAdVEC.

A blood sample is obtained from participants on Day 1 of treatment before participants receive CAdVEC intra-tumor injection. Depending on the location of the participant's tumor, different techniques are used for intra-tumor injection. The most common route of injection is ultrasound-guided percutaneous (needle puncture in the skin) injection, but endoscopic (examination of the inside of the body by using a lighted, flexible instrument called an endoscope) ultrasound-guidance are used for some patients as appropriate. Participants' doctors describe the procedures in more detail. Prior to percutaneous injection, participants may receive an anti-anxiety medicine such as diazepam (Valium) 5-10 mg to calm participants down, to relieve muscle spasms, and provide sedation. If the participant's tumor is injected during an endoscopic procedure, the procedure may be done under sedation.

This study looks at different doses of CAdVEC. The decision about the dose participants receive is determined when enrolled on the study.

Treatment with HER2-AdVST:
Participants provide blood samples for the production of Adenovirus-specific HER2 targeting cytotoxic T-lymphocytes (HER2-AdVSTs) in the lab. Investigators make the cells by first combining a special type of cells called dendritic cells (DCs) or monocytes with the T cells in the presence of produced mixtures of adenoviral proteins. Investigators then put a new gene in to those T cells to make them specifically attract to and kill HER2 positive tumors. As the T cells grow, investigators culture them by adding adenoviral proteins for stimulation and expansion. Investigators call those T cells: adenovirus-specific HER2-targeting T cells (HER2-AdVST). These cells are grown and frozen for participants.

Participants receive HER2-AdVST cells infusion between day 3 and day 14 after receiving CAdVEC intra-tumor injections. Before receiving the T cell infusion, participants may be given Benadryl (diphenhydramine) and Tylenol (acetaminophen). Tylenol and Benadryl are given to prevent a possible allergic reaction to the T cell administration.

This study looks at different doses of HER2-AdVST. The decision about the dose participants receive is determined when participants are enrolled on the study.

Standard Medical Tests Before Treatment:

Before being treated, participants receive a series of standard medical tests:
Physical exam and History
Blood tests to measure blood cells, kidney and liver function.
Pregnancy testing for female patients of childbearing potential (on blood or urine)
Measurements of participants tumor by scans will be performed as standard of care procedures
Color photography of skin lesions to estimate the size of the lesion, if present

Standard Medical Tests During and after Treatment:

Participants receive standard medical tests during and after study treatment:
Physical exams and History
Blood tests to measure blood cells, kidney and liver function.
Measurements of participants tumor by PET scans or CT scans 12 weeks after CAdVEC intra-tumor injection and then every 3 months up to 24 months per standard of care evaluation.
Color photography of skin lesions to estimate the size of the lesion, if present.

Study Specific Evaluations:

Investigators follow participants closely after treatment for any side effects for at least 2 years after the participants last infusion.

To learn more about the way the T cells are working in the participants body, an extra 20-50 mL (4-10 teaspoons) of blood is taken before CAdVEC injection, before the HER2-AdVST infusion, and at week 1, 2, 4 and 6 and 3, 6, 9 and 12 months after HER2-AdVST infusion. The blood may be drawn from participants central line at the time of the regular blood tests. Investigators use this blood to see how long the T cells last and to look at the immune response to the participants cancer. Investigators may also ask participants for a tumor tissue biopsy at week 12 if necessary.

14.3 Study Details

Study type: Interventional

Study Phase: Phase 1

Study Design:
Intervention Model: Single Group Assignment;
Masking: None (Open Label)
Primary Purpose: Treatment

14.4 Conditions

Bladder Cancer

Head and Neck Squamous Cell Carcinoma

Cancer of the Salivary Gland

Lung Cancer

Breast Cancer

Gastric Cancer

Esophageal Cancer

Colorectal Cancer

Pancreatic Adenocarcinoma

14.5 Intervention
CAdVEC
HER2.CAR AdVST

The administration of CAdVEC may create a pro-inflammatory tumor microenvironment and will promote the recruitment and expansion of adoptively transferred CAR AdVSTs via both CAR recognition of tumor antigen and TCR recognition of viral antigen. The CAR AdVSTs expanded at primary tumor sites may re-circulate and target metastasized tumors. The combination this study proposes to test has the potential to overcome each of the established individual limitations of oncolytic viruses and of CAR T-cell/VST and testing each element separately would not be beneficial or informative.

14.6 Study Arms

Experimental: Treatment Phase Six dose levels will be evaluated using the BOIN design. Cohorts of size 3 will be enrolled at each dose level (see section 11.1) until 9 evaluable patients have been studied at a single dose. Each patient receives an intratumoral injection of CAdVEC alone or combined with an injection of HER2.CAR.AdVST 3 days later (Day 4), according to the following dose levels.

Dose Level 1:
CAdVEC=1×10¹⁰ viral particles

Dose Level 2:
CAdVEC=1×10¹⁰ viral particles+1×10⁶ HER2.CAR-AdVSTs

Dose Level 3:
CAdVEC=1×10¹¹ viral particles+1×10⁶ HER2.CAR-AdVSTs

Dose Level 4:
CAdVEC=1×10¹¹ viral particles+1×10⁷ HER2.CAR-AdVSTs

Dose Level 5:
CAdVEC=1×10¹² viral particles+1×10 HER2.CAR-AdVSTs

Dose Level 6:
CAdVEC=1×10¹² viral particles+1×10⁸ HER2.CAR-AdVSTs

14.7 Eligibility Criteria

Inclusion/Exclusion Criteria

1. Histologically confirmed HER2 positive solid tumors, including but not limited to: head and neck squamous cell carcinoma; cancer of the salivary glands; lung cancer; breast cancer; bladder cancer; gastric cancer; esophageal cancer; colorectal cancer; and pancreatic adenocarcinoma. HER2 positivity is defined as ≥2+ staining by IHC with the FDA-approved 4B5 antibody (Ventana), which refers to greater than weak-to-moderate staining intensity in >10% tumor cells.

2. The disease must be deemed by the appropriate multidisciplinary tumor board unsuitable for curative surgery, radiotherapy, systemic therapy or any combination of the above modalities.

3. Disease must have progressed after standard first line therapy, or without available effective treatment options.

4. The patient must have at least one tumor site appropriate for intratumoral injection.

5. The patient must have radiographically measurable disease as per RECIST 1.1.

6. The patient must have adequate organ function within 7 days prior to consent for treatment as indicated by following measures:
Hematologic: Absolute neutrophil count (ANC) ≥1.0×10\hat{ }9/l; Hemoglobin 29 g/dl;
Platelet count ≥100×10\hat{ }9/l; PT or PTT ≤1.5×ULN unless the subject is receiving anticoagulation.
Hepatic function: bilirubin <2 ULN, and AST and ALT <3 ULN
Renal Function: serum creatinine <2× the ULN or creatinine clearance >30 mL/min.

7. Prior HER2 targeted therapy is allowed if delivered at least 4 weeks prior to the enrollment.

8. Prior immunotherapy is allowed if it was delivered at least 4 weeks prior to the enrollment.

9. Eastern Cooperative Oncology Group (ECOG) performance status 2 or less.

10. Females of childbearing potential must have a negative pregnancy test and agree to use contraception during on-study protocol therapy.

11. Male subjects with pregnant partner/female partner of childbearing potential agree to use barrier contraceptive during the study to minimize the risk of embryo-fetal exposure.

12. The patient is 18 years of age, and able to understand and give informed consent to study related procedures and treatments.

Sex/Gender: All sexes eligible for study

Ages: 18 years and older

Healthy volunteers not accepted.

14.8 Outcome Measures

Primary Outcome Measures

1. Number of patients with dose limiting toxicity (DLT) by CTCAE 5.0 [Time Frame: 6 weeks after the HER2.CAR AdVST infusion or 6 weeks+3 days after the CAdVEC injection.]
Incidence of dose limiting toxicities (DLT) of CAdVEC intratumoral injection in combination with HER2.CAR AdVST cells in patients with advanced refractory HER2 positive solid tumors.

Secondary Outcome Measures
1. Overall Response Rate (ORR) according to RECIST1.1 criteria [Time Frame: 13 weeks]
   Overall response rate is defined as the number of patients experiencing PR or better (i.e. PR+CR) divided by the number evaluable for efficacy. Tumor regression or progression will be evaluated accordingly with RECIST 1.1 criteria.
2. Disease Control Rate (DCR) [Time Frame: 13 weeks]
   Disease Control Rate is defined as the number of patients experiencing SD or better (i.e. SD+PR+CR) divided by the number evaluable for efficacy. Tumor regression or progression will be evaluated accordingly with RECIST 1.1 criteria.
3. Progression Free Survival (PFS) [Time Frame: 15 years]
   Progression-Free Survival is defined as the time from start of treatment to disease progression or death.
4. Overall Survival (OS) [Time Frame: 15 years]
   Overall survival is defined as the time from the start of treatment to death due to any cause.
5. Number of treatment related adverse events with grade 3 or greater severity by CTCAE 5.0 [Time Frame: 30 days]
   Treatment related adverse events with grade 3 or greater severity by CTCAE 5.0

1. A method of treating a cancer, comprising administering to a subject:
(i) an oncolytic virus and a virus comprising nucleic acid encoding IL-12 and antagonist anti-PD-L1 antibody; and
(ii) cells comprising a chimeric antigen receptor (CAR) specific for a cancer cell antigen.

2. A combination of (i) an oncolytic virus and a virus comprising nucleic acid encoding IL-12 and antagonist anti-PD-L1 antibody, and (ii) cells comprising a chimeric antigen receptor (CAR) specific for a cancer cell antigen, for use in a method of treating a cancer.

3. Use of (i) an oncolytic virus and a virus comprising nucleic acid encoding IL-12 and antagonist anti-PD-L1 antibody, and (ii) cells comprising a chimeric antigen receptor (CAR) specific for a cancer cell antigen, in the manufacture of a medicament for use in a method of treating a cancer.

4. The method according to claim 1, the combination for use according to claim 2, or the use according to claim 3, wherein the method of treating a cancer comprises administering the oncolytic virus and virus comprising nucleic acid encoding IL-12 and antagonist anti-PD-L1 antibody at a total viral particle dose of 1×10¹⁰, 1×10¹¹, or 1×10¹² particles.

5. The method, combination or use according to any one of claims 1 to 4, wherein the method of treating a cancer comprises administering a dose of 1×10⁶, 1×10⁷, or 1×10⁸ cells comprising a CAR.

6. The method, combination or use according to any one of claims 1 to 5, wherein the method of treating a cancer comprises: (a) administering the oncolytic virus and virus comprising nucleic acid encoding IL-12 and antagonist anti-PD-L1 antibody; and (b) administering cells comprising a CAR three days after administration according to (a).

7. The method, combination or use according to any one of claims 1 to 6, wherein the method of treating a cancer comprises administering the oncolytic virus and virus comprising nucleic acid encoding IL-12 and antagonist anti-PD-L1 antibody via intratumoral administration.

8. The method, combination or use according to claims 1 to 7, wherein the method of treating a cancer comprises administering the cells comprising a CAR via intravenous administration.

9. The method, combination or use according to claims 1 to 8, wherein the method of treating a cancer comprises administering the oncolytic virus and virus comprising nucleic acid encoding IL-12 and antagonist anti-PD-L1 antibody at a viral particle ratio of oncolytic virus:virus comprising nucleic acid of from 1:0.5 to 1:25.

10. The method, combination or use according to claims 1 to 9, wherein the method of treating a cancer comprises administering the oncolytic virus and virus comprising nucleic acid encoding IL-12 and antagonist anti-PD-L1 antibody at a viral particle ratio of oncolytic virus:virus comprising nucleic acid of 1:1.

11. The method, combination or use according to any one of claims 1 to 10, wherein the oncolytic virus is an oncolytic adenovirus (OncAd).

12. The method, combination or use according to any one of claims 1 to 11, wherein the oncolytic virus is derived from adenovirus 5 (Ad5).

13. The method, combination or use according to any one of claims 1 to 12, wherein the oncolytic virus encodes an E1A protein which displays reduced binding to Rb protein as compared to E1A protein encoded by Ad5.

14. The method, combination or use according to any one of claims 1 to 13, wherein the oncolytic virus encodes an E1A protein lacking the amino acid sequence LTCHEACF (SEQ ID NO:52).

15. The method, combination or use according to any one of claims 1 to 14, wherein the oncolytic virus encodes an E1A protein comprising, or consisting of, the amino acid sequence SEQ ID NO:34.

16. The method, combination or use according to claim any one of claims 1 to 15, wherein the virus comprising nucleic acid encoding IL-12 and antagonist anti-PD-L1 antibody is a helper-dependent adenovirus (HDAd).

17. The method, combination or use according to claim any one of claims 1 to 16, wherein the virus comprising nucleic acid encoding IL-12 and antagonist anti-PD-L1 antibody comprises nucleic acid encoding an enzyme capable of catalysing conversion of a non-toxic factor to a cytotoxic form.

18. The method, combination or use according to claim 17, wherein the enzyme is selected from: thymidine kinase, cytosine deaminase, nitroreductase, cytochrome P450, carboxypeptidase G2, purine nucleoside phosphorylase, horseradish peroxidase and carboxylesterase.

19. The method, combination or use according to any one of claims 1 to 18, wherein the cells comprising a CAR specific for a cancer cell antigen are T cells.

20. The method, combination or use according to any one of claims 1 to 19, wherein the CAR comprises an antigen binding domain capable of specific binding to HER2.

21. The method, combination or use according to any one of claims 1 to 20, wherein the CAR comprises an antigen-binding domain comprising:
a VL domain comprising:
LC-CRD1: SEQ ID NO:10;
LC-CRD2: SEQ ID NO:11;
LC-CRD3: SEQ ID NO:12;
and a VH domain comprising:
HC-CRD1: SEQ ID NO:13;
HC-CRD2: SEQ ID NO:14;
HC-CRD3: SEQ ID NO:15;
or
a VL domain comprising:
LC-CRD1: SEQ ID NO:18;
LC-CRD2: SEQ ID NO:19;
LC-CRD3: SEQ ID NO:20;
and a VH domain comprising:
HC-CRD1: SEQ ID NO:21;
HC-CRD2: SEQ ID NO:22;
HC-CRD3: SEQ ID NO:23;
or
a VL domain comprising:
LC-CRD1: SEQ ID NO:26;
LC-CRD2: SEQ ID NO:27;
LC-CRD3: SEQ ID NO:28;
and a VH domain comprising:
HC-CRD1: SEQ ID NO:29;
HC-CRD2: SEQ ID NO:30;
HC-CRD3: SEQ ID NO:31;
or
a VL domain comprising:
LC-CRD1: SEQ ID NO:57;
LC-CRD2: SEQ ID NO:58;
LC-CRD3: SEQ ID NO:59;
and a VH domain comprising:
HC-CRD1: SEQ ID NO:60;
HC-CRD2: SEQ ID NO:61;
HC-CRD3: SEQ ID NO:62.

22. The method, combination or use according to any one of claims 1 to 21, wherein the CAR comprises an antigen binding domain comprising:
a VL comprising, or consisting of, an amino acid sequence having at least 75% sequence identity to SEQ ID NO:16 and a VH comprising, or consisting of, an amino acid sequence having at least 75% sequence identity to SEQ ID NO:17;
or
a VL comprising, or consisting of, an amino acid sequence having at least 75% sequence identity to SEQ ID NO:24 and a VH comprising, or consisting of, an amino acid sequence having at least 75% sequence identity to SEQ ID NO:25;
or
a VL comprising, or consisting of, an amino acid sequence having at least 75% sequence identity to SEQ ID NO:32 and a VH comprising, or consisting of, an amino acid sequence having at least 75% sequence identity to SEQ ID NO:33;
or
a VL comprising, or consisting of, an amino acid sequence having at least 75% sequence identity to SEQ ID NO:63 and a VH comprising, or consisting of, an amino acid sequence having at least 75% sequence identity to SEQ ID NO:64.

23. The method, combination or use according to any one of claims 1 to 22, wherein the method of treating a cancer comprises:
(a) isolating at least one cell from a subject;
(b) modifying the at least one cell to express or comprise a CAR specific for a cancer cell antigen, or a nucleic acid encoding a CAR specific for a cancer cell antigen,
(c) optionally expanding the modified at least one cell, and;
(d) administering the modified at least one cell to a subject.

24. The method, combination or use according to any one of claims 1 to 23, wherein the cancer expresses HER2.

25. The method, combination or use according to any one of claims 1 to 24, wherein the cancer is selected from head and neck cancer, head and neck squamous cell carcinoma, nasopharyngeal carcinoma (NPC), breast cancer, bladder cancer, cervical carcinoma (CC), oropharyngeal carcinoma (OPC), oesophageal cancer, colorectal cancer, gastric carcinoma (GC), hepatocellular carcinoma (HCC), salivary gland cancer, lung cancer, and pancreatic adenocarcinoma.

26. The method, combination or use according to any one of claims 1 to 25, wherein the cancer comprises a HER2-positive tumour.
